(19) United States
(12) Patent Application Publication
(10) Pub. No.: US 2026/0144899 A1
(43) Pub. Date: May 28, 2026

(54) CIRCULAR RNAS FOR CELLULAR THERAPY

(71) Applicant: Flagship Pioneering Innovations VI, LLC, Cambridge, MA (US)

(72) Inventors: Alexandra Sophie DE BOER, Somerville, MA (US); Erica Gabrielle WEINSTEIN, Newton, MA (US); Nicholas McCartney PLUGIS, Cohasset, MA (US); Catherine CIFUENTES-ROJAS, Brookline, MA (US); Morag Helen STEWART, Tiburon, CA (US); Avak KAHVEJIAN, Lexington, MA (US)

(21) Appl. No.: 19/380,596

(22) Filed: Nov. 5, 2025

Related U.S. Application Data
(63) Continuation of application No. 17/617,632, now abandoned.
(60) Provisional application No. 62/861,805, filed on Jun. 14, 2019.
(60) Provisional application No. 62/967,537, filed on Jan. 29, 2020.

(51) Int. Cl. A61K 48/00; A61K 39/00; A61K 40/11; A61K 40/15; A61K 40/31; A61K 40/42; A61K 40/50; A61P 35/00; C07K 14/725; C07K 16/28; C12N 5/0783; C12N 9/22; C12N 15/11; C12N 15/115; C12N 15/85; C12N 15/90

(52) U.S. Cl. CPC A61K 48/0058; A61K 40/11; A61K 40/15; A61K 40/31; A61K 40/4211; A61P 35/00; C07K 14/7051; C07K 16/2803; C12N 5/0636; C12N 9/22; C12N 15/11; C12N 15/115; C12N 15/85; C12N 15/907; A61K 2039/54; A61K 2039/545; A61K 40/50; A61K 2239/48; C07K 2317/31; C07K 2317/76; C07K 2319/02; C07K 2319/03; C07K 2319/33; C12N 2310/16; C12N 2310/20; C12N 2320/32; C12N 2800/80

(57) ABSTRACT

This invention relates generally to pharmaceutical compositions and preparations of circular polyribonucleotides and uses thereof in cellular therapy.

### SEQUENCE LISTING

The instant application contains a Sequence Listing which has been submitted electronically in XML format and is hereby incorporated by reference in its entirety. Said XML copy, created on Nov. 4, 2025, is named “51509-013004_Sequence_Listing_11_4_25” and is 88,560 bytes in size.

### BACKGROUND

Certain circular polyribonucleotides are ubiquitously present in human tissues and cells, including tissues and cells of healthy individuals.

### SUMMARY

The present disclosure generally relates to compositions comprising isolated cells and cell preparations, and methods of using such cells and cell preparations, for cell therapy in mammals, e.g., humans. The compositions include, and the methods use, isolated cells comprising circular polyribonucleotides (e.g., isolated mammalian cells comprising exogenous, synthetic circular RNAs) where the circular polyribonucleotides (a) comprise at least one binding site, (b) encode a protein, or both (a) and (b). The cells (e.g., isolated mammalian cells) can be selected, inter alia, from an immune cell (such as a T cell, B cell, or NK cell), a macrophage, a dendritic cell, a red blood cell, a reticulocyte, a myeloid progenitor, and a megakaryocyte. The protein can be a secreted protein, membrane protein, or intracellular protein. The methods of cellular therapy can comprise administering the isolated cells or preparations to a subject (e.g., a human) in need thereof.

In one aspect, the invention features a pharmaceutical composition comprising a pharmaceutically acceptable carrier or excipient and a circular polyribonucleotide comprising at least one binding site, an encoded protein or a combination thereof. In one embodiment of this aspect, the circular polyribonucleotide (1) comprises at least one binding site, (2) encodes a secreted protein or an intracellular protein, or (3) a combination of (1) and (2). In another embodiment of this aspect, the circular polyribonucleotide (1) comprises at least one binding site, (2) encodes a membrane protein, or (3) a combination of (1) and (2), wherein the membrane protein is not a chimeric antigen receptor, T cell receptor, or T cell receptor fusion protein. In another embodiment of this aspect, the circular polyribonucleotide comprises at least one binding site and encodes a protein, wherein the protein is a secreted protein, membrane protein, or an intracellular protein.

In another aspect, the invention features an isolated cell or preparation of such cells comprising a circular polyribonucleotide comprising at least one binding site, an encoded protein or a combination thereof, wherein the isolated cell is administered to a subject. In one embodiment of this aspect, the circular polyribonucleotide (1) comprises at least one binding site, (2) encodes a secreted protein or an intracellular protein, or (3) a combination of (1) and (2). In another embodiment of this aspect, the circular polyribonucleotide (1) comprises at least one binding site, (2) encodes a membrane protein, or (3) a combination of (1) and (2), wherein the membrane protein is not a chimeric antigen receptor, T cell receptor, or T cell receptor fusion protein. In another embodiment of this aspect, the circular polyribonucleotide comprises at least one binding site and encodes a protein, wherein the protein is a secreted protein, membrane protein, or an intracellular protein.

In some embodiments, the circular polyribonucleotide lacks a poly-A tail, a replication element, or both.

In some embodiments, the intracellular protein, membrane protein, or secreted protein is a therapeutic protein. In some embodiments, the intracellular protein, membrane protein, or secreted protein promotes cell expansion, cell differentiation, and/or localization of the cell to a target. In some embodiments, the intracellular protein, membrane protein, and/or secreted protein has binding activity, or transcription regulator activity.

In some embodiments, the protein is a membrane protein and the cell is a non-immune cell.

In some embodiments, the membrane protein is a transmembrane protein or extracellular matrix protein. In some embodiments, the membrane protein is a chimeric antigen receptor.

In some embodiments, the at least one binding site confers at least one therapeutic characteristic to the cell. In some embodiments, the at least one binding site confers nucleic acid localization to the cell or isolated cell. In some embodiments, the at least one binding site confers nucleic acid activity in the cell or isolated cell. In some embodiments, the at least one binding site is an aptamer. In some embodiments, the at least one binding site is a protein binding site, DNA binding site, or RNA binding site. In some embodiments, the at least one binding site is an miRNA binding site. In some embodiments, the at least one binding site binds to a cell receptor on a surface of the cell. In some embodiments, the circular polyribonucleotide is internalized into the cell after the at least one binding site binds to a cell receptor on the surface of the cell.

In some embodiments, the cell is a eukaryotic cell, animal cell, mammalian cell, or human cell. In some embodiments, the cell is an immune cell. In some embodiments, the cell is a peripheral blood mononuclear cell, peripheral blood lymphocyte, or lymphocyte. In some embodiments, the cell is selected from a group consisting of a T cell (e.g., a regulatory T cell, γδT cell, αβT cell, CD8+ T cell, or CD4+ T cell), a B cell, or a Natural Killer cell. In some embodiments, the cell is replication incompetent.

In some embodiments of any aspect described herein, the pharmaceutical composition comprises a plurality or preparation of the cells, wherein the preparation comprise or the plurality is at least 10⁵ cells, e.g. at least 10⁶ or at least 10⁷ or at least 10⁸ or at least 10⁹ or at least 10¹⁰ or at least 10¹¹ cells, e.g., between from 5×10⁵ cells to 1×10⁷ cells. In some embodiments, the plurality is from 12.5×10⁵ cells to 4.4×10¹¹ cells. In some embodiments, the pharmaceutical composition comprises a plurality or preparation of the cells that is a unit dose for a target subject, e.g., the pharmaceutical composition comprises between 10⁵-10⁹ cells/kg of the target subject, e.g., between 10⁶-10⁸ cells/kg of the target subject. For example, a unit dose for a target subject weighing 50 kg may be a pharmaceutical composition that comprises between 5×10⁷ and 2.5×10¹⁰ cells, e.g., between 5×10⁷ and 2.5×10⁹ cells, e.g., between 5×10⁸ and 5×10⁹ cells.

In some embodiments, the pharmaceutical composition is for administration to a subject. In some embodiments, the subject is a human or non-human animal. The human may be a juvenile, a young adult (from 18-25 years), an adult, or a neonate. In some embodiments, the subject has a disease or disorder. In some embodiments, the subject has a hyperproliferative disease or cancer. In some embodiments, the cell or isolated cell is allogeneic to the treated subject. In some embodiments, the cell or isolated cell is autologous to the treated subject.

In some embodiments, the isolated cell is formulated with a pharmaceutically acceptable excipient (e.g., a diluent).

In a third aspect, the invention provides a pharmaceutical composition comprising a cell, wherein the cell comprises a circular polyribonucleotide encoding an antigen-binding domain, a transmembrane domain, and an intracellular signaling domain and comprising at least one binding site.

In a fourth aspect, the invention provides an isolated cell comprising a circular polyribonucleotide encoding a chimeric antigen receptor and comprises at least one binding site, wherein the isolated cell is for administration (e.g., intravenous administration to a subject).

In a fifth aspect, the invention provides a cell comprising: (a) a circular polyribonucleotide comprising i) at least one target binding sequence encoding an antigen-binding protein that binds to an antigen or ii) a sequence encoding an antigen-binding domain, a transmembrane domain, and an intracellular signaling domain and, optionally, comprising at least one binding site; and (b) a second nucleotide sequence encoding a protein, wherein expression of the protein is activated upon binding of the antigen to the antigen-binding protein.

In a sixth aspect, the invention provides a cell comprising a circular polyribonucleotide encoding a T cell receptor (TCR) comprising affinity for an antigen and a circular polyribonucleotide encoding a bispecific antibody, wherein the cell expresses the TCR and bispecific antibody on a surface of the cell.

In some embodiments of any aspect described herein, the chimeric antigen receptor comprises an antigen-binding domain, a transmembrane domain, and an intracellular domain. In some embodiments, the antigen-binding protein comprises an antigen-binding domain, a transmembrane domain, and an intracellular signaling domain. In some embodiments, the antigen-binding domain is linked to the transmembrane domain, which is linked to the intracellular signaling domain to produce a chimeric antigen receptor. In some embodiments, the antigen-binding domain binds to a tumor antigen, a tolerogen, or a pathogen antigen, or the antigen is a tumor antigen, or a pathogen antigen. In some embodiments, the antigen-binding domain is an antibody or antibody fragment thereof (e.g., scFv, Fv, Fab). In some embodiments, the antigen binding domain is a bispecific antibody. In some embodiments, the bispecific antibody has first immunoglobulin variable domain that binds a first epitope and a second immunoglobulin variable domain that binds a second epitope. In some embodiments, the first epitope and the second epitope are the same. In some embodiments, the first epitope and the second epitope are different.

In some embodiments, the transmembrane domain links the binding domain and the intracellular signaling domain. In some embodiments, the transmembrane domain is a hinge protein (e.g., immunglobuline hinge), a polypeptide linker (e.g., GS linker), a KIR2DS2 hinge, a CD8a hinge, or a spacer.

In some embodiments, the intracellular signaling domain comprises at least a portion of a T-cell signaling molecule. In some embodiments, the intracellular signaling domain comprises an immunoreceptor tyrosine-based activation motif. In some embodiments, the intracellular signaling domain comprises at least a portion of CD3zeta, common FcRgamma (FCER1G), Fc gamma RIIa, FcRbeta (Fc Epsilon Rib), CD3 gamma, CD3delta, CD3epsilon, CD79a, CD79b, DAP10, DAP12, or any combination thereof. In some embodiments, the intracellular signaling domain further comprises a costimulatory intracellular signaling domain.

In some embodiments, the costimulatory intracellular signaling domain comprises at least one or more of a TNF receptor protein, immunoglobulin-like protein, a cytokine receptor, an integrin, a signaling lymphocytic activation molecule, or an activating NK cell receptor protein. In some embodiments, the costimulatory intracellular signaling domain comprises at least one or more of CD27, CD28, 4-1BB, OX40, GITR, CD30, CD40, PD-1, ICOS, BAFFR, HVEM, ICAM-1, LFA-1, CD2, CDS, CD7, CD287, LIGHT, NKG2C, NKG2D, SLAMF7, NKp80, NKp30, NKp44, NKp46, CD160, CD19, CD4, CD8alpha, CD8beta, IL2R beta, IL2R gamma, IL7R alpha, ITGA4, VLA1, CD49a, IA4, CD49D, ITGA6, VLA6, CD49f, ITGAD, CD103, ITGAL, ITGAM, ITGAX, ITGB1, CD29, ITGB2, CD18, ITGB7, TNFR2, TRANCE/TRANKL, CD226, SLAMF4, CD84, CD96, CEACAM1, CRTAM, CD229, CD160, PSGL1, CD100, CD69, SLAMF6, SLAMF1, SLAMF8, CD162, LTBR, LAT, GADS, SLP-76, PAG/Cbp, CD19a, B7-H3, or a ligand that binds to CD83.

In some embodiments, the circular polyribonucleotide lacks a poly-A tail, a replication element, or combination thereof.

In some embodiments, cell is an immune effector cell. In some embodiments, the cell is a T cell (e.g., a αβ T cell, or γδ T cell) or an NK cell. In some embodiments, the cell is an allogeneic cell or autologous cell. In some embodiments, the antigen is expressed from a tumor or cancer. In some embodiments, the protein is a cytokine (e.g., IL-12) or a costimulatory ligand (e.g., CD40L or 4-1BBL). In some embodiments, the protein is a secreted protein.

In a seventh aspect, the invention provides a preparation of from 1×10⁵ to 9×10¹¹ cells, e.g., between 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, the preparation configured for parenteral delivery to a subject, wherein the preparation comprises a plurality (e.g., at least 1% of the cells in the preparation) of cells or isolated cells as described herein. For example, at least 50% of the cells, at least 60% of the cells, e.g., between 50-70% of the cells in the preparation are cells comprising a synthetic, exogenous circular RNA as described herein. In some embodiments of this aspect, the preparation is in a unit dose form described herein. In some embodiments of this aspect, the delivery is injection or infusion (e.g., IV injection or infusion).

In an eighth aspect, the invention provides an intravenous bag or other infusion product comprising a suspension of isolated cells, wherein a plurality of the cells in the suspension (e.g., at least 1% of the cells in the preparation) is any cell or isolated cell described herein. In some embodiments, the suspension comprises from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, the IV bag being configured for parenteral delivery to a subject. In some embodiments, at least 50% of the cells, at least 60% of the cells, e.g., between 50-70% of the cells in the suspension are cells comprising a synthetic, exogenous circular RNA as described herein. In some embodiments of this aspect, the IV bag comprises a unit dose of cells described herein.

In a ninth aspect, the invention provides a medical device comprising a plurality of cells, e.g., from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, the medical device being configured for implantation into a subject, wherein at least 40% of the cells in the medical device are cells or isolated cells as described herein. For example, at least 50% of the cells, at least 60% of the cells, e.g., between 50-70% of the cells in the medical device are cells comprising a synthetic, exogenous circular RNA as described herein.

In a tenth aspect, the invention provides a biocompatible matrix comprising a plurality of cells, wherein the biocompatible matrix is configured for implantation into a subject. The biocompatible matrix can comprise from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, wherein at least 50% of the cells, at least 60% of the cells, e.g., between 50-70% of the cells in the biocompatible matrix are cells comprising a synthetic, exogenous circular RNA as described herein. For example, the biocompatible matrix is an Afibromer™ matrix. For example, the biocompatible matrix may be that described in Bose et al. 2020. Nat Biomed Eng. 2020. doi:10.1038/s41551-020-0538-5, which is incorporated herein by reference.

In an eleventh aspect, the invention provides a bioreactor comprising a plurality of cells, e.g., from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, wherein at least 50% of the cells, at least 60% of the cells, e.g., between 50-70% of the cells in the bioreactcor are cells comprising a synthetic, exogenous circular RNA as described herein. In some embodiments of this aspect, the bioreactor comprises a 2D cell culture. In some embodiments of this aspect, the bioreactor comprises a 3D cell culture.

In some embodiments, the medical device or biocompatible matrix disclosed herein is configured to produce and release the plurality of cells when implanted into the subject.

In some embodiments of the above aspects, the subject is a human or non-human animal.

In some embodiments, the plurality of cells is formulated with a pharmaceutically acceptable carrier or excipient.

In a twelfth aspect, the invention provides a method of producing the cell or plurality of cells, comprising providing an isolated cell or a plurality of isolated cells; providing a preparation of circular polyribonucleotide as described herein, and contacting the circular polyribonucleotide to the isolated cell or plurality of isolated cells, wherein the isolated cell or plurality of isolated cells is capable of expressing the circular polyribonucleotide. In some embodiments, the preparation of circular polyribonucleotide contacted to the cells comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules. In some embodiments, the preparation of circular polyribonucleotide contacted to the cells comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to the total ribonucleotide molecules in the pharmaceutical preparation. In embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) of total ribonucleotide molecules in the preparation are circular polyribonucleotide molecules. In some embodiments of this aspect, viability of the isolated cell or plurality of isolated cells after the contacting is at least 40% compared to a normalized uncontacted isolates cell or plurality of normalized uncontacted isolated cells. In some embodiments of this aspect, the method further comprises administering the cell or plurality of cells after the contacting to a subject.

In a thirteenth aspect, the invention provides a method of producing a cell for administration to a subject comprising a) providing an isolated cell, and b) contacting the isolated cell to a circular polyribonucleotide described herein; thereby producing the cell for administration to the subject. In one embodiment of this aspect, the circular polyribonucleotide in the cell is degraded prior to administration to the subject.

In a fourteenth aspect, the invention provides a method of cellular therapy comprising administering a pharmaceutical composition, cell, plurality of cells, preparation, a plurality of cells in an intravenous bag, a plurality of cells in a medical device, a plurality of cells in a biocompatible matrix, or a plurality of cells from a bioreactor as described herein to a subject in need thereof. In some embodiments, the administered pharmaceutical composition, plurality of cells, cell preparation, plurality of cells in an intravenous bag, plurality of cells in a medical device, or plurality of cells in a biocompatible matrix comprises a unit dose for the subject, e.g., comprises between 10⁵-10⁹ cells/kg of the subject, e.g., between 10⁶-10⁸ cells/kg of the subject. For example, a unit dose for a target subject weighing 50 kg may be a pharmaceutical composition that comprises between 5×10⁷ and 2.5×10¹⁰ cells, e.g., between 5×10⁷ and 2.5×10⁹ cells, e.g., between 5×10⁸ and 5×10⁹ cells.

In some embodiments of this aspect, the pharmaceutical composition, plurality of cells, preparation, intravenous bag, medical device, or biocompatible matrix comprises a dose of, e.g., 1×10⁵ to 9×10¹¹ cells, e.g., between 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g. from 5×10⁵ cells to 4.4×10¹¹ cells, wherein at least 1% of the cells are cells or isolated cells as described herein. For example, at least 50% of the cells, at least 60% of the cells, e.g., between 50-70% of the cells in the plurality, cell preparation, intravenous bag, medical device, or biocompatible matrix are cells comprising a synthetic, exogenous circular RNA as described herein. In some embodiments of this aspect, the method comprises administering the pharmaceutical composition, plurality of cells, or preparation at a dose of 1×10⁵ to 9×10¹¹ cells, e.g., between 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., from 5×10⁵ cells/kg to 6×10⁸ cells/kg. In some embodiments of this aspect, the method comprises administering the pharmaceutical composition, plurality of cells, or preparation in a plurality of administrations or doses. In some embodiments of this aspect, the plurality, e.g., two, subsequent doses are administered at least about a week, 2 weeks, 28 days, 35 days, 42 days, or 60 days apart or more.

In another aspect, the invention provides a method of editing a nucleic acid of an isolated cell or plurality of isolated cells comprising a) providing an isolated cell or a plurality of isolated cells; b) contacting the isolated cell or plurality of isolated cells to a circular polyribonucleotide encoding a nuclease and/or comprising a guide nucleic acid; and thereby producing an edited cell or plurality of edited cells for administration to a subject. In some embodiments of this aspect, the method further comprises formulating the edited cell or the plurality of edited cells with a pharmaceutically acceptable excipient. In some embodiments of this aspect, the nuclease is a zinc finger nuclease, transcription activator like effector nuclease, or Cas protein. In some embodiments of this aspect, the nuclease is a Cas9 protein, Cas12 protein, Cas14 protein, or Cas13 protein.

In another aspect, the invention provides an isolated cell for use in a cellular therapy comprising a circular polyribonucleotide comprising at least one binding site, encoding a protein or a combination thereof. In one embodiment of this aspect, the circular polyribonucleotide (1) comprises at least one binding site, (2) encodes a secreted protein or an intracellular protein, or (3) a combination of (1) and (2). In one embodiment of this aspect, the circular polyribonucleotide (1) comprises at least one binding site, (2) encodes a membrane protein, or (3) a combination of (1) or (2), wherein the membrane protein is not a chimeric antigen receptor, T cell receptor, or T cell receptor fusion protein. In one embodiment of this aspect, the circular polyribonucleotide comprises at least one binding site and encodes a protein, wherein the protein is a secreted protein, membrane protein, or an intracellular protein.

The invention also provides a preparation of between 1×10⁶-1×10¹¹ human cells (e.g., T cells), e.g., between 1×10⁷ to 5×10¹⁰ human cells, e.g., between 1×10⁸-1×10⁹ human cells, formulated with a excipient suitable for parenteral administration, wherein at least 50% (e.g., between 50%-70%) of the cells of the preparation comprise an exogenous circular RNA that expresses a chimeric antigen receptor described herein, and wherein the preparation is in a medical device such as an infusion bag, which is configured for parenteral delivery to a human. The invention also provides a method of treating a human subject diagnosed with cancer, e.g., a leukemia or lymphoma (e.g., acute lymphoblastic leukemia or relapsed or refractory diffuse large B-cell lymphoma), comprising administering to the subject a preparation of autologous T cells formulated with an excipient suitable for parenteral administration, wherein at least 50% (e.g., between 50%-70%) of the cells of the preparation comprise an exogenous circular RNA that expresses a chimeric antigen receptor described herein, wherein the preparation is administered at a dose of between 1×10⁵ to 1×10⁹ cells/kg of the subject, via a medical device such as an infusion bag, which is configured for parenteral delivery to the human.

The invention also provides a preparation of between 1×10⁶-1×10¹¹ human cells (e.g., CD34+ hematopoietic stem cells or HSCs, e.g., NK cells), e.g., between 1×10⁷ to 5×10¹⁰ human cells, e.g., between 1×10⁸-1×10⁹ human cells, formulated with a excipient suitable for parenteral administration, wherein at least 50% (e.g., between 50%-70%) of the cells of the preparation comprise an exogenous circular RNA that expresses hemoglobin Subunit Beta (Beta Globin or Hemoglobin Beta Chain or HBB) for treatment of thalassemia or for sickle cell disease, or express an ABC transporter for treatment of cerebral adrenoleukodystrophy, and wherein the preparation is in a medical device such as an infusion bag, which is configured for parenteral delivery to a human, and wherein the preparation is administered at a dose of between 1×10⁵ to 1×10⁹ cells/kg of the subject, via a medical device such as an infusion bag, which is configured for parenteral delivery to the human.

The invention also provides a preparation of between 1×10⁶-1×10¹¹ human cells (e.g., CD34+ hematopoietic stem cells or HSCs, e.g., NK cells), e.g., between 1×10⁷ to 5×10¹⁰ human cells, e.g., between 1×10⁸-1×10⁹ human cells, formulated with a excipient suitable for parenteral administration, wherein at least 50% (e.g., between 50%-70%) of the cells of the preparation comprise an exogenous circular RNA that expresses (a) hemoglobin Subunit Beta (Beta Globin or Hemoglobin Beta Chain or HBB) for treatment of thalassemia or for sickle cell disease, or (b) an ABC transporter for treatment of cerebral adrenoleukodystrophy, or (c) adenosine deaminase (ADA) for treatment of ADA-SCID, or (d) WAS protein for treatment of Wiskott-Aldrich, or (e) CYBB protein for treatment of X-Linked chronic granulomatous disease or (f) ARSA for treatment of metachromatic leukodystrophy, or (g) α-L-iduronidase for treatment of MPS-I, or (h) N-sulfoglucosamine sulfohydrolase for treatment of MPS-IIIA or (i) N-acetyl-alpha-glucosaminidase for treatment of MPS-IIIB, and wherein the preparation is in a medical device such as an infusion bag, which is configured for parenteral delivery to a human, and wherein the preparation is administered at a dose of between 1×10⁵ to 1×10⁹ cells/kg of the subject, via a medical device such as an infusion bag, which is configured for parenteral delivery to the human. In some embodiments, the dose is an IV dose, e.g., a single IV dose, e.g., of 1-5 million cells.

### Definitions

The present invention will be described with respect to particular embodiments and with reference to certain figures but the invention is not limited thereto but only by the claims. Terms as set forth hereinafter are generally to be understood in their common sense unless indicated otherwise.

As used herein, the terms “circRNA” or “circular polyribonucleotide” or “circular RNA” are used interchangeably and mean a polyribonucleotide molecule that has a structure having no free ends (i.e., no free 3′ and/or 5′ ends), for example a polyribonucleotide molecule that forms a circular or end-less structure through covalent or non-covalent bonds.

As used herein, the term “aptamer sequence” is a non-naturally occurring or synthetic oligonucleotide that specifically binds to a target molecule. Typically an aptamer is from 20 to 500 nucleotides. Typically an aptamer binds to its target through secondary structure rather than sequence homology.

As used herein, the term “therapeutic characteristic” is any characteristic that beneficially affects the course of a condition or disease, including promoting delivery of a therapeutic molecule, such as a circular RNA, to a cell or the effects of the therapeutic molecule on a cell.

As used herein, an “isolated cell” means a cell that has been obtained and separated from a tissue or fluid of a subject. An isolated cell is a cell obtained and separated from a tissue or fluid of a subject, or is a progeny cell of a cell obtained and separated from a tissue or fluid of a subject, for example, an isolated cell can be a primary cell from a subject which is placed in in vitro or ex vivo culture, a progeny of such cell, or a cell from a cell line. In some embodiments, the isolated cell is derived from a subject's own cells (for autologous transfer) or derived from a subject other than the treated subject (for allogenic transfer).

As used herein, the term “encryptogen” is a nucleic acid sequence or structure of the circular polyribonucleotide that aids in reducing, evading, and/or avoiding detection by an immune cell and/or reduces induction of an immune response against the circular polyribonucleotide.

As used herein, the term “expression sequence” is a nucleic acid sequence that encodes a product, e.g., a peptide or polypeptide, or a regulatory nucleic acid. An exemplary expression sequence that codes for a peptide or polypeptide comprises a plurality of nucleotide triads, each of which code for an amino acid and is termed as a “codon”.

As used herein the term “exogenous”, when used with reference to a biomolecule (such as a circular RNA) means that the biomolecule was introduced into a host genome, cell or organism by the hand of man. For example, a circular RNA that is added into an existing genome, cell, tissue or subject using recombinant DNA techniques and/or methods for internalizing a biomolecule into a cell, is exogenous to the existing nucleic acid sequence, cell, tissue or subject, and any progeny of the nucleic acid sequence, cell, tissue or subject that retain the biomolecule.

As used herein, the term “immunoprotein binding site” is a nucleotide sequence that binds to an immunoprotein. In some embodiments, the immunoprotein binding site aids in masking the circular polyribonucleotide as exogenous, for example, the immunoprotein binding site is bound by a protein (e.g., a competitive inhibitor) that prevents the circular polyribonucleotide from being recognized and bound by an immunoprotein, thereby reducing or avoiding an immune response against the circular polyribonucleotide.

As used herein, the term “immunoprotein” is any protein or peptide that is associated with an immune response, e.g., such as against an immunogen, e.g., the circular polyribonucleotide. Non-limiting examples of immunoprotein include T cell receptors (TCRs), antibodies (immunoglobulins), major histocompatibility complex (MHC) proteins, complement proteins, and RNA binding proteins.

As used herein, the term “modified ribonucleotide” means any ribonucleotide analog or derivative that has one or more chemical modifications to the chemical composition of an unmodified natural ribonucleotide, such as a natural unmodified nucleotide adenosine (A), uridine (U), guanine (G), cytidine (C). In some embodiments, the chemical modifications of the modified ribonucleotide are modifications to any one or more functional groups of the ribonucleotide, such as, the sugar the nucleobase, or the internucleoside linkage (e.g. to a linking phosphate/to a phosphodiester linkage/to the phosphodiester backbone).

As used herein, the phrase “quasi-helical structure” is a higher order structure of the circular polyribonucleotide, wherein at least a portion of the circular polyribonucleotide folds into a helical structure.

As used herein, the phrase “quasi-double-stranded secondary structure” is a higher order structure of the circular polyribonucleotide, wherein at least a portion of the circular polyribonucleotide creates an internal double strand.

As used herein, the term “regulatory element” is a moiety, such as a nucleic acid sequence, that modifies expression of an expression sequence within the circular polyribonucleotide.

As used herein, the term “repetitive nucleotide sequence” is a repetitive nucleic acid sequence within a stretch of DNA or RNA or throughout a genome. In some embodiments, the repetitive nucleotide sequence includes poly CA or poly TG (UG) sequences. In some embodiments, the repetitive nucleotide sequence includes repeated sequences in the Alu family of introns.

As used herein, the term “replication element” is a sequence and/or motif(s) necessary or useful for replication or that initiate transcription of the circular polyribonucleotide.

As used herein, the term “stagger element” is a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPG P, where x=any amino acid. In some embodiments, the stagger element may include a chemical moiety, such as glycerol, a non nucleic acid linking moiety, a chemical modification, a modified nucleic acid, or any combination thereof.

As used herein, the term “substantially resistant” means one that has at least 50%, 55%, 60%, 65%, 70%, 75%, 80%, 85%, 90%, 95%, 96%, 97%, 98% or 99% resistance as compared to a reference.

As used herein, the term “stoichiometric translation” means a substantially equivalent production of expression products translated from the circular polyribonucleotide. For example, for a circular polyribonucleotide having two expression sequences, stoichiometric translation of the circular polyribonucleotide can mean that the expression products of the two expression sequences can have substantially equivalent amounts, e.g., amount difference between the two expression sequences (e.g., molar difference) can be about 0, or less than 1%, 2%, 3%, 4%, 5%, 6%, 7%, 8%, 9%, 10%, 15%, or 20%.

As used herein, the term “translation initiation sequence” is a nucleic acid sequence that initiates translation of an expression sequence in the circular polyribonucleotide.

As used herein, the term “termination element” is a moiety, such as a nucleic acid sequence, that terminates translation of the expression sequence in the circular polyribonucleotide.

As used herein, the term “translation efficiency” means a rate or amount of protein or peptide production from a ribonucleotide transcript. In some embodiments, translation efficiency can be expressed as amount of protein or peptide produced per given amount of transcript that codes for the protein or peptide, e.g., in a given period of time, e.g., in a given translation system, e.g., an in vitro translation system like rabbit reticulocyte lysate, or an in vivo translation system like a eukaryotic cell or a prokaryotic cell.

As used herein, the term “circularization efficiency” is a measurement of resultant circular polyribonucleotide versus its starting material.

As used herein, the term “immunogenic” is a potential to induce an immune response to a substance. In some embodiments, an immune response may be induced when an immune system of an organism or a certain type of immune cells is exposed to an immunogenic substance. The term “non-immunogenic” is a lack of or absence of an immune response above a detectable threshold to a substance. In some embodiments, no immune response is detected when an immune system of an organism or a certain type of immune cells is exposed to a non-immunogenic substance. In some embodiments, a non-immunogenic circular polyribonucleotide as provided herein, does not induce an immune response above a pre-determined threshold when measured by an immunogenicity assay. For example, when an immunogenicity assay is used to measure an innate immune response against a circular polyribonucleotide (such as measuring inflammatory markers), a non-immunogenic polyribonucleotide as provided herein can lead to production of an innate immune response at a level lower than a predetermined threshold. The predetermined threshold can be, for instance, at most 1.5 times, 2 times, 3 times, 4 times, 5 times, 6 times, 7 times, 8 times, 9 times, or 10 times the level of a marker(s) produced by an innate immune response for a control reference.

As used herein, the term “linear counterpart” is a polyribonucleotide molecule (and its fragments) having the same or similar nucleotide sequence (e.g., 100%, 95%, 90%, 85%, 80%, 75%, or any percentage therebetween of sequence similarity) as a circular polyribonucleotide and having two free ends (i.e., the uncircularized version (and its fragments) of the circularized polyribonucleotide). In some embodiments, the linear counterpart (e.g., a pre-circularized version) is a polyribonucleotide molecule (and its fragments) having the same or similar nucleotide sequence (e.g., 100%, 95%, 90%, 85%, 80%, 75%, or any percentage therebetween sequence similarity) and same or similar nucleic acid modifications as a circular polyribonucleotide and having two free ends (i.e., the uncircularized version (and its fragments) of the circularized polyribonucleotide). In some embodiments, the linear counterpart is a polyribonucleotide molecule (and its fragments) having the same or similar nucleotide sequence (e.g., 100%, 95%, 90%, 85%, 80%, 75%, or any percentage therebetween of sequence similarity) and different or no nucleic acid modifications as a circular polyribonucleotide and having two free ends (i.e., the uncircularized version (and its fragments) of the circularized polyribonucleotide). In some embodiments, a fragment of the polyribonucleotide molecule that is the linear counterpart is any portion of linear counterpart polyribonucleotide molecule that is shorter than the linear counterpart polyribonucleotide molecule. In some embodiments, the linear counterpart further comprises a 5′ cap. In some embodiments, the linear counterpart further comprises a poly adenosine tail. In some embodiments, the linear counterpart further comprises a 3′ UTR. In some embodiments, the linear counterpart further comprises a 5′ UTR.

As used herein, the term “carrier” means a compound, composition, reagent, or molecule that facilitates the transport or delivery of a composition (e.g., a circular polyribonucleotide) into a cell by a covalent modification of the circular polyribonucleotide, via a partially or completely encapsulating agent, or a combination thereof. Non-limiting examples of carriers include carbohydrate carriers (e.g., an anhydride-modified phytoglycogen or glycogen-type material), nanoparticles (e.g., a nanoparticle that encapsulates or is covalently linked binds to the circular polyribonucleotide), liposomes, fusosomes, ex vivo differentiated reticulocytes, exosomes, protein carriers (e.g., a protein covalently linked to the circular polyribonucleotide), or cationic carriers (e.g., a cationic lipopolymer or transfection reagent).

As used herein, the term “naked delivery” means a formulation for delivery to a cell without the aid of a carrier and without covalent modification to a moiety that aids in delivery to a cell. A naked delivery formulation is free from any transfection reagents, cationic carriers, carbohydrate carriers, nanoparticle carriers, or protein carriers. For example, naked delivery formulation of a circular polyribonucleotide is a formulation that comprises a circular polyribonucleotide without covalent modification and is free from a carrier.

The term “diluent” means a vehicle comprising an inactive solvent in which a composition described herein (e.g., a composition comprising a circular polyribonucleotide) may be diluted or dissolved. A diluent can be an RNA solubilizing agent, a buffer, an isotonic agent, or a mixture thereof. A diluent can be a liquid diluent or a solid diluent. Non-limiting examples of liquid diluents include water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and 1,3-butanediol. Non-limiting examples of solid diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, or powdered sugar.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments, which are presently exemplified. It should be understood, however, that the invention is not limited to the precise arrangement and instrumentalities of the embodiments shown in the drawings.

FIG. 1 shows experimental data demonstrating that expression of a GFP protein encoded by a circular polyribonucleotide (“Endless RNA”) persists in a cell following electroporation for longer than expression of a GFP protein encoded by a linear polyribonucleotide counterpart (“Linear RNA”).

FIG. 2 shows experimental data demonstrating the surface expression of a CAR protein following introduction of circular (“C”) or linear (“L”) polyribonucleotide encoding the CAR protein into the cells.

FIG. 3 shows experimental data demonstrating the expression of Gaussia luciferase encoded by different circular polyribonucleotide constructs or linear polyribonucleotide constructs in HeLa cells as a function of the amount of nucleotide.

FIG. 4 shows that CD19 CAR was expressed on primary human T cells electroporated with circular RNA constructs encoding a CD19 CAR sequence or with linear RNA construct encoding a CD19 CAR sequence. No expression was observed from primary human T cells electroporated with vehicle alone (negative control).

FIG. 5 is a schematic showing a T cells expressing CD19 CAR from a circular RNA construct encoding a CD19 CAR sequence in a tumor killing assay.

FIG. 6 shows T cells expressing a CD19 CAR from a circular RNA construct encoding a CD19 CAR sequence kills tumor cells.

FIG. 7 shows a Western blot of PAH protein expressed in cells from circular polyribonucleotides.

FIG. 8 shows PAH protein expressed in cells by both circular RNAs tested was functional and able to convert phenylalanine to tyrosine

FIG. 9 shows experimental data demonstrating the stability of a circular polyribonucleotide (“GLuc-Circular”) over time as compared to linear polyribonucleotides (“GLuc-Linear” and “GLuc-Linear-Modified-Globin”)

FIG. 10 shows experimental data showing reduced toxicity of a circular polyribonucleotide (“GLuc-Circular”) over time as compared to linear polyribonucleotides (“GLuc-Linear”) or a transfection reagent negative control (“Lipofectamine (−) RNA”).

FIG. 11 shows schematic of circular RNAs. The bottom left schematic shows a circular RNA comprising a C2 min aptamer sequence that binds the transferrin receptor. The bottom middle schematic shows a circular RNA comprising a 36a aptamer sequence that binds the transferrin receptor. The bottom right schematic shows a circular RNA comprising a non-binding sequence that does not bind the transferrin receptor. All three circular RNAs also comprise a sequence that binds to an AF488 labelled DNA oligonucleotide (annealing sequence).

FIG. 12 shows circular polyribonucleotides comprising an aptamer sequence (C2.min or 36a) that binds the transferrin receptor were internalized into cells that express the transferrin receptor based on fluorescence. The circular polyribonucleotides comprising the non-binding aptamer were not internalized into cells that express the transferrin receptor based on fluorescence.

FIG. 13 shows a schematic of a single-stranded RNA oligonucleotide and a circular RNA. The single-stranded RNA oligonucleotide comprises an aptamer sequence and a sequence that binds to the circular polyribonucleotide (binding motif). The circular RNA comprises a sequence that binds to a binding sequence in the single-stranded RNA oligonucleotide. The bottom left schematic shows a single-stranded RNA oligonucleotide comprising a C2 min aptamer sequence that binds the transferrin receptor and a sequence that binds to the circular polyribonucleotide, which is bound to the circular polyribonucleotide. The bottom middle schematic shows a single-stranded RNA oligonucleotide comprising a 36a aptamer sequence that binds the transferrin receptor and a sequence that binds to the circular polyribonucleotide, which is bound to the circular polyribonucleotide. The bottom right schematic shows a single-stranded RNA oligonucleotide comprising a aptamer sequence that is non-binding for the transferrin receptor and a sequence that binds to the circular polyribonucleotide, which is bound to the circular polyribonucleotide.

FIG. 14 is a denaturing PAGE gel image demonstrating exemplary circular RNA after an exemplary purification process.

FIG. 15A is a graph showing qRT-PCR analysis of linear and circular RNA levels 24 hours after delivery to cells using primers that captured both linear and circular RNA.

FIG. 15B is a graph showing qRT-PCR analysis of linear and circular RNA levels using a primer specific for the circular RNA.

FIG. 16 is a graph showing qRT-PCR analysis of immune related genes from 293T cells transfected with circular RNA or linear RNA.

FIG. 17 is a graph showing luciferase activity of protein expressed from circular RNA via rolling circle translation.

FIG. 18 is an image showing a protein blot of expression products from circular RNA or linear RNA.

FIG. 19 shows experimental data demonstrating the higher stability of circular RNA in a dividing cell as compared to linear controls.

FIG. 20 shows experimental data demonstrating the reduced toxicity to transfected cells of an exemplary circular RNA as compared to linear control.

FIG. 21 shows a schematic of an exemplary in vitro production process of a circular RNA that contains a start-codon, an ORF (open reading frame) coding for GFP, a stagger element (2A), an encryptogen, and an IRES (internal ribosome entry site).

FIG. 22 shows a schematic of an exemplary in vivo production process of a circular RNA.

FIG. 23 shows design of an exemplary circular RNA that comprises a start-codon, an ORF coding for GFP, a stagger element (2A), and an encryptogen.

FIG. 24A and FIG. 24B are schematics demonstrating in vivo stoichiometric protein expression of two different circular RNAs.

### DETAILED DESCRIPTION

The present disclosure generally relates to compositions for cell therapy and methods of using the compositions in cellular therapy. The compositions include and the methods use cells (e.g., isolated cells) comprising exogenous circular polyribonucleotides comprising at least one binding site, encoding a protein, or a combination thereof. The protein can be a secreted protein, membrane protein, or intracellular protein. In some embodiments, the protein is a therapeutic protein. In some embodiments, the circular polyribonucleotide lacks a poly-A tail, a replication element, or a combination thereof. The methods of cellular therapy can comprise administering the isolated cells to a subject in need thereof.

The disclosure relates to isolated cells comprising exogenous circular polyribonucleotides. In some embodiments pharmaceutical compositions, preparations, suspensions, medical devices, or biocompatible matrixes comprise the isolated cells for use in cellular therapy. In some embodiments, a bioreactor comprises the isolated cells for use in cellular therapy. In some embodiments, the at least one binding site confer cellular localization to the circular polyribonucleotide. In some aspects, the isolated cell is an edited cell.

The disclosure further relates to producing an isolated cell for cellular therapy. In one embodiment, a method of producing the cell or plurality of cells comprises providing an isolated cell or a plurality of isolated cells as described herein; providing the circular polyribonucleotide as described herein, and contacting the circular polyribonucleotide to the isolated cell or plurality of isolated cells. In some embodiments, the method further comprises administering the cell or plurality of cells after the contacting to a subject.

The disclosure further relates to administering the isolated cells comprising the circular polyribonucleotides as disclosed herein. In one embodiment, a method of cell therapy comprises administering to a subject in need thereof the pharmaceutical composition comprising the isolated cells, a plurality of isolated cells, a preparation comprising the isolated cells, the plurality of isolated cells in an intravenous bag, the plurality of isolated cells from a bioreactor, or implanting the medical device or biocompatible matrix comprising the plurality of isolated cells to a subject.

The disclosure further relates to a method of editing a nucleic acid of an isolated cell or plurality of isolated cells comprises a) providing an isolated cell or plurality of isolated cells; b) contacting the isolated cell or plurality of isolated cells to a circular polyribonucleotide encoding a nuclease and/or comprising a guide nucleic acid; and thereby producing an edited cell or plurality of edited cells for administration to a subject. In some embodiments, the nuclease is a zinc finger nuclease, TALEN, or Cas protein.

In some aspects, the invention relates to a cellular therapy comprising a cell, wherein the cell comprises an exogenous circular polyribonucleotide comprising at least one expression sequence encoding a protein (e.g., a therapeutic protein). In some embodiments, the cell comprises a protein (e.g., a therapeutic protein) and a circular polyribonucleotide, wherein the circular polyribonucleotide comprises at least one expression sequence encoding the protein. In some embodiments, the cell is a therapeutic cell, wherein the therapeutic cell comprises a protein and a circular polyribonucleotide, and wherein the circular polyribonucleotide comprises at least one expression sequence encoding the protein that confers at least one therapeutic characteristic to the cell. The cell may be an ex vivo cell (e.g., an isolated cell). The cell may be an isolated cell. In some embodiments, the cellular therapy is a pharmaceutical composition and further comprises a pharmaceutically acceptable carrier or excipient.

The cells described herein may be used in methods of cell therapy. A method of cell therapy may comprise providing a circular polyribonucleotide, e.g., any of the circular polyribonucleotides disclosed herein or compositions thereof, and contacting the circular polyribonucleotide to a cell ex vivo (e.g., an isolated cell). The circular polyribonucleotide may comprise one or more expression sequences. The expression product of one or more expression sequences may be a protein, e.g., a therapeutic protein. In some embodiments, the method of cellular therapy further comprises administering the cell to a subject in need thereof, e.g., a human subject. In some aspects, a method of cell therapy comprises providing a circular polyribonucleotide comprising one or more expression sequences and contacting the circular polyribonucleotide to a cell ex vivo (e.g., an isolated cell). In some embodiments, an expression product of the one or more expression sequences comprises a protein for treating a subject in need thereof. In further aspects, the invention relates to a method of cell therapy comprising administering the cell or therapeutic cell as disclosed herein, or pharmaceutical compostions thereof, to a subject in need thereof.

### Cells for Cellular Therapy

In some aspects, cell therapy comprises a cell (e.g., an isolated cell), wherein the cell comprises a circular polyribonucleotide, where the circular polyribonucleotide (a) comprises at least one binding site, (b) encodes a protein, or both (a) and (b). The circular polyribonucleotide can comprise at least one expression sequence encoding a protein (e.g., a therapeutic protein), at least one binding site, or a combination thereof. In some embodiments, the cell is a therapeutic cell, wherein the therapeutic cell comprises a protein and a circular polyribonucleotide, and wherein the circular polyribonucleotide comprises at least one expression sequence encoding the protein that confers at least one therapeutic characteristic to the cell. In some embodiments, the cell is a therapeutic cell, wherein the therapeutic cell comprises a circular polyribonucleotide, and wherein the circular polyribonucleotide comprises at least one binding site that confers at least one therapeutic characteristic to the cell. In some embodiments, the circular polyribonucleotide is contacted to a cell. The cell may be an isolated cell. In some embodiments, the cell (e.g., isolated cell) is an isolated mammalian cell comprising an exogenous, synthetic circular polyribonucleotide.

In some embodiments, the cell (e.g., an isolated cell) comprises an exogenous, synthetic circular polyribonucleotide comprising at least one binding site, an encoded protein or a combination thereof, wherein the cell is administered to a subject. In some embodiments, the circular polyribonucleotide (1) comprises at least one binding site, (2) encodes a secreted protein or an intracellular protein, or (3) a combination of (1) and (2). In embodiments, the circular polyribonucleotide (1) comprises at least one binding site, (2) encodes a membrane protein, or (3) a combination of (1) and (2), wherein the membrane protein is not a chimeric antigen receptor, T cell receptor, or T cell receptor fusion protein. In some embodiments, the circular polyribonucleotide comprises at least one binding site and encodes a protein, wherein the protein is a secreted protein, membrane protein, or an intracellular protein.

In some embodiments, a cell for cellular therapy comprise a chimeric antigen receptor (CAR) encoded by an exogenous circular polyribonucleotide as described herein. For example, a cell comprising a circular polyribonucleotide encoding an antigen-binding domain, a transmembrane domain, and an intracellular signaling domain and comprising at least one binding site. In some embodiments, an isolated cell comprises a circular polyribonucleotide encoding a chimeric antigen receptor and comprises at least one binding site, wherein the isolated cell is for administration (e.g., intravenous administration to a subject).

In some embodiments, a cell comprises: (a) a circular polyribonucleotide comprising i) at least one target binding sequence encoding an antigen-binding protein that binds to an antigen or ii) a sequence encoding an antigen-binding domain, a transmembrane domain, and an intracellular signaling domain; and (b) a second nucleotide sequence encoding a protein, wherein expression of the protein is activated upon binding of the antigen to the antigen-binding protein. In some embodiments, the sequence of ii) further comprises at least one binding site. In some embodiments, the protein is a secreted protein. In some embodiments, the protein is a cytokine (e.g., IL-12) or a costimulatory ligand (e.g., CD40 or 4-1BBL).

In particular embodiments, a cell for cellular therapy is a modified T cell. For example, cell comprises a circular polyribonucleotide encoding a T cell receptor (TCR) comprising affinity for an antigen and a circular polyribonucleotide encoding a bispecific antibody, wherein the cell expresses the TCR and bispecific antibody on a surface of the cell.

### Cell Types

In some embodiments, the cell (e.g., an isolated cell) is a eukaryotic cell. In some embodiments, the cell is an animal cell. In some embodiments, a cell is from an aquaculture animal (fish, crabs, shrimp, oysters etc.), a mammal, e.g., a cell from a pet or zoo animal (cats, dogs, lizards, birds, lions, tigers and bears etc.), a cell from a farm or working animal (horses, cows, pigs, chickens etc.), or is a human cell, a cultured cell, a primary cell or from a cell line, a stem cell, a progenitor cell, a differentiated cell, a germ cell, a cancer cell (e.g., tumorigenic, metastic), a non-tumorigenic cell (normal cell), a fetal cell, an embryonic cell, an adult cell, a mitotic cell, or a non-mitotic cell.

In some embodiments, a cell (e.g., an isolated cell) is an immune cell. In some embodiments, a cell is non-immune cell. In some embodiments the cell is a peripheral blood mononuclear cell. In some embodiments, a cell is a lymphocyte. In some embodiments, the cell is a neurological cell. In some embodiments, the cell is a cardiological cell. In some embodiments, the cell is an adipocyte. In some embodiments, the cell is a liver cell. In some embodiments, the cell is a beta cell. A cell can be a cell selected from the group consisting of a T cell (e.g., a regulatory T cell, γδ T cell, αβ cell, CD8+ T cell, or CD4+ T cell), a B cell, a Natural Killer cell, a Natural Killer T cell, a macrophage, a dendritic cell, a red blood cell, a reticulocyte, a myeloid progenitor, and a megakaryocyte.

In some embodiments, the cell (e.g., an isolated cell) is selected from a group consisting of a mesenchymal stem cell, an embryological stem cell, a fetal stem cell, a placental derived stem cell, a induced pluripotent stem cell, an adipose stem cell, a hematopoietic stem cell (e.g., CD34+ cell), a skin stem cell, an adult stem cell, a bone marrow stem cell, a cord blood stem cell, an umbilical cord stem cell, a corneal limbal stem cell, a progenitor stem cell, and a neural stem cell.

In some embodiments, the cell (e.g., the isolated cell) is a peripheral blood lymphocyte. In some embodiments, a cell is a fibroblast. A cell can be a chondrocyte. A cell can be a cardiomyocyte. A cell can be a dopaminergic neuron. A cell can be a microglia. A cell can be an oligodendrocyte. A cell can be an enteric neuron. A cell can be a hepatocyte.

In some embodiments, a cell (e.g., an isolated cell) is replication incompetent, e.g., the cell is post mitotic, or treated with a mitogen or irradiation.

A cell (e.g., an isolated cell) can be removed from subject (e.g., an animal) using any methods known in the art. In some embodiments, a cell is removed from an organ, tissue, blood, or lymph from a subject. In some embodiments, a cell is a removed or isolated cell that was expanded or cultured in vitro. In some embodiments, a cell is from a cell line, e.g., an immortalized laboratory cell line. A cell can be autologous to a subject. A cell can be allogeneic to a subject. A cell can be immunogenic in a subject. In some embodiments, the cell is not immunogenic in a subject. In some embodiments, a plurality of cells (e.g., a plurality of isolated cells), are a homogenous cell population. In some embodiments, a plurality of cells (e.g., a plurality of isolated cells), are a heterogenous population. A heterogenous population, for example, is a heterogenous population of immune cells.

In some embodiments, a cell is in a tissue or an organ removed from a subject to be used for an organ transplant. For example, a cell is in a liver, heart, kidney, skin, cornea, adipose, pancreas, lung, intestine, middle ear, bone, bone marrow, heart valve, connective tissue, or vascularized composite allografts (e.g., a composite of several tissues such as skin, bone, muscle, blood vessels, nerves, and connective tissue).

### Circular Polyribonucleotides

In some aspects, the cell as described herein comprises a circular polyribonucleotide. In some embodiments, the circular polyribonucleotide is an exogenous, synthetic circular polyribonucleotide. In some embodiments, the cellular therapy comprises a cell, wherein the cell comprises a circular polyribonucleotide. In some embodiments, the circular polyribonucleotide (1) comprises at least one binding site, (2) encodes a secreted protein or an intracellular protein, or (3) a combination of (1) and (2). In some embodiments, the circular polyribonucleotide (1) comprises at least one binding site, (2) encodes a membrane protein, or (3) a combination of (1) and (2), wherein the membrane protein is not a chimeric antigen receptor, T cell receptor, or T cell receptor fusion protein. In some embodiments, the circular polyribonucleotide comprises at least one binding site and encodes a protein, wherein the protein is a secreted protein, membrane protein, or an intracellular protein.

The circular polyribonucleotide can comprise at least one expression sequence encoding a protein (e.g., a therapeutic protein) or at least one binding site. In some embodiments, the cell is a therapeutic cell, wherein the therapeutic cell comprises a protein and a circular polyribonucleotide, and wherein the circular polyribonucleotide comprises at least one expression sequence encoding the protein that confers at least one therapeutic characteristic to the cell. In some embodiments, the circular polyribonucleotide is contacted to a cell as described herein.

### Protein

In some embodiments, the circular polyribonucleotide as described herein encodes a protein. The protein can be a secreted protein, membrane protein, or an intracellular protein. In some embodiments, the circular polyribonucleotide encodes an expression sequence that produces an expression product upon translation in the cell. The expression sequence can encode a protein, such as a therapeutic protein. The expression sequence can encode a protein that confers at least one therapeutic characteristic to the cell. The circular polyribonucleotide can comprise one or more expression sequences encoding a protein or therapeutic protein.

In some embodiments, the circular polyribonucleotide comprises an expression sequence encoding a peptide or polypeptide of expression sequence, e.g., a therapeutic protein, for use as a cellular therapy. The protein may treat the disease in the subject in need thereof. In some embodiments, a peptide or polypeptide of expression sequence is any peptide or polypeptide that confers a therapeutic characteristic to cell, e.g., promotes cell expansion, cell immortalization, cell differentiation, and/or localization of the cell to a target. The therapeutic protein can compensate for a mutated, under-expressed, or absent protein in the subject in need thereof. The therapeutic protein can target, interact with, or bind to a cell, tissue, or virus in the subject in need thereof.

In some embodiments, the circular polyribonucleotide comprises one or more RNA expression sequences, each of which may encode a polypeptide. The polypeptide may be produced in substantial amounts. As such, the polypeptide may be any proteinaceous molecule that can be produced.

A polypeptide can be a polypeptide that can be secreted from a cell, or localized to the cytoplasm, nucleus or membrane compartment of a cell. Some polypeptides include, but are not limited to, at least a portion of a viral envelope protein, metabolic regulatory enzymes (e.g., that regulate lipid or steroid production), an antigen, a tolerogen, a cytokine, a toxin, enzymes whose absence is associated with a disease, and polypeptides that are not active in an animal until cleaved (e.g., in the gut of an animal), and a hormone. In some embodiments, the polypeptide is a protein or a therapeutic protein that compensates for a deficiency in the cell (e.g., a mutated protein, a defective protein, a poorly expressed protein, or an absent protein).

In some embodiments, a protein or a therapeutic protein that can be expressed from the circular polyribonucleotide disclosed herein has antioxidant activity, binding activity, cargo receptor activity, catalytic activity, molecular carrier activity, molecular transducer activity, nutrient reservoir activity, structural molecule activity, toxin activity, transcription regulator activity, translation regulator activity, tolerogenic activity, or transporter activity. In some embodiments, the protein is a molecular function regulator. In some embodiments, the protein functions as a protein tag. Some examples of proteins or therapeutic proteins include, but are not limited to, an enzyme replacement protein, a protein for supplementation, a protein vaccination, antigens (e.g. tumor antigens, viral, bacterial), hormones, cytokines, antibodies, immunotherapy (e.g., cancer), cellular reprogramming/transdifferentiation factor, transcription factors, chimeric antigen receptor, transposase or nuclease, immune effector (e.g., influences susceptibility to an immune response/signal), a regulated death effector protein (e.g., an inducer of apoptosis or necrosis), a non-lytic inhibitor of a tumor (e.g., an inhibitor of an oncoprotein), an epigenetic modifying agent, epigenetic enzyme, a transcription factor, a DNA or protein modification enzyme, a DNA-intercalating agent, an efflux pump inhibitor, a nuclear receptor activator or inhibitor, a proteasome inhibitor, a competitive inhibitor for an enzyme, a protein synthesis effector or inhibitor, a nuclease, a protein fragment or domain, a ligand or a receptor, a Cas protein, and a CRISPR system or component thereof. In some embodiments, the protein is a tolerogenic factor, such as HLA-G, PD-L1, CD47, or CD24.

In some embodiments the protein or the therapeutic protein encoded by the circular polyribonucleotide and, optionally, expressed in the cell, is an intracellular protein or a cytosolic protein. The protein or the therapeutic protein may be, for example, phenylalanine hydroxylase, a G-protein, a kinase, a phosphatase, a nuclease, a chimeric antigen receptor, a zinc finger nuclease protein, a transcription activator like protein nuclease, or a Cas protein. In some embodiments, the Cas protein is a Cas9, Cas12, Cas14, or Cas13.

In some embodiments the protein or the therapeutic protein encoded by the circular polyribonucleotide and, optionally, expressed in the cell, is a membrane protein. In some embodiments, the membrane protein is a transmembrane protein. In some embodiments, a membrane protein is an extracellular matrix protein. The protein or the therapeutic protein may be, for example, a chimeric antigen receptor (CAR), a transmembrane receptor, a G-protein-coupled receptor (GPCR), a receptor tyrosine kinase (RTK), an antigen receptor, an ion channel, or a membrane transporter.

In some embodiments, the protein or therapeutic protein is a membrane protein. In some embodiments, the membrane protein is an extracellular matrix protein. In some embodiments, the membrane protein is a chimeric antigen receptor (CAR). In some embodiments, the protein or therapeutic protein comprises an antigen-binding domain, a transmembrane domain, and an intracellular signaling domain. In some embodiments, the antigen-binding domain is linked to the transmembrane domain, which is linked to the intracellular signaling domain to produce a CAR.

In some embodiments, the antigen-binding domain binds a tumor antigen, a tolerogen, or a pathogen, or the antigen is a tumor antigen or pathogen antigen. In some embodiment, the antigen-binding domain is an antibody or antibody fragment thereof. For example, the antigen binding domain is an single chain variable fragment (scFv), variable fragment, or Fab. In some embodiments the antigen binding domain is a bispecific antibody. In some embodiments, the bispecific antibody has a first immunoglobulin variable domain that binds a first epitope and a second immunoglobulin variable domain that binds a second epitope. In some embodiments, the first epitope and the second epitope are the same. In some embodiments, the first epitope and the second epitope are different. In some embodiments, the transmembrane domain links the antigen binding domain and the intracellular signaling domain.

In some embodiments, the transmembrane domain is a hinge protein (e.g., immunglobuline hinge), a polypeptide linker (e.g., GS linker), a KIR2DS2 hinge, a CD8a hinge, or a spacer. In some embodiments, the intracellular signaling domain comprises at least a portion of a T-cell signaling molecule.

In some embodiments, the intracellular signaling domain comprises an immunoreceptor tyrosine-based activation motif. In some embodiments, the intracellular signaling domain comprises at least a portion of CD3zeta, common FcRgamma (FCER1G), Fc gamma RIIa, FcRbeta (Fc Epsilon Rib), CD3 gamma, CD3delta, CD3epsilon, CD79a, CD79b, DAP10, DAP12, or any combination thereof. In some embodiments, the intracellular signaling domain further comprises a costimulatory intracellular signaling domain. In some embodiments, the costimulatory intracellular signaling domain comprises at least one or more of a TNF receptor protein, immunoglobulin-like protein, a cytokine receptor, an integrin, a signaling lymphocytic activation molecule, or an activating NK cell receptor protein. In some embodiments, the costimulatory intracellular signaling domain comprises at least one or more of CD27, CD28, 4-1BB, OX40, GITR, CD30, CD40, PD-1, ICOS, BAFFR, HVEM, ICAM-1, LFA-1, CD2, CDS, CD7, CD287, LIGHT, NKG2C, NKG2D, SLAMF7, NKp80, NKp30, NKp44, NKp46, CD160, CD19, CD4, CD8alpha, CD8beta, IL2R beta, IL2R gamma, IL7R alpha, ITGA4, VLA1, CD49a, IA4, CD49D, ITGA6, VLA6, CD49f, ITGAD, CD103, ITGAL, ITGAM, ITGAX, ITGB1, CD29, ITGB2, CD18, ITGB7, TNFR2, TRANCE/TRANKL, CD226, SLAMF4, CD84, CD96, CEACAM1, CRTAM, CD229, CD160, PSGL1, CD100, CD69, SLAMF6, SLAMF1, SLAMF8, CD162, LTBR, LAT, GADS, SLP-76, PAG/Cbp, CD19a, B7-H3, or a ligand that binds to CD83.

In some embodiments, the chimeric antigen receptor is a CD19 specific chimeric antigen receptor, a TAA specific chimeric antigen receptor, a BCMA specific chimeric antigen receptor, a HER2 specific chimeric antigen receptor, a CD2 specific chimeric antigen receptor, a NY-ESO-1 specific chimeric antigen receptor, a CD20 specific chimeric antigen receptor, a Mesothelina specific chimeric antigen receptor, a EBV specific chimeric antigen receptor, or a CD33 specific chimeric antigen receptor.

In some embodiments, the protein or the therapeutic protein encoded by the circular polyribonucleotide and, optionally, expressed in the cell, is a secreted protein. The secreted protein may be, for example, an erythropoietin, a cytokine, insulin, oxytocin, a secretary enzyme, a hormone, or a neurotransmitter.

In some embodiments, the protein or the therapeutic protein may have an activity. For example, the activity may be an antioxidant activity, a binding activity, a cargo receptor activity, a catalytic activity, a molecular carrier activity, a molecular transducer activity, a nutrient reservoir activity, a structural molecule activity, a toxin activity, a transcription regulator activity, a translation regulator activity, or a transporter activity. In some embodiments, the activity may confer a characteristic to the cell (e.g., immortalization, cell differentiation, localization to a target site, expansion, and/or increased replication). In some embodiments, the protein or therapeutic protein for cell differentiation is Oct4, Klf4, Sox2, cMyc, or a combination thereof. In some embodiments, these proteins are used to reprogram a cell, e.g., to produce an induced pluripotent stem cell.

In some embodiments, exemplary proteins that can be expressed from the circular polyribonucleotide disclosed herein include human proteins, for instance, receptor binding protein, hormone, growth factor, growth factor receptor modulator, and regenerative protein (e.g., proteins implicated in proliferation and differentiation, e.g., therapeutic protein, for wound healing). In some embodiments, exemplary proteins that can be expressed from the circular polyribonucleotide disclosed herein include EGF (epidermal growth factor). In some embodiments, exemplary proteins that can be expressed from the circular polyribonucleotide disclosed herein include enzymes, for instance, oxidoreductase enzymes, metabolic enzymes, mitochondrial enzymes, oxygenases, dehydrogenases, ATP-independent enzyme, and desaturases. In some embodiments, exemplary proteins that can be expressed from the circular polyribonucleotide disclosed herein include an intracellular protein or cytosolic protein. In some embodiments, the circular polyribonucleotide expresses a phenylalanine hydroxylase. In some embodiments, exemplary proteins that can be expressed from the circular polyribonucleotide disclosed herein include a secreted protein, for instance, a secretary enzyme. In some embodiments, the circular polyribonucleotide expresses an erythropoietin. In some embodiments, the circular polyribonucleotide expresses an epidermal growth factor (EGF). In some cases, the circular polyribonucleotide expresses a secretory protein that can have a short half-life therapeutic in the blood, or can be a protein with a subcellular localization signal, or protein with secretory signal peptide.

In some embodiments, the protein or the therapeutic protein specifically binds an antigen. For example, peptides useful in the invention described herein include antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such antigen binding peptides may bind a cytosolic antigen, a nuclear antigen, an intra-organellar antigen. In some embodiment, the antigen is a tumor antigen, toleragen, or pathogen antigen. In some embodiments, the antigen is expressed from a tumor or cancer.

In some embodiments, the circular polyribonucleotide expresses an antibody, e.g., an full-length antibody, an antibody fragment, or a portion thereof. In some embodiments, the antibody expressed by the circular polyribonucleotide can be of any isotype, such as IgA, IgD, IgE, IgG, IgM. In some embodiments, the circular polyribonucleotide expresses a portion of an antibody, such as a light chain, a heavy chain, a Fc fragment, a CDR (complementary determining region), a Fv fragment, or a Fab fragment, a further portion thereof. In some embodiments, the circular polyribonucleotide expresses one or more portions of an antibody. For instance, the circular polyribonucleotide can comprise more than one expression sequence, each of which expresses a portion of an antibody, and the sum of which can constitute the antibody. In some cases, the circular polyribonucleotide comprises one expression sequence coding for the heavy chain of an antibody, and another expression sequence coding for the light chain of the antibody. When the circular polyribonucleotide is expressed in a cell, the light chain and heavy chain can be subject to appropriate modification, folding, or other post-translation modification to form a functional antibody.

A peptide may include, but is not limited to, a neurotransmitter, a hormone, a drug, a toxin, a viral or microbial particle, a synthetic molecule, and agonists or antagonists thereof.

The polypeptide may be linear or branched. The polypeptide may have a length from about 5 to about 40,000 amino acids, about 15 to about 35,000 amino acids, about 20 to about 30,000 amino acids, about 25 to about 25,000 amino acids, about 50 to about 20,000 amino acids, about 100 to about 15,000 amino acids, about 200 to about 10,000 amino acids, about 500 to about 5,000 amino acids, about 1,000 to about 2,500 amino acids, or any range therebetween. In some embodiments, the polypeptide has a length of less than about 40,000 amino acids, less than about 35,000 amino acids, less than about 30,000 amino acids, less than about 25,000 amino acids, less than about 20,000 amino acids, less than about 15,000 amino acids, less than about 10,000 amino acids, less than about 9,000 amino acids, less than about 8,000 amino acids, less than about 7,000 amino acids, less than about 6,000 amino acids, less than about 5,000 amino acids, less than about 4,000 amino acids, less than about 3,000 amino acids, less than about 2,500 amino acids, less than about 2,000 amino acids, less than about 1,500 amino acids, less than about 1,000 amino acids, less than about 900 amino acids, less than about 800 amino acids, less than about 700 amino acids, less than about 600 amino acids, less than about 500 amino acids, less than about 400 amino acids, less than about 300 amino acids, or less may be useful.

In some embodiments, the expression of a protein (e.g., a therapeutic protein or a protein that confers a therapeutic characteristic) from the circular polyribonucleotide is transient or long term. The expression can result in a therapeutic effect on the cell, in the cell, or of the cell. In certain embodiments, the therapeutic effect persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the therapeutic effect persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the one or more expression sequences generates at least 1.5 fold greater expression product than a linear counterpart in the cell for a time period of at least at 3, 4, 5, 6, 7, 8, 9, 10, 12, 14, or 16 days in the cell. In some embodiments, expression of the one or more expression sequences in the cell is maintained at a level that does not vary by more than about 10%, 20%, 30%, 40%, 50%, 60%, 70%, 80%, 90%, or 95% for time period of at least 3, 4, 5, 6, 7, 8, 9, 10, 12, 14, or 16 days. In some embodiments, the expression of the one or more expression sequences in the cell over a time period of at least 3, 4, 5, 6, 7, 8, 9, 10, 12, 14, or 16 days does not decrease by greater than about 10%, 20%, 30%, 40%, 50%, 60%, 70%, 80%, 90%, or 95%.

In some embodiments, the circular polyribonucleotide comprises one or more expression sequences and is configured for persistent expression in a cell of a subject in vivo. In some embodiments, the circular polyribonucleotide is configured such that protein expression of the one or more expression sequences in the cell at a later time point is equal to or higher than an earlier time point. In such embodiments, the protein expression of the one or more expression sequences can be either maintained at a relatively stable level or can increase over time. The protein expression of the expression sequences can be relatively stable for an extended period of time. For instance, in some cases, the protein expression of the one or more expression sequences in the cell over a time period of at least 3, 4, 5, 6, 7, 8, 9, 10, 12, 14, 16, 18, 20, 22, 23 or more days does not decrease by 50%, 45%, 40%, 35%, 30%, 25%, 20%, 15%, 10%, or 5%. In some cases, the protein expression of the one or more expression sequences in the cell is maintained at a level that does not vary by more than 50%, 45%, 40%, 35%, 30%, 25%, 20%, 15%, 10%, or 5% for at least 3, 4, 5, 6, 7, 8, 9, 10, 12, 14, 16, 18, 20, 22, 23 or more days.

The present invention includes expression of the peptides or polypeptides, protein expression, comprising translating at least a region of the circular polyribonucleotide provided herein. Protein expression can occur from a circular polyribonucleotide as disclosed herein that encodes a protein (e.g, a therapeutic protein or a protein that confers a therapeutic characteristic to a therapeutic cell). Protein expression may occur after contacting the cell with the circular polyribonucleotide. Protein expression may occur in a cell, for example an ex vivo cell (e.g., an isolated cell). Protein expression may occur in a cell after administration of the cell to a subject in need thereof.

In some embodiments, the methods for protein expression comprises translation of at least 10%, at least 20%, at least 30%, at least 40%, at least 50%, at least 60%, at least 70%, at least 80%, at least 90%, or at least 95% of the total length of the circular polyribonucleotide into polypeptides. In some embodiments, the methods for protein expression comprises translation of the circular polyribonucleotide into polypeptides of at least 5 amino acids, at least 10 amino acids, at least 15 amino acids, at least 20 amino acids, at least 50 amino acids, at least 100 amino acids, at least 150 amino acids, at least 200 amino acids, at least 250 amino acids, at least 300 amino acids, at least 400 amino acids, at least 500 amino acids, at least 600 amino acids, at least 700 amino acids, at least 800 amino acids, at least 900 amino acids, or at least 1000 amino acids. In some embodiments, the methods for protein expression comprises translation of the circular polyribonucleotide into polypeptides of about 5 amino acids, about 10 amino acids, about 15 amino acids, about 20 amino acids, about 50 amino acids, about 100 amino acids, about 150 amino acids, about 200 amino acids, about 250 amino acids, about 300 amino acids, about 400 amino acids, about 500 amino acids, about 600 amino acids, about 700 amino acids, about 800 amino acids, about 900 amino acids, or about 1000 amino acids. In some embodiments, the methods comprise translation of the circular polyribonucleotide into continuous polypeptides as provided herein, discrete polypeptides as provided herein, or both.

In some embodiments, the translation of the at least a region of the circular polyribonucleotide takes place in vivo, for instance, after transfection of a eukaryotic cell, or transformation of a prokaryotic cell such as a bacteria, or after contacting a cell such as an ex vivo cell (e.g., an isolated cell) to a circular polyribonucleotide.

In some embodiments, the methods for protein expression comprise modification, folding, or other post-translation modification of the translation product. In some embodiments, the methods for protein expression comprise post-translation modification in vivo or in an ex vivo cell, e.g., via cellular machinery.

In some embodiments, the protein expression results in the production of an intracellular protein, membrane protein, or a secreted protein.

In some embodiments, the one or more expression sequences generates an amount of discrete polypeptides as compared to total polypeptides, wherein the amount is a percent of the total amount of polypeptides by moles of polypeptide. The polypeptides may be generated during rolling circle translation of a circular polyribonucleotide. Each of the discrete polypeptides may be generated from a single expression sequence. In some embodiments, the amount of discrete polypeptides is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 94%, at least 95%, at least 96%, at least 97%, or at least 98% of total polypeptides (molar/molar). In some embodiments, the amount of discrete polypeptides is from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% of total polypeptides (molar/molar).

In some embodiments, the circular polyribonucleotide comprises an expression sequence that generates a greater amount of an expression product than a linear polyribonucleotide counterpart. In some embodiments, the greater amount of the expression product is at least 1-fold, at least 1.2-fold, at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, at least 20-fold, or at least 25-fold greater than that of the linear polyribonucleotide counterpart. In some embodiments, the greater amount of the expression product is from 1.5-fold to 1.6-fold, from 1.6-fold to 1.7-fold, from 1.7-fold to 1.8-fold, from 1.8-fold to 1.9-fold, from 1.9-fold to 2-fold, from 2-fold to 2.5-fold, from 2.5-fold to 3-fold, from 3-fold to 3.5-fold, from 3.5-fold to 4-fold, from 4-fold to 4.5-fold, from 4.5-fold to 5-fold, from 5-fold to 6-fold, from 6-fold to 7-fold, from 7-fold to 8-fold, from 8-fold to 9-fold, from 9-fold to 10-fold, from 10-fold to 15-fold, from 15-fold to 20-fold, from 20-fold to 25-fold, from 2-fold to 5-fold, from 2-fold to 6-fold, from 2-fold to 7-fold, from 2-fold to 10-fold, from 2-fold to 20-fold, from 4-fold to 5-fold, from 4-fold to 6-fold, from 4-fold to 7-fold, from 4-fold to 10-fold, from 4-fold to 20-fold, from 5-fold to 6-fold, from 5-fold to 7-fold, from 5-fold to 10-fold, from 5-fold to 20-fold, or from 10-fold to 20-fold greater than that of the linear polyribonucleotide counterpart. In some embodiments, the greater amount of the expression product is generated in a cell for at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days. In some embodiments, the greater amount of the expression product is generated in a cell for from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days.

In some embodiments, the one or more expression sequences generates at least 1.5 fold greater expression product than a linear counterpart in the cell for a time period of at least at 3, 4, 5, 6, 7, 8, 9, 10, 12, 14, or 16 days in the cell. In some embodiments, the time period begins one day after contacting the cell with the circular polyribonucleotide. In some embodiments, expression of the one or more expression sequences in the cell is maintained at a level that does not vary by more than about 10%, 20%, 30%, 40%, 50%, 60%, 70%, 80%, 90%, or 95% for time period of at least 3, 4, 5, 6, 7, 8, 9, 10, 12, 14, or 16 days. In some embodiments, the time period begins one day after contacting the cell with the circular polyribonucleotide. In some embodiments, the level of the expression that is maintained is the level of the expression at the beginning of the time period, e.g., the level of expression one day after contacting the cell with the circular polyribonucleotide. In some embodiments, the level of the expression that is maintained is the highest level of the expression one day after contacting the cell with the circular polyribonucleotide. In some embodiments, the expression of the one or more expression sequences in the cell over a time period of at least 3, 4, 5, 6, 7, 8, 9, 10, 12, 14, or 16 days does not decrease by greater than about 10%, 20%, 30%, 40%, 50%, 60%, 70%, 80%, 90%, or 95%. In some embodiments, the time period begins one day after contacting the cell with the circular polyribonucleotide. In some embodiments, the level of the expression that does not decrease by greater than about 10%, 20%, 30%, 40%, 50%, 60%, 70%, 80%, 90%, or 95% is the level of the expression at the beginning of the time period. In some embodiments, the level of the expression that does not decrease by greater than about 10%, 20%, 30%, 40%, 50%, 60%, 70%, 80%, 90%, or 95% is the highest level of the expression at the beginning of time period, e.g., the level of expression one day after contacting the cell with the circular polyribonucleotide. In some embodiments, the level of the expression does not decrease by greater than about 10%, 20%, 30%, 40%, 50%, 60%, 70%, 80%, 90%, or 95% compared to the highest level of the expression one day after contacting the cell with the circular polyribonucleotide.

After translation, the protein can be detected in the cell or as a secreted protein. In some embodiments, the protein is detected in the cell over a time period of at least 3, 4, 5, 6, 7, 8, 9, 10, 12, 14, 16, 20, 30, 40, 50, 60, or more days. In some embodiments, the protein is detected on surface of the cell over a time period of at least 3, 4, 5, 6, 7, 8, 9, 10, 12, 14, 16, 20, 30, 40, 50, 60, or more days. In some embodiments, the secreted protein is detected over a time period of at least 3, 4, 5, 6, 7, 8, 9, 10, 12, 14, 16, 20, 30, 40, 50, 60, or more days. In some embodiments, the time period begins one day after contacting the cell with the circular polyribonucleotide encoding the protein. The protein can be detected using any technique known in the art for protein detection, such as by flow cytometry.

The peptide may include, but is not limited to, small peptide, peptidomimetic (e.g., peptoid), amino acids, and amino acid analogs. The peptide may be linear or branched. Such peptide may have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. A peptide can be a therapeutic peptide.

### Binding Site

In some embodiments, the circular polyribonucleotide encodes at least one binding site. The at least one binding site can bind a target, such as protein, RNA, or DNA. The at least one binding site be a protein binding site, an RNA binding site, or a DNA binding site. The at least one binding site confers at least one therapeutic characteristic to the cell. In some embodiments, the at least one binding site confers nucleic acid (e.g., the circular polyribonucleotide as described herein) localization to a cell. In some embodiments, the at least one binding site confers nucleic acid activity (e.g., is a miRNA binding site that results in nucleic acid degradation in cells comprising the miRNA) to the cell comprising the circular polyribonucleotide. In some embodiments, the at least one binding site binds to a cell receptor on a surface of a cell. In some embodiments, a circular polyribonucleotide is internalized into the cell as described herein when the at least one binding site binds to a cell receptor on the surface of the cell. In some embodiments, the at least binding site hybridizes to a linear polynucleotide that aids in internalization of the circular polyribonucleotide into a cell. For example, the linear polynucleotide comprises a region that hybridizes to the at least one binding site of the circular polyribonucleotide and a region that binds to a cell receptor on the surface of the cell. In some embodiments, the region of the linear polyribonucleotide that binds to the cell receptor results in internalization of the linear polyribonucleotide hybridized to the circular polyribonucleotide after binding.

In some embodiments, a circRNA comprises one binding site. A binding site can comprise an aptamer. In some instances, a circRNA comprises at least two binding sites. For example, a circRNA can comprise 2, 3, 4, 5, 6, 7, 8, 9, 10, 11, 12, 13, 14, 15, 16, 17, 18, 19, 20, or more binding sites. In some embodiments, a circRNA described herein is a molecular scaffold that binds one or more targets, or one or more binding moieties of one or more targets. Each target may be, but is not limited to, a different or the same nucleic acids (e.g., RNAs, DNAs, RNA-DNA hybrids), small molecules (e.g., drugs), aptamers, polypeptides, proteins, lipids, carbohydrates, antibodies, viruses, virus particles, membranes, multi-component complexes, cells, cellular moieties, any fragments thereof, and any combination thereof. In some embodiments, the one or more binding sites binds to the same target. In some embodiments, the one or more binding sites bind to one or more binding moieties of the same target. In some embodiments, the one or more binding sites bind to one or more different targets. In some embodiments, the one or more binding sites bind to one or more binding moieties of different targets. In some embodiments, a circRNA acts as a scaffold for one or more binding one or more targets. In some embodiments, a circRNA acts as a scaffold for one or more binding moieties of one or more targets. In some embodiments, a circRNA modulates cellular processes by specifically binding to one or more one or more targets. In some embodiments, a circRNA modulates cellular processes by specifically binding to one or more binding moieties of one or more targets. In some embodiments, a circRNA modulates cellular processes by specifically binding to one or more targets. In some embodiments, a circRNA described herein includes binding sites for one or more specific targets of interest. In some embodiments, a circRNA includes multiple binding sites or a combination of binding sites for each target of interest. In some embodiments, a circRNA includes multiple binding sites or a combination of binding sites for each binding moiety of interest. For example, a circRNA can include one or more binding sites for a polypeptide target. In some embodiments, a circRNA includes one or more binding sites for a polynucleotide target, such as a DNA or RNA, an mRNA target, an rRNA target, a tRNA target, or a genomic DNA target.

In some embodiments, a circRNA comprises a binding site for a single-stranded DNA. In some instances, a circRNA comprises a binding site for double-stranded DNA. In some instances, a circRNA comprises a binding site for an antibody. In some instances, a circRNA comprises a binding site for a virus particle. In some instances, a circRNA comprises a binding site for a small molecule. In some instances, a circRNA comprises a binding site that binds in or on a cell. In some instances, a circRNA comprises a binding site for a RNA-DNA hybrid. In some instances, a circRNA comprises a binding site for a methylated polynucleotide. In some instances, a circRNA comprises a binding site for an unmethylated polynucleotide. In some instances, a circRNA comprises a binding site for an aptamer. In some instances, a circRNA comprises a binding site for a polypeptide. In some instances, a circRNA comprises a binding site for a polypeptide, a protein, a protein fragment, a tagged protein, an antibody, an antibody fragment, a small molecule, a virus particle (e.g., a virus particle comprising a transmembrane protein), or a cell. In some instances, a circRNA comprises a binding site for a binding moiety on a single-stranded DNA. In some instances, a circRNA comprises a binding site for a binding moiety on a double-stranded DNA. In some instances, a circRNA comprises a binding site for a binding moiety on an antibody. In some instances, a circRNA comprises a binding site for a binding moiety on a virus particle. In some instances, a circRNA comprises a binding site for a binding moiety on a small molecule. In some instances, a circRNA comprises a binding site for a binding moiety in or on a cell. In some instances, a circRNA comprises a binding site for a binding moiety on a RNA-DNA hybrid. In some instances, a circRNA comprises a binding site for a binding moiety on a methylated polynucleotide. In some instances, a circRNA comprises a binding site for a binding moiety on an unmethylated polynucleotide. In some instances, a circRNA comprises a binding site for a binding moiety on an aptamer. In some instances, a circRNA comprises a binding site for a binding moiety on a polypeptide. In some instances, a circRNA comprises a binding site for a binding moiety on a polypeptide, a protein, a protein fragment, a tagged protein, an antibody, an antibody fragment, a small molecule, a virus particle (e.g., a virus particle comprising a transmembrane protein), or a cell.

In some embodiments, a binding site binds to a portion of a target comprising at least two amide bonds. In some instances, a binding site does not bind to a portion of a target comprising a phosphodiester linkage. In some instances, a portion of the target is not DNA or RNA. In some instances, a binding moiety comprises at least two amide bonds. In some instances, a binding moiety does not comprise a phosphodiester linkage. In some instances, a binding moiety is not DNA or RNA.

The circRNAs provided herein can include one or more binding sites for binding moieties on a complex. The circRNAs provided herein can include one or more binding sites for targets to form a complex. For example, the circRNAs provided herein can act as a scaffold to form a complex between a circRNA and a target. In some embodiments, a circRNA forms a complex with a single target. In some embodiments, a circRNA forms a complex with two targets. In some embodiments, a circRNA forms a complex with three targets. In some embodiments, a circRNA forms a complex with four targets. In some embodiments, a circRNA forms a complex with five or more targets. In some embodiments, a circRNA forms a complex with a complex of two or more targets. In some embodiments, a circRNA forms a complex with a complex of three or more targets. In some embodiments, two or more circRNAs form a complex with a single target. In some embodiments, two or more circRNAs form a complex with two or more targets. In some embodiments, a first circRNA forms a complex with a first binding moiety of a first target and a second different binding moiety of a second target. In some embodiments, a first circRNA forms a complex with a first binding moiety of a first target and a second circRNA forms a complex with a second binding moiety of a second target.

In some embodiments, a circRNA can include a binding site for one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some embodiments, a circRNA can include a binding site for one or more binding moieties on one or more antibody-polypeptide complexes, polypeptide-polypeptide complexes, polypeptide-DNA complexes, polypeptide-RNA complexes, polypeptide-aptamer complexes, virus particle-antibody complexes, virus particle-polypeptide complexes, virus particle-DNA complexes, virus particle-RNA complexes, virus particle-aptamer complexes, cell-antibody complexes, cell-polypeptide complexes, cell-DNA complexes, cell-RNA complexes, cell-aptamer complexes, small molecule-polypeptide complexes, small molecule-DNA complexes, small molecule-aptamer complexes, small molecule-cell complexes, small molecule-virus particle complexes, and combinations thereof.

In some embodiments, a binding site binds to a polypeptide, protein, or fragment thereof. In some embodiments, a binding site binds to a domain, a fragment, an epitope, a region, or a portion of a polypeptide, protein, or fragment thereof of a target. For example, a binding site binds to a domain, a fragment, an epitope, a region, or a portion of an isolated polypeptide, a polypeptide of a cell, a purified polypeptide, or a recombinant polypeptide. For example, a binding site binds to a domain, a fragment, an epitope, a region, or a portion of an antibody or fragment thereof. For example, a binding site binds to a domain, a fragment, an epitope, a region, or a portion of a transcription factor. For example, a binding site binds to a domain, a fragment, an epitope, a region, or a portion of a receptor. For example, a binding site binds to a domain, a fragment, an epitope, a region, or a portion of a transmembrane receptor. Binding sites may bind to a domain, a fragment, an epitope, a region, or a portion of isolated, purified, and/or recombinant polypeptides. Binding sites can bind to a domain, a fragment, an epitope, a region, or a portion of a mixture of analytes (e.g., a lysate). For example, a binding site binds to a domain, a fragment, an epitope, a region, or a portion of from a plurality of cells or from a lysate of a single cell. A binding site can bind to a binding moiety of a target. In some embodiments, a binding moiety is on a polypeptide, protein, or fragment thereof. In some embodiments, a binding moiety comprises a domain, a fragment, an epitope, a region, or a portion of a polypeptide, protein, or fragment thereof. For example, a binding moiety comprises a domain, a fragment, an epitope, a region, or a portion of an isolated polypeptide, a polypeptide of a cell, a purified polypeptide, or a recombinant polypeptide. For example, a binding moiety comprises a domain, a fragment, an epitope, a region, or a portion of an antibody or fragment thereof. For example, a binding moiety comprises a domain, a fragment, an epitope, a region, or a portion of a transcription factor. For example, a binding moiety comprises a domain, a fragment, an epitope, a region, or a portion of a receptor. For example, a binding moiety comprises a domain, a fragment, an epitope, a region, or a portion of a transmembrane receptor. Binding moieties may be on or comprise a domain, a fragment, an epitope, a region, or a portion of isolated, purified, and/or recombinant polypeptides. Binding moieties include binding moieties on or a domain, a fragment, an epitope, a region, or a portion of a mixture of analytes (e.g., a lysate). For example, binding moieties are on or comprise a domain, a fragment, an epitope, a region, or a portion of from a plurality of cells or from a lysate of a single cell.

In some embodiments, a binding site binds to a domain, a fragment, an epitope, a region, or a portion of a chemical compound (e.g., small molecule). For example, a binding binds to a domain, a fragment, an epitope, a region, or a portion of a drug. For example, a binding site binds to a domain, a fragment, an epitope, a region, or a portion of a compound. For example, a binding moiety binds to a domain, a fragment, an epitope, a region, or a portion of an organic compound. In some instances, a binding site binds to a domain, a fragment, an epitope, a region, or a portion of a small molecule with a molecular weight of 900 Daltons or less. In some instances, a binding site binds to a domain, a fragment, an epitope, a region, or a portion of a small molecule with a molecular weight of 500 Daltons or more. The portion the small molecule that the binding site binds to may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference. A binding site can bind to a binding moiety of a small molecule. In some instances, a binding moiety is on or comprises a domain, a fragment, an epitope, a region, or a portion of a small molecule. For example, a binding moiety is on or comprises a domain, a fragment, an epitope, a region, or a portion of a drug. For example, a binding moiety is on or comprises a domain, a fragment, an epitope, a region, or a portion of a compound. For example, a binding moiety is on or comprises a domain, a fragment, an epitope, a region, or a portion of an organic compound. In some instances, a binding moiety is on or comprises a domain, a fragment, an epitope, a region, or a portion of a small molecule with a molecular weight of 900 Daltons or less. In some instances, a binding moiety is on or comprises a domain, a fragment, an epitope, a region, or a portion of a small molecule with a molecular weight of 500 Daltons or more. Binding moieties may be obtained, for example, from a library of naturally occurring or synthetic molecules, including a library of compounds produced through combinatorial means, i.e. a compound diversity combinatorial library. Combinatorial libraries, as well as methods for their production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

A binding site can bind to a domain, a fragment, an epitope, a region, or a portion of a member of a specific binding pair (e.g., a ligand). A binding site can bind to a domain, a fragment, an epitope, a region, or a portion of monovalent (monoepitopic) or polyvalent (polyepitopic). A binding site can bind to an antigenic or haptenic portion of a target. A binding site can bind to a domain, a fragment, an epitope, a region, or a portion of a single molecule or a plurality of molecules that share at least one common epitope or determinant site. A binding site can bind to a domain, a fragment, an epitope, a region, or a portion of a part of a cell (e.g., a bacteria cell, a plant cell, or an animal cell). A binding site can bind to a target that is in a natural environment (e.g., tissue), a cultured cell, or a microorganism (e.g., a bacterium, fungus, protozoan, or virus), or a lysed cell. A binding site can bind to a portion of a target that is modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group. A binding site can bind to a binding moiety of a member of a specific binding pair. A binding moiety can be on or comprise a domain, a fragment, an epitope, a region, or a portion of a member of a specific binding pair (e.g., a ligand). A binding moiety can be on or comprise a domain, a fragment, an epitope, a region, or a portion of monovalent (monoepitopic) or polyvalent (polyepitopic). A binding moiety can be antigenic or haptenic. A binding moiety can be on or comprise a domain, a fragment, an epitope, a region, or a portion of a single molecule or a plurality of molecules that share at least one common epitope or determinant site. A binding moiety can be on or comprise a domain, a fragment, an epitope, a region, or a portion of a part of a cell (e.g., a bacteria cell, a plant cell, or an animal cell). A binding moiety can be either in a natural environment (e.g., tissue), a cultured cell, or a microorganism (e.g., a bacterium, fungus, protozoan, or virus), or a lysed cell. A binding moiety can be modified (e.g., chemically), to provide one or more additional binding sites such as, but not limited to, a dye (e.g., a fluorescent dye), a polypeptide modifying moiety such as a phosphate group, a carbohydrate group, and the like, or a polynucleotide modifying moiety such as a methyl group.

In some instances, a binding site binds to a domain, a fragment, an epitope, a region, or a portion of a molecule found in a sample from a host. A binding site can bind to a binding moeity of a molecule found in a sample from a host. In some instances, a binding moiety is on or comprises a domain, a fragment, an epitope, a region, or a portion of a molecule found in a sample from a host. A sample from a host includes a body fluid (e.g., urine, blood, plasma, serum, saliva, semen, stool, sputum, cerebral spinal fluid, tears, mucus, and the like). A sample can be examined directly or may be pretreated to render a binding moiety more readily detectible. Samples include a quantity of a substance from a living thing or formerly living things. A sample can be natural, recombinant, synthetic, or not naturally occurring. A binding site can bind to any of the above that is expressed from a cell naturally or recombinantly, in a cell lysate or cell culture medium, an in vitro translated sample, or an immunoprecipitation from a sample (e.g., a cell lysate). A binding moiety can be any of the above that is expressed from a cell naturally or recombinantly, in a cell lysate or cell culture medium, an in vitro translated sample, or an immunoprecipitation from a sample (e.g., a cell lysate).

In some instances, a binding site binds to a target expressed in a cell-free system or in vitro. For example, a binding site binds to a target in a cell extract. In some instances, a binding site binds to a target in a cell extract with a DNA template, and reagents for transcription and translation. A binding site can bind to a binding moiety of a a target expressed in a cell-free system or in vitro. In some instances, a binding moiety of a target is expressed in a cell-free system or in vitro. For example, a binding moiety of a target is in a cell extract. In some instances, a binding moiety of a target is in a cell extract with a DNA template, and reagents for transcription and translation. Exemplary sources of cell extracts that can be used include wheat germ, Escherichia coli, rabbit reticulocyte, hyperthermophiles, hybridomas, Xenopus oocytes, insect cells, and mammalian cells (e.g., human cells). Exemplary cell-free methods that can be used to express target polypeptides (e.g., to produce target polypeptides on an array) include Protein in situ arrays (PISA), Multiple spotting technique (MIST), Self-assembled mRNA translation, Nucleic acid programmable protein array (NAPPA), nanowell NAPPA, DNA array to protein array (DAPA), membrane-free DAPA, nanowell copying and pIP-microintaglio printing, and pMAC-protein microarray copying (See Kilb et al., Eng. Life Sci. 2014, 14, 352-364).

In some instances, a binding site binds to a target that is synthesized in situ (e.g., on a solid substrate of an array) from a DNA template. A binding site can bind to binding moiety of a target that is synthesized in situ. In some instances, a binding moiety of a target is synthesized in situ (e.g., on a solid substrate of an array) from a DNA template. In some instances, a plurality of binding moieties is synthesized in situ from a plurality of corresponding DNA templates in parallel or in a single reaction. Exemplary methods for in situ target polypeptide expression include those described in Stevens, Structure 8(9): R177-R185 (2000); Katzen et al., Trends Biotechnol. 23(3):150-6. (2005); He et al., Curr. Opin. Biotechnol. 19(1):4-9. (2008); Ramachandran et al., Science 305(5680):86-90. (2004); He et al., Nucleic Acids Res. 29(15):E73-3 (2001); Angenendt et al., Mol. Cell Proteomics 5(9): 1658-66 (2006); Tao et al, Nat Biotechnol 24(10):1253-4 (2006); Angenendt et al., Anal. Chem. 76(7):1844-9 (2004); Kinpara et al., J. Biochem. 136(2):149-54 (2004); Takulapalli et al., J. Proteome Res. 11(8):4382-91 (2012); He et al., Nat. Methods 5(2):175-7 (2008); Chatterjee and J. LaBaer, Curr Opin Biotech 17(4):334-336 (2006); He and Wang, Biomol Eng 24(4):375-80 (2007); and He and Taussig, J. Immunol. Methods 274(1-2):265-70 (2003).

In some instances, a binding site binds to a nucleic acid target comprising a span of at least 6 nucleotides, for example, least 8, 9, 10, 12, 15, 20, 25, 30, 40, 50, or 100 nucleotides. In some instances, a binding site binds to a protein target comprising a contiguous stretch of nucleotides. In some instances, a binding site binds to a protein target comprising a non-contiguous stretch of nucleotides. In some instances, a binding site binds to a nucleic acid target comprising a site of a mutation or functional mutation, including a deletion, addition, swap, or truncation of the nucleotides in a nucleic acid sequence. A binding site can bind to a binding moiety of a nucleic acid target. In some instances, a binding moiety of a nucleic acid target comprises a span of at least 6 nucleotides, for example, least 8, 9, 10, 12, 15, 20, 25, 30, 40, 50, or 100 nucleotides. In some instances, a binding moiety of a protein target comprises a contiguous stretch of nucleotides. In some instances, a binding moiety of a protein target comprises a non-contiguous stretch of nucleotides. In some instances, a binding moiety of a nucleic acid target comprises a site of a mutation or functional mutation, including a deletion, addition, swap, or truncation of the nucleotides in a nucleic acid sequence.

In some instances, a binding site binds to a protein target comprising a span of at least 6 amino acids, for example, least 8, 9, 10, 12, 15, 20, 25, 30, 40, 50, or 100 amino acids. In some instances, a binding site binds to a protein target comprising a contiguous stretch of amino acids. In some instances, a binding site binds to a protein target comprising a non-contiguous stretch of amino acids. In some instances, a binding site binds to a protein target comprising a site of a mutation or functional mutation, including a deletion, addition, swap, or truncation of the amino acids in a polypeptide sequence. A binding site can bind to a binding moiety of a protein target. In some instances, a binding moiety of a protein target comprises a span of at least 6 amino acids, for example, least 8, 9, 10, 12, 15, 20, 25, 30, 40, 50, or 100 amino acids. In some instances, a binding moiety of a protein target comprises a contiguous stretch of amino acids. In some instances, a binding moiety of a protein target comprises a non-contiguous stretch of amino acids. In some instances, a binding moiety of a protein target comprises a site of a mutation or functional mutation, including a deletion, addition, swap, or truncation of the amino acids in a polypeptide sequence.

In some embodiments, a binding site binds to a domain, a fragment, an epitope, a region, or a portion of a membrane bound protein. A binding site can bind to a binding moiety of a membrane bound protein. In some embodiments, a binding moiety is on or comprises a domain, a fragment, an epitope, a region, or a portion of a membrane bound protein. Exemplary membrane bound proteins include, but are not limited to, GPCRs (e.g., adrenergic receptors, angiotensin receptors, cholecystokinin receptors, muscarinic acetylcholine receptors, neurotensin receptors, galanin receptors, dopamine receptors, opioid receptors, erotonin receptors, somatostatin receptors, etc.), ion channels (e.g., nicotinic acetylcholine receptors, sodium channels, potassium channels, etc.), non-excitable and excitable channels, receptor tyrosine kinases, receptor serine/threonine kinases, receptor guanylate cyclases, growth factor and hormone receptors (e.g., epidermal growth factor (EGF) receptor), and others. The binding site can bind to a domain, a fragment, an epitope, a region, or a portion of a mutant or modified variants of membrane-bound proteins. The binding site can bind to a binding moiety of a mutant or modified variant of membrane-bound protein. The binding moiety may also be on or comprise a domain, a fragment, an epitope, a region, or a portion of a mutant or modified variants of membrane-bound proteins. For example, some single or multiple point mutations of GPCRs retain function and are involved in disease (See, e.g., Stadel et al., (1997) Trends in Pharmacological Review 18:430-37).

A binding site binds to, for example, a domain, a fragment, an epitope, a region, or a portion of a ubiquitin ligase. A binding site binds to, for example, a domain, a fragment, an epitope, a region, or a portion of a ubiquitin adaptor, proteasome adaptor, or proteasome protein. A binding site binds to, for example, a domain, a fragment, an epitope, a region, or a portion of a protein involved in endocytosis, phagocytosis, a lysosomal pathway, an autophagic pathway, macroautophagy, microautophagy, chaperone-mediated autophagy, the multivesicular body pathway, or a combination thereof.

### RNA Binding Sites

In some embodiments, the circular polyribonucleotide comprises one or more RNA binding sites. In some embodiments, the circular polyribonucleotide includes RNA binding sites that modify expression of an endogenous gene and/or an exogenous gene. In some embodiments, the RNA binding site modulates expression of a host gene. The RNA binding site can include a sequence that hybridizes to an endogenous gene (e.g., a sequence for a miRNA, siRNA, mRNA, lncRNA, RNA, DNA, an antisense RNA, gRNA as described herein), a sequence that hybridizes to an exogenous nucleic acid such as a viral DNA or RNA, a sequence that hybridizes to an RNA, a sequence that interferes with gene transcription, a sequence that interferes with RNA translation, a sequence that stabilizes RNA or destabilizes RNA such as through targeting for degradation, or a sequence that modulates a DNA- or RNA-binding factor. In some embodiments, the circular polyribonucleotide comprises an aptamer sequence that binds to an RNA. The aptamer sequence can bind to an endogenous gene (e.g., a sequence for a miRNA, siRNA, mRNA, lncRNA, RNA, DNA, an antisense RNA, gRNA as described herein), to an exogenous nucleic acid such as a viral DNA or RNA, to an RNA, to a sequence that interferes with gene transcription, to a sequence that interferes with RNA translation, to a sequence that stabilizes RNA or destabilizes RNA such as through targeting for degradation, or to a sequence that modulates a DNA- or RNA-binding factor. The secondary structure of the aptamer sequence can bind to the RNA. The circular RNA can form a complex with the RNA by binding of the aptamer sequence to the RNA.

In some embodiments, the RNA binding site can be one of a tRNA, lncRNA, lincRNA, miRNA, rRNA, snRNA, microRNA, siRNA, piRNA, snoRNA, snRNA, exRNA, scaRNA, Y RNA, and hnRNA binding site. RNA binding sites are well-known to persons of ordinary skill in the art.

Certain RNA binding sites can inhibit gene expression through the biological process of RNA interference (RNAi). In some embodiments, the circular polyribonucleotides comprises an RNAi molecule with RNA or RNA-like structures typically having 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNA (siRNA), double-strand RNA (dsRNA), microRNA (miRNA), short hairpin RNA (shRNA), meroduplexes, and dicer substrates.

In some embodiments, the RNA binding site comprises an siRNA or an shRNA. siRNA and shRNA resemble intermediates in the processing pathway of the endogenous miRNA genes. In some embodiments, siRNA can function as miRNA and vice versa. MicroRNA, like siRNA, can use RISC to downregulate target genes, but unlike siRNA, most animal miRNA do not cleave the mRNA. Instead, miRNA reduce protein output through translational suppression or polyA removal and mRNA degradation. Known miRNA binding sites are within mRNA 3′-UTRs; miRNA seem to target sites with near-perfect complementarity to nucleotides 2-8 from the miRNA's 5′ end. This region is known as the seed region. Because siRNA and miRNA are interchangeable, exogenous siRNA can downregulate mRNA with seed complementarity to the siRNA. Multiple target sites within a 3′-UTR can give stronger downregulation.

MicroRNA (miRNA) are short noncoding RNA that bind to the 3′-UTR of nucleic acid molecules and down-regulate gene expression either by reducing nucleic acid molecule stability or by inhibiting translation. The circular polyribonucleotide can comprise one or more miRNA target sequences, miRNA sequences, or miRNA seeds. Such sequences can correspond to any miRNA.

A miRNA sequence comprises a “seed” region, i.e., a sequence in the region of positions 2-8 of the mature miRNA, which sequence has Watson-Crick complementarity to the miRNA target sequence. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA target is flanked by an adenine (A) opposed to miRNA position 1. In some embodiments, a miRNA seed can comprise 6 nucleotides (e.g., nucleotides 2-7 of the mature miRNA), wherein the seed-complementary site in the corresponding miRNA target is flanked by an adenine (A) opposed to miRNA at position 1.

The bases of the miRNA seed can be substantially complementary with the target sequence. By engineering miRNA target sequences into the circular polyribonucleotide, the circular polyribonucleotide can evade or be detected by the host's immune system, have modulated degradation, or modulated translation. This process can reduce the hazard of off target effects upon circular polyribonucleotide delivery.

The circular polyribonucleotide can include an miRNA sequence identical to about 5 to about 25 contiguous nucleotides of a target gene. In some embodiments, the miRNA sequence targets a mRNA and commences with the dinucleotide AA, comprises a GC-content of about 30%-70%, about 30%-60%, about 40%-60%, or about 45%-55%, and does not have a high percentage identity to any nucleotide sequence other than the target in the genome of the mammal in which it is to be introduced, for example, as determined by standard BLAST search.

Conversely, miRNA binding sites can be engineered out of (i.e., removed from) the circular polyribonucleotide to modulate protein expression in specific tissues. Regulation of expression in multiple tissues can be accomplished through introduction or removal or one or several miRNA binding sites (e.g., the miRNA binding site confers nucleic acid activity in a cell).

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-ld, miR-149), kidney (miR-192, miR-194, miR-204), and lung epithelial cells (let-7, miR-133, miR-126). MViRNA can also regulate complex biological processes, such as angiogenesis (miR-132). In the circular polyribonucleotides described herein, binding sites for miRNA that are involved in such processes can be removed or introduced, in order to tailor the expression from the circular polyribonucleotide to biologically relevant cell types or to the context of relevant biological processes. In some embodiments, the miRNA binding site includes, e.g., miR-7.

Through an understanding of the expression patterns of miRNA in different cell types, the circular polyribonucleotide described herein can be engineered for more targeted expression in specific cell types or only under specific biological conditions. Through introduction of tissue-specific miRNA binding sites, the circular polyribonucleotide can be designed for optimal protein expression in a tissue or in the context of a biological condition.

In addition, miRNA seed sites can be incorporated into the circular polyribonucleotide to modulate expression in certain cells which results in a biological improvement. An example of this is incorporation of miR-142 sites. Incorporation of miR-142 sites into the circular polyribonucleotide described herein can modulate expression in hematopoietic cells, but also reduce or abolish immune responses to a protein encoded in the circular polyribonucleotide.

In some embodiments, the circular polyribonucleotide comprises at least one miRNA, e.g., 2, 3, 4, 5, 6, or more. In some embodiments, the circular polyribonucleotide comprises an miRNA having at least about 75%, about 80%, about 85%, about 90%, about 95%, about 96%, about 97%, about 98%, about 99%, or 100% nucleotide sequence identity to any one of the nucleotide sequences or a sequence that is complementary to a target sequence.

Lists of known miRNA sequences can be found in databases maintained by research organizations, for example, Wellcome Trust Sanger Institute, Penn Center for Bioinformatics, Memorial Sloan Kettering Cancer Center, and European Molecule Biology Laboratory. RNAi molecules can be readily designed and produced by technologies known in the art. In addition, computational tools can be used to determine effective and specific sequence motifs.

In some embodiments, a circular polyribonucleotide comprises a long non-coding RNA. Long non-coding RNA (lncRNA) include non-protein coding transcripts longer than 100 nucleotides. The longer length distinguishes lncRNA from small regulatory RNA, such as miRNA, siRNA, and other short RNA. In general, the majority (˜78%) of lncRNA are characterized as tissue-specific. Divergent lncRNA that are transcribed in the opposite direction to nearby protein-coding genes (comprise a significant proportion ˜20% of total lncRNA in mammalian genomes) can regulate the transcription of the nearby gene.

The length of the RNA binding site may be between about 5 to 30 nucleotides, between about 10 to 30 nucleotides, or about 11, 12, 13, 14, 15, 16, 17, 18, 19, 20, 21, 22, 23, 24, 25, 26, 27, 28, 29, 30, or more nucleotides. The degree of identity of the RNA binding site to a target of interest can be at least 75%, at least 80%, at least 85%, at least 90%, or at least 95%.

In some embodiments, the circular polyribonucleotide includes one or more large intergenic non-coding RNA (lincRNA) binding sites. LincRNA make up most of the long non-coding RNA. LincRNA are non-coding transcripts and, in some embodiments, are more than about 200 nucleotides long. In some embodiments, lincRNA have an exon-intron-exon structure, similar to protein-coding genes, but do not encompass open-reading frames and do not code for proteins. LincRNA expression can be strikingly tissue-specific compared to coding genes. LincRNA are typically co-expressed with their neighboring genes to a similar extent to that of pairs of neighboring protein-coding genes. In some embodiments, the circular polyribonucleotide comprises a circularized lincRNA.

In some embodiments, the circular polyribonucleotides disclosed herein include one or more lincRNA, for example, FIRRE, LINC00969, PVT1, LINC01608, JPX, LINC01572, LINC00355, C1orf132, C3orf35, RP11-734, LINC01608, CC-499B15.5, CASC15, LINC00937, and RP11-191.

Lists of known lincRNA and lncRNA sequences can be found in databases maintained by research organizations, for example, Institute of Genomics and Integrative Biology, Diamantina Institute at the University of Queensland, Ghent University, and Sun Yat-sen University. LincRNA and lncRNA molecules can be readily designed and produced by technologies known in the art. In addition, computational tools can be used to determine effective and specific sequence motifs.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

The RNA binding site can comprise a sequence that is substantially complementary, or fully complementary, to all or a fragment of an endogenous gene or gene product (e.g., mRNA). The complementary sequence can complement sequences at the boundary between introns and exons to prevent the maturation of newly-generated nuclear RNA transcripts of specific genes into mRNA for transcription. The complementary sequence may be specific to genes by hybridizing with the mRNA for that gene and prevent its translation. The RNA binding site can comprise a sequence that is antisense or substantially antisense to all or a fragment of an endogenous gene or gene product, such as DNA, RNA, or a derivative or hybrid thereof.

The RNA binding site can comprise a sequence that is substantially complementary, or fully complementary, to a region of a linear polyribonucleotide. The complementary sequence may be specific to the region of the linear polyribonucleotide for hybridization of the circular polyribonucleotide to the linear polyribonucleotide. In some embodiments, the linear polyribonucleotide also comprises a region for binding to a protein, such as a receptor, on a cell. In some embodiments, the region of the linear polyribonucleotide that binds to a cell receptor results in internalization of the linear polyribonucleotide hybridized to the circular polyribonucleotide into the cell after binding.

In some embodiments, the circular polyribonucleotide comprises a RNA binding site that has an RNA or RNA-like structure typically between about 5-5000 base pairs (depending on the specific RNA structure, e.g., miRNA 5-30 bps, lncRNA 200-500 bps) and has a nucleobase sequence identical (complementary) or nearly identical (substantially complementary) to a coding sequence in an expressed target gene within the cell.

### DNA Binding Sites

In some embodiments, the circular polyribonucleotide comprises a DNA binding site, such as a sequence for a guide RNA (gRNA). In some embodiments, the circular polyribonucleotide comprises a guide RNA or a complement to a gRNA sequence. A gRNA short synthetic RNA composed of a “scaffold” sequence necessary for binding to the incomplete effector moiety and a user-defined ˜20 nucleotide targeting sequence for a genomic target. Guide RNA sequences can have a length of between 17-24 nucleotides (e.g., 19, 20, or 21 nucleotides) and complementary to the targeted nucleic acid sequence. Custom gRNA generators and algorithms can be used in the design of effective guide RNA. Gene editing can be achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNA can be effective in genome editing.

The gRNA can recognize specific DNA sequences (e.g., sequences adjacent to or within a promoter, enhancer, silencer, or repressor of a gene).

In some embodiments, the gRNA is part of a CRISPR system for gene editing. For gene editing, the circular polyribonucleotide can be designed to include one or multiple guide RNA sequences corresponding to a desired target DNA sequence. The gRNA sequences may include at least 10, 11, 12, 13, 14, 15, 16, 17, 18, 19, 20, 21, 22, 23, 24, 25, 26, 27, 28, 29, 30 or more nucleotides for interaction with Cas9 or other exonuclease to cleave DNA, e.g., Cpf1 interacts with at least about 16 nucleotides of gRNA sequence for detectable DNA cleavage.

In some embodiments, the circular polyribonucleotide comprises an aptamer sequence that can bind to DNA. The secondary structure of the aptamer sequence can bind to DNA. In some embodiments, the circular polyribonucleotide forms a complex with the DNA by binding of the aptamer sequence to the DNA.

In some embodiments, the circular polyribonucleotide includes sequences that bind a major groove of in duplex DNA. In one such instance, the specificity and stability of a triplex structure created by the circular polyribonucleotide and duplex DNA is afforded via Hoogsteen hydrogen bonds, which are different from those formed in classical Watson-Crick base pairing in duplex DNA. In one instance, the circular polyribonucleotide binds to the purine-rich strand of a target duplex through the major groove.

In some embodiments, triplex formation occurs in two motifs, distinguished by the orientation of the circular polyribonucleotide with respect to the purine-rich strand of the target duplex. In some instances, polypyrimidine sequence stretches in a circular polyribonucleotides bind to the polypurine sequence stretches of a duplex DNA via Hoogsteen hydrogen bonding in a parallel fashion (i.e., in the same 5′ to 3′, orientation as the purine-rich strand of the duplex), whereas the polypurine stretches (R) bind in an antiparallel fashion to the purine strand of the duplex via reverse-Hoogsteen hydrogen bonds. In the antiparallel, a purine motif comprises triplets of G:G-C, A:A-T, or T:A-T; whereas in the parallel, a pyrimidine motif comprises canonical triples of C+:G-C or T:A-T triplets (where C+ represents a protonated cytosine on the N3 position). Antiparallel GA and GT sequences in a circular polyribonucleotide may form stable triplexes at neutral pH, while parallel CT sequences in a circular polyribonucleotide may bind at acidic pH. N3 on cytosine in the circular polyribonucleotide may be protonated. Substitution of C with 5-methyl-C may permit binding of CT sequences in the circular polyribonucleotide at physiological pH as 5-methyl-C has a higher pK than does cytosine. For both purine and pyrimidine motifs, contiguous homopurine-homopyrimidine sequence stretches of at least 10 base pairs aid circular polyribonucleotide binding to duplex DNA, since shorter triplexes may be unstable under physiological conditions, and interruptions in sequences can destabilize the triplex structure. In some embodiments, the DNA duplex target for triplex formation includes consecutive purine bases in one strand. In some embodiments, a target for triplex formation comprises a homopurine sequence in one strand of the DNA duplex and a homopyrimidine sequence in the complementary strand.

In some embodiments, a triplex comprising a circular polyribonucleotide is a stable structure. In some embodiments, a triplex comprising a circular polyribonucleotide exhibits an increased half-life, e.g., increased by about 5%, 10%, 15%, 20%, 25%, 30%, 35%, 40%, 45%, 50%, or greater, e.g., persistence for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time there between.

### Protein Binding Sites

In some embodiments, the circular polyribonucleotide includes one or more protein binding sites. In some embodiments, a protein binding site comprises an aptamer sequence. In one embodiment, the circular polyribonucleotide includes a protein binding site to reduce an immune response from the host as compared to the response triggered by a reference compound, e.g., a circular polyribonucleotide lacking the protein binding site, e.g., linear RNA.

In some embodiments, circular polyribonucleotides disclosed herein include one or more protein binding sites to bind a protein, e.g., a ribosome. By engineering protein binding sites, e.g., ribosome binding sites, into the circular polyribonucleotide, the circular polyribonucleotide can evade or have reduced detection by the host's immune system, have modulated degradation, or modulated translation.

In some embodiments, the circular polyribonucleotide comprises at least one immunoprotein binding site, for example, to mask the circular polyribonucleotide from components of the host's immune system, e.g., evade CTL responses. In some embodiments, the immunoprotein binding site is a nucleotide sequence that binds to an immunoprotein and aids in masking the circular polyribonucleotide as non-endogenous.

Traditional mechanisms of ribosome engagement to linear RNA involve ribosome binding to the capped 5′ end of an RNA. From the 5′ end, the ribosome migrates to an initiation codon, whereupon the first peptide bond is formed. According to the present invention, internal initiation (i.e., cap-independent) or translation of the circular polyribonucleotide does not require a free end or a capped end. Rather, a ribosome binds to a non-capped internal site, whereby the ribosome begins polypeptide elongation at an initiation codon. In some embodiments, the circular polyribonucleotide includes one or more RNA sequences comprising a ribosome binding site, e.g., an initiation codon.

In some embodiments, circular polyribonucleotides disclosed herein comprise a protein binding sequence that binds to a protein. In some embodiments, the protein binding sequence targets or localizes a circular polyribonucleotide to a specific target. In some embodiments, the protein binding sequence specifically binds an arginine-rich region of a protein.

In some embodiments, circular polyribonucleotides disclosed herein include one or more protein binding sites that each bind a target protein, e.g., acting as a scaffold to bring two or more proteins in close proximity. In some embodiments, circular polynucleotides disclosed herein comprise two protein binding sites that each bind a target protein, thereby bringing the target proteins into close proximity. In some embodiments, circular polynucleotides disclosed herein comprise three protein binding sites that each bind a target protein, thereby bringing the three target proteins into close proximity. In some embodiments, circular polynucleotides disclosed herein comprise four protein binding sites that each bind a target protein, thereby bringing the four target proteins into close proximity. In some embodiments, circular polynucleotides disclosed herein comprise five or more protein binding sites that each bind a target protein, thereby bringing five or more target proteins into close proximity. In some embodiments, the target proteins are the same. In some embodiments, the target proteins are different. In some embodiments, bringing target proteins into close proximity promotes formation of a protein complex. For example, a circular polyribonucleotide of the disclosure can act as a scaffold to promote the formation of a complex comprising one, two, three, four, five, six, seven, eight, nine, or ten target proteins, or more. In some embodiments, bringing two or more target proteins into close proximity promotes interaction of the two or more target proteins. In some embodiments, bringing two or more target proteins into close proximity modulates, promotes, or inhibits of an enzymatic rection. In some embodiments, bringing two or more target proteins into close proximity modulates, promotes, or inhibits a signal transduction pathway.

In some embodiments, the protein binding site includes, but is not limited to, a binding site to the protein, such as ACIN1, AGO, APOBEC3F, APOBEC3G, ATXN2, AUH, BCCIP, CAPRIN1, CELF2, CPSF1, CPSF2, CPSF6, CPSF7, CSTF2, CSTF2T, CTCF, DDX21, DDX3, DDX3X, DDX42, DGCR8, EIF3A, EIF4A3, EIF4G2, ELAVL1, ELAVL3, FAM120A, FBL, FIP1L1, FKBP4, FMR1, FUS, FXR1, FXR2, GNL3, GTF2F1, HNRNPA1, HNRNPA2B1, HNRNPC, HNRNPK, HNRNPL, HNRNPM, HNRNPU, HNRNPUL1, IGF2BP1, IGF2BP2, IGF2BP3, ILF3, KHDRBS1, LARP7, LIN28A, LIN28B, m6A, MBNL2, METIL3, MOV10, MSI1, MSI2, NONO, NONO−, NOP58, NPM1, NUDT21, p53, PCBP2, POLR2A, PRPF8, PTBP1, RBFOX1, RBFOX2, RBFOX3, RBM10, RBM22, RBM27, RBM47, RNPS1, SAFB2, SBDS, SF3A3, SF3B4, SIRT7, SLBP, SLTM, SMNDC1, SND1, SRRM4, SRSF1, SRSF3, SRSF7, SRSF9, TAF15, TARDBP, TIA1, TNRC6A, TOP3B, TRA2A, TRA2B, U2AF1, U2AF2, UNK, UPF1, WDR33, XRN2, YBX1, YTHDC1, YTHDF1, YTHDF2, YWHAG, ZC3H7B, PDK1, AKT1, and any other protein that binds RNA.

In some embodiments, a protein binding site is a nucleic acid sequence that binds to a protein, e.g., a sequence that can bind a transcription factor, enhancer, repressor, polymerase, nuclease, histone, or any other protein that binds DNA. In some embodiments, a protein binding site is an aptamer sequence that binds to a protein. In some embodiments, the secondary structure of the aptamer sequence binds the protein. In some embodiments, the circular RNA forms a complex with the protein by binding of the aptamer sequence to the protein.

In some embodiments, a circular RNA is conjugated to a small molecule or a part thereof, wherein the small molecule or part thereof binds to a target such as a protein. A small molecule can be conjugated to a circular RNA via a modified nucleotide, e.g., by click chemistry. Examples of small molecules that can bind to proteins include, but are not limited to 4-hydroxytamoxifen (4-OHT), AC220, Afatinib, an aminopyrazole analog, an AR antagonist, BI-7273, Bosutinib, Ceritinib, Chloroalkane, Dasatinib, Foretinib, Gefitinib, a HIF-1α-derived (R)-hydroxyproline, HJB97, a hydroxyproline-based ligand, IACS-7e, Ibrutinib, an ibrutinib derivative, JQ1, Lapatinib, an LCL161 derivative, Lenalidomide, a nutlin small molecule, OTX015, a PDE4 inhibitor, Pomalidomide, a ripk2 inhibitor, RN486, Sirt2 inhibitor 3b, SNS-032, Steel factor, a TBK1 inhibitor, Thalidomide, a thalidomide derivative, a Thiazolidinedione-based ligand, a VH032 derivative, VHL ligand 2, VHL-1, VL-269, and derivatives thereof.

In some embodiments, a circular RNA is conjugated to more than one small molecule, for instance, 2, 3, 4, 5, 6, 7, 8, 9, 10, or more small molecules. In some embodiments, a circular RNA is conjugated to more than one different small molecules, for instance, 2, 3, 4, 5, 6, 7, 8, 9, 10, or more different small molecules. In some embodiments, the more than one small molecule conjugated to the circular RNA are configured to recruit their respective target proteins into proximity, which can lead to interaction between the target proteins, and/or other molecular and cellular changes. For instance, a circular RNA can be conjugated to both JQ1 and thalidomide, or derivative thereof, which can thus recruit a target protein of JQ1, e.g., BET family proteins, and a target protein of thalidomide, e.g., E3 ligase. In some cases, the circular RNA conjugated with JQ1 and thalidomide recruits a BET family protein via JQ1, or derivative thereof, tags the BET family protein with ubiquitin by E3 ligase that is recruited through thalidomide or derivative thereof, and thus leads to degradation of the tagged BET family protein.

### Other Binding Sites

In some embodiments, the circular polyribonucleotide comprises one or more binding sites to a non-RNA or non-DNA target. In some embodiments, the binding site can be one of a small molecule, an aptamer, a lipid, a carbohydrate, a virus particle, a membrane, a multi-component complex, a cell, a cellular moiety, or any fragment thereof binding site. In some embodiments, the circular polyribonucleotide comprises one or more binding sites to a lipid. In some embodiments, the circular polyribonucleotide comprises one or more binding sites to a carbohydrate. In some embodiments, the circular polyribonucleotide comprises one or more binding sites to a carbohydrate. In some embodiments, the circular polyribonucleotide comprises one or more binding sites to a membrane. In some embodiments, the circular polyribonucleotide comprises one or more binding sites to a multi-component complex, e.g., ribosome, nucleosome, transcription machinery, etc.

In some embodiments, the circular polyribonucleotide comprises an aptamer sequence. The aptamer sequence can bind to any target as described herein (e.g., a nucleic acid molecule, a small molecule, a protein, a carbohydrate, a lipid, etc.). The aptamer sequence has a secondary structure that can bind the target. In some embodiments, the aptamer sequence has a tertiary structure that can bind the target. In some embodiments, the aptamer sequence has a quaternary structure that can bind the target. The circular polyribonucleotide can bind to the target via the aptamer sequence to form a complex. In some embodiments, the complex is detectable for at least 5 days. In some embodiments, the complex is detectable for at least 2 days, 3, days, 4 days, 5 days, 6 days, 7 days, 8 days, 9 days, 10 days, 11 days, 12 days, 13 days, 14 days, 15 days, 16 days.

### Targets

The least one binding site can bind to a target. The at least one binding site can comprise at least one aptamer sequence that binds to a target. In some embodiments, the circRNA comprises one or more binding sites for one or more targets. Targets include, but are not limited to, nucleic acids (e.g., RNAs, DNAs, RNA-DNA hybrids), small molecules (e.g., drugs, fluorophores, metabolites), aptamers, polypeptides, proteins, lipids, carbohydrates, antibodies, viruses, virus particles, membranes, multi-component complexes, organelles, cells, other cellular moieties, any fragments thereof, and any combination thereof. (See, e.g., Fredriksson et al., (2002) Nat Biotech 20:473-77; Gullberg et al., (2004) PNAS, 101:8420-24). For example, a target is a single-stranded RNA, a double-stranded RNA, a single-stranded DNA, a double-stranded DNA, a DNA or RNA comprising one or more double stranded regions and one or more single stranded regions, an RNA-DNA hybrid, a small molecule, an aptamer, a polypeptide, a protein, a lipid, a carbohydrate, an antibody, an antibody fragment, a mixture of antibodies, a virus particle, a membrane, a multi-component complex, a cell, a cellular moiety, any fragment thereof, or any combination thereof.

In some embodiments, a target is a polypeptide, a protein, or any fragment thereof. For example, a target can be a purified polypeptide, an isolated polypeptide, a fusion tagged polypeptide, a polypeptide attached to or spanning the membrane of a cell or a virus or virion, a cytoplasmic protein, an intracellular protein, an extracellular protein, a kinase, a tyrosine kinase, a serine/threonine kinase, a phosphatase, an aromatase, a phosphodiesterase, a cyclase, a helicase, a protease, an oxidoreductase, a reductase, a transferase, a hydrolase, a lyase, an isomerase, a glycosylase, a extracellular matrix protein, a ligase, a ubiquitin ligase, any ligase that affects post-translational modification, an ion transporter, a channel, a pore, an apoptotic protein, a cell adhesion protein, a pathogenic protein, an aberrantly expressed protein, a transcription factor, a transcription regulator, a translation protein, an epigenetic factor, an epigenetic regulator, a chromatin regulator, a chaperone, a secreted protein, a ligand, a hormone, a cytokine, a chemokine, a nuclear protein, a receptor, a transmembrane receptor, a receptor tyrosine kinase, a G-protein coupled receptor, a growth factor receptor, a nuclear receptor, a hormone receptor, a signal transducer, an antibody, a membrane protein, an integral membrane protein, a peripheral membrane protein, a cell wall protein, a globular protein, a fibrous protein, a glycoprotein, a lipoprotein, a chromosomal protein, a proto-oncogene, an oncogene, a tumor-suppressor gene, any fragment thereof, or any combination thereof. In some embodiments, a target is a heterologous polypeptide. In some embodiments, a target is a protein overexpressed in a cell using molecular techniques, such as transfection. In some embodiments, a target is a recombinant polypeptide. For example, a target is in a sample produced from bacterial (e.g., E. coli), yeast, mammalian, or insect cells (e.g., proteins overexpressed by the organisms). In some embodiments, a target is a polypeptide with a mutation, insertion, deletion, or polymorphism. In some embodiments, a target is a polypeptide naturally expressed by a cell (e.g., a healthy cell or a cell associated with a disease or condition). In some embodiments, a target is an antigen, such as a polypeptide used to immunize an organism or to generate an immune response in an organism, such as for antibody production.

In some embodiments, a target is an antibody. An antibody can specifically bind to a particular spatial and polar organization of another molecule. An antibody can be monoclonal, polyclonal, or a recombinant antibody, and can be prepared by techniques that are well known in the art such as immunization of a host and collection of sera (polyclonal) or by preparing continuous hybrid cell lines and collecting the secreted protein (monoclonal), or by cloning and expressing nucleotide sequences, or mutagenized versions thereof, coding at least for the amino acid sequences required for specific binding of natural antibodies. A naturally occurring antibody can be a protein comprising at least two heavy (H) chains and two light (L) chains inter-connected by disulfide bonds. Each heavy chain can be comprised of a heavy chain variable region (V_{H}) and a heavy chain constant region. The heavy chain constant region can comprise three domains, C_{H1}, C_{H2}, and C_{H3}. Each light chain can comprise a light chain variable region (V_{L}) and a light chain constant region. The light chain constant region can comprise one domain, C_{L}. The V_{H} and V_{L} regions can be further subdivided into regions of hypervariability, termed complementary determining regions (CDR), interspersed with regions that are more conserved, termed framework regions (FR). Each V_{H} and V_{L} can be composed of three CDRs and four FRs arranged from amino-terminus to carboxy-terminus in the following order: FR₁, CDR₁, FR₂, CDR₂, FR₃, CDR₃, and FR4. The constant regions of the antibodies may mediate the binding of the immunoglobulin to host tissues or factors, including various cells of the immune system (e.g., effector cells) and the first component (C1 q) of the classical complement system. The antibodies can be of any isotype (e.g., IgG, IgE, IgM, IgD, IgA and IgY), class (e.g., IgG₁, IgG2, IgG₃, IgG₄, IgA₁ and IgA₂), subclass or modified version thereof. Antibodies may include a complete immunoglobulin or fragments thereof. An antibody fragment can refer to one or more fragments of an antibody that retain the ability to specifically bind to a binding moiety, such as an antigen. In addition, aggregates, polymers, and conjugates of immunoglobulins or their fragments are also included so long as binding affinity for a particular molecule is maintained. Examples of antibody fragments include a Fab fragment, a monovalent fragment consisting of the V_{L}, V_{H}, C_{L} and C_{H1} domains; a F(ab)₂ fragment, a bivalent fragment comprising two Fab fragments linked by a disulfide bridge at the hinge region; an Fd fragment consisting of the V_{H} and C_{H1} domains; an Fv fragment consisting of the V_{L} and V_{H} domains of a single arm of an antibody; a single domain antibody (dAb) fragment (Ward et al., (1989) Nature 341:544-46), which consists of a V_{H} domain; and an isolated CDR and a single chain Fragment (scFv) in which the V_{L} and V_{H} regions pair to form monovalent molecules (known as single chain Fv (scFv); See, e.g., Bird et al., (1988) Science 242:423-26; and Huston et al., (1988) PNAS 85:5879-83). Thus, antibody fragments include Fab, F(ab)₂, scFv, Fv, dAb, and the like. Although the two domains V_{L} and V_{H} are coded for by separate genes, they can be joined, using recombinant methods, by an artificial peptide linker that enables them to be made as a single protein chain. Such single chain antibodies include one or more antigen binding moieties. An antibody can be a polyvalent antibody, for example, bivalent, trivalent, tetravalent, pentavalent, hexavalanet, heptavalent, or octavalent antibodies. An antibody can be a multi-specific antibody. For example, bispecific, trispecific, tetraspecific, pentaspecific, hexaspecific, heptaspecific, or octaspecific antibodies can be generated, e.g., by recombinantly joining a combination of any two or more antigen binding agents (e.g., Fab, F(ab)₂, scFv, Fv, IgG). Multi-specific antibodies can be used to bring two or more targets into close proximitiy, e.g., degradation machinery and a target substrate to degrade, or a ubiquitin ligase and a substrate to ubiquitinate. These antibody fragments can be obtained using conventional techniques known to those of skill in the art, and the fragments can be screened for utility in the same manner as are intact antibodies. Antibodies can be human, humanized, chimeric, isolated, dog, cat, donkey, sheep, any plant, animal, or mammal.

In some embodiments, a target is a polymeric form of ribonucleotides and/or deoxyribonucleotides (adenine, guanine, thymine, or cytosine), such as DNA or RNA (e.g., mRNA). DNA includes double-stranded DNA found in linear DNA molecules (e.g., restriction fragments), viruses, plasmids, and chromosomes. In some embodiments, a polynucleotide target is single-stranded, double stranded, small interfering RNA (siRNA), messenger RNA (mRNA), transfer RNA (tRNA), a chromosome, a gene, a noncoding genomic sequence, genomic DNA (e.g., fragmented genomic DNA), a purified polynucleotide, an isolated polynucleotide, a hybridized polynucleotide, a transcription factor binding site, mitochondrial DNA, ribosomal RNA, a eukaryotic polynucleotide, a prokaryotic polynucleotide, a synthesized polynucleotide, a ligated polynucleotide, a recombinant polynucleotide, a polynucleotide containing a nucleic acid analogue, a methylated polynucleotide, a demethylated polynucleotide, any fragment thereof, or any combination thereof. In some embodiments, a target is a recombinant polynucleotide. In some embodiments, a target is a heterologous polynucleotide. For example, a target is a polynucleotide produced from bacterial (e.g., E. coli), yeast, mammalian, or insect cells (e.g., polynucleotides heterologous to the organisms). In some embodiments, a target is a polynucleotide with a mutation, insertion, deletion, or polymorphism.

In some embodiments, a target is an aptamer. An aptamer is an isolated nucleic acid molecule that binds with high specificity and affinity to a binding moiety or target molecule, such as a protein. An aptamer is a three dimensional structure held in certain conformation(s) that provides chemical contacts to specifically bind its given target. Although aptamers are nucleic acid based molecules, there is a fundamental difference between aptamers and other nucleic acid molecules such as genes and mRNA. In the latter, the nucleic acid structure encodes information through its linear base sequence and thus this sequence is of importance to the function of information storage. In complete contrast, aptamer function, which is based upon the specific binding of a target molecule, is not entirely dependent on a conserved linear base sequence (a non-coding sequence), but rather a particular secondary/tertiary/quaternary structure. Any coding potential that an aptamer may possess is fortuitous and is not thought to play a role in the binding of an aptamer to its cognate target. Aptamers are differentiated from naturally occurring nucleic acid sequences that bind to certain proteins. These latter sequences are naturally occurring sequences embedded within the genome of the organism that bind to a specialized sub-group of proteins that are involved in the transcription, translation, and transportation of naturally occurring nucleic acids (e.g., nucleic acid-binding proteins). Aptamers on the other hand non-naturally occurring nucleic acid molecules. While aptamers can be identified that bind nucleic acid-binding proteins, in most cases such aptamers have little or no sequence identity to the sequences recognized by the nucleic acid-binding proteins in nature. More importantly, aptamers can bind virtually any protein (not just nucleic acid-binding proteins) as well as almost any partner of interest including small molecules, carbohydrates, peptides, etc. For most partners, even proteins, a naturally occurring nucleic acid sequence to which it binds does not exist. For those partners that do have such a sequence, e.g., nucleic acid-binding proteins, such sequences will differ from aptamers as a result of the relatively low binding affinity used in nature as compared to tightly binding aptamers. Aptamers are capable of specifically binding to selected partners and modulating the partner's activity or binding interactions, e.g., through binding, aptamers may block their partner's ability to function. The functional property of specific binding to a partner is an inherent property an aptamer. An aptamer can be 6-35 kDa. An aptamer can be from 20 to 500 nucleotides. An aptamer can bind its partner with micromolar to sub-nanomolar affinity, and may discriminate against closely related targets (e.g., aptamers may selectively bind related proteins from the same gene family). In some cases, an aptamer only binds one molecule. In some cases, an aptamer binds family members of a molecule of interest. An aptamer, in some cases, binds to multiple different molecules. Aptamers are capable of using commonly seen intermolecular interactions such as hydrogen bonding, electrostatic complementarities, hydrophobic contacts, and steric exclusion to bind with a specific partner. Aptamers have a number of desirable characteristics for use as therapeutics and diagnostics including high specificity and affinity, low immunogenicity, biological efficacy, and excellent pharmacokinetic properties. An aptamer can comprise a molecular stem and loop structure formed from the hybridization of complementary polynucleotides that are covalently linked (e.g., a hairpin loop structure). The stem comprises the hybridized polynucleotides and the loop is the region that covalently links the two complementary polynucleotides. An aptamer can be a linear ribonucleic acid (e.g., linear aptamer) comprising an aptamer sequence or a circular polyribonucleic acid comprising an aptamer sequence (e.g., a circular aptamer).

In some embodiments, a target is a small molecule. For example, a small molecule can be a macrocyclic molecule, an inhibitor, a drug, or chemical compound. In some embodiments, a small molecule contains no more than five hydrogen bond donors. In some embodiments, a small molecule contains no more than ten hydrogen bond acceptors. In some embodiments, a small molecule has a molecular weight of 500 Daltons or less. In some embodiments, a small molecule has a molecular weight of from about 180 to 500 Daltons. In some embodiments, a small molecule contains an octanol-water partition coefficient lop P of no more than five. In some embodiments, a small molecule has a partition coefficient log P of from −0.4 to 5.6. In some embodiments, a small molecule has a molar refractivity of from 40 to 130. In some embodiments, a small molecule contains from about 20 to about 70 atoms. In some embodiments, a small molecule has a polar surface area of 140 Angstroms² or less.

In some embodiments, a circRNA comprises a binding site to a single target or a plurality of (e.g., two or more) targets. In one embodiment, the single circRNA comprises 2, 3, 4, 5, 6, 7, 8, 9, 10, or more different binding sites for a single target. In one embodiment, the single circRNA comprises 2, 3, 4, 5, 6, 7, 8, 9, 10, or more of the same binding sites for a single target. In one embodiment, the single circRNA comprises 2, 3, 4, 5, 6, 7, 8, 9, 10, or more different binding sites for one or more different targets. In one embodiment, two or more targets are in a sample, such as a mixture or library of targets, and the sample comprises circRNA comprising two or more binding sites that bind to the two or more targets.

In some embodiments, a single target or a plurality of (e.g., two or more) targets have a plurality of binding moieties. In one embodiment, the single target may have 2, 3, 4, 5, 6, 7, 8, 9, 10, or more binding moieties. In one embodiment, two or more targets are in a sample, such as a mixture or library of targets, and the sample comprises two or more binding moieties. In some embodiments, a single target or a plurality of targets comprise a plurality of different binding moieties. For example, a plurality may include at least about 2, 3, 4, 5, 6, 7, 8, 9, 10, 15, 20, 30, 40, 50, 60, 70, 80, 90, 100, 200, 500, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 25,000, or 30,000 binding moieties.

A target can comprise a plurality of binding moieties comprising at least 2 different binding moieties. For example, a binding moiety can comprise a plurality of binding moieties comprising at least 3, 4, 5, 6, 7, 8, 9, 10, 11, 12, 13, 14, 15, 16, 17, 18, 19, 20, 25, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, or 25,000 different binding moieties.

### Circular Polyribonucleotide Elements

In some embodiments, the circular polyribonucleotide comprises one or more of the elements as described herein in addition to comprising a sequence encoding a protein (e.g., a therapeutic protein) and/or at least one binding site. In some embodiments, the circular polyribonucleotide lacks a poly-A tail. In some embodiments, the circular polyribonucleotide lacks a replication element. In some embodiments, the circular polyribonucleotide lacks an IRES. In some embodiments, the circular polyribonucleotide lacks a cap. In some embodiments, the circular polyribonucleotide comprises any feature or any combination of features as disclosed in WO2019/118919, which is hereby incorporated by reference in its entirety.

For example, the circular polyribonucleotide comprises sequences encoding one or more polypeptides or peptides in addition to those disclosed above. Some examples include, but are not limited to, fluorescent tag or marker, antigen, peptide therapeutic, synthetic or analog peptide from naturally-bioactive peptide, agonist or antagonist peptide, anti-microbial peptide, pore-forming peptide, a bicyclic peptide, a targeting or cytotoxic peptide, a degradation or self-destruction peptide, and degradation or self-destruction peptides. In some embodiments, the circular polyribonucleotide further comprises an expression sequence encoding an additional therapeutic protein as described herein. Further examples of regulatory elements are described in paragraphs [0151]-[0153] of WO2019/118919, which is hereby incorporated by reference in its entirety.

For example, the circular polyribonucleotide comprises a regulatory element, e.g., a sequence that modifies expression of an expression sequence within the circular polyribonucleotide. A regulatory element may include a sequence that is located adjacent to an expression sequence that encodes an expression product. A regulatory element may be operably linked to the adjacent sequence. A regulatory element may increase an amount of product expressed as compared to an amount of the expressed product when no regulatory element is present. In addition, one regulatory element can increase an amount of products expressed for multiple expression sequences attached in tandem. Hence, one regulatory element can enhance the expression of one or more expression sequences. Multiple regulatory elements can also be used, for example, to differentially regulate expression of different expression sequences. In some embodiments, a regulatory element as provided herein can include a selective translation sequence. As used herein, the term “selective translation sequence” refers to a nucleic acid sequence that selectively initiates or activates translation of an expression sequence in the circular polyribonucleotide, for instance, certain riboswitch aptazymes. A regulatory element can also include a selective degradation sequence. As used herein, the term “selective degradation sequence” refers to a nucleic acid sequence that initiates degradation of the circular polyribonucleotide, or an expression product of the circular polyribonucleotide. In some embodiments, the regulatory element is a translation modulator. A translation modulator can modulate translation of the expression sequence in the circular polyribonucleotide. A translation modulator can be a translation enhancer or suppressor. In some embodiments, a translation initiation sequence can function as a regulatory element. Further examples of regulatory elements are described in paragraphs [0154]-[0161] of WO2019/118919, which is hereby incorporated by reference in its entirety.

In some embodiments, the circular polyribonucleotide comprises a sequence encoding a protein (e.g., a therapeutic protein) and/or at least one binding site, and comprises a translation initiation sequence, e.g., a start codon. In some embodiments, the translation initiation sequence includes a Kozak or Shine-Dalgarno sequence. In some embodiments, the circular polyribonucleotide includes the translation initiation sequence, e.g., Kozak sequence, adjacent to an expression sequence. In some embodiments, the translation initiation sequence is a non-coding start codon. In some embodiments, the translation initiation sequence, e.g., Kozak sequence, is present on one or both sides of each expression sequence, leading to separation of the expression products. In some embodiments, the circular polyribonucleotide includes at least one translation initiation sequence adjacent to an expression sequence. In some embodiments, the translation initiation sequence provides conformational flexibility to the circular polyribonucleotide. In some embodiments, the translation initiation sequence is within a substantially single stranded region of the circular polyribonucleotide. Further examples of translation initiation sequences are described in paragraphs [0163]-[0165] of WO2019/118919, which is hereby incorporated by reference in its entirety.

In some embodiments, a circular polyribonucleotide described herein comprises an internal ribosome entry site (IRES) element. A suitable IRES element to include in a circular polyribonucleotide can be an RNA sequence capable of engaging an eukaryotic ribosome. Further examples of an IRES are described in paragraphs [0166]-[0168] of WO2019/118919, which is hereby incorporated by reference in its entirety.

A circular polyribonucleotide can include one or more expression sequences (e.g., a therapeutic protein), and each expression sequence may or may not have a termination element. Further examples of termination elements are described in paragraphs [0169]-[0170] of WO2019/118919, which is hereby incorporated by reference in its entirety.

A circular polyribonucleotide of the disclosure can comprise a stagger element. The term “stagger element” refers to a moiety, such as a nucleotide sequence, that induces ribosomal pausing during translation. In some embodiments, the stagger element is a non-conserved sequence of amino-acids with a strong alpha-helical propensity followed by the consensus sequence −D(V/I)ExNPGP, where x=any amino acid. In some embodiments, the stagger element may include a chemical moiety, such as glycerol, a non nucleic acid linking moiety, a chemical modification, a modified nucleic acid, or any combination thereof.

In some embodiments, the circular polyribonucleotide includes at least one stagger element adjacent to an expression sequence. In some embodiments, the circular polyribonucleotide includes a stagger element adjacent to each expression sequence. In some embodiments, the stagger element is present on one or both sides of each expression sequence, leading to separation of the expression products, e.g., peptide(s) and/or polypeptide(s). In some embodiments, the stagger element is a portion of the one or more expression sequences. In some embodiments, the circular polyribonucleotide comprises one or more expression sequences, and each of the one or more expression sequences is separated from a succeeding expression sequence by a stagger element on the circular polyribonucleotide. In some embodiments, the stagger element prevents generation of a single polypeptide (a) from two rounds of translation of a single expression sequence or (b) from one or more rounds of translation of two or more expression sequences. In some embodiments, the stagger element is a sequence separate from the one or more expression sequences. In some embodiments, the stagger element comprises a portion of an expression sequence of the one or more expression sequences.

Examples of stagger elements are described in paragraphs [0172]-[0175] of WO2019/118919, which is hereby incorporated by reference in its entirety.

In some embodiments, the circular polyribonucleotide comprises one or more regulatory nucleic acid sequences or comprises one or more expression sequences that encode regulatory nucleic acid, e.g., a nucleic acid that modifies expression of an endogenous gene and/or an exogenous gene. In some embodiments, the expression sequence of a circular polyribonucleotide as provided herein can comprise a sequence that is antisense to a regulatory nucleic acid like a non-coding RNA, such as, but not limited to, tRNA, lncRNA, miRNA, rRNA, snRNA, microRNA, siRNA, piRNA, snoRNA, snRNA, exRNA, scaRNA, Y RNA, and hnRNA.

Exemplary regulatory nucleic acids are described in paragraphs [0177]-[0194] of WO2019/118919, which is hereby incorporated by reference in its entirety.

In some embodiments, the translation efficiency of a circular polyribonucleotide as provided herein is greater than a reference, e.g., a linear counterpart, a linear expression sequence, or a linear circular polyribonucleotide. In some embodiments, a circular polyribonucleotide as provided herein has the translation efficiency that is at least about 5%, 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 450%, 500%, 600%, 70%, 800%, 900%, 1000%, 2000%, 5000%, 10000%, 100000%, or more greater than that of a reference. In some embodiments, a circular polyribonucleotide has a translation efficiency 10% greater than that of a linear counterpart. In some embodiments, a circular polyribonucleotide has a translation efficiency 300% greater than that of a linear counterpart.

In some embodiments, the circular polyribonucleotide produces stoichiometric ratios of expression products. Rolling circle translation continuously produces expression products at substantially equivalent ratios. In some embodiments, the circular polyribonucleotide has a stoichiometric translation efficiency, such that expression products are produced at substantially equivalent ratios. In some embodiments, the circular polyribonucleotide has a stoichiometric translation efficiency of multiple expression products, e.g., products from 2, 3, 4, 5, 6, 7, 8, 9, 10, 11, 12, or more expression sequences.

In some embodiments, once translation of the circular polyribonucleotide is initiated, the ribosome bound to the circular polyribonucleotide does not disengage from the circular polyribonucleotide before finishing at least one round of translation of the circular polyribonucleotide. In some embodiments, the circular polyribonucleotide as described herein is competent for rolling circle translation. In some embodiments, during rolling circle translation, once translation of the circular polyribonucleotide is initiated, the ribosome bound to the circular polyribonucleotide does not disengage from the circular polyribonucleotide before finishing at least 2 rounds, at least 3 rounds, at least 4 rounds, at least 5 rounds, at least 6 rounds, at least 7 rounds, at least 8 rounds, at least 9 rounds, at least 10 rounds, at least 11 rounds, at least 12 rounds, at least 13 rounds, at least 14 rounds, at least 15 rounds, at least 20 rounds, at least 30 rounds, at least 40 rounds, at least 50 rounds, at least 60 rounds, at least 70 rounds, at least 80 rounds, at least 90 rounds, at least 100 rounds, at least 150 rounds, at least 200 rounds, at least 250 rounds, at least 500 rounds, at least 1000 rounds, at least 1500 rounds, at least 2000 rounds, at least 5000 rounds, at least 10000 rounds, at least 105 rounds, or at least 106 rounds of translation of the circular polyribonucleotide.

In some embodiments, the rolling circle translation of the circular polyribonucleotide leads to generation of polypeptide product that is translated from more than one round of translation of the circular polyribonucleotide (“continuous” expression product). In some embodiments, the circular polyribonucleotide comprises a stagger element, and rolling circle translation of the circular polyribonucleotide leads to generation of polypeptide product that is generated from a single round of translation or less than a single round of translation of the circular polyribonucleotide (“discrete” expression product). In some embodiments, the circular polyribonucleotide is configured such that at least 10%, 20%, 30%, 40%, 50%, at least 60%, at least 70%, at least 80%, at least 90%, at least 95%, at least 96%, at least 97%, at least 98%, at least 99%, or 100% of total polypeptides (molar/molar) generated during the rolling circle translation of the circular polyribonucleotide are discrete polypeptides. In some embodiments, the amount ratio of the discrete products over the total polypeptides is tested in an in vitro translation system. In some embodiments, the in vitro translation system used for the test of amount ratio comprises rabbit reticulocyte lysate. In some embodiments, the amount ratio is tested in an in vivo translation system, such as a eukaryotic cell or a prokaryotic cell, a cultured cell or a cell in an organism.

In some embodiments, the circular polyribonucleotide comprises untranslated regions (UTRs). UTRs of a genomic region comprising a gene may be transcribed but not translated. In some embodiments, a UTR may be included upstream of the translation initiation sequence of an expression sequence described herein. In some embodiments, a UTR may be included downstream of an expression sequence described herein. In some instances, one UTR for first expression sequence is the same as or continuous with or overlapping with another UTR for a second expression sequence. In some embodiments, the intron is a human intron. In some embodiments, the intron is a full-length human intron, e.g., ZKSCAN1.

Exemplary untranslated regions are described in paragraphs [0197]-[201] of WO2019/118919, which is hereby incorporated by reference in its entirety.

In some embodiments, the circular polyribonucleotide may include a poly-A sequence. Exemplary poly-A sequences are described in paragraphs [0202]-[0205] of WO2019/118919, which is hereby incorporated by reference in its entirety. In some embodiments, the circular polyribonucleotide lacks a poly-A sequence.

In some embodiments, the circular polyribonucleotide comprises one or more riboswitches. Exemplary riboswitches are described in paragraphs [0232]-[0252] of WO2019/118919, which is hereby incorporated by reference in its entirety.

In some embodiments, the circular polyribonucleotide comprises an aptazyme. Exemplary aptazymes are described in paragraphs [0253]-[0259] of WO2019/118919, which is hereby incorporated by reference in its entirety.

In some embodiments, the circular polyribonucleotide comprises one or more RNA binding sites. microRNAs (or miRNA) can be short noncoding RNAs that bind to the 3′UTR of nucleic acid molecules and down-regulate gene expression either by reducing nucleic acid molecule stability or by inhibiting translation. The circular polyribonucleotide may comprise one or more microRNA target sequences, microRNA sequences, or microRNA seeds. Such sequences may correspond to any known microRNA, such as those taught in US Publication US2005/0261218 and US Publication US2005/0059005, the contents of which are incorporated herein by reference in their entirety. Further examples of RNA binding sites are described in paragraphs [0206]-[0215] of WO2019/118919, which is hereby incorporated by reference in its entirety.

In some embodiments, the circular polyribonucleotide includes one or more protein binding sites that enable a protein, e.g., a ribosome, to bind to an internal site in the RNA sequence. Further examples of protein binding sites are described in paragraphs [0218]-[0221] of WO2019/118919, which is hereby incorporated by reference in its entirety.

In some embodiments, the circular polyribonucleotide comprises an encryptogen to reduce, evade or avoid the innate immune response of a cell. In one aspect, provided herein are circular polyribonucleotide which when delivered to cells (e.g., contacting), results in a reduced immune response from the host as compared to the response triggered by a reference compound, e.g. a linear polynucleotide corresponding to the described circular polyribonucleotide or a circular polyribonucleotide lacking an encryptogen. In some embodiments, the circular polyribonucleotide has less immunogenicity than a counterpart lacking an encryptogen.

In some embodiments, an encryptogen enhances stability. There is growing body of evidence about the regulatory roles played by the UTRs in terms of stability of a nucleic acid molecule and translation. The regulatory features of a UTR may be included in the encryptogen to enhance the stability of the circular polyribonucleotide.

In some embodiments, 5′ or 3′UTRs can constitute encryptogens in a circular polyribonucleotide. For example, removal or modification of UTR AU rich elements (AREs) may be useful to modulate the stability or immunogenicity of the circular polyribonucleotide.

In some embodiments, removal of modification of AU rich elements (AREs) in expression sequence, e.g., translatable regions, can be useful to modulate the stability or immunogenicity of the circular polyribonucleotide

In some embodiments, an encryptogen comprises miRNA binding site or binding site to any other non-coding RNAs. For example, incorporation of miR-142 sites into the circular polyribonucleotide described herein may not only modulate expression in hematopoietic cells, but also reduce or abolish immune responses to a protein encoded in the circular polyribonucleotide.

In some embodiments, an encyptogen comprises one or more protein binding sites that enable a protein, e.g., an immunoprotein, to bind to the RNA sequence. By engineering protein binding sites into the circular polyribonucleotide, the circular polyribonucleotide may evade or have reduced detection by the host's immune system, have modulated degradation, or modulated translation, by masking the circular polyribonucleotide from components of the host's immune system. In some embodiments, the circular polyribonucleotide comprises at least one immunoprotein binding site, for example to evade immune responses, e.g., CTL responses. In some embodiments, the immunoprotein binding site is a nucleotide sequence that binds to an immunoprotein and aids in masking the circular polyribonucleotide as exogenous.

In some embodiments, an encryptogen comprises one or more modified nucleotides. Exemplary modifications can include any modification to the sugar, the nucleobase, the internucleoside linkage (e.g. to a linking phosphate/to a phosphodiester linkage/to the phosphodiester backbone), and any combination thereof that can prevent or reduce immune response against the circular polyribonucleotide. Some of the exemplary modifications provided herein are described in details below.

In some embodiments, the circular polyribonucleotide includes one or more modifications as described elsewhere herein to reduce an immune response from the host as compared to the response triggered by a reference compound, e.g. a circular polyribonucleotide lacking the modifications. In particular, the addition of one or more inosine has been shown to discriminate RNA as endogenous versus viral. See for example, Yu, Z. et al. (2015) RNA editing by ADAR1 marks dsRNA as “self”. Cell Res. 25, 1283-1284, which is incorporated by reference in its entirety.

In some embodiments, the circular polyribonucleotide includes one or more expression sequences for shRNA or an RNA sequence that can be processed into siRNA, and the shRNA or siRNA targets RIG-I and reduces expression of RIG-I. RIG-I can sense foreign circular RNA and leads to degradation of foreign circular RNA. Therefore, a circular polynucleotide harboring sequences for RIG-I-targeting shRNA, siRNA or any other regulatory nucleic acids can reduce immunity, e.g., host cell immunity, against the circular polyribonucleotide.

In some embodiments, the circular polyribonucleotide lacks a sequence, element or structure, that aids the circular polyribonucleotide in reducing, evading or avoiding an innate immune response of a cell. In some such embodiments, the circular polyribonucleotide may lack a polyA sequence, a 5′ end, a 3′ end, phosphate group, hydroxyl group, or any combination thereof.

In some embodiments, the circular polyribonucleotide comprises a spacer sequence. In some embodiments, elements of a polyribonucleotide may be separated from one another by a spacer sequence or linker. Exemplary of spacer sequences are described in paragraphs [0293]-[0302] of WO2019/118919, which is hereby incorporated by reference in its entirety.

The circular polyribonucleotide described herein may also comprise a non-nucleic acid linker. Exemplary non-nucleic acid linkers are described in paragraphs [0303]-[0307] of WO2019/118919, which is hereby incorporated by reference in its entirety.

In some embodiments, the circular polyribonucleotide further includes another nucleic acid sequence. In some embodiments, the circular polyribonucleotide may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In some embodiments, the circular polyribonucleotide includes an siRNA to target a different locus of the same gene expression product as the circular polyribonucleotide. In some embodiments, the circular polyribonucleotide includes an siRNA to target a different gene expression product than a gene expression product that is present in the circular polyribonucleotide.

In some embodiments, the circular polyribonucleotide lacks a 5′-UTR. In some embodiments, the circular polyribonucleotide lacks a 3′-UTR. In some embodiments, the circular polyribonucleotide lacks a poly-A sequence. In some embodiments, the circular polyribonucleotide lacks a termination element. In some embodiments, the circular polyribonucleotide lacks an internal ribosomal entry site. In some embodiments, the circular polyribonucleotide lacks degradation susceptibility by exonucleases. In some embodiments, the fact that the circular polyribonucleotide lacks degradation susceptibility can mean that the circular polyribonucleotide is not degraded by an exonuclease, or only degraded in the presence of an exonuclease to a limited extent, e.g., that is comparable to or similar to in the absence of exonuclease. In some embodiments, the circular polyribonucleotide is not degraded by exonucleases. In some embodiments, the circular polyribonucleotide has reduced degradation when exposed to exonuclease. In some embodiments, the circular polyribonucleotide lacks binding to a cap-binding protein In some embodiments, the circular polyribonucleotide lacks a 5′ cap.

In some embodiments, the circular polyribonucleotide lacks a 5′-UTR and is competent for protein expression from its one or more expression sequences. In some embodiments, the circular polyribonucleotide lacks a 3′-UTR and is competent for protein expression from its one or more expression sequences. In some embodiments, the circular polyribonucleotide lacks a poly-A sequence and is competent for protein expression from its one or more expression sequences. In some embodiments, the circular polyribonucleotide lacks a termination element and is competent for protein expression from its one or more expression sequences. In some embodiments, the circular polyribonucleotide lacks an internal ribosomal entry site and is competent for protein expression from its one or more expression sequences. In some embodiments, the circular polyribonucleotide lacks a cap and is competent for protein expression from its one or more expression sequences. In some embodiments, the circular polyribonucleotide lacks a 5′-UTR, a 3′-UTR, and an IRES, and is competent for protein expression from its one or more expression sequences. In some embodiments, the circular polyribonucleotide comprises one or more of the following sequences: a sequence that encodes one or more miRNAs, a sequence that encodes one or more replication proteins, a sequence that encodes an exogenous gene, a sequence that encodes a therapeutic, a regulatory element (e.g., translation modulator, e.g., translation enhancer or suppressor), a translation initiation sequence, one or more regulatory nucleic acids that targets endogenous genes (e.g., siRNA, lncRNAs, shRNA), and a sequence that encodes a therapeutic mRNA or protein.

As a result of its circularization, the circular polyribonucleotide may include certain characteristics that distinguish it from linear RNA. For example, the circular polyribonucleotide is less susceptible to degradation by exonuclease as compared to linear RNA. As such, the circular polyribonucleotide can be more stable than a linear RNA, especially when incubated in the presence of an exonuclease. The increased stability of the circular polyribonucleotide compared with linear RNA can make the circular polyribonucleotide more useful as a cell transforming reagent to produce polypeptides (e.g., antigens and/or epitopes to elicit antibody responses). The increased stability of the circular polyribonucleotide compared with linear RNA can make the circular polyribonucleotide easier to store for long than linear RNA. The stability of the circular polyribonucleotide treated with exonuclease can be tested using methods standard in art which determine whether RNA degradation has occurred (e.g., by gel electrophoresis).

Moreover, unlike linear RNA, the circular polyribonucleotide can be less susceptible to dephosphorylation when the circular polyribonucleotide is incubated with phosphatase, such as calf intestine phosphatase.

In some embodiments, the circular polyribonucleotide comprises particular sequence characteristics. For example, the circular polyribonucleotide may comprise a particular nucleotide composition. In some such embodiments, the circular polyribonucleotide may include one or more purine (adenine and/or guanosine) rich regions. In some such embodiments, the circular polyribonucleotide may include one or more purine poor regions. In some embodiments, the circular polyribonucleotide may include one or more AU rich regions or elements (AREs). In some embodiments, the circular polyribonucleotide may include one or more adenine rich regions.

In some embodiments, the circular polyribonucleotide may include one or more repetitive elements described elsewhere herein. In some embodiments, the circular polyribonucleotide comprises one or more modifications described elsewhere herein.

A circular polyribonucleotide may include one or more substitutions, insertions and/or additions, deletions, and covalent modifications with respect to reference sequences. For example, circular polyribonucleotides with one or more insertions, additions, deletions, and/or covalent modifications relative to a parent polyribonucleotide are included within the scope of this disclosure. Exemplary modifications are described in paragraphs [0310]-[0325] of WO2019/118919, which is hereby incorporated by reference in its entirety.

In some embodiments, the circular polyribonucleotide comprises a higher order structure, e.g., a secondary or tertiary structure. In some embodiments, complementary segments of the circular polyribonucleotide fold itself into a double stranded segment, held together with hydrogen bonds between pairs, e.g., A-U and C-G. In some embodiments, helices, also known as stems, are formed intra-molecularly, having a double-stranded segment connected to an end loop. In some embodiments, the circular polyribonucleotide has at least one segment with a quasi-double-stranded secondary structure.

In some embodiments, one or more sequences of the circular polyribonucleotide include substantially single stranded vs double stranded regions. In some embodiments, the ratio of single stranded to double stranded may influence the functionality of the circular polyribonucleotide.

In some embodiments, one or more sequences of the circular polyribonucleotide that are substantially single stranded. In some embodiments, one or more sequences of the circular polyribonucleotide that are substantially single stranded may include a protein- or RNA-binding site. In some embodiments, the circular polyribonucleotide sequences that are substantially single stranded may be conformationally flexible to allow for increased interactions. In some embodiments, the sequence of the circular polyribonucleotide is purposefully engineered to include such secondary structures to bind or increase protein or nucleic acid binding.

In some embodiments, the circular polyribonucleotide sequences that are substantially double stranded. In some embodiments, one or more sequences of the circular polyribonucleotide that are substantially double stranded may include a conformational recognition site, e.g., a riboswitch or aptazyme. In some embodiments, the circular polyribonucleotide sequences that are substantially double stranded may be conformationally rigid. In some such instances, the conformationally rigid sequence may sterically hinder the circular polyribonucleotide from binding a protein or a nucleic acid. In some embodiments, the sequence of the circular polyribonucleotide is purposefully engineered to include such secondary structures to avoid or reduce protein or nucleic acid binding.

There are 16 possible base-pairings, however of these, six (AU, GU, GC, UA, UG, CG) may form actual base-pairs. The rest are called mismatches and occur at very low frequencies in helices. In some embodiments, the structure of the circular polyribonucleotide cannot easily be disrupted without impact on its function and lethal consequences, which provide a selection to maintain the secondary structure. In some embodiments, the primary structure of the stems (i.e., their nucleotide sequence) can still vary, while still maintaining helical regions. The nature of the bases is secondary to the higher structure, and substitutions are possible as long as they preserve the secondary structure. In some embodiments, the circular polyribonucleotide has a quasi-helical structure. In some embodiments, the circular polyribonucleotide has at least one segment with a quasi-helical structure. In some embodiments, the circular polyribonucleotide includes at least one of a U-rich or A-rich sequence or a combination thereof. In some embodiments, the U-rich and/or A-rich sequences are arranged in a manner that would produce a triple quasi-helix structure. In some embodiments, the circular polyribonucleotide has a double quasi-helical structure. In some embodiments, the circular polyribonucleotide has one or more segments (e.g., 2, 3, 4, 5, 6, or more) having a double quasi-helical structure. In some embodiments, the circular polyribonucleotide includes at least one of a C-rich and/or G-rich sequence. In some embodiments, the C-rich and/or G-rich sequences are arranged in a manner that would produce triple quasi-helix structure. In some embodiments, the circular polyribonucleotide has an intramolecular triple quasi-helix structure that aids in stabilization.

In some embodiments, the circular polyribonucleotide has two quasi-helical structure (e.g., separated by a phosphodiester linkage), such that their terminal base pairs stack, and the quasi-helical structures become colinear, resulting in a “coaxially stacked” substructure.

In some embodiments, the circular polyribonucleotide comprises a tertiary structure with one or more motifs, e.g., a pseudoknot, a g-quadruplex, a helix, and coaxial stacking.

Further examples of structure of circular polyribonucleotides as disclosed herein are described in paragraphs [0326]-[0333] of WO2019/118919, which is hereby incorporated by reference in its entirety.

In some embodiments, the circular polyribonucleotide is at least about 20 nucleotides, at least about 30 nucleotides, at least about 40 nucleotides, at least about 50 nucleotides, at least about 75 nucleotides, at least about 100 nucleotides, at least about 200 nucleotides, at least about 300 nucleotides, at least about 400 nucleotides, at least about 500 nucleotides, at least about 1,000 nucleotides, at least about 2,000 nucleotides, at least about 5,000 nucleotides, at least about 6,000 nucleotides, at least about 7,000 nucleotides, at least about 8,000 nucleotides, at least about 9,000 nucleotides, at least about 10,000 nucleotides, at least about 12,000 nucleotides, at least about 14,000 nucleotides, at least about 15,000 nucleotides, at least about 16,000 nucleotides, at least about 17,000 nucleotides, at least about 18,000 nucleotides, at least about 19,000 nucleotides, or at least about 20,000 nucleotides. In some embodiments, the circular polyribonucleotide may be of a sufficient size to accommodate a binding site for a ribosome. One of skill in the art can appreciate that the maximum size of a circular polyribonucleotide can be as large as is within the technical constraints of producing a circular polyribonucleotide, and/or using the circular polyribonucleotide. While not being bound by theory, it is possible that multiple segments of RNA may be produced from DNA and their 5′ and 3′ free ends annealed to produce a “string” of RNA, which ultimately may be circularized when only one 5′ and one 3′ free end remains. In some embodiments, the maximum size of a circular polyribonucleotide may be limited by the ability of packaging and delivering the RNA to a target. In some embodiments, the size of a circular polyribonucleotide is a length sufficient to encode useful polypeptides, and thus, lengths of at least 20,000 nucleotides, at least 15,000 nucleotides, at least 10,000 nucleotides, at least 7,500 nucleotides, or at least 5,000 nucleotides, at least 4,000 nucleotides, at least 3,000 nucleotides, at least 2,000 nucleotides, at least 1,000 nucleotides, at least 500 nucleotides, at least 400 nucleotides, at least 300 nucleotides, at least 200 nucleotides, at least 100 nucleotides may be useful.

In some embodiments, the circular polyribonucleotide is capable of replicating or replicates in a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, cultured cells, primary cells or cell lines, stem cells, progenitor cells, differentiated cells, germ cells, cancer cells (e.g., tumorigenic, metastic), non-tumorigenic cells (normal cells), fetal cells, embryonic cells, adult cells, mitotic cells, non-mitotic cells, or any combination thereof. In some embodiments, the invention includes a cell comprising the circular polyribonucleotide described herein, wherein the cell is a cell from an aquaculture animal (fish, crabs, shrimp, oysters etc.), a mammalian cell, e.g., a cell from a pet or zoo animal (cats, dogs, lizards, birds, lions, tigers and bears etc.), a cell from a farm or working animal (horses, cows, pigs, chickens etc.), a human cell, a cultured cell, a primary cell or a cell line, a stem cell, a progenitor cell, a differentiated cell, a germ cell, a cancer cell (e.g., tumorigenic, metastic), a non-tumorigenic cell (normal cells), a fetal cell, an embryonic cell, an adult cell, a mitotic cell, a non-mitotic cell, or any combination thereof.

### Stability and Half Life

In some embodiments, a circular polyribonucleotide provided herein has increased half-life over a reference, e.g., a linear polyribonucleotide having the same nucleotide sequence that is not circularized (linear counterpart). In some embodiments, the circular polyribonucleotide is substantially resistant to degradation, e.g., exonuclease degradation. In some embodiments, the circular polyribonucleotide is resistant to self-degradation. In some embodiments, the circular polyribonucleotide lacks an enzymatic cleavage site, e.g., a dicer cleavage site. Further examples of stability and half life of circular polyribonucleotides as disclosed herein are described in paragraphs [0308]-[0309] of WO2019/118919, which is hereby incorporated by reference in its entirety.

In some embodiments, the circular polyribonucleotide has a half-life of at least that of a linear counterpart, e.g., linear expression sequence, or linear circular polyribonucleotide. In some embodiments, the circular polyribonucleotide has a half-life that is increased over that of a linear counterpart. In some embodiments, the half-life is increased by about 5%, 10%, 15%, 20%, 25%, 30%, 35%, 40%, 45%, 50%, or greater. In some embodiments, the circular polyribonucleotide has a half-life or persistence in a cell for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the circular polyribonucleotide has a half-life or persistence in a cell for no more than about 10 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween. In some embodiments, the circular polyribonucleotide has a half-life or persistence in a cell while the cell is dividing. In some embodiments, the circular polyribonucleotide has a half-life or persistence in a cell post division. In certain embodiments, the circular polyribonucleotide has a half-life or persistence in a dividing cell for greater than about about 10 minutes to about 30 days, or at least about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween.

In some embodiments, at least about 10%, 20%, 30%, 40%, 50%, 60%, 70%, 80%, 90%, or 95% of an amount of the circular polyribonucleotide persists for a time period of at least about 3, 4, 5, 6, 7, 8, 9, 10, 12, 14, or 16 days in a cell.

In some embodiments, the circular polyribonucleotide is non-immunogenic in a mammal, e.g., a human.

### Production Methods

In some embodiments, the circular polyribonucleotide includes a deoxyribonucleic acid sequence that is non-naturally occurring and can be produced using recombinant technology (e.g., derived in vitro using a DNA plasmid), chemical synthesis, or a combination thereof.

It is within the scope of the disclosure that a DNA molecule used to produce an RNA circle can comprise a DNA sequence of a naturally-occurring original nucleic acid sequence, a modified version thereof, or a DNA sequence encoding a synthetic polypeptide not normally found in nature (e.g., chimeric molecules or fusion proteins, such as fusion proteins comprising multiple antigens and/or epitopes). DNA and RNA molecules can be modified using a variety of techniques including, but not limited to, classic mutagenesis techniques and recombinant techniques, such as site-directed mutagenesis, chemical treatment of a nucleic acid molecule to induce mutations, restriction enzyme cleavage of a nucleic acid fragment, ligation of nucleic acid fragments, polymerase chain reaction (PCR) amplification and/or mutagenesis of selected regions of a nucleic acid sequence, synthesis of oligonucleotide mixtures and ligation of mixture groups to “build” a mixture of nucleic acid molecules and combinations thereof.

The circular polyribonucleotide may be prepared according to any available technique including, but not limited to chemical synthesis and enzymatic synthesis. In some embodiments, a linear primary construct or linear mRNA may be cyclized, or concatemerized to create a circular polyribonucleotide described herein. The mechanism of cyclization or concatemerization may occur through methods such as, but not limited to, chemical, enzymatic, splint ligation), or ribozyme catalyzed methods. The newly formed 5′-/3′-linkage may be an intramolecular linkage or an intermolecular linkage.

Methods of making the circular polyribonucleotides described herein are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

Various methods of synthesizing circular polyribonucleotides are also described in the art (see, e.g., U.S. Pat. Nos. 6,210,931, 5,773,244, 5,766,903, 5,712,128, 5,426,180, US Publication No. US20100137407, International Publication No. WO1992001813 and International Publication No. WO2010084371; the contents of each of which are herein incorporated by reference in their entireties).

In some embodiments, the circular polyribonucleotides is purified, e.g., free ribonucleic acids, linear or nicked RNA, DNA, proteins, etc are removed. In some embodiments, the circular polyribonucleotides may be purified by any known method commonly used in the art. Examples of nonlimiting purification methods include, column chromatography, gel excision, size exclusion, etc.

### Circularization

In some embodiments, a linear circular polyribonucleotide may be cyclized, or concatemerized. In some embodiments, the linear circular polyribonucleotide may be cyclized in vitro prior to formulation and/or delivery. In some embodiments, the linear circular polyribonucleotide may be cyclized within a cell.

### Extracellular Circularization

In some embodiments, the linear circular polyribonucleotide is cyclized, or concatemerized using a chemical method to form a circular polyribonucleotide. In some chemical methods, the 5′-end and the 3′-end of the nucleic acid (e.g., a linear circular polyribonucleotide) includes chemically reactive groups that, when close together, may form a new covalent linkage between the 5′-end and the 3′-end of the molecule. The 5′-end may contain an NHS-ester reactive group and the 3′-end may contain a 3′-amino-terminated nucleotide such that in an organic solvent the 3′-amino-terminated nucleotide on the 3′-end of a linear RNA molecule will undergo a nucleophilic attack on the 5′-NHS-ester moiety forming a new 5′-/3′-amide bond.

In some embodiments, a DNA or RNA ligase may be used to enzymatically link a 5′-phosphorylated nucleic acid molecule (e.g., a linear circular polyribonucleotide) to the 3′-hydroxyl group of a nucleic acid (e.g., a linear nucleic acid) forming a new phosphorodiester linkage. In an example reaction, a linear circular polyribonucleotide is incubated at 37° C. for 1 hour with 1-10 units of T4 RNA ligase (New England Biolabs, Ipswich, MA) according to the manufacturer's protocol. The ligation reaction may occur in the presence of a linear nucleic acid capable of base-pairing with both the 5′- and 3′-region in juxtaposition to assist the enzymatic ligation reaction. In some embodiments, the ligation is splint ligation. For example, a splint ligase, like SplintR® ligase, can be used for splint ligation. For splint ligation, a single stranded polynucleotide (splint), like a single stranded RNA, can be designed to hybridize with both termini of a linear polyribonucleotide, so that the two termini can be juxtaposed upon hybridization with the single-stranded splint. Splint ligase can thus catalyze the ligation of the juxtaposed two termini of the linear polyribonucleotide, generating a circular polyribonucleotide.

In some embodiments, a DNA or RNA ligase may be used in the synthesis of the circular polynucleotides. As a non-limiting example, the ligase may be a circ ligase or circular ligase.

In some embodiments, either the 5′- or 3′-end of the linear circular polyribonucleotide can encode a ligase ribozyme sequence such that during in vitro transcription, the resultant linear circular polyribonucleotide includes an active ribozyme sequence capable of ligating the 5′-end of the linear circular polyribonucleotide to the 3′-end of the linear circular polyribonucleotide. The ligase ribozyme may be derived from the Group I Intron, Hepatitis Delta Virus, Hairpin ribozyme or may be selected by SELEX (systematic evolution of ligands by exponential enrichment). The ribozyme ligase reaction may take 1 to 24 hours at temperatures between 0 and 37° C.

In some embodiments, a linear circular polyribonucleotide may be cyclized or concatermerized by using at least one non-nucleic acid moiety. In one aspect, the at least one non-nucleic acid moiety may react with regions or features near the 5′ terminus and/or near the 3′ terminus of the linear circular polyribonucleotide in order to cyclize or concatermerize the linear circular polyribonucleotide. In another aspect, the at least one non-nucleic acid moiety may be located in or linked to or near the 5′ terminus and/or the 3′ terminus of the linear circular polyribonucleotide. The non-nucleic acid moieties contemplated may be homologous or heterologous. As a non-limiting example, the non-nucleic acid moiety may be a linkage such as a hydrophobic linkage, ionic linkage, a biodegradable linkage and/or a cleavable linkage. As another non-limiting example, the non-nucleic acid moiety is a ligation moiety. As yet another non-limiting example, the non-nucleic acid moiety may be an oligonucleotide or a peptide moiety, such as an aptamer or a non-nucleic acid linker as described herein.

In some embodiments, a linear circular polyribonucleotide may be cyclized or concatermerized due to a non-nucleic acid moiety that causes an attraction between atoms, molecular surfaces at, near or linked to the 5′ and 3′ ends of the linear circular polyribonucleotide. As a non-limiting example, one or more linear circular polyribonucleotides may be cyclized or concatermized by intermolecular forces or intramolecular forces. Non-limiting examples of intermolecular forces include dipole-dipole forces, dipole-induced dipole forces, induced dipole-induced dipole forces, Van der Waals forces, and London dispersion forces. Non-limiting examples of intramolecular forces include covalent bonds, metallic bonds, ionic bonds, resonant bonds, agnostic bonds, dipolar bonds, conjugation, hyperconjugation and antibonding.

In some embodiments, the linear circular polyribonucleotide may comprise a ribozyme RNA sequence near the 5′ terminus and near the 3′ terminus. The ribozyme RNA sequence may covalently link to a peptide when the sequence is exposed to the remainder of the ribozyme. In one aspect, the peptides covalently linked to the ribozyme RNA sequence near the 5′ terminus and the 3′ terminus may associate with each other causing a linear circular polyribonucleotide to cyclize or concatemerize. In another aspect, the peptides covalently linked to the ribozyme RNA near the 5′ terminus and the 3′ terminus may cause the linear primary construct or linear mRNA to cyclize or concatemerize after being subjected to ligated using various methods known in the art such as, but not limited to, protein ligation. Non-limiting examples of ribozymes for use in the linear primary constructs or linear RNA of the present invention or a non-exhaustive listing of methods to incorporate and/or covalently link peptides are described in US patent application No. US20030082768, the contents of which is here in incorporated by reference in its entirety.

In some embodiments, the linear circular polyribonucleotide may include a 5′ triphosphate of the nucleic acid converted into a 5′ monophosphate, e.g., by contacting the 5′ triphosphate with RNA 5′ pyrophosphohydrolase (RppH) or an ATP diphosphohydrolase (apyrase). Alternately, converting the 5′ triphosphate of the linear circular polyribonucleotide into a 5′ monophosphate may occur by a two-step reaction comprising: (a) contacting the 5′ nucleotide of the linear circular polyribonucleotide with a phosphatase (e.g., Antarctic Phosphatase, Shrimp Alkaline Phosphatase, or Calf Intestinal Phosphatase) to remove all three phosphates; and (b) contacting the 5′ nucleotide after step (a) with a kinase (e.g., Polynucleotide Kinase) that adds a single phosphate.

In some embodiments, the circularization efficiency of the circularization methods provided herein is at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, at least about 95%, or 100%. In some embodiments, the circularization efficiency of the circularization methods provided herein is at least about 40%.

In some embodiment, the circular polyribonucleotide includes at least one splicing element. Exemplary splicing elements are described in paragraphs [0270]-[0275] of WO2019/118919, which is hereby incorporated by reference in its entirety.

### Other Circularization Methods

In some embodiments, linear circular polyribonucleotides may include complementary sequences, including either repetitive or nonrepetitive nucleic acid sequences within individual introns or across flanking introns. Repetitive nucleic acid sequence are sequences that occur within a segment of the circular polyribonucleotide. In some embodiments, the circular polyribonucleotide includes a repetitive nucleic acid sequence. In some embodiments, the repetitive nucleotide sequence includes poly CA or poly UG sequences. In some embodiments, the circular polyribonucleotide includes at least one repetitive nucleic acid sequence that hybridizes to a complementary repetitive nucleic acid sequence in another segment of the circular polyribonucleotide, with the hybridized segment forming an internal double strand. In some embodiments, repetitive nucleic acid sequences and complementary repetitive nucleic acid sequences from two separate circular polyribonucleotides hybridize to generate a single circularized polyribonucleotide, with the hybridized segments forming internal double strands. In some embodiments, the complementary sequences are found at the 5′ and 3′ ends of the linear circular polyribonucleotides. In some embodiments, the complementary sequences include about 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more paired nucleotides.

In some embodiments, chemical methods of circularization may be used to generate the circular polyribonucleotide. Such methods may include, but are not limited to click chemistry (e.g., alkyne and azide based methods, or clickable bases), olefin metathesis, phosphoramidate ligation, hemiaminal-imine crosslinking, base modification, and any combination thereof.

In some embodiments, enzymatic methods of circularization may be used to generate the circular polyribonucleotide. In some embodiments, a ligation enzyme, e.g., DNA or RNA ligase, may be used to generate a template of the circular polyribonuclease or complement, a complementary strand of the circular polyribonuclease, or the circular polyribonuclease.

Circularization of the circular polyribonucleotide may be accomplished by methods known in the art, for example, those described in “RNA circularization strategies in vivo and in vitro” by Petkovic and Muller from Nucleic Acids Res, 2015, 43(4): 2454-2465, and “In vitro circularization of RNA” by Muller and Appel, from RNA Biol, 2017, 14(8):1018-1027.

The circular polyribonucleotide may encode a sequence and/or motifs useful for replication. Exemplary replication elements include binding sites for RNA polymerase. Other types of replication elements are described in paragraphs [0280]-[0286] of WO2019/118919, which is hereby incorporated by reference in its entirety. In some embodiments, the circular polyribonucleotide as disclosed herein lacks a replication element, e.g., lacks an RNA-dependent RNA polymerase binding site.

In some embodiments, the circular polyribonucleotide lacks a poly-A sequence and a replication element.

### Compositions for Administration to a Subject

The cell comprising a circular polyribonucleotide described herein may be included in various compositions, preparations, suspensions, or medical devices for administration to a subject.

For example, a cell (e.g., an isolated cell) as described herein is in a pharmaceutical composition for administration to a subject. The present invention includes compositions in combination with one or more pharmaceutically acceptable excipients.

A pharmaceutically acceptable excipient can be a non-carrier excipient. A non-carrier excipient serves as a vehicle or medium for a composition, such as a circular polyribonucleotide as described herein. A non-carrier excipient serves as a vehicle or medium for a composition, such as a linear polyribonucleotide as described herein. Non-limiting examples of a non-carrier excipient include solvents, aqueous solvents, non-aqueous solvents, dispersion media, diluents, dispersions, suspension aids, surface active agents, isotonic agents, thickening agents, emulsifying agents, preservatives, polymers, peptides, proteins, cells, hyaluronidases, dispersing agents, granulating agents, disintegrating agents, binding agents, buffering agents (e.g., phosphate buffered saline (PBS)), lubricating agents, oils, and mixtures thereof. A non-carrier excipient can be any one of the inactive ingredients approved by the United States Food and Drug Administration (FDA) and listed in the Inactive Ingredient Database that does not exhibit a cell-penetrating effect. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

In some embodiments, pharmaceutical compositions (e.g., a cell comprising a circular polyribonucleotide as described herein) provided herein are suitable for administration to a subject, wherein the subject is a non-human animal, for example, suitable for veterinary use. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, any animals, such as humans and/or other primates; mammals, including commercially relevant mammals, e.g., pet and live-stock animals, such as cattle, pigs, horses, sheep, cats, dogs, mice, and/or rats; and/or birds, including commercially relevant birds such as poultry, chickens, ducks, geese, and/or turkeys; zoo animals, e.g., a feline; non-mammal animals, e.g., reptiles, fish, amphibians, etc.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

The cellular compositions described herein may be used or administered as a pharmaceutical composition. In some embodiments, the pharmaceutical composition comprises a cell comprising a circular polyribonucletoide. The pharmaceutical composition may further comprise one or more pharmaceutically acceptable carriers or excipients.

In some embodiments, the pharmaceutically acceptable carrier or excipient is a sugar (e.g., sucrose, lactose, mannitol, maltose, sorbitol or fructose), a neutral salt (e.g., sodium chloride, magnesium sulfate, magnesium chloride, potassium sulfate, sodium carbonate, sodium sulfite, potassium acid phosphate, or sodium acetate), an acidic component (e.g., fumaric acid, maleic acid, adipic acid, citric acid or ascorbic acid), an alkaline component (e.g., tris(hydroxymethyl) aminomethane (TRIS), meglumine, tribasic or dibasic phosphates of sodium or potassium), or an amino acid (e.g., glycine or arginine).

In some embodiments, the pharmaceutical composition comprises a plurality or preparation of the cells, wherein the preparation comprise or the plurality is at least 10⁵ cells, e.g. at least 10⁶ or at least 10⁷ or at least 10⁸ or at least 10⁹ or at least 10¹⁰ or at least 10¹¹ cells, e.g., between from 5×10⁵ cells to 1×10⁷ cells. In some embodiments, the plurality is from 12.5×10⁵ cells to 4.4×10¹¹ cells. In some embodiments, the pharmaceutical composition comprises a plurality or preparation of the cells that is a unit dose for a target subject, e.g., the pharmaceutical composition comprises between 10⁵-10⁹ cells/kg of the target subject, e.g., between 10⁶-10⁸ cells/kg of the subject (e.g., a target subject, such as subject in need thereof). For example, a unit dose for a target subject weighing 50 kg may be a pharmaceutical composition that comprises between 5×10⁷ and 2.5×10¹⁰ cells, e.g., between 5×10⁷ and 2.5×10⁹ cells, e.g., between 5×10⁸ and 5×10⁹ cells.

As another example, the cells (e.g., isolated cells) for a cellular therapy as described herein are in a preparation. A preparation can comprise of from 1×10⁵ to 9×10¹¹ cells, e.g., between 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, the preparation configured for parenteral delivery to a subject, wherein the preparation comprises a plurality (e.g., at least 1% of the cells in the preparation) of cells or isolated cells as described herein. For example, at least 50% of the cells, at least 60% of the cells, e.g., between 50-70% of the cells in the preparation are cells comprising a synthetic, exogenous circular RNA as described herein. In some embodiments, the preparation is in a unit dose form described herein. In some embodiments, the delivery is injection or infusion (e.g., IV injection or infusion). A preparation can comprise from 5×10⁵ cells to 4.4×10¹¹ cells as disclosed herein configured for delivery (e.g., intravenous administration) to a subject. In some embodiments, the preparation comprises from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹ cells as disclosed herein, or any range of cells therebetween. In some embodiments, the preparation is configured for injection or infusion. In some embodiments, the preparation is in a unit dose form of from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the preparation comprises a dose of from 5×10⁵ cells/kg to 6×10⁸ cells/kg. In some embodiments, the preparation comprises a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween.

In some embodiments, the cells for a cellular therapy as described herein are in an intravenous bag or infusion product. An intravenous bag or other infusion product can comprise a suspension of isolated cells, wherein a plurality of the cells in the suspension (e.g., at least 1% of the cells in the preparation) is any cell or isolated cell described herein. In embodiments, the suspension comprises from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, the IV bag being configured for parenteral delivery to a subject. In some embodiments, at least 50% of the cells, at least 60% of the cells, e.g., between 50-70% of the cells in the suspension are cells comprising a synthetic, exogenous circular RNA as described herein. In some embodiments, the IV bag comprises a unit dose of cells described herein. An intravenous bag or infusion product can comprise a suspension of cells as described herein comprising from 5×10⁵ cells to 1×10⁷ cells as disclosed herein configured for delivery to a subject. In some embodiments, the suspension comprises from 12.5×10⁵ cells to 4.4×10¹¹ cells as disclosed herein. In some embodiments, the suspension of cells comprises from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the suspension comprises a dose of from 5×10⁵ cells/kg to 6×10⁸ cells/kg. In some embodiments, the suspension comprises a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween.

In some embodiments, the cells (e.g., isolated cells) for a cellular therapy as described herein are in a medical device. A medical device can comprise a plurality of cells, e.g., from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, the medical device being configured for implantation into a subject, wherein at least 40% of the cells in the medical device are cells or isolated cells as described herein. For example, at least 50% of the cells, at least 60% of the cells, e.g., between 50-70% of the cells in the medical device are cells comprising a synthetic, exogenous circular RNA as described herein. A medical device can comprise the cells as disclosed herein configured for implantation into a subject. In some embodiments, the medical device comprises from 5×10⁵ cells to 1×10⁷ cells as disclosed herein. In some embodiments, the medical device comprises from 12.5×10⁵ cells to 4.4×10¹¹ cells as disclosed herein. In some embodiments, the medical device comprises from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the medical device comprises a dose of from 5×10⁵ cells/kg to 6×10⁸ cells/kg. In some embodiments, the medical device comprises a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween. In some embodiments, the medical device is configured to produce and release the plurality of cells when implanted in the subject. In some embodiments, the medical device is configured to produce and release the protein (e.g., secreted protein or cleavable protein) when implanted into the subject.

In some embodiments, the cells (e.g., isolated cells) for a cellular therapy as described herein are in a biocompatible matrix. A biocompatible matrix can comprise a plurality of cells, wherein the biocompatible matrix is configured for implantation into a subject. The biocompatible matrix can comprise from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, wherein at least 50% of the cells, at least 60% of the cells, e.g., between 50-70% of the cells in the biocompatible matrix are cells comprising a synthetic, exogenous circular RNA as described herein. For example, the biocompatible matrix is an Afibromer™ matrix. For example, the biocompatible matrix may be that described in Bose et al. 2020. Nat Biomed Eng. 2020. doi:10.1038/s41551-020-0538-5, which is incorporated herein by reference. A biocompatible matrix can comprise the cells as disclosed herein configured for implantation into a subject. In some embodiments, the biocompatible matrix comprises from 5×10⁵ cells to 1×10⁷ cells as disclosed herein. In some embodiments, the biocompatible matrix comprises from 12.5×10⁵ cells to 4.4×10¹¹ cells as disclosed herein. In some embodiments, the biocompatible matrix comprises from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the biocompatible matrix comprises a dose of from 5×10⁵ cells/kg to 6×10⁸ cells/kg. In some embodiments, the biocompatible matrix comprises a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween. In some embodiments, the biocompatible matrix is configured to produce and release the plurality of cells when implanted in the subject. In some embodiments, the biocompatible matrix is configured to produce and release the protein (e.g., secreted protein or cleavable protein) when implanted into the subject.

In some embodiments, the cells (e.g., isolated cells) for a cellular therapy as described herein are in a bioreactor before administration to a subject. A bioreactor can comprise a plurality of cells, e.g., from 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., between 5×10⁵ cells to 4.4×10¹¹ cells, wherein at least 50% of the cells, at least 60% of the cells, e.g., between 50-70% of the cells in the bioreactcor are cells comprising a synthetic, exogenous circular RNA as described herein. A bioreactor can comprise the cells as described herein in a culture. In some embodiments, the bioreactor comprises a 2D cell culture. In some embodiments, the bioreactor comprises a 3D cell culture. In some embodiments, the cells from the bioreactor are in a pharmaceutical composition for administration to a subject, and the pharmaceutical composition comprises from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the cells from the bioreactor are in a pharmaceutical composition for administration to a subject, and the pharmaceutical composition comprises a dose of from 5×10⁵ cells/kg to 6×10⁸ cells/kg. In some embodiments, the cells from the bioreactor are in a pharmaceutical composition for administration to a subject, and the pharmaceutical composition comprises a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween.

In some embodiments, the cell for cellular therapy are cells exhibit a phenotype or genotype associated with the protein and/at least one binding site of the circular polyribonucleotide. For example, the cell expresses a protein (e.g., a CAR), is sensitized to a drug due to sequestration of a target in the cell by a binding to a binding site of a circular polyribonucleotide, or the cell is an edited cell. For example, cells as described herein comprising a circular polyribonucleotide encoding a nuclease that is capable of editing a nucleic acid in the cell. In some embodiments, a method of editing a nucleic acid of an isolated cell or plurality of isolated cells comprises providing an isolated cell or plurality of isolated cells, and contacting the isolated cell or plurality of isolated cells to a circular polyribonucleotide encoding a nuclease and/or comprising a guide nucleic acid, thereby producing an edited cell or plurality of edited cells for administration to a subject. The nuclease can be a zinc finger nuclease, transcription activator like effector nuclease or a Cas protein. In some embodiments, the Cas protein is a Cas9 protein, Cas12 protein, Cas14 protein, or Cas13 protein. In some embodiments, the nuclease edits a target sequence, wherein the target sequence is in the isolated cell. In some embodiments, the guide nucleic acid comprises a first region having a sequence that is complementary to a target sequence and a second region that hybridizes to the nuclease. The isolated cell or plurality of isolated cells can be any cell as described herein. In some embodiments, the method further comprise formulating the edited cell or plurality of edited cells with a pharmaceutically acceptable excipient. In some embodiments, the method further comprises administering the edited or plurality of edited cells to the subject. In some embodiments, the method further comprises administering the plurality of edited cells at a dose of from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the method further comprises administering the plurality of edited cells at a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween. In some embodiments, the method further comprises administering the plurality of edited cells at a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween, in two subsequent doses. In some embodiments, the two subsequent doses are at least about 28 days, 35 day, 42 days, or 60 days apart, or any day therebetween. As another example, cells as described herein comprising a circular polyribonucleotide encoding transcription factor, such as Oct4, Klf4, Sox2, cMyc, or a combination thereof, that is capable of reprogramming in the cell (e.g., reprogramming to produce an induce pluripotent stem cell). In some embodiments, a method of reprogramming a nucleic acid of an isolated cell or plurality of isolated cells comprises providing an isolated cell or plurality of isolated cells, and contacting the isolated cell or plurality of isolated cells to a circular polyribonucleotide encoding a transcription factor, thereby producing a reprogrammed cell or plurality of reprogrammed cells for administration to a subject. The transcription factor can be a as Oct4, Klf4, Sox2, or cMyc. In some embodiments, the circular polyribonucleotide encodes one or more transcription factors. In some embodiments, the transcription factors are each encoded by separate circular polyribonucleotides and these circular polyribonucleotides (e.g., a plurality of circular polyribonucleotides) are contacted to the isolated cell or plurality of isolated cells. The isolated cell or plurality of isolated cells can be any cell as described herein. In some embodiments, the method further comprise formulating the reprogrammed cell or plurality of reprogrammed cells with a pharmaceutically acceptable excipient. In some embodiments, the method further comprises administering the reprogrammed cell or plurality of reprogrammed cells to the subject. In some embodiments, method further comprising differentiating the reprogrammed cell or plurality of differentiated cells to into a cell type (e.g., beta cell, hemopoietic stem cell, etc.) to produce a differentiated cell or plurality of differentiated cells and then administering the differentiated cell or plurality of differentiated cells to a subject. In some embodiments, the method further comprises administering the plurality of reprogrammed cells or the plurality of differentiated cells at a dose of from from 5×10⁵ cells to 1×10⁷ cells, 5×10⁵ cells to 1×10⁸ cells, 5×10⁵ cells to 1×10⁹ cells, 5×10⁵ cells to 1×10¹⁰ cells, 5×10⁵ cells to 1×10¹¹ cells, 5×10⁵ cells to 2×10¹¹ cells, 5×10⁵ cells to 3×10¹¹ cells, 5×10⁵ cells to 4×10¹¹ cells, 1×10⁶ cells to 1×10⁷ cells, 1×10⁶ cells to 1×10⁸ cells, 1×10⁶ cells to 1×10⁹ cells, 1×10⁶ cells to 1×10¹⁰ cells, 1×10⁶ cells to 1×10¹¹ cells, 1×10⁶ cells to 2×10¹¹ cells, 1×10⁶ cells to 3×10¹¹ cells, 1×10⁶ cells to 4×10¹¹ cells, 1×10⁷ cells to 1×10⁸ cells, 1×10⁷ cells to 1×10⁹ cells, 1×10⁷ cells to 1×10¹⁰ cells, 1×10⁷ cells to 1×10¹¹ cells, 1×10⁷ cells to 2×10¹¹ cells, 1×10⁷ cells to 3×10¹¹ cells, 1×10⁷ cells to 4×10¹¹ cells, 1×10⁸ cells to 1×10⁹ cells, 1×10⁸ cells to 1×10¹⁰ cells, 1×10⁸ cells to 1×10¹¹ cells, 1×10⁸ cells to 2×10¹¹ cells, 1×10⁸ cells to 3×10¹¹ cells, 1×10⁸ cells to 4×10¹¹, 12.5×10⁵ cells to 4.4×10¹¹ cells as disclosed herein, or any range of cells therebetween. In some embodiments, the method further comprises administering the plurality of reprogrammed cells or the plurality of differentiated cells at a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween. In some embodiments, the method further comprises administering the plurality of edited cells at a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween, in two subsequent doses. In some embodiments, the two subsequent doses are at least about 28 days, 35 day, 42 days, or 60 days apart, or any day therebetween. The subject can be any subject as described herein.

### Methods of Producing and Administering a Cellular Therapy

Cells for cell therapy can be produced by contacting an isolated cell or plurality of isolated cells as described herein to a plurality of circular polyribonucleotides as described herein under conditions in which the circular polyribonucleotides are internalized into the isolated cell or plurality. In some embodiments, a method of producing a cell comprises providing an isolated cell or a plurality of isolated cells as described herein, providing the circular polyribonucleotide as described herein, and contacting the circular polyribonucleotide to the isolated cell or plurality of isolated cells. In some embodiments, a method of producing the cell or plurality of cells, comprises providing an isolated cell or a plurality of isolated cells; providing a preparation of circular polyribonucleotide as described herein, and contacting the circular polyribonucleotide to the isolated cell or plurality of isolated cells, wherein the isolated cell or plurality of isolated cells is capable of expressing the circular polyribonucleotide. In some embodiments, the preparation of circular polyribonucleotide contacted to the cells comprises no more than 1 ng/ml, 5 ng/ml, 10 ng/ml, 15 ng/ml, 20 ng/ml, 25 ng/ml, 30 ng/ml, 35 ng/ml, 40 ng/ml, 50 ng/ml, 60 ng/ml, 70 ng/ml, 80 ng/ml, 90 ng/ml, 100 ng/ml, 200 ng/ml, 300 ng/ml, 400 ng/ml, 500 ng/ml, 600 ng/ml, 1 μg/ml, 10 μg/ml, 50 μg/ml, 100 μg/ml, 200 g/ml, 300 μg/ml, 400 μg/ml, 500 μg/ml, 600 μg/ml, 700 μg/ml, 800 μg/ml, 900 μg/ml, 1 mg/ml, 1.5 mg/ml, or 2 mg/ml of linear polyribonucleotide molecules. In some embodiments, the preparation of circular polyribonucleotide contacted to the cells comprises at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) circular polyribonucleotide molecules relative to the total ribonucleotide molecules in the preparation of circular polyribonucleotides (e.g., a pharmaceutical preparation). In some embodiments, at least 30% (w/w), 40% (w/w), 50% (w/w), 60% (w/w), 70% (w/w), 80% (w/w), 85% (w/w), 90% (w/w), 91% (w/w), 92% (w/w), 93% (w/w), 94% (w/w), 95% (w/w), 96% (w/w), 97% (w/w), 98% (w/w), or 99% (w/w) of total ribonucleotide molecules in the preparation are circular polyribonucleotide molecules. In some embodiments, viability of the isolated cell or plurality of isolated cells after the contacting is at least 40% compared to a normalized uncontacted isolated cell or plurality of normalized uncontacted isolated cells. In some embodiments, the method further comprises administering the cell or plurality of cells after the contacting to a subject.

In some embodiments, viability of the isolated cell or plurality of isolated cells is at least 30%, 40%, 50%, 60%, 70%, 80% 90% 95%, 99% or 100% compared to a normalized uncontacted isolated cell or plurality of normalized uncontacted isolated cells. In some embodiments, a method of producing a cell or a plurality of cells for a transplant comprises providing a cell or plurality of cells in a tissue or an organ for transplant, providing the circular polyribonucleotide as described herein, and contacting the circular polyribonucleotide to the cell or the plurality of cells in a tissue or an organ for transplant, thereby producing the cell or plurality of cells for transplant. In some embodiments, the tissue or organ for transplant is removed from the subject, e.g., surgically removed, before the contacting. In some embodiments, after the contacting, the method comprises transplanting the cell or plurality of cells for transplant into a subject. In some embodiments, the tissue or organ for transplant is removed from a subject and transplanted back into the subject. In some embodiments, the tissue or organ for transplant is removed from a subject and transplanted into a different subject.

In some embodiments, the cells for cellular therapy are configured (e.g., in a medical device) or are suitable for parenteral administration in a subject, e.g., as an infusion product or injection product. A method of producing an infusion product can comprise enriching for a cell type from a plurality of cells, expanding the cell type, contacting a plurality of cells of the cell type to a plurality of circular polyribonucleotides sufficient to internalize the circular polyribonucleotides into the plurality of cells, wherein a circular polyribonucleotide of the plurality comprises at least one expression sequence encoding a protein that confers at least one therapeutic characteristic to the cell, at least one binding site that confers at least one therapeutic characteristic to the cell, or a combination thereof, and providing the contacted plurality of cells as an infusion product. A method of producing an injection product can comprise enriching for a cell type from a plurality of cells, expanding the cell type, contacting a plurality of cells of the cell type to a plurality of circular polyribonucleotides sufficient to internalize the circular polyribonucleotides into the plurality of cells, wherein a circular polyribonucleotide of the plurality comprises at least one expression sequence encoding a protein that confers at least one therapeutic characteristic to the cell, at least one binding site that confers at least one therapeutic characteristic to the cell, or a combination thereof, and providing the contacted plurality of cells as an injection product. In some embodiments, a method of producing an injection product comprises expanding an isolated cell to produce a plurality of isolated cells, contacting the plurality of isolated cells to a plurality of circular polyribonucleotides, wherein a circular polyribonucleotide of the plurality comprises at least one expression sequence encoding a protein that confers at least one therapeutic characteristic to the cell, at least one binding site that confers at least one therapeutic characteristic to the cell, or a combination thereof, and providing the contacted plurality of cells as an injection product. In some embodiments, the therapeutic characteristic of the at least one binding site confer nucleic acid activity (e.g., the at least one binding site is a miRNA binding site that results in nucleic acid degradation in a cell comprising the miRNA) in the isolated cell.

The produced cells for cellular therapy can then be administered to a subject in need thereof as the cellular therapy. In some embodiments, the circular polyribonucleotide is absent in the produced cells after a period of time (e.g., by degradation or lack of replication) and this produced cell is administered to a subject. For example, at least 50% of the cells, at least 60% of the cells, e.g., between 50-70% of the produced cells in the preparation are cells comprising a synthetic, exogenous circular polyribonucleotide as described herein. In some embodiments, the circular polyribonucleotide is present in the produced cells and this produced cell is administered to a subject. In some embodiments, cellular therapy as disclosed herein comprises a cell comprising a circular polyribonucleotide. In some aspects, the cellular therapy comprises a cell, wherein the cell comprises a circular polyribonucleotide as described herein. The cellular therapy can be used as a method of treating a subject in need thereof or as a method of treatment. In some embodiments, a method of cellular therapy comprises providing a circular polyribonucleotide as disclosed herein, and contacting the circular polyribonucleotide to an ex vivo cell (e.g., an isolated cell). In some embodiments, a method of cellular therapy comprises administering a cell as disclosed herein comprising a circular polyribonucleotide as disclosed herein to a subject in need thereof. In some embodiments, a method of treating a subject in need thereof comprises providing a cell as disclosed herein, contacting the cell ex vivo (e.g., isolated cell) to a circular polyribonucleotide as disclosed herein comprising one or more expression sequences, wherein an expression product of the one or more expression sequences comprises a protein for treating the subject. In some embodiments, a method of treatment comprises providing a cell as disclosed herein, and contacting the cell ex vivo (e.g., an isolated cell) to a circular polyribonucleotide as disclosed herein comprising one or more expression sequences, wherein at least one of the one or more expression sequences encodes a protein for treating a subject in need thereof. In further embodiments, the cell is administered to a subject in need thereof after the contacting.

### Contacting

In some embodiments, the contacting comprises contacting an isolated cell or plurality of isolated cells as described herein to a plurality of circular polyribonucleotides as described herein. In some embodiments, the contacting comprises contacting a cell ex vivo (e.g., an isolated cell) to a circular polyribonucleotide. In some embodiments, the contacting comprises contacting a cell ex vivo (e.g., an isolated cell) to a circular polyribonucleotide in a manner sufficient to internalize the circular polyribonucleotide or the circular polyribonucleotide into the cell. In some embodiments, the contacting comprises using cationic lipids, electroporation, naked circular RNA, aptamers, cationic polymers (e.g., PEI, polybrene, DEAE-dextran), virus-like particles (e.g., L1 from HPV, VP1 from polyomavirus), exosomes; nanostructured calcium phosphate; peptide transduction domains (e.g., TAT, polyR, SP, pVEC, SynB1, etc.); exosomes; vesicles (e.g., VSV-G, TAMEL); cell squeezing; nanoparticles; magnetofection; or any combination thereof; or any method of internalizing biomolecules into cells.

In some embodiments, viability of the cell after the contacting is at least 20%, 30%, 40%, 50%, 60%, 70%, 80%, 90%, 95%, 99%, or 100% compared to a normalized uncontacted cell.

The circular polyribonucleotide can persist in the cell after the contacting. The circular polyribonucleotide can persist for at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days after the contacting. The circular polyribonucleotide may persist for from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days after the contacting.

In some embodiments, at least about 10%, 20%, 30%, 40%, 50%, 60%, 70%, 80%, 90%, or 95% of an amount of the circular polyribonucleotide persists for a time period of at least about 3, 4, 5, 6, 7, 8, 9, 10, 12, 14, or 16 days in a cell after the contacting.

In some embodiments, persisting comprises maintaining at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 92%, at least about 94%, at least about 95%, at least about 96%, at least about 97%, at least about 98%, or at least about 99% of an amount of the polyribonucleotide as compared to the amount of the polyribonucleotide immediately following the contacting. In some embodiments, persisting comprises maintaining from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% of an amount of the polyribonucleotide as compared to the amount of the polyribonucleotide immediately following the contacting.

In some embodiments, the one or more expression sequences generates an amount of discrete polypeptides as compared to total polypeptides, wherein the amount is a percent of the total amount of polypeptides by moles of polypeptide. The polypeptides may be generated during rolling circle translation of a circular polyribonucleotide. Each of the discrete polypeptides may be generated from a single expression sequence. In some embodiments, the amount of discrete polypeptides is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 94%, at least 95%, at least 96%, at least 97%, or at least 98% of total polypeptides (molar/molar). In some embodiments, the amount of discrete polypeptides is from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% of total polypeptides (molar/molar).

In some embodiments, the circular polyribonucleotide comprises an expression sequence that generates greater amount of an expression product than a linear polyribonucleotide counterpart. In some embodiments, the greater amount of the expression product is at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, at least 20-fold, or at least 25-fold greater than that of the linear polyribonucleotide counterpart. In some embodiments, the greater amount of the expression product is from 1.5-fold to 1.6-fold, from 1.6-fold to 1.7-fold, from 1.7-fold to 1.8-fold, from 1.8-fold to 1.9-fold, from 1.9-fold to 2-fold, from 2-fold to 2.5-fold, from 2.5-fold to 3-fold, from 3-fold to 3.5-fold, from 3.5-fold to 4-fold, from 4-fold to 4.5-fold, from 4.5-fold to 5-fold, from 5-fold to 6-fold, from 6-fold to 7-fold, from 7-fold to 8-fold, from 8-fold to 9-fold, from 9-fold to 10-fold, from 10-fold to 15-fold, from 15-fold to 20-fold, from 20-fold to 25-fold, from 2-fold to 5-fold, from 2-fold to 6-fold, from 2-fold to 7-fold, from 2-fold to 10-fold, from 2-fold to 20-fold, from 4-fold to 5-fold, from 4-fold to 6-fold, from 4-fold to 7-fold, from 4-fold to 10-fold, from 4-fold to 20-fold, from 5-fold to 6-fold, from 5-fold to 7-fold, from 5-fold to 10-fold, from 5-fold to 20-fold, or from 10-fold to 20-fold greater than that of the linear polyribonucleotide counterpart. In some embodiments, the greater amount of the expression product is generated in a cell for at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days after the contacting. In some embodiments, the greater amount of the expression product is generated in a cell for from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days after the contacting.

The circular polyribonucleotide may express one or more expression sequences, wherein the expression level of the one or more expression sequences is maintained over a period of time after the contacting. In some embodiments, the expression is maintained at a level that does not vary by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 92%, about 94%, about 95%, about 96%, about 97%, or about 98% over the period of time. In some embodiments, the expression is maintained at a level that does not vary by more than from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% over the period of time. In some embodiments, the period of time over which the expression is maintained is up to 1 day, at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days after the contacting. In some embodiments, the period of time over which the expression is maintained is from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days after the contacting. In some embodiments the time period begins 1 day after the contacting.

In some embodiments, the expression does not decrease by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 92%, about 94%, about 95%, about 96%, about 97%, or about 98% over the period of time. In some embodiments the time period is 1 day after the contacting. In some embodiments, the expression does not decrease by greater than from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% over the period of time. In some embodiments the time period is 1 day after the contacting.

In some embodiments, the one or more expression sequences generates at least 1.5 fold greater expression product in the cell than a linear counterpart for a time period of at least at 3, 4, 5, 6, 7, 8, 9, 10, 12, 14, or 16 days in the cell after the contacting. In some embodiments, expression of the one or more expression sequences in the cell is maintained at a level that does not vary by more than about 10%, 20%, 30%, 40%, 50%, 60%, 70%, 80%, 90%, or 95% for time period of at least 3, 4, 5, 6, 7, 8, 9, 10, 12, 14, or 16 days after contacting the cell with the circular polyribonucleotide. In some embodiments, the level of the expression that is maintained is the level of the expression one day after the contacting. In some embodiments, the level of the expression that is maintained is the highest level of the expression one day after the contacting. In some embodiments, the level of expression of the one or more expression sequences in the cell does not decrease by greater than about 10%, 20%, 30%, 40%, 50%, 60%, 70%, 80%, 90%, or 95% over a time period of at least 3, 4, 5, 6, 7, 8, 9, 10, 12, 14, or 16 days after contacting the cell with the circular polyribonucleotide. In some embodiments, the level of the expression that does not decrease by greater than about 10%, 20%, 30%, 40%, 50%, 60%, 70%, 80%, 90%, or 95% is the level of the expression one day after the contacting. In some embodiments, the level of the expression does not decrease by greater than about 10%, 20%, 30%, 40%, 50%, 60%, 70%, 80%, 90%, or 95% compared to the highest level of the expression day one after contacting the cell with the circular polyribonucleotide.

After translation, the protein can be detected in the cell (e.g., also includes in a membrane of the cell) or outside the cell (e.g., as a secreted protein). In some embodiments, the protein is detected in the cell over a time period of at least 3, 4, 5, 6, 7, 8, 9, 10, 12, 14, 16, 20, 30, 40, 50, 60, or more days after the contacting. In some embodiments, the protein is detected on surface of the cell over a time period of at least 3, 4, 5, 6, 7, 8, 9, 10, 12, 14, 16, 20, 30, 40, 50, 60, or more days after the contacting. In some embodiments, the secreted protein is detected over a time period of at least 3, 4, 5, 6, 7, 8, 9, 10, 12, 14, 16, 20, 30, 40, 50, 60, or more days. In some embodiments, the time period begins one day after contacting the cell with the circular polyribonucleotide encoding the protein. The protein can be detected using any technique known in the art for protein detection, such as by flow cytometry.

### Circular Polyribonucleotide Composition

A circular polyribonucleotide described herein may be included in a composition for contacting a cell as described herein. The composition may be a pharmaceutical composition. The pharmaceutical composition can be free of any carrier. The pharmaceutical composition can comprise a carrier.

In some embodiments, the circular polyribonucleotide or a pharmaceutical composition thereof is delivered to (e.g., by contacting) a cell (e.g., an isolated cell) as a naked delivery formulation. A naked delivery formulation delivers a circular polyribonucleotide to a cell without the aid of a carrier and without covalent modification or partial or complete encapsulation of the circular polyribonucleotide.

A naked delivery formulation is a formulation that is free from a carrier and wherein the circular polyribonucleotide is without a covalent modification that binds a moiety that aids in delivery to a cell or without partial or complete encapsulation of the circular polyribonucleotide. In some embodiments, a circular polyribonucleotide without covalent modification bound to a moiety that aids in delivery to a cell is not covalently bound to a protein, small molecule, a particle, a polymer, or a biopolymer that aids in delivery to a cell. An unmodified circular polyribonucleotide without bound to a moiety that aids in delivery to a cell may not contain a modified phosphate group. For example, an circular polyribonucleotide without bound to a moiety that aids in delivery to a cell may not contain phosphorothioate, phosphoroselenates, boranophosphates, boranophosphate esters, hydrogen phosphonates, phosphoramidates, phosphorodiamidates, alkyl or aryl phosphonates, or phosphotriesters.

In some embodiments, a naked delivery formulation may be free of any or all of: transfection reagents, cationic carriers, carbohydrate carriers, nanoparticle carriers, or protein carriers. For example, a naked delivery formulation may be free from phtoglycogen octenyl succinate, phytoglycogen beta-dextrin, anhydride-modified phytoglycogen beta-dextrin, lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), N,N-dioleyl-N,N-dimethylammonium chloride (DODAC), human serum albumin (HSA), low-density lipoprotein (LDL), high-density lipoprotein (HDL), or globulin.

A naked delivery formulation may comprise a non-carrier excipient. In some embodiments, a non-carrier excipient may comprise an inactive ingredient. In some embodiments, a non-carrier excipient may comprise a buffer, for example PBS. In some embodiments, a non-carrier excipient may be a solvent, a non-aqueous solvent, a diluent, a suspension aid, a surface active agent, an isotonic agent, a thickening agent, an emulsifying agent, a preservative, a polymer, a peptide, a protein, a cell, a hyaluronidase, a dispersing agent, a granulating agent, a disintegrating agent, a binding agent, a buffering agent, a lubricating agent, or an oil.

In some embodiments, a naked delivery formulation may comprise a diluent. A diluent may be a liquid diluent or a solid diluent. In some embodiments, a diluent may be an RNA solubilizing agent, a buffer, or an isotonic agent. Examples of an RNA solubilizing agent include water, ethanol, methanol, acetone, formamide, and 2-propanol. Examples of a buffer include 2-(N-morpholino)ethanesulfonic acid (MES), Bis-Tris, 2-[(2-amino-2-oxoethyl)-(carboxymethyl)amino]acetic acid (ADA), N-(2-Acetamido)-2-aminoethanesulfonic acid (ACES), piperazine-N,N′-bis(2-ethanesulfonic acid) (PIPES), 2-[[1,3-dihydroxy-2-(hydroxymethyl)propan-2-yl]amino]ethanesulfonic acid (TES), 3-(N-morpholino)propanesulfonic acid (MOPS), 4-(2-hydroxyethyl)-1-piperazineethanesulfonic acid (HEPES), Tris, Tricine, Gly-Gly, Bicine, or phosphate. Examples of an isotonic agent include glycerin, mannitol, polyethylene glycol, propylene glycol, trehalose, or sucrose.

In some embodiments, the circular polyribonucleotide or a pharmaceutical composition thereof may be delivered to a cell (e.g., an isolated cell) with a carrier. Pharmaceutical compositions described herein may be formulated, for example, to include a carrier, such as a pharmaceutical carrier, e.g., a membrane, lipid bilayer, and/or a polymeric carrier, e.g., a liposome or particle such as a nano particle, e.g., a lipid nanoparticle, and delivered by known methods, such as via partial or complete encapsulation of the circular polyribonucleotide, to a cell for use in a subject in need thereof (e.g., a human or non-human agricultural or domestic animal, e.g., cattle, dog, cat, horse, poultry). Such methods include, but are not limited to, transfection (e.g., lipid-mediated, cationic polymers, calcium phosphate, dendrimers); viral delivery (e.g., lentivirus, retrovirus, adenovirus, AAV), fugene, protoplast fusion, exosome-mediated transfer, lipid nanoparticle-mediated transfer, and any combination thereof. Cationic lipid-mediated delivery of proteins enables efficient protein-based genome editing in vitro and in vivo. Nat Biotechnol. 2014 Oct. 30; 33(1):73-80. Methods of delivery are also described, e.g., in Gori et al., Delivery and Specificity of CRISPR/Cas9 Genome Editing Technologies for Human Gene Therapy. Human Gene Therapy. July 2015, 26(7): 443-451. doi:10.1089/hum.2015.074; and Zuris et al.

Additional methods of delivery include electroporation (e.g., using a flow electroporation device) or other methods of membrane disruption (e.g., nucleofection), microinjection, microprojectile bombardment (“gene gun”), direct sonic loading, cell squeezing, optical transfection, impalefection, magnetofection, and any combination thereof. A flow electroporation device, for example, comprises a chamber for containing a suspension of cells to be electorporated, such as the cells (e.g., isolated cells) as described herein, the chamber being at least partially defined by oppositely chargeable electrodes, wherein the thermal resistance of the chamber is less than approximately 110° C. per Watt.

### Cell and Vesicle-Based Carriers

A circular polyribonucleotide described herein may be included in a composition for contacting a cell as described herein, wherein the composition (e.g., a pharmaceutical composition) comprises in a vesicle or other membrane-based carrier.

In some embodiments, the circular polyribonucleotide, composition thereof, or pharmaceutical composition thereof is delivered (e.g., by contacting) to a cell as described herein, in or via a cell, vesicle or other membrane-based carrier. In some embodiments, the circular polyribonucleotide, composition thereof, or pharmaceutical composition thereof is formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for a circular polyribonucleotide or the pharmaceutical composition thereof as described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N\N′-Dimethylaminoethane)-carbamoyl]Cholesterol Hydrochloride (DC-Cholesterol HCl), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a circular polyribonucleotide or a pharmaceutical composition thereof described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a circular polyribonucleotide or a pharmaceutical composition thereof described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the circular polyribonucleotide or pharmaceutical composition thereof described herein.

Virosomes and virus-like particles (VLPs) can also be used as carriers to the circular polyribonucleotide or pharmaceutical composition thereof described herein to a cell (e.g., an isolated cell).

The invention is further directed to a host or host cell comprising the circular polyribonucleotide described herein. In some embodiments, the host or host cell is a plant, insect, bacteria, fungus, vertebrate, mammal (e.g., human), or other organism or cell.

In some embodiments, the circular polyribonucleotide is non-immunogenic in the host. In some embodiments, the circular polyribonucleotide has a decreased or fails to produce a response by the host's immune system as compared to the response triggered by a reference compound, e.g. a linear polynucleotide corresponding to the described circular polyribonucleotide or a circular polyribonucleotide lacking an encryptogen. Some immune responses include, but are not limited to, humoral immune responses (e.g. production of antigen-specific antibodies) and cell-mediated immune responses (e.g. lymphocyte proliferation).

In some embodiments, a host or a host cell is contacted with (e.g., delivered to or administered to) the circular polyribonucleotide. In some embodiments, the host is a mammal, such as a human. The amount of the circular polyribonucleotide, expression product, or both in the host can be measured at any time after administration. In certain embodiments, a time course of host growth in a culture is determined. If the growth is increased or reduced in the presence of the circular polyribonucleotide, the circular polyribonucleotide or expression product or both is identified as being effective in increasing or reducing the growth of the host.

### Administering

In some embodiments, the administration of a cell after the contacting to a subject in need thereof is conducted using any delivery method described herein. In some embodiments, the cell is administered parenterally. In some embodiments, the cell is administered to the subject via intravenous injection. In some embodiments, the administration of the cell, comprising a circular polyribonucleotide, includes, but is not limited to, prenatal administration, neonatal administration, postnatal administration, oral, by injection (e.g., intravenous, intraarterial, intraperotoneal, intradermal, subcutaneous and intramuscular), by ophthalmic administration and by intranasal administration. In some embodiments, the delivery is administration of a cell as described herein, a plurality of cells as described herein, a pharmaceutical composition of the cells as described herein, a preparation of the cells as described herein, by a medical device comprising the cells as described herein, by a biocompatible matrix comprising the cells as described herein, or cells as described herein from a bioreactor.

In some embodiments, a method of cellular therapy comprising administering a cell as described herein, a plurality of cells as described herein, a pharmaceutical composition of the cells as described herein, a preparation of the cells as described herein, implanting a medical device comprising the cells as described herein, implanting a biocompatible matrix comprising the cells as described herein, or administering cells as described herein from a bioreactor. In some embodiments, a method of cellular therapy comprises administering a pharmaceutical composition, cell, plurality of cells, preparation, a plurality of cells in an intravenous bag, a plurality of cells in a medical device, a plurality of cells in a biocompatible matrix, or a plurality of cells from a bioreactor as described herein to a subject in need thereof. In some embodiments, the administered pharmaceutical composition, plurality of cells, cell preparation, plurality of cells in an intravenous bag, plurality of cells in a medical device, or plurality of cells in a biocompatible matrix comprises a unit dose for the subject, e.g., comprises between 10⁵-10⁹ cells/kg of the subject, e.g., between 10⁶-10⁸ cells/kg of the subject. For example, a unit dose for a target subject weighing 50 kg may be a pharmaceutical composition that comprises between 5×10⁷ and 2.5×10¹⁰ cells, e.g., between 5×10⁷ and 2.5×10⁹ cells, e.g., between 5×10⁸ and 5×10⁹ cells.

In some embodiments, the pharmaceutical composition, plurality of cells, preparation, intravenous bag, medical device, or biocompatible matrix comprises a dose of, e.g., 1×10⁵ to 9×10¹¹ cells, e.g., between 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g. from 5×10⁵ cells to 4.4×10¹¹ cells, wherein at least 1% of the cells are cells or isolated cells as described herein. For example, at least 50% of the cells, at least 60% of the cells, e.g., between 50-70% of the cells in the plurality, cell preparation, intravenous bag, medical device, or biocompatible matrix are cells comprising a synthetic, exogenous circular RNA as described herein. In some embodiments, the method comprises administering the pharmaceutical composition, plurality of cells, or preparation at a dose of 1×10⁵ to 9×10¹¹ cells, e.g., between 1×10⁵-9×10⁵ cells, between 1×10⁶-9×10⁶ cells, between 1×10⁷-9×10⁷ cells, between 1×10⁸-9×10⁸ cells, between 1×10⁹-9×10⁹ cells, between 1×10¹⁰-9×10¹⁰ cells, between 1×10¹¹-9×10¹¹ cells, e.g., from 5×10⁵ cells/kg to 6×10⁸ cells/kg. In some embodiments, the method comprises administering the pharmaceutical composition, plurality of cells, or preparation in a plurality of administrations or doses. In some embodiments, the plurality, e.g., two, subsequent doses are administered at least about 7 days, 14 weeks, 28 days, 35 days, 42 days, or 60 days apart or more, or any day therebetween.

In some embodiments, the pharmaceutical composition, plurality of cells, preparation, plurality of cells in the intravenous bag, medical device, or biocompatible matrix, or plurality of cells from the bioreactor comprises a dose of from 5×10⁵ cells/kg to 6×10⁸ cells/kg. In some embodiments, the pharmaceutical composition, plurality of cells, preparation, plurality of cells in the intravenous bag, medical device, or biocompatible matrix, or plurality of cells from the bioreactor comprises a dose of from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween. In some embodiments, the method of cellular therapy comprises administering the pharmaceutical composition, plurality of cells, or preparation at a dose of from 5×10⁵ cells/kg to 6×10⁸ cells/kg in two subsequent doses. In some embodiments, the method of cellular therapy comprises administering the pharmaceutical composition, plurality of cells, or preparation at from 5×10⁵ cells/kg to 6×10⁸ cells/kg, 5×10⁵ cells/kg to 6×10⁹ cells/kg, 5×10⁴ cells/kg to 6×10⁸ cells/kg, 5×10⁴ cells/kg to 6×10⁹ cells/kg, 5×10⁵ cells/kg to 6×10⁶ cells/kg, 5×10⁵ cells/kg to 6×10⁷ cells/kg, or any range of cell/kg therebetween, in two subsequent doses. In some embodiments, the two subsequent doses are administered at least about 7 days, 14 day, 28 days, 35 day, 42 days, or 60 days apart, or more, or any day therebetween.

The circular polyribonucleotide can persist in the cell after the administering. The circular polyribonucleotide can persist for at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days after the administering. The circular polyribonucleotide may persist for from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days after the administering.

In some embodiments, at least about 10%, 20%, 30%, 40%, 50%, 60%, 70%, 80%, 90%, or 95% of an amount of the circular polyribonucleotide persists for a time period of at least about 3, 4, 5, 6, 7, 8, 9, 10, 12, 14, or 16 days in a cell after the administering.

In some embodiments, persisting comprises maintaining at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 92%, at least about 94%, at least about 95%, at least about 96%, at least about 97%, at least about 98%, or at least about 99% of an amount of the polyribonucleotide as compared to the amount of the polyribonucleotide immediately following the contacting. In some embodiments, persisting comprises maintaining from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% of an amount of the polyribonucleotide as compared to the amount of the polyribonucleotide immediately following the administering.

In some embodiments, the one or more expression sequences generates an amount of discrete polypeptides as compared to total polypeptides, wherein the amount is a percent of the total amount of polypeptides by moles of polypeptide. The polypeptides may be generated during rolling circle translation of a circular polyribonucleotide. Each of the discrete polypeptides may be generated from a single expression sequence. In some embodiments, the amount of discrete polypeptides is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 92%, at least 94%, at least 95%, at least 96%, at least 97%, or at least 98% of total polypeptides (molar/molar). In some embodiments, the amount of discrete polypeptides is from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% of total polypeptides (molar/molar).

In some embodiments, the circular polyribonucleotide comprises an expression sequence that generates greater amount of an expression product than a linear polyribonucleotide counterpart in a cell as described herein. In some embodiments, the greater amount of the expression product is at least 1.5-fold, at least 1.6-fold, at least 1.7-fold, at least 1.8-fold, at least 1.9-fold, at least 2-fold, at least 2.5-fold, at least 3-fold, at least 3.5-fold, at least 4-fold, at least 4.5-fold, at least 5-fold, at least 6-fold, at least 7-fold, at least 8-fold, at least 9-fold, at least 10-fold, at least 15-fold, at least 20-fold, or at least 25-fold greater than that of the linear polyribonucleotide counterpart in a cell. In some embodiments, the greater amount of the expression product is from 1.5-fold to 1.6-fold, from 1.6-fold to 1.7-fold, from 1.7-fold to 1.8-fold, from 1.8-fold to 1.9-fold, from 1.9-fold to 2-fold, from 2-fold to 2.5-fold, from 2.5-fold to 3-fold, from 3-fold to 3.5-fold, from 3.5-fold to 4-fold, from 4-fold to 4.5-fold, from 4.5-fold to 5-fold, from 5-fold to 6-fold, from 6-fold to 7-fold, from 7-fold to 8-fold, from 8-fold to 9-fold, from 9-fold to 10-fold, from 10-fold to 15-fold, from 15-fold to 20-fold, from 20-fold to 25-fold, from 2-fold to 5-fold, from 2-fold to 6-fold, from 2-fold to 7-fold, from 2-fold to 10-fold, from 2-fold to 20-fold, from 4-fold to 5-fold, from 4-fold to 6-fold, from 4-fold to 7-fold, from 4-fold to 10-fold, from 4-fold to 20-fold, from 5-fold to 6-fold, from 5-fold to 7-fold, from 5-fold to 10-fold, from 5-fold to 20-fold, or from 10-fold to 20-fold greater than that of the linear polyribonucleotide counterpart in a cell. In some embodiments, the greater amount of the expression product is generated in a cell for at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days after the contacting. In some embodiments, the greater amount of the expression product is generated in a cell for from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days after the administering.

The circular polyribonucleotide may express one or more expression sequences, wherein the expression level of the one or more expression sequences is maintained over a period of time after the contacting to a cell as described herein and after administering the cell. In some embodiments, the expression is maintained at a level that does not vary by more than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 92%, about 94%, about 95%, about 96%, about 97%, or about 98% over the period of time. In some embodiments, the expression is maintained at a level that does not vary by more than from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% over the period of time. In some embodiments, the period of time over which the expression is maintained is up to 1 day, at least about 1 day, at least about 2 days, at least about 3 days, at least about 4 days, at least about 5 days, at least about 6 days, at least about 7 days, at least about 8 days, at least about 9 days, at least about 10 days, at least about 12 days, at least about 14 days, at least about 16 days, at least about 18 days, at least about 20 days, at least about 25 days, at least about 30 days, at least about 40 days, or at least about 50 days after the administering. In some embodiments, the period of time over which the expression is maintained is from 1 day to 2 days, from 2 days to 3 days, from 3 days to 4 days, from 4 days to 5 days, from 5 days to 6 days, from 6 days to 7 days, from 7 days to 8 days, from 8 days to 9 days, from 9 days to 10 days, from 10 days to 12 days, from 12 days to 14 days, from 14 days to 16 days, from 16 days to 18 days, from 18 days to 20 days, from 20 days to 25 days, from 25 days to 30 days, from 30 days to 40 days, from 40 days to 50 days, from 1 day to 14 days, from 1 days to 30 days, from 7 days to 14 days, from 7 days to 30 days, or from 14 days to 30 days after the administering. In some embodiments the time period begins 1 day after the administering.

In some embodiments, the expression does not decrease by greater than about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 92%, about 94%, about 95%, about 96%, about 97%, or about 98% over the period of time. In some embodiments the time period is 1 day after the administering. In some embodiments, the expression does not decrease by greater than from 5% to 10%, from 10% to 15%, from 15% to 20%, from 20% to 25%, from 25% to 30%, from 30% to 35%, from 35% to 40%, from 40% to 45%, from 45% to 50%, from 50% to 55%, from 55% to 60%, from 60% to 65%, from 65% to 70%, from 70% to 75%, from 75% to 80%, from 80% to 85%, from 85% to 90%, from 90% to 92%, from 92% to 94%, from 94% to 95%, from 95% to 96%, from 96% to 97%, from 97% to 98%, from 98% to 99%, from 10% to 30%, from 10% to 40%, from 10% to 50%, from 10% to 60%, from 10% to 70%, from 10% to 80%, from 10% to 90%, from 10% to 95%, from 40% to 50%, from 40% to 60%, from 40% to 70%, from 40% to 80%, from 40% to 90%, from 40% to 95%, from 60% to 80%, from 60% to 90%, from 60% to 95%, or from 60% to 98% over the period of time. In some embodiments the time period is 1 day after the administering.

In some embodiments, the one or more expression sequences generates at least 1.5 fold greater expression product than a linear counterpart in the cell for a time period of at least at 3, 4, 5, 6, 7, 8, 9, 10, 12, 14, or 16 days in the cell after the administering. In some embodiments, expression of the one or more expression sequences in the cell is maintained at a level that does not vary by more than about 10%, 20%, 30%, 40%, 50%, 60%, 70%, 80%, 90%, or 95% for time period of at least 3, 4, 5, 6, 7, 8, 9, 10, 12, 14, or 16 days after the administering. In some embodiments, the time period begins one day after administering the cell. In some embodiments, the level of the expression that is maintained is the level of the expression one day after the administering. In some embodiments, the level of the expression that is maintained is the level of the highest level of the expression one day after the administering. In some embodiments, the expression of the one or more expression sequences in the cell over a time period of at least 3, 4, 5, 6, 7, 8, 9, 10, 12, 14, or 16 days does not decrease by greater than about 10%, 20%, 30%, 40%, 50%, 60%, 70%, 80%, 90%, or 95% after the administering. In some embodiments, the level of the expression that does not decrease by greater than about 10%, 20%, 30%, 40%, 50%, 60%, 70%, 80%, 90%, or 95% is the level of the expression one day after the administering. In some embodiments, the level of the expression does not decrease by greater than about 10%, 20%, 30%, 40%, 50%, 60%, 70%, 80%, 90%, or 95% compared to the highest level of the expression one day after administering.

After translation, the protein can be detected in the cell or as a secreted protein. In some embodiments, the protein is detected in the cell over a time period of at least 3, 4, 5, 6, 7, 8, 9, 10, 12, 14, 16, 20, 30, 40, 50, 60, or more days after the administering. In some embodiments, the protein is detected on surface of the cell over a time period of at least 3, 4, 5, 6, 7, 8, 9, 10, 12, 14, 16, 20, 30, 40, 50, 60, or more days after the administering. In some embodiments, the secreted protein is detected over a time period of at least 3, 4, 5, 6, 7, 8, 9, 10, 12, 14, 16, 20, 30, 40, 50, 60, or more days. In some embodiments, the secreted protein is detected over a time period of at least 3, 4, 5, 6, 7, 8, 9, 10, 12, 14, 16, 20, 30, 40, 50, 60, or more days. In some embodiments, the time period begins one day after administering the cell expressing the protein. The protein can be detected using any technique known in the art for protein detection, such as by flow cytometry.

### Subject

A subject in need thereof can be a human or a non-human animal. The human may be a juvenile, a young adult, (between 18-25 years), an adult, or a neonate.

The subject in need thereof can have a disease or disorder. In some embodiments, the subject has a hyperproliferative disease. In some embodiments, the subject has cancer. In some embodiments, the subject has a neurodegenerative disease. In some embodiments, the subject has a metabolic disease. In some embodiments, the subject has a metabolic disease. In some embodiments, the subject has an inflammatory disease. In some embodiments, the subject has an autoimmune disease. In some embodiments, the subject has an infectious disease. In some embodiments, the subject has a genetic disease.

In some embodiments, the cell for cellular therapy and the subject administered the cell are allogeneic. In some embodiments, the cell for cellular therapy and the subject administered the cell are autologous.

### Exemplary Cell Therapies

A cell therapy can be the combination of cells, compositions, or methods as described herein for the treatment of a subject need thereof. An exemplary cell therapy comprises a preparation of between 1×10⁶-1×10¹¹ human cells (e.g., T cells), e.g., between 1×10⁷ to 5×10¹⁰ human cells, e.g., between 1×10⁸-1×10⁹ human cells, is formulated with a excipient suitable for parenteral administration, wherein at least 50% (e.g., between 50%-70%) of the cells of the preparation comprise an exogenous circular RNA that expresses a chimeric antigen receptor described herein, and wherein the preparation is in a medical device such as an infusion bag, which is configured for parenteral delivery to a human. The cell therapy further comprises a method of treating a human subject diagnosed with cancer, e.g., a leukemia or lymphoma (e.g., acute lymphoblastic leukemia or relapsed or refractory diffuse large B-cell lymphoma), comprising administering to the subject a preparation of autologous T cells formulated with an excipient suitable for parenteral administration, wherein at least 50% (e.g., between 50%-70%) of the cells of the preparation comprise an exogenous circular RNA that expresses a chimeric antigen receptor described herein, wherein the preparation is administered at a dose of between 1×10⁵ to 1×10⁹ cells/kg of the subject, via a medical device such as an infusion bag, which is configured for parenteral delivery to the human.

A second exemplary cell therapy comprises a preparation of between 1×10⁶-1×10¹¹ human cells (e.g., CD34+ hematopoietic stem cells or HSCs, e.g., NK cells), e.g., between 1×10⁷ to 5×10¹⁰ human cells, e.g., between 1×10⁸-1×10⁹ human cells, formulated with a excipient suitable for parenteral administration, wherein at least 50% (e.g., between 50%-70%) of the cells of the preparation comprise an exogenous circular RNA that expresses hemoglobin Subunit Beta (Beta Globin or Hemoglobin Beta Chain or HBB) for treatment of thalassemia or for sickle cell disease, or express an ABC transporter for treatment of cerebral adrenoleukodystrophy, and wherein the preparation is in a medical device such as an infusion bag, which is configured for parenteral delivery to a human, and wherein the preparation is administered at a dose of between 1×10⁵ to 1×10⁹ cells/kg of the subject, via a medical device such as an infusion bag, which is configured for parenteral delivery to the human.

Another exemplary cell therapy comprises preparation of between 1×10⁶-1×10¹¹ human cells (e.g., CD34+ hematopoietic stem cells or HSCs, e.g., NK cells), e.g., between 1×10⁷ to 5×10¹⁰ human cells, e.g., between 1×10⁸-1×10⁹ human cells, formulated with a excipient suitable for parenteral administration, wherein at least 50% (e.g., between 50%-70%) of the cells of the preparation comprise an exogenous circular RNA that expresses (a) hemoglobin Subunit Beta (Beta Globin or Hemoglobin Beta Chain or HBB) for treatment of thalassemia or for sickle cell disease, or (b) an ABC transporter for treatment of cerebral adrenoleukodystrophy, or (c) adenosine deaminase (ADA) for treatment of ADA-SCID, or (d) WAS protein for treatment of Wiskott-Aldrich, or (e) CYBB protein for treatment of X-Linked chronic granulomatous disease or (f) ARSA for treatment of metachromatic leukodystrophy, or (g) α-L-iduronidase for treatment of MPS-I, or (h) N-sulfoglucosamine sulfohydrolase for treatment of MPS-IIIA or (i) N-acetyl-alpha-glucosaminidase for treatment of MPS-IIIB, and wherein the preparation is in a medical device such as an infusion bag, which is configured for parenteral delivery to a human, and wherein the preparation is administered at a dose of between 1×10⁵ to 1×10⁹ cells/kg of the subject, via a medical device such as an infusion bag, which is configured for parenteral delivery to the human. In some embodiments, the dose is an IV dose, e.g., a single IV dose, e.g., of 1-5 million cells.

All references and publications cited herein are hereby incorporated by reference. The above described embodiments can be combined to achieve the afore-mentioned functional characteristics.

### Numbered Embodiments #1

[1] A cell comprising a circular polyribonucleotide, wherein the circular polyribonucleotide comprises at least one expression sequence encoding a therapeutic protein.
[2] A cell comprising a therapeutic protein and a circular polyribonucleotide, wherein the circular polyribonucleotide comprises at least one expression sequence encoding the therapeutic protein.
[3] A therapeutic cell comprising a protein and a circular polyribonucleotide, wherein the circular polyribonucleotide comprises at least one expression sequence encoding the protein that confers at least one therapeutic characteristic to the cell.
[4] A therapeutic cell comprising a circular polyribonucleotide, wherein the circular polyribonucleotide comprises at least one binding site that confers at least one therapeutic characteristic to the cell.
[5] A therapeutic cell comprising a circular polyribonucleotide, wherein the circular polyribonucleotide comprises at least one binding site that confers at least one therapeutic characteristic to the cell.
[6] The cell of any one of the preceding embodiments, wherein the cell is a therapeutic cell.
[7] The cell or therapeutic cell of any one of the preceding embodiments, wherein the cell is an ex vivo cell.
[8] The cell or therapeutic cell of any one of the preceding embodiments, wherein the cell is a eukaryotic cell.
[9] The cell or therapeutic cell of any one of the preceding embodiments, wherein the cell is an animal cell.
[10] The cell or therapeutic cell of any one of the preceding embodiments, wherein the cell is a mammalian cell.
[11] The cell or therapeutic cell of any one of the preceding embodiments, wherein the cell is a human cell.
[12] The cell or therapeutic cell of any one of the preceding embodiments, wherein the cell is an immune cell, a cancer cell, a progenitor cell, or a stem cell.
[13] The cell or therapeutic cell of any one of the preceding embodiments, wherein the cell is a peripheral blood mononuclear cell.
[14] The cell or therapeutic cell of any one of the preceding embodiments, wherein the cell is a lymphocyte.
[15] The cell or therapeutic cell of any one of the preceding embodiments, wherein the cell is a peripheral blood lymphocyte.
[16] The cell or therapeutic cell of any one of the preceding embodiments, wherein the cell is selected from a group consisting of a T cell, a B cell, a Natural Killer cell, a Natural Killer T cell, a macrophage, a dendritic cell, a red a red blood cell reticulocyte, a myeloid progenitor, and a megakaryocyte.
[17] The cell or therapeutic cell of any one of the preceding embodiments, wherein the cell is selected from a group consisting of a mesenchymal stem cell, an embryological stem cell, a fetal stem cell, a placental derived stem cell, a induced pluripotent stem cell, an adipose stem cell, a hematopoietic stem cell, a skin stem cell, an adult stem cell, a bone marrow stem cell, a cord blood stem cell, an umbilical cord stem cell, a corneal limbal stem cell, a progenitor stem cell, and a neural stem cell.
[18] The cell or therapeutic cell of any one of the preceding embodiments, wherein the cell is a fibroblast.
[19] The cell or therapeutic cell of any one of the preceding embodiments, wherein the cell is a chondrocyte.
[20] The cell or therapeutic cell of any one of the preceding embodiments, wherein the protein is a therapeutic protein.
[21] The cell or therapeutic cell of any one of the preceding embodiments, wherein the protein is a protein that promotes cell expansion, cell immortalization, and/or localization of the cell to a target.
[22] The cell or therapeutic cell of any one of the preceding embodiments, wherein the protein or the therapeutic protein is an intracellular protein, a membrane protein, or a secreted protein.
[23] The cell or therapeutic cell of any one of the preceding embodiments, wherein the protein or the therapeutic protein has antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity.
[24] The cell or therapeutic cell of any one of the preceding embodiments, wherein the therapeutic protein is a chimeric antigen receptor.
[25] The cell or therapeutic cell of any one of the preceding embodiments, wherein the chimeric antigen receptor is a CD19 specific chimeric antigen receptor, a TAA specific chimeric antigen receptor, a BCMA specific chimeric antigen receptor, a HER2 specific chimeric antigen receptor, a CD2 specific chimeric antigen receptor, a NY-ESO-1 specific chimeric antigen receptor, a CD20 specific chimeric antigen receptor, a Mesothelina specific chimeric antigen receptor, a EBV specific chimeric antigen receptor, or a CD33 specific chimeric antigen receptor.
[26] The cell or therapeutic cell of any one of the preceding embodiments, wherein the therapeutic protein is epidermal growth factor, erythropoietin, or phenylalanine hydroxylase.
[27] The cell or therapeutic cell of any one of the preceding embodiments, wherein the protein or the therapeutic protein specifically binds an antigen.
[28] The cell or therapeutic cell of any one of the preceding embodiments, wherein the protein or the therapeutic protein is detected in the cell over a time period of at least 3, 4, 5, 6, 7, 8, 9, 10, 12, 14, or 16 days.
[29] The cell or therapeutic cell of any one of the preceding embodiments, wherein the protein or the therapeutic protein is detected on a surface of the cell over a time period of at least 3, 4, 5, 6, 7, 8, 9, 10, 12, 14, or 16 days.
[30] The cell or therapeutic cell of any one of the preceding embodiments, wherein the protein or the therapeutic protein is a secreted protein detected over a time period of at least 3, 4, 5, 6, 7, 8, 9, 10, 12, 14, or 16 days.
[31] The cell or therapeutic cell of any one of the preceding embodiments, wherein the at least one binding site is an aptamer.
[32] The cell or therapeutic cell of any one of the preceding embodiments, wherein the at least one binding site binds to a cell receptor on a surface of the cell.
[33] The cell or therapeutic cell of any one of the preceding embodiments, wherein the circular polyribonucleotide is internalized into the cell when the at least one binding site is bound to a cell receptor on the surface of the cell.
[34] The cell or therapeutic cell of any one of the preceding embodiments, wherein the circular polyribonucleotide comprises at least one expression sequence encoding a therapeutic protein and at least one binding site.
[35] The cell or therapeutic cell of any one of the preceding embodiments, wherein the circular polyribonucleotide is competent for rolling circle translation and lacks a termination element.
[36] The cell or therapeutic cell of any one of the preceding embodiments, wherein the circular polyribonucleotide further comprises a stagger element at a 3′ end of at least one of the expression sequences, and lacks a termination element.
[37] The cell or therapeutic cell of embodiment [36], wherein the stagger element stalls a ribosome during rolling circle translation of the circular polyribonucleotide.
[38] The cell or therapeutic cell of embodiment [36] or [37], wherein the stagger element encodes a sequence with a C-terminal consensus sequence that is D(V/I)ExNPGP, where x=any amino acid.
[39] The cell or therapeutic cell of any one of the preceding embodiments, wherein the circular polyribonucleotide lacks a cap, an internal ribosomal entry site, a poly-A tail, a replication element, or both.
[40] The cell or therapeutic cell of any one of the preceding embodiments, wherein the one or more expression sequences comprise a Kozak initiation sequence.
[41] The cell or therapeutic cell of any one of the preceding embodiments, wherein the circular polyribonucleotide further comprises at least one structural element selected from:
   (a) an encryptogen;
   (b) a regulatory element;
   (c) a replication element; and
   (d) quasi-double-stranded secondary structure.
[42] The cell or therapeutic cell of any one of the preceding embodiments, wherein the circular polyribonucleotide comprises at least one functional characteristic selected from:
   (i) greater translation efficiency than a linear counterpart;
   (ii) a stoichiometric translation efficiency of multiple translation products;
   (iii) less immunogenicity than a counterpart lacking an encryptogen;
   (iv) increased half-life over a linear counterpart; and
   (v) persistence during cell division.
[43] The cell or therapeutic cell of any one of embodiments [33]-[42], wherein the termination element comprises a stop codon.
[44] The cell or therapeutic cell of any one of the preceding embodiments, wherein the circular polyribonucleotide further comprises a replication domain configured to mediate self-replication of the circular polyribonucleotide.
[45] The cell or therapeutic cell of any one of the preceding embodiments, wherein the circular polyribonucleotide persists during cell division.
[46] The cell or therapeutic cell of any one of the preceding embodiments, wherein at least about 10%, 20%, 30%, 40%, 50%, 60%, 70%, 80%, 90%, or 95% of an amount of the circular polyribonucleotide persists for a time period of at least about 3, 4, 5, 6, 7, 8, 9, 10, 12, 14, or 16 days in the cell.
[47] The cell or therapeutic cell of any one of the preceding embodiments, wherein expressing the one or more expression sequences generates at least 10%, 20%, 30%, 40%, 50%, 60%, 70%, 80%, 90%, or 95% discrete polypeptides of total polypeptides (molar/molar) generated during rolling circle translation of the circular polyribonucleotide, and wherein each of the discrete polypeptides is generated from a single expression sequence.
[48] The cell or therapeutic cell of any one of the preceding embodiments, wherein the one or more expression sequences generates at least 1.5 fold greater expression product than a linear counterpart in the cell for a time period of at least at 3, 4, 5, 6, 7, 8, 9, 10, 12, 14, or 16 days in the cell.
[49] The cell or therapeutic cell of any one of the preceding embodiments, wherein expression of the one or more expression sequences in the cell is maintained at a level that does not vary by more than about 10%, 20%, 30%, 40%, 50%, 60%, 70%, 80%, 90%, or 95% for time period of at least 3, 4, 5, 6, 7, 8, 9, 10, 12, 14, or 16 days.
[50] The cell or therapeutic cell of any one of the preceding embodiments, wherein the expression of the one or more expression sequences in the cell over a time period of at least 3, 4, 5, 6, 7, 8, 9, 10, 12, 14, or 16 days does not decrease by greater than about 10%, 20%, 30%, 40%, 50%, 60%, 70%, 80%, 90%, or 95%.
[51] A pharmaceutical composition comprising:
   the cell or therapeutic cell of any one of any one of the preceding embodiments; and a pharmaceutically acceptable carrier or excipient.
[52] A method of cellular therapy comprising administering the cell or therapeutic cell of any one of the preceding embodiments or the pharmaceutical composition of embodiment [48] to a subject in need thereof.
[53] A method of cellular therapy, comprising:
   providing a circular polyribonucleotide comprising one or more expression sequences, at least one binding site, or a combination thereof, and
   contacting the circular polyribonucleotide to a cell ex vivo.
[54] A method of a treating a subject in need thereof, comprising:
   providing a cell, and
   contacting the cell ex vivo to a circular polyribonucleotide comprising one or more expression sequences, at least one binding site, or a combination thereof,
   wherein an expression product of the one or more expression sequences comprises a protein for treating the subject.
[55] A method of treatment comprising:
   providing a cell; and
   contacting the cell ex vivo to a circular polyribonucleotide comprising one or more expression sequences, at least one binding site, or a combination thereof,
   wherein at least one of the one or more expression sequences encodes a protein for treating a subject in need thereof.
[56] The method of any one of the preceding embodiments further comprising administering the cell after the contacting to a subject in need thereof.
[57] The method of any one of the preceding embodiments, wherein the contacting further comprises the cell internalizing the circular polyribonucleotide.
[58] The method of any one of the preceding embodiments, wherein the contacting comprises using cationic lipids, electroporation (e.g., using a flow electroporation device), naked circular RNA, aptamers, cationic polymers (e.g., PEI, polybrene, DEAE-dextran), virus-like particles (e.g., L1 from HPV, VP1 from polyomavirus), exosomes; nanostructured calcium phosphate; peptide transduction domains (e.g., TAT, polyR, SP, pVEC, SynB1, etc.); vesicles (e.g., VSV-G, TAMEL); exosomes; cell squeezing; nanoparticles; magnetofection, or any combination thereof.
[59] The method of any one of the preceding embodiments, wherein viability of the cell after the contacting is at least 40% compared to a normalized uncontacted cell.
[60] The method of any one of the preceding embodiments, wherein the subject in need thereof has a disease or disorder.
[61] The method of any one of the preceding embodiments, wherein the subject in need thereof has a hyperproliferative disease.
[62] The method of any one of the preceding embodiments, wherein the subject in need thereof has cancer.
[63] The method of any one of the preceding embodiments, wherein the subject in need thereof has a neurodegenerative disease.
[64] The method of any one of the preceding embodiments, wherein the subject in need thereof has a a metabolic disease.
[65] The method of any one of the preceding embodiments, wherein the subject in need thereof has an inflammatory disease.
[66] The method of any one of the preceding embodiments, wherein the subject in need thereof has a an autoimmune disease.
[67] The method of any one of the preceding embodiments, wherein the subject in need thereof has an infectious disease.
[68] The method of any one of the preceding embodiments, wherein the subject in need thereof has a genetic disease.
[69] The method of any one of the preceding embodiments, wherein the circular polyribonucleotide persists during cell division.
[70] The method of any one of the preceding embodiments, wherein at least about 10%, 20%, 30%, 40%, 50%, 60%, 70%, 80%, 90%, or 95% of an amount of the circular polyribonucleotide persists for at least about 3, 4, 5, 6, 7, 8, 9, 10, 12, 14, or 16 days in the cell after the contacting.
[71] The method of any one of the preceding embodiments, wherein at least about 10%, 20%, 30%, 40%, 50%, 60%, 70%, 80%, 90%, or 95% of an amount of the circular polyribonucleotide persists for at least about 3, 4, 5, 6, 7, 8, 9, 10, 12, 14, or 16 days in the cell after the administering.
[72] The method of any one of the preceding embodiments, wherein expressing the one or more expression sequences generates at least 10%, 20%, 30%, 40%, 50%, 60%, 70%, 80%, 90%, or 95% discrete polypeptides of total polypeptides (molar/molar) generated during the rolling circle translation of the circular polyribonucleotide, and wherein each of the discrete polypeptides is generated from a single expression sequence.
[73] The method of any one of the preceding embodiments, wherein the one or more expression sequences generates at least 1.5 fold greater expression product than a linear counterpart in the cell at least at day 3, 4, 5, 6, 7, 8, 9, 10, 12, 14, or 16 after the contacting.
[74] The method of any one of the preceding embodiments, wherein the one or more expression sequences generates at least 1.5 fold greater expression product than a linear counterpart in the cell at least at day 3, 4, 5, 6, 7, 8, 9, 10, 12, 14, or 16 after the administering.
[75] The method of any one of the preceding embodiments, wherein expression of the one or more expression sequences in the cell is maintained at a level that does not vary by more than about 10%, 20%, 30%, 40%, 50%, 60%, 70%, 80%, 90%, or 95% for at least 3, 4, 5, 6, 7, 8, 9, 10, 12, 14, or 16 days.
[76] The method of embodiment 69, wherein the level that does not vary by more than about 10%, 20%, 30%, 40%, 50%, 60%, 70%, 80%, 90%, or 95% is a level of expression of the one or more expression sequences 1 day after the administering.
[77] The method of embodiment 69, wherein the level that does not vary by more than about 10%, 20%, 30%, 40%, 50%, 60%, 70%, 80%, 90%, or 95% is a level of expression of the one or more expression sequences 1 day after the contacting.
[78] The method of any one of the preceding embodiments, wherein the expression of the one or more expression sequences in the cell over a time period of at least 3, 4, 5, 6, 7, 8, 9, 10, 12, 14, or 16 days does not decrease by greater than about 10%, 20%, 30%, 40%, 50%, 60%, 70%, 80%, 90%, or 95%.
[79] The method of embodiment [78], wherein the time period of at least 3, 4, 5, 6, 7, 8, 9, 10, 12, 14, or 16 days begins 1 day after the contacting.
[80] The method of embodiment [78], wherein the time period of at least 3, 4, 5, 6, 7, 8, 9, 10, 12, 14, or 16 days begins 1 day after the administering.
[81] The method of any one of the preceding embodiments, wherein the cell is a therapeutic cell.
[82] The method of any one of the preceding embodiments, wherein the cell is a eukaryotic cell.
[83] The method of any one of the preceding embodiments, wherein the cell is an animal cell.
[84] The method of any one of the preceding embodiments, wherein the cell is a mammalian cell.
[85] The method of any one of the preceding embodiments, wherein the cell is a human cell.
[86] The method of any one of the preceding embodiments, wherein the cell is an immune cell, a cancer cell, a progenitor cell, or a stem cell.
[87] The method of any one of the preceding embodiments, wherein the cell is a peripheral blood mononuclear cell.
[88] The method of any one of the preceding embodiments, wherein the cell is a lymphocyte.
[89] The method of any one of the preceding embodiments, wherein the cell is a peripheral blood lymphocyte.
[90] The method of any one of the preceding embodiments, wherein the cell is selected from a group consisting of a T cell, a B cell, a Natural Killer cell, a Natural Killer T cell, a macrophage, a dendritic cell, a megakaryocyte, a red blood cell reticulocyte, and a myeloid progenitor.
[91] The method of any one of the preceding embodiments or the cell of any one of the preceding embodiments, wherein the cell is selected from a group consisting of a mesenchymal stem cell, an embryological stem cell, a fetal stem cell, a placental derived stem cell, a induced pluripotent stem cell, an adipose stem cell, a hematopoietic stem cell (e.g., CD34⁺ cell), a skin stem cell, an adult stem cell, a bone marrow stem cell, a cord blood stem cell, an umbilical cord stem cell, a corneal limbal stem cell, a progenitor stem cell, and a neural stem cell.
[92] The method of any one of the preceding embodiments, wherein the cell is a fibroblast.
[93] The method of any one of the preceding embodiments, wherein the cell is a chondrocyte.
[94] The method of any one of the preceding embodiments, wherein the cell is autologous to the subject.
[95] The method of any one of the preceding embodiments, wherein the cell is allogeneic to the subject.
[96] The method of any one of the preceding embodiments, wherein an expression product of the one or more expression sequences comprises a therapeutic protein or a protein that confers a therapeutic characteristic to the cell.
[97] The method of any one of the preceding embodiments, wherein the protein promotes cell expansion, cell immortalization, and/or localization of the cell to a target.
[98] The method of any one of the preceding embodiments, wherein the protein or the therapeutic protein is an intracellular protein, a membrane protein, or a secreted protein.
[99] The method of any one of the preceding embodiments, wherein the protein or the therapeutic protein has antioxidant activity, binding, cargo receptor activity, catalytic activity, molecular carrier activity, molecular function regulator, molecular transducer activity, nutrient reservoir activity, protein tag, structural molecule activity, toxin activity, transcription regulator activity, translation regulator activity, or transporter activity.
[100] The method of any one of the preceding embodiments, wherein the therapeutic protein is a chimeric antigen receptor.
[101] The method of any one of the preceding embodiments or the cell of any one of the preceding embodiments, wherein the chimeric antigen receptor is a CD19 specific chimeric antigen receptor, a TAA specific chimeric antigen receptor, a BCMA specific chimeric antigen receptor, a HER2 specific chimeric antigen receptor, a CD2 specific chimeric antigen receptor, a NY-ESO-1 specific chimeric antigen receptor, a CD20 specific chimeric antigen receptor, a Mesothelina specific chimeric antigen receptor, a EBV specific chimeric antigen receptor, or a CD33 specific chimeric antigen receptor.
[102] The method of any one of the preceding embodiments, wherein the therapeutic protein is erythropoietin, epidermal growth factor, phenylalanine hydroxylase, or chimeric antigen receptor.
[103] The method of any one of the preceding embodiments, wherein the protein or therapeutic protein specifically binds an antigen.
[104] The method of any one of the preceding embodiments, wherein the protein or the therapeutic protein is detected in the cell over a time period of at least 3, 4, 5, 6, 7, 8, 9, 10, 12, 14, or 16 days after the contacting.
[105] The method of any one of the preceding embodiments, wherein the protein or the therapeutic protein is detected on a surface of the cell over a time period of at least 3, 4, 5, 6, 7, 8, 9, 10, 12, 14, or 16 days after the contacting.
[106] The method of any one of the preceding embodiments, wherein the protein or the therapeutic protein is a secreted protein detected over a time period of at least 3, 4, 5, 6, 7, 8, 9, 10, 12, 14, or 16 days after the contacting.
[107] The method any one of the preceding embodiments, wherein the at least one binding site is an aptamer.
[108] The method any one of the preceding embodiments, wherein the at least one binding site binds to a cell receptor on a surface of the cell.
[109] The method of any one of the preceding embodiments, wherein the circular polyribonucleotide is internalized into the cell when the at least one binding site is bound to a cell receptor on the surface of the cell.
[110] The method of any one of the preceding embodiments, wherein the circular polyribonucleotide is competent for rolling circle translation and lacks a termination element.
[111] The method of any one of the preceding embodiments, wherein the circular polyribonucleotide further comprises a stagger element at a 3′ end of at least one of the expression sequences, and lacks a termination element.
[112] The method of embodiment [111], wherein the stagger element stalls a ribosome during the rolling circle translation of the circular polyribonucleotide.
[113] The method of embodiment [111] or [112], wherein the stagger element encodes a sequence with a C-terminal consensus sequence that is D(V/I)ExNPGP, where x=any amino acid.
[114] The method of any one of the preceding embodiments, wherein the circular polyribonucleotide lacks an internal ribosomal entry site.
[115] The method of any one of the preceding embodiments, wherein the one or more expression sequences comprise a Kozak initiation sequence.
[116] The method of any one of the preceding embodiments, wherein the circular polyribonucleotide further comprises at least one structural element selected from:
   (a) an encryptogen;
   (b) a regulatory element;
   (c) a replication element; and
   (d) quasi-double-stranded secondary structure.
[117] The method of any one of the preceding embodiments, wherein the circular polyribonucleotide comprises at least one functional characteristic selected from:
   (i) greater translation efficiency than a linear counterpart;
   (ii) a stoichiometric translation efficiency of multiple translation products;
   (iii) less immunogenicity than a counterpart lacking an encryptogen;
   (iv) increased half-life over a linear counterpart; and
   (v) persistence during cell division.
[118] The method of any one of embodiments [110]-[117], wherein the termination element comprises a stop codon.
[119] The method of any one of the preceding embodiments, wherein the circular polyribonucleotide further comprises a replication domain configured to mediate self-replication of the circular polyribonucleotide

### Numbered Embodiments #2

[1] A pharmaceutical composition comprising
   a) a pharmaceutically acceptable carrier or excipient; and
   b) a cell comprising a circular polyribonucleotide, wherein the circular polyribonucleotide (1) comprises at least one binding site, (2) encodes a protein, wherein the protein is a secreted protein or an intracellular protein, or (3) a combination of (1) and (2).
[2] A pharmaceutical composition comprising
   a) a pharmaceutically acceptable carrier or excipient; and
   b) a cell comprising a circular polyribonucleotide, wherein the circular polyribonucleotide (1) comprises at least one binding site, (2) encodes a membrane protein, or (3) a combination of (1) and (2), wherein the membrane protein is not a chimeric antigen receptor, T cell receptor, or T cell receptor fusion protein or the cell is not an immune cell.
[3] A pharmaceutical composition comprising
   a) a pharmaceutically acceptable carrier or excipient; and
   b) a cell comprising a circular polyribonucleotide, wherein the circular polyribonucleotide comprises at least one binding site and encodes a protein, wherein the protein is a secreted protein, membrane protein, or an intracellular protein.
[4] An isolated cell comprising a circular polyribonucleotide, wherein the circular polyribonucleotide (1) comprises at least one binding site, (2) encodes a protein, wherein the protein is a secreted protein or an intracellular protein, or (3) a combination of (1) and (2) and wherein the isolated cell is administered to a subject.
[5] An isolated cell or a preparation comprising a circular polyribonucleotide, wherein the circular polyribonucleotide (1) comprises at least one binding site, (2) encodes a membrane protein, or (3) a combination of (1) and (2), wherein the membrane protein is not a chimeric antigen receptor, T cell receptor, or T cell receptor fusion protein or the isolated cell is not an immune cell, and wherein the isolated cell is administered to a subject.
[6] An isolated cell comprising a circular polyribonucleotide, wherein the circular polyribonucleotide comprises at least one binding site and encodes a protein, wherein the protein is a secreted protein, membrane protein, or an intracellular protein and wherein the isolated cell is administered to a subject.
[7] The pharmaceutical composition of embodiment [1] or the isolated cell of embodiments [4], wherein the protein is a membrane protein and the cell is a non-immune cell.
[8] The pharmaceutical composition or the isolated cell of any one of the preceding embodiments, wherein the intracellular protein, membrane protein, or secreted protein is a therapeutic protein.
[9] The pharmaceutical composition or the isolated cell of any one of the preceding embodiments, wherein the membrane protein is a transmembrane protein.
[10] The pharmaceutical composition or the isolated cell of any one of the preceding embodiments, wherein the membrane protein is an extracellular matrix protein.
[11] The pharmaceutical composition or the isolated cell of any one of the preceding embodiments, wherein the intracellular protein, membrane protein, or secreted protein promotes cell expansion, cell differentiation, cell immortalization, and/or localization of the cell to a target.
[12] The pharmaceutical composition or the isolated cell of any one of the preceding embodiments, wherein intracellular protein, membrane protein, or secreted protein has antioxidant activity, binding activity, cargo receptor activity, catalytic activity, molecular carrier activity, molecular transducer activity, nutrient reservoir activity, structural molecule activity, toxin activity, transcription regulator activity, translation regulator activity, tolerogenic activity, or transporter activity.
[13] The pharmaceutical composition or the isolated cell of any one of the preceding embodiments, wherein the intracellular protein, membrane protein, or secreted protein functions as a protein tag.
[14] The pharmaceutical composition or the isolated cell of any one of the preceding embodiments, wherein intracellular protein, membrane protein, or secreted protein is a molecular function regulator.
[15] The pharmaceutical composition or the isolated cell of any one of the preceding embodiments, wherein the intracellular protein, membrane protein, or secreted protein is a tolerogenic factor (e.g., HLA-G, PD-L1, CD47, or CD24).
[16] The pharmaceutical composition or the isolated cell of any one of the preceding embodiments, wherein the intracellular protein, membrane protein, or secreted protein is an epidermal growth factor, an erythropoietin, a phenylalanine hydroxylase, a chimeric antigen receptor, a nuclease, a zinc finger nuclease protein, a transcription activator like effector nuclease, or a Cas protein.
[17] The pharmaceutical composition or the isolated cell of any one of the preceding embodiments, wherein the at least one binding site confers at least one therapeutic characteristic to the cell.
[18] The pharmaceutical composition or the isolated cell of any one of the preceding embodiments, wherein the at least one binding site confers nuceleic acid localization to the cell or isolated cell.
[19] The pharmaceutical composition or the isolated cell of any one of the preceding embodiments, wherein the at least one binding site is an aptamer.
[20] The pharmaceutical composition or the isolated cell of any one of the preceding embodiments, wherein the at least one binding site is a protein binding site, DNA binding site, or RNA binding site.
[21] The pharmaceutical composition or the isolated cell of any one of the preceding embodiments, wherein the at least one binding site is an miRNA binding site.
[22] The pharmaceutical composition or the isolated cell of any one of the preceding embodiments, wherein the at least one binding site binds to a cell receptor on a surface of the cell.
[23] The pharmaceutical composition or the isolated cell of any one of the preceding embodiments, wherein the circular polyribonucleotide is internalized into the cell after the at least one binding site binds to a cell receptor on the surface of the cell.
[24] The pharmaceutical composition or the isolated cell of any one of the preceding embodiments, wherein the cell or isolated cell is a eukaryotic cell, animal cell, mammalian cell, or human cell.
[25] The pharmaceutical composition or the isolated cell of any one of the preceding embodiments, wherein the cell or isolated cell is an immune cell, progenitor cell, stem cell, neurological cell, cardiological cell, an adipocyte, liver cell, or beta cell.
[26] The pharmaceutical composition or the isolated cell of any one of the preceding embodiments, wherein the cell or isolated cell is a peripheral blood mononuclear cell, peripheral blood lymphocyte, or lymphocyte.
[27] The pharmaceutical composition or the isolated cell of any one of the preceding embodiments, wherein the cell or isolated cell is selected from a group consisting of a T cell (e.g., a regulatory T cell, γδ T cell, αβ cell, CD8+ T cell, or CD4+ T cell), a B cell, a Natural Killer cell, a Natural Killer T cell, a macrophage, a dendritic cell, a red blood cell, a reticulocyte, a myeloid progenitor, and a megakaryocyte.
[28] The pharmaceutical composition or the isolated cell of any one of the preceding embodiments, wherein the cell or isolated cell is selected from a group consisting of a mesenchymal stem cell, an embryological stem cell, a fetal stem cell, a placental derived stem cell, an induced pluripotent stem cell, an adipose stem cell, a hematopoietic stem cell (e.g., CD34+ cell), a skin stem cell, an adult stem cell, a bone marrow stem cell, a cord blood stem cell, an umbilical cord stem cell, a corneal limbal stem cell, a progenitor stem cell, and a neural stem cell.
[29] The pharmaceutical composition or the isolated cell of any one of the preceding embodiments, wherein the cell or isolated cell is selected from a group consisting of a fibroblast, a chondrocyte, a cardiomyocyte, a dopaminergic neuron, a microglia, a oligodendrocyte, a enteric neuron, and a hepatocyte.
[30] The pharmaceutical composition or the isolated cell of any one of the preceding embodiments, wherein the cell or isolated cell is replication incompetent.
[31] The pharmaceutical composition of any one of the preceding embodiments comprising a plurality of the cells, wherein the plurality is from 5×10⁵ cells to 1×10⁷ cells.
[32] The pharmaceutical composition of any one of the preceding embodiments comprising a plurality of the isolated cell (e.g., a preparation of comprising a plurality of the isolated cell) of any one of the preceding embodiments, wherein the plurality is from 5×10⁵ cells to 1×10⁷ cells.
[33] The pharmaceutical composition of any one of the preceding embodiments comprising a plurality of the cells or isolated cells, wherein the plurality is from 12.5×10⁵ cells to 4.4×10¹¹ cells.
[34] The pharmaceutical composition of any one of the preceding embodiments comprising a plurality of the isolated cell of any one of the preceding embodiments, wherein the plurality is from 12.5×10⁵ cells to 4.4×10¹¹ cells.
[35] The pharmaceutical composition of any one of the preceding embodiments for administration (e.g., by intravenous administration) to a subject.
[36] The pharmaceutical composition or the isolated cell of any one of the preceding embodiments, wherein the subject is a human or non-human animal.
[37] The pharmaceutical composition or isolated cell of any one of the preceding embodiments, wherein the subject has a disease or disorder.
[38] The pharmaceutical composition or the isolated cell of any one of the preceding embodiments, wherein the subject has a hyperproliferative disease, cancer, a neurodegenerative disease, a metabolic disease, an inflammatory disease, an autoimmune disease, an infectious disease, or a genetic disease.
[39] The pharmaceutical composition or the isolated cell of any one of the preceding embodiments, wherein the subject and the cell or isolated cell are allogeneic or are autologous.
[40] The pharmaceutical composition or the isolated cell of any one of the preceding embodiments, wherein the circular polyribonucleotide lacks a cap, an internal ribosome entry site, a poly-A tail, a replication element, or combination thereof.
[41] The isolated cell of any one of the preceding embodiments formulated with a pharmaceutically acceptable excipient (e.g., a diluent).
[42] A pharmaceutical composition comprising a cell, wherein the cell comprises a circular polyribonucleotide that comprises a sequence encoding an antigen-binding domain, a transmembrane domain, and an intracellular signaling domain, and comprises at least one binding site.
[43] An isolated cell comprising a circular polyribonucleotide that comprises a sequence encoding a chimeric antigen receptor and comprises at least one binding site, wherein the isolated cell is for administration (e.g., intravenous administration) to a subject.
[44] A cell comprising:
   a) a circular polyribonucleotide comprising
      i) at least one target binding sequence encoding an antigen-binding protein that binds to an antigen, or
      ii) a sequence encoding an antigen-binding domain, a transmembrane domain, and an intracellular signaling domain and, optionally, comprising at least one binding site; and
   b) a second nucleotide sequence encoding a protein, wherein expression of the protein is activated upon binding of the antigen to the antigen-binding protein.
[45] A cell comprising a circular polyribonucleotide encoding a T cell receptor (TCR) comprising affinity for an antigen and a circular polyribonucleotide encoding a bispecific antibody, wherein the cell expresses the TCR and bispecific antibody on a surface of the cell.
[46] The isolated cell of embodiment [43], wherein the chimeric antigen receptor comprises an antigen binding domain, a transmembrane domain, and an intracellular domain.
[47] The cell of embodiment [44], wherein the antigen-binding protein comprises an antigen-binding domain, a transmembrane domain, and an intracellular signaling domain.
[48] The pharmaceutical composition of embodiment [42], the isolated cell of embodiment [46], or the cell of embodiments [44] or [47], wherein the antigen-binding domain is linked to the transmembrane domain, which is linked to the intracellular signaling domain to produce a chimeric antigen receptor.
[49] The pharmaceutical composition of embodiments [42] or [48], the isolated cell of embodiments [46] or [48], or the cell of embodiments [44] or [47]-[48], wherein the antigen-binding domain binds to a tumor antigen, a tolerogen, or a pathogen antigen, or the antigen is a tumor antigen or a pathogen antigen.
[50] The pharmaceutical composition of any one of embodiments [42] or [48]-[49], the isolated cell of any one of embodiments [46] or [48]-[49], or the cell of embodiments [44] or [47]-[49], wherein the antigen-binding domain is an antibody or antibody fragment thereof (e.g., scFv, Fv, Fab).
[51] The pharmaceutical composition of any one of embodiments [42] or [48]-[50], the isolated cell of any one of embodiments [46] or [48]-[50], or the cell of embodiments [44] or [47]-[50], wherein the antigen-binding domain is a bispecific antibody.
[52] The cell of embodiment [45] or the pharmaceutical composition, cell, or isolated cell of embodiment [51], wherein the bispecific antibody has first immunoglobulin variable domain that binds a first epitope and a second immunoglobulin variable domain that binds a second epitope.
[53] The pharmaceutical composition, cell, or isolated cell of embodiment [52], wherein the first epitope and the second epitope are the same.
[54] The pharmaceutical composition, cell, or isolated cell of embodiment [52], wherein the first epitope and the second epitope are different.
[55] The pharmaceutical composition of any one of embodiments [42] or [48]-[54], the isolated cell of any one of embodiments [46] or [48]-[54], or the cell of embodiments [44] or [47]-[54], wherein the transmembrane domain links the antigen-binding domain and the intracellular signaling domain.
[56] The pharmaceutical composition of any one of embodiments [42] or [48]-[55], the isolated cell of any one of embodiments [46] or [48]-[55], or the cell of embodiments [44] or [47]-[55], wherein the transmembrane domain is a hinge protein (e.g., immunglobuline hinge), a polypeptide linker (e.g., GS linker), a KIR2DS2 hinge, a CD8a hinge, or a spacer.
[57] The pharmaceutical composition of any one of embodiments [42] or [48]-[56], the isolated cell of any one of embodiments [46] or [48]-[56], or the cell of embodiments [44] or [47]-[56], wherein the intracellular signaling domain comprises at least a portion of a T-cell signaling molecule.
[58] The pharmaceutical composition of any one of embodiments [42] or [48]-[57], the isolated cell of any one of embodiments [46] or [48]-[57], or the cell of embodiments [44] or [47]-[57], wherein the intracellular signaling domain comprises an immunoreceptor tyrosine-based activation motif.
[59] The pharmaceutical composition of any one of embodiments [42] or [48]-[58], the isolated cell of any one of embodiments [46] or [48]-[58], or the cell of embodiments [44] or [47]-[58], wherein the intracellular signaling domain comprises at least a portion of CD3zeta, common FcRgamma (FCER1G), Fc gamma RIIa, FcRbeta (Fc Epsilon Rib), CD3 gamma, CD3delta, CD3epsilon, CD79a, CD79b, DAP10, DAP12, or any combination thereof.
[60] The pharmaceutical composition of any one of embodiments [42] or [48]-[59], the isolated cell of any one of embodiments [46] or [48]-[59], or the cell of embodiments [44] or [47]-[59], wherein the intracellular signaling domain further comprises a costimulatory intracellular signaling domain.
[61] The pharmaceutical composition, cell, or isolated cell of any one of embodiment [60], wherein the costimulatory intracellular signaling domain comprises at least one or more of a TNF receptor protein, immunoglobulin-like protein, a cytokine receptor, an integrin, a signaling lymphocytic activation molecule, or an activating NK cell receptor protein.
[62] The pharmaceutical composition, cell, or isolated cell of embodiment [60] or [61], wherein the costimulatory intracellular signaling domain comprises at least one or more of CD27, CD28, 4-1BB, OX40, GITR, CD30, CD40, PD-1, ICOS, BAFFR, HVEM, ICAM-1, LFA-1, CD2, CDS, CD7, CD287, LIGHT, NKG2C, NKG2D, SLAMF7, NKp80, NKp30, NKp44, NKp46, CD160, CD19, CD4, CD8alpha, CD8beta, IL2R beta, IL2R gamma, IL7R alpha, ITGA4, VLA1, CD49a, IA4, CD49D, ITGA6, VLA6, CD49f, ITGAD, CD103, ITGAL, ITGAM, ITGAX, ITGB1, CD29, ITGB2, CD18, ITGB7, TNFR2, TRANCE/TRANKL, CD226, SLAMF4, CD84, CD96, CEACAM1, CRTAM, CD229, CD160, PSGL1, CD100, CD69, SLAMF6, SLAMF1, SLAMF8, CD162, LTBR, LAT, GADS, SLP-76, PAG/Cbp, CD19a, B7-H3, or a ligand that binds to CD83.
[63] The pharmaceutical composition, cell, or isolated cell of any one of embodiments [42]-[62], wherein the circular polyribonucleotide lacks a cap, an internal ribosome entry site, a poly-A tail, a replication element, or combination thereof.
[64] The pharmaceutical composition, cell, or isolated cell of any one of embodiments [42]-[63], wherein cell is an immune effector cell.
[65] The pharmaceutical composition, cell, or isolated cell of any one of embodiments [42]-[64], wherein the cell or isolated cell is a T cell (e.g., a αβ T cell, or γδ T cell) or an NK cell.
[66] The pharmaceutical composition, cell, or isolated cell of any one of embodiments [42]-[65], wherein the cell or isolated cell is an allogeneic cell or autologous cell.
[67] The cell of any one of embodiments [44], [45], or [47]-[66], wherein the antigen is expressed from a tumor or cancer.
[68] The cell of any one of embodiments [44] or [47]-[67], wherein the protein is a cytokine (e.g., IL-12) or a costimulatory ligand (e.g., CD40L or 4-1BBL).
[69] The cell of any one of embodiments [44] or [47]-[68], wherein the protein is a secreted protein.
[70] A preparation of from 5×10⁵ cells to 4.4×10¹¹ cells configured for delivery (e.g. by injection or infusion) to a subject, wherein a cell of the 5×10⁵ cells to 4.4×10¹¹ cells is the cell or the isolated cell of any one of the preceding embodiments; wherein the preparation is optionally in unit dose form.
[71] An intravenous bag or infusion product comprising a suspension of a plurality of cells configured for delivery (e.g. by injection or infusion) to a subject, wherein a cell of the plurality is the cell or the isolated cell of any one of the preceding embodiments.
[72] A medical device comprising a plurality of cells, wherein a cell of the plurality is the cell or the isolated cell of any one of the preceding embodiments, and wherein the medical device is configured for implantation into a subject.
[73] A biocompatible matrix comprising a plurality of cells, wherein a cell of the plurality is the cell or the isolated cell of any one of the preceding embodiments, and wherein the biocompatible matrix is configured for implantation into a subject.
[74] A bioreactor comprising a plurality of cells, wherein a cell of the plurality is the cell or isolated cell of any one of the preceding embodiments.
[75] The bioreactor of any one of the preceding embodiments, wherein the bioreactor comprises a 2D cell culture.
[76] The bioreactor of any one of the preceding embodiments, wherein the bioreactor comprises a 3D cell culture.
[77] The medical device of embodiment [72] or the biocompatible matrix of embodiment [73] configured to produce and release the plurality of cells when implanted into the subject.
[78] The medical device of embodiment [72] or the biocompatible matrix of embodiment [73] configured to produce and release the protein (e.g., secreted protein or cleavable protein) when implanted into the subject.
[79] The preparation, the intravenous bag, medical device, or biocompatible matrix of any one of embodiments [70]-[73] or [77]-[78], wherein the subject is a human or non-human animal.
[80] The preparation, intravenous bag, medical device, biocompatible matrix, or bioreactor of any one of embodiments [70]-[79], wherein the plurality of cells is formulated with a pharmaceutically acceptable carrier or excipient.
[81] A method of producing a cell or a plurality of cells, comprising: providing an isolated cell or a plurality of isolated cells; providing the circular polyribonucleotide of any one of the preceding embodiments, and contacting the circular polyribonucleotide to the isolated cell or plurality of isolated cells.
[82] The method of any one of the preceding embodiments, wherein viability of the isolated cell or plurality of isolated cells after the contacting is at least 40% compared to a normalized uncontacted isolated cell or a plurality of normalized uncontacted isolated cells.
[83] The method any one of embodiments [81] or [82], further comprising administering the cell or plurality of cells after the contacting to a subject.
[84] A method of producing a cell for administration to a subject comprising:
   a) providing an isolated cell, and
   b) contacting the isolated cell to the circular polyribonucleotide of any one the preceding embodiments;
      thereby producing the cell for administration to the subject.
[85] The method of embodiment [84], wherein the circular polyribonucleotide in the cell is degraded prior to administration to the subject.
[86] A method of producing an infusion product comprising:
   a) enriching for a cell type from a plurality of cells;
   b) expanding the cell type;
   c) contacting a plurality of cells of the cell type with a plurality of circular polyribonucleotides, wherein a circular polyribonucleotide of the plurality is the circular polyribonucleotide of any one of the preceding embodiments; and
   d) providing the contacted plurality of cells in a suspension as an infusion product.
[87] A method of producing an injection product comprising:
   a) enriching for a cell type from a plurality of cells;
   b) expanding the cell type;
   c) contacting a plurality of cells of the cell type with a plurality of circular polyribonucleotides, wherein a circular polyribonucleotide of the plurality is the circular polyribonucleotide of any one of the preceding embodiments; and
   d) providing the contacted plurality of cells in a suspension as an injection product.
[88] A method of cellular therapy comprising administering the pharmaceutical composition, the cell, plurality of cells, preparation, a plurality of cells in the intravenous bag, a plurality of cells in the medical device, a plurality of cells in the biocompatible matrix, or a plurality of cells from the bioreactor of any one of the preceding embodiments to a subject.
[89] The method of embodiment [88], wherein the pharmaceutical composition, the plurality of cells, the preparation, the plurality of cells in the intravenous bag, the plurality of cells in the medical device, the plurality of cells in the biocompatible matrix or the plurality of cells from the bioreactor comprises a dose of from 5×10⁵ cells to 4.4×10¹¹ cells.
[90] The method of embodiment [88] or [89], comprising administering the pharmaceutical composition, plurality of cells, preparation, the plurality of cells in the intravenous bag, the plurality of cells in the medical device, the plurality of cells in the biocompatible matrix or the plurality of cells from the bioreactor at a dose of from 5×10⁵ cells/kg to 6×10⁸ cells/kg.
[91] The method of any one of embodiments [88]-[90], comprising administering the pharmaceutical composition, plurality of cells, preparation, the plurality of cells in the intravenous bag, the plurality of cells in the medical device, the plurality of cells in the biocompatible matrix or the plurality of cells from the bioreactor at a dose of from 5×10⁵ cells/kg to 6×10⁸ cells/kg in two subsequent doses.
[92] The method of embodiment [91], wherein the two subsequent doses are administered at least about 28 days, 35 day, 42 days, or 60 days apart.
[93] A method of editing a nucleic acid of an isolated cell or plurality of isolated cells comprising
   a) providing an isolated cell or a plurality of isolated cells;
   b) contacting the isolated cell or the plurality of isolated cells to a circular polyribonucleotide encoding a nuclease and/or comprising a guide nucleic acid; thereby producing an edited cell or plurality of edited cells for administration to a subject.
[94] The method of embodiment [93], further comprising formulating the edited cell or the plurality of edited cells with a pharmaceutically acceptable excipient.
[95] The method of embodiments [93] or [94], further comprising administering the edited cell or the plurality of edited cells to the subject.
[96] The method of any one of embodiments [93]-[95], further comprising administering the plurality of edited cells at a dose of from 5×10⁵ cells/kg to 6×10⁸ cells/kg.
[97] The method of any one of embodiments [93]-[96], further comprising administering the plurality of edited cells at a dose of from 5×10⁵ cells/kg to 6×10⁸ cells/kg in two subsequent doses.
[98] The method of embodiment [97], wherein the two subsequent doses are administered at least about 28 days, 35 day, 42 days, or 60 days apart.
[99] The method of any one of embodiments [93]-[98], wherein the nuclease is a zinc finger nuclease, transcription activator like effector nuclease, or Cas protein.
[100] The method of any one of embodiments [93]-[99], wherein the nuclease is a Cas9 protein, Cas12 protein, Cas14 protein, or Cas13 protein.
[101] The method of any one of embodiments [93]-[100], wherein the nuclease edits a target sequence.
[102] The method of any one of embodiments [93]-[101], wherein the guide nucleic acid comprises a first region having a sequence that is complementary to a target sequence and a second region that hybrizes to the nuclease.
[103] The method of embodiment [101], wherein the target sequence is a sequence of the isolated cell or plurality of isolated cells.
[104] The method of any one of embodiments [93]-[103], wherein the isolated cell is a eukaryotic cell, animal cell, mammalian cell, or human cell.
[105] The method of any one of embodiments [93]-[104], wherein the isolated cell is an immune cell, progenitor cell, stem cell, neurological cell, cardiological cell, liver cell, or beta cell.
[106] The method of any one of embodiments [93]-[105], wherein the isolated cell is a peripheral blood mononuclear cell, peripheral blood lymphocyte, or lymphocyte.
[107] The method of any one of embodiments [93]-[106], wherein the isolated cell is selected from a group consisting of a T cell (e.g., a regulatory T cell, γδ T cell, αβ T cell, CD8+ T cell, or CD4+ T cell), a B cell, a Natural Killer cell, a Natural Killer T cell, a macrophage, a dendritic cell, a red blood cell, a reticulocyte, a myeloid progenitor, and a megakaryocyte.
[108] The method of any one of embodiments [93]-[104], wherein the isolated cell is selected from a group consisting of a mesenchymal stem cell, an embryological stem cell, a fetal stem cell, a placental derived stem cell, an induced pluripotent stem cell, an adipose stem cell, a hematopoietic stem cell (e.g., CD34+ cell), a skin stem cell, an adult stem cell, a bone marrow stem cell, a cord blood stem cell, an umbilical cord stem cell, a corneal limbal stem cell, a progenitor stem cell, and a neural stem cell.
[109] The method of any one of embodiments [93]-[104], wherein the isolated cell is selected from a group consisting of a fibroblast, a chondrocyte, a cardiomyocyte, a dopaminergic neuron, a microglia, a oligodendrocyte, a enteric neuron, and a hepatocyte.
[110] The method of any one of embodiments [93]-[109], wherein the isolated cell is replication incompetent.
[111] The method of any one of embodiments [93]-[110], wherein the plurality of edited cells is from 5×10⁵ cells to 1×10⁷ cells.
[112] The method of any one of embodiments [93]-[111], wherein the plurality of edited cells is from 12.5×10⁵ cells to 4.4×10¹¹ cells.
[113] The method of any one of embodiments [83]-[85] or [88]-[112], wherein the subject is a human or non-human animal.
[114] The method of any one of embodiments [83]-[85] or [88]-[113], wherein the cell or isolated cell is autologous to the subject (e.g., a treated subject or a subject in need thereof) or the cell or isolated cell is allogeneic to the subject (e.g., a treated subject or a subject in need thereof).
[115] The method of any one of embodiments [83]-[85] or [88]-[114], wherein the subject has a disease or disorder.
[116] The method of any one of embodiments [83]-[85] or [88]-[115], wherein the subject has a hyperproliferative disease, cancer, a neurodegenerative disease, a metabolic disease, an inflammatory disease, an autoimmune disease, an infectious disease, or a genetic disease.

### EXAMPLES

The following examples are provided to further illustrate some embodiments of the present invention, but are not intended to limit the scope of the invention; it will be understood by their exemplary nature that other procedures, methodologies, or techniques known to those skilled in the art may alternatively be used.

### Example 1: Expression of an Intra-Cellular Protein from a Circular RNA in Cells

This Example demonstrates in vitro assessment of expression of an intra-cellular protein of circular RNA in cells.

In this Example, circular RNAs were designed to include an IRES and an ORF encoding GFP. The circular RNA was generated in vitro via splint mediated ligation using T4 RNA Ligase 2. Primary human T-cells were activated using CD3/CD28 Dynabeads (for 3 days in T cell OpTimizer media). After bead removal, activated T cells were electroporated with 0.6 pmoles (˜250 ng) of circular RNAs using an electroporation system (Thermo Scientific).

At each time point, starting at 24 hrs post-electroporation, T cells were resuspended and a fraction of the sample was assayed for GFP expression by flow cytometry. In short, the cells were pelleted (300 g, 5 min RT) and resuspended in Flow Buffer (PBS+5% FBS) containing Dapi (1:1000 dilution) for 5 mm in the dark. After 2 washes in Flow Buffer, samples were run on a flow cytometer (Thermo Scientific) to assay for GFP expression. Dead cells and doublets were removed from the target population prior to GFP expression measurements.

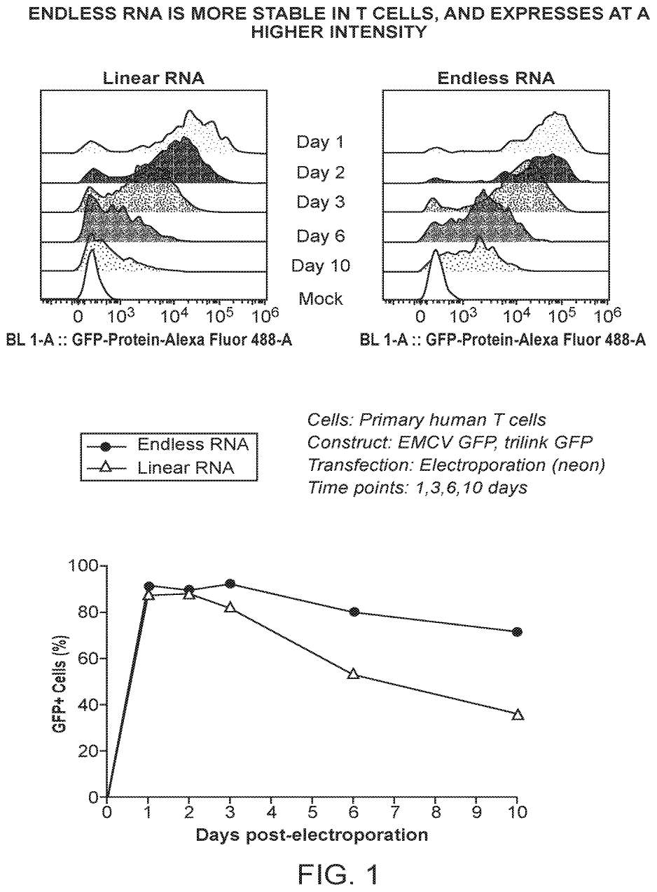

As shown in FIG. 1, GFP expression from both circular and linear RNA was detected at 1 day post electroporation (˜90% GFP+ cells). The percentage of GFP+ cells was maintained at 2 days post administration for both linear and circular mRNA-electroporated cells. However, the mean fluorescence intensity (MFI) of GFP+ cells electroporated with linear mRNA dropped by approximately 54% by Day 2, while circular mRNA-electroporated cells only dropped by about 16%. At days 3, 6, and 10 linear RNA expression decreased, while circular RNA expression remained steady (81% linear vs. 92% circular at day 3, 53% linear vs. 80% circular at day 6, and 36% linear vs. 72% circular at day 10).

Overall, the results demonstrate that cells transfected with circular RNA show prolonged expression of intra-cellular proteins compared to cells treated with linear RNA. These results further demonstrate reduced toxicity of the circular RNA compared to linear RNA.

### Example 2: Expression of a Therapeutic Membrane Protein from a Circular RNA on Cells

This Example demonstrates in vitro assessment of expression of a membrane protein from circular RNA in cells.

In this Example, circular RNAs were designed to include an IRES and an ORF encoding a CD19 chimeric antigen receptor (CAR). The circular RNA was generated in vitro via intronic self-splicing. Primary human T-cells were activated using CD3/CD28 beads for 3 days in T Cell Media and electroporated with 0.6 pmoles (˜400 ng) of circular RNAs. At each time point, starting at 24 hrs post-electroporation, the T cells were resuspended and a fraction of the cells were assayed for CD19 CAR surface expression as well as target antigen binding by flow cytometry. In short, expression of CD19 CAR was detected by first staining cells with biotinylated rabbit anti-murine IgG (H+L) antibody (1:1000 dilution, 1 hour at room temperature in the dark), washed two times, and then incubated with a streptavidin-APC secondary antibody (1:500 dilution, 1 hour at room temperature in the dark). After 2 washes in flow buffer, samples were run on a flow cytometer (Thermo Scientific) to assay for CD19 CAR expression and antigen binding. Dead cells and doublets were removed from the target population prior to CD19 CAR expression measurements.

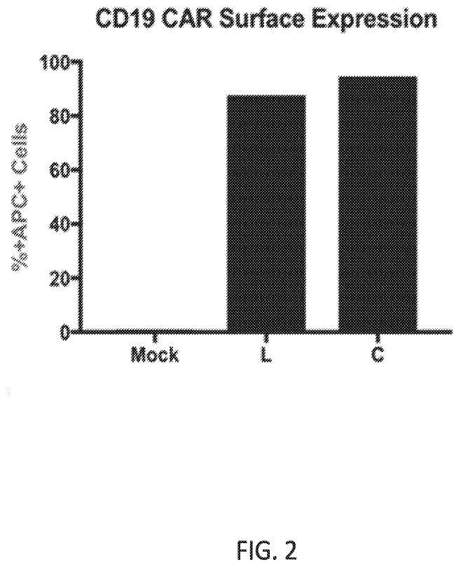

As shown in FIG. 2, CD19 CAR expression from both circular (C) and linear (L) RNA was detected at 1-day post electroporation (97% and 95%, respectively). Intensity of CD19 CAR surface expression was about 3 times higher in circular RNA-electroporated cells than linear RNA-electroporated cells.

Overall, the results demonstrate that cells transfected with circular RNA show expression of membrane proteins.

### Example 3: Circular RNA Expression of a Secreted Protein in Cells

This Example demonstrates increased half-life of circular RNA expressing a secreted protein when delivered into cells compared with linear RNA.

A non-naturally occurring circular RNA was engineered to express a biologically active secreted protein in cells. As shown in the following Example, protein expression from the circular RNA was present at higher levels compared to expression from linear RNA encoding the same protein, demonstrating a longer half-life for circular RNA in cells.

In this Example, circular RNA and linear RNA were designed to include an IRES and an ORF encoding Gaussia Luciferase and two spacer elements flanking the IRES-ORF. The circular RNA was generated in vitro as follows: unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

To monitor expression of protein from RNA in cells, 5×10³ cells were successfully reverse transfected with a lipid-based transfection reagent (Invitrogen) and 2 nM of linear or circular RNA. Gaussia Luciferase activity was monitored daily for up to 14 days in cell culture supernatants, as a measure of expression, using a Gaussia Luciferase Flash Assay Kit and following manufacturer's instructions.

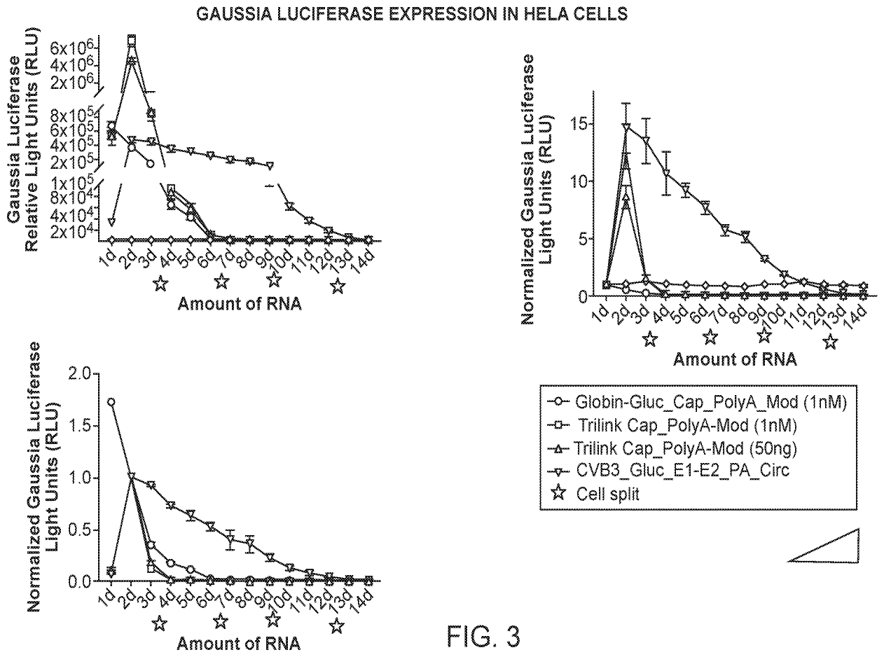

FIG. 3 shows longer secreted protein expression from circular RNA, for more than 9 days, in HeLa cells compared to 4-6 days for linear RNA.

### Example 4: Human Primary T Cells Expressed CD19 CAR from Circular and Linear RNA Constructs Encoding CD19 CAR

This Example demonstrates the ability of circular RNA to express a functional chimeric antigen receptor (CAR) as a membrane protein in human primary T cells.

In this example, circular RNAs were designed to include an IRES with an ORF encoding a CD19 chimeric antigen receptor (CAR) flanked upstream and downstream by self-splicing motifs derived from the Anabaena pre-tRNA. The circular RNA was generated by in vitro transcription using T7 RNA polymerase from a DNA segment. Transcribed RNA was purified with an RNA purification system (New England Biolabs, Inc.). Self-splicing reactions contained GTP (final concentration: 2 mM) and NEBuffer 4 (NEB, Cat #B7004S) and were purified using an RNA purification system (New England Biolabs, Inc.). To remove residual linear RNA samples were treated with RNase R (Lucigen, Cat #RNR07250). Circular RNA was Urea-PAGE purified, eluted in a buffer (0.5M Sodium Acetate, 0.1% SDS, 1 mM EDTA), ethanol precipitated and resuspended in RNase storage solution (Thermo Fisher Scientific, cat #AM7000). RNA was diluted to a concentration of 1 pmole/uL prior to use. In addition, linear RNA counterparts were generated and included the same CD19 CAR ORF, flanked upstream and downstream by the human alpha globin 5′ and 3′ UTRs, respectively.

Primary human T-cells were activated using CD3/CD28 Dynabeads (Thermo Fisher Scientific, Cat #11132D) for 3 days in T cell OpTimizer media (Thermo Fisher Scientific, Cat #A1048501). After bead removal, activated T cells (100,000 cells) were electroporated with 0.65 pmoles (˜500 ng) of circular or linear RNA using the Neon electroporation system (Thermo Fisher Scientific). RNA Storage Solution alone was used as a vehicle only control.

At 24 hrs post-electroporation, T cells were resuspended and a fraction of the sample was assayed for CD19 CAR surface expression by flow cytometry. In short, the cells were stained with FITC-conjugated recombinant CD19 (Acro Biosystems, Cat #CD9-HF2H2) resuspended in Flow Buffer (PBS+5% FBS), and was incubated at 4 degrees for 1 hour in the dark. Cells were washed two times with Flow Buffer and were stained with Dapi (diluted 1:1000 in Flow Buffer) for 5 mm in the dark. After a final wash in Flow Buffer, samples were run on an Attune NxT Flow Cytometer (Thermo Scientific) to measure for CD19-FITC binding. Cell debris, doublets, and dead cells were removed from the target population prior to CD19 binding measurements.

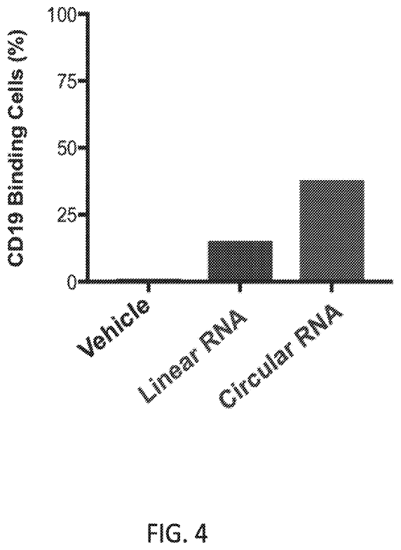

As shown in FIG. 4, CD19 CAR expression from both circular and linear RNA was detected at 24 hours post-electroporation and was observed to be higher than the vehicle only control. CD19 CAR expression from circular RNA-electroporated cells was observed to be roughly three times higher than linear RNA-electroporated cells.

This Example demonstrated that CD19 CAR was successfully expressed as a membrane protein on primary human T cells electroporated with circular and linear RNA constructs encoding the CD19 CAR protein.

### Example 5: T Cells Expressing CD19 CAR from Circular RNA Constructs can Kill Tumor Cells

This Example demonstrates the ability of circular RNA to express a functional chimeric antigen receptor as a membrane protein in human primary T cells.

In this example, circular RNAs were designed to include an IRES with an ORF encoding a CD19 chimeric antigen receptor (CAR) flanked upstream and downstream by self-splicing motifs derived from the Anabaena pre-tRNA. The circular RNA was generated by in vitro transcription using T7 RNA polymerase from a DNA segment. Transcribed RNA was purified with an RNA purification system (New England Biolabs, Inc.). Self-splicing reactions contained GTP (final concentration: 2 mM) and NEBuffer 4 (NEB, Cat #B7004S) and were purified using an RNA purification system (New England Biolabs, Inc.). To remove residual linear RNA, samples were treated with RNase R (Lucigen, Cat #RNR07250). Circular RNA was Urea-PAGE purified, eluted in a buffer (0.5M Sodium Acetate, 0.1% SDS, 1 mM EDTA), ethanol precipitated and resuspended in RNase storage solution (Thermo Fisher Scientific, cat #AM7000). RNA was diluted to a concentration of 1 pmole/uL prior to use.

Primary human T-cells were activated using CD3/CD28 Dynabeads (Thermo Fisher Scientific, Cat #11132D) for 3 days in T cell OpTimizer media (Thermo Fisher Scientific, Cat #A1048501). After bead removal, activated T cells (100,000 cells) were electroporated with 0.65 pmoles (˜500 ng) of circular RNA using the Neon electroporation system (Thermo Fisher Scientific). RNA Storage Solution alone was used as a vehicle only control.

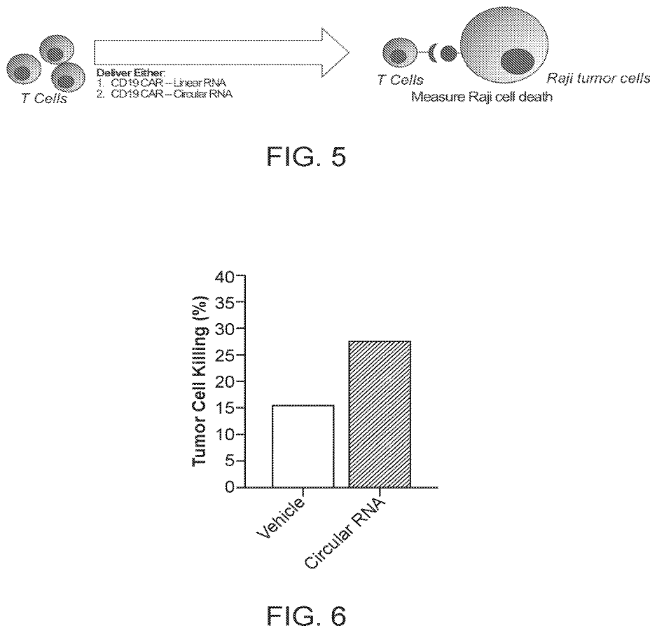

The ability of T cells expressing CD19 CAR to kill tumor cells was determined by tumor cell killing assay (FIG. 5). Briefly, Raji tumor cells expressing CD19 surface antigen were stained with the membrane dye PHK26 (Sigma, Cat #MINI26) according to manufacturer's instructions and then were incubated with CD19 CAR expressing T cells at an effector-target ratio ranging from 1:1 to 20:1 for 18 hours at 37 degrees. Afterwards, the cell suspension was stained with Dapi (diluted 1:1000 in Flow Buffer) for 5 min in the dark. Cell suspensions were directly transferred to FACS tubes and run on the Attune NxT Flow Cytometer (Thermo Scientific) to measure tumor cell killing by gating on double positive (PKH+, Dapi+) cells, which represented the % of dead Raji (tumor cells) in the total cell population. Cell debris and doublets were removed from the target population prior to tumor cell killing assay measurements.

As shown in FIG. 6, T cells expressing CD19 CAR derived from transfected circular RNA exhibited greater Raji tumor cell killing capacity compared to the vehicle only control. This suggests CD19 CAR-dependent killing of the Raji tumor cells.

This Example demonstrated that functional CD19 CAR was successfully expressed as a membrane protein on primary human T cells electroporated with circular RNA constructs encoding the CD19 CAR sequence. It further demonstrated CD19 CAR-dependent downstream effector function of the electroporated T cell with therapeutic implications. T cells carrying the CD19 CAR expressed from circular RNA were able to kill tumor cells.

### Example 6: Delivery of Circular RNA or Modified Linear RNA with a Carrier to Human Retina Cell Line and Translation into Protein

This example demonstrates delivery of unmodified circular RNA to human retinal pigmented epithelial cell line ARPE-19.

In this example, eGFP mRNA was purchased (Trilink Biotechnologies, L-7201) and contains a codon optimized eGFP ORF distinct from the circular RNA template. The mRNA contained the conventional modifications necessary for optimal cap-dependent translation (5′ and 3′ human beta-globin UTRs, 5′ Cap, 3′ Poly(A) tail, 100% methoxy-pseudouridine nucleotide substitutions).

In this example, circular RNA was designed with an encephalomyocarditis virus (EMCV) internal ribosome entry site (IRES) and an open reading frame (ORF) encoding enhance green fluorescent protein (eGFP).

The circular RNA was generated in vitro. Unmodified linear RNA was transcribed in vitro (Lucigen, ASF3507) from a DNA template including all the motifs listed above, as well as a T7 RNA polymerase promoter to drive transcription. Transcribed RNA was purified with an RNA cleanup kit (New England Biolabs, T2050), treated with RNA 5′ phosphohydrolase (RppH) (New England Biolabs, M0356), and purified again with the same type of RNA purification column. RNA was circularized using a splint DNA (5′-GGCTATTCCCAATAGCCGTT-3′) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in water under sterile conditions.

RNA was diluted in water to a concentration of 45 g/L (1 uM) and then complexed with a lipofectamine carrier (Thermo Fisher Scientific, LMRNA003) in a total volume of 10 uL. A total of 0.1 pmoles of RNA was transfected into 5,000 ARPE-19 cells, plated in Dulbecco's Modified Eagle's Medium (DMEM):F12 (American Type Cell Culture, 30-2006) supplemented with 10% fetal bovine serum (FBS) at 37° C. All reagents were brought to room temperature prior to mixing and mixtures were prepared immediately prior to use following the manufacturer's instructions. As a negative control, untreated controls (without carrier and without RNA) were used.

To determine RNA translation persistence in cells, culture plates were daily analyzed for green fluorescence by using an EVOS Cell Imaging System M7000 (Thermo Fisher Scientific). Cultures images were taken in bright field (visible wavelengths) and green fluorescence (“GFP channel”, 510 nm), at 4×, 10× and 20× magnification. Fluorescence signal was considered positive when colocalizing with an intact cell as images from bright field and fluorescence were superimposed.

Fluorescence signal by eGFP was detected in cells at 16 hours after transfection with circular RNA and also after transfection with modified mRNA.

This example demonstrated that circular RNA was successfully delivered and efficiently translated in human retina cell culture, ARPE-19 cells, via transfection in presence of a carrier.

### Example 7: Circular RNA Expression of Functional Phenylalanine Hydroxylase in Cells, Converting Phenylalanine to Tyrosine

This Example demonstrates the ability of circular RNA to express a functional enzyme with therapeutic effects in cells.

Phenylalanine hydroxylase (PAH) is an enzyme that catalyzes the hydroxylation of phenylalanine to generate tyrosine. The principal source of phenylalanine in humans is ingested proteins, the majority of which is then catabolized through PAH to form tyrosine which can then be broken down in subsequent catabolic steps. Mutations in the PAH encoding gene can lead to phenylketonuria, a severe metabolic disorder, where phenylalanine levels are elevated in the body. Expression of functional PAH in disease can reduce phenylalanine levels in the body and therefore have therapeutic benefit.

In this example, circular RNAs were designed to include CVB3 IRES with an ORF encoding mouse phenylalanine hydrolyase (mPAH) and a spacer (either IS or E1E2). To generate circular RNA, linear RNA was generated by in vitro transcription using T7 RNA polymerase from a DNA segment. Transcribed RNA was purified with an RNA purification column (New England Biolabs, T2050), treated with RNA 5′ Pyrophosphohydrolase (RppH) (New England Biolabs, M0356) following the manufacturer's instructions, and purified again with the RNA purification column (New England Biolabs, T2050). RppH treated linear RNA was circularized using a splint DNA (5′-GTTTTTCGGCTATTCCCAATAGCCGTTTTG-3′ for IS, 5′-GTCAACGGATTTTCCCAAGTCCGTAGCGTCTC-3′ for E1E2) and T4 RNA ligase 2 (New England Biolabs, M0239). Circular RNA was Urea-PAGE purified, eluted in a buffer (0.5M Sodium Acetate, 0.1% SDS, 1 mM EDTA), ethanol precipitated and resuspended in RNA storage solution (Thermo Fisher Scientific, AM7000).

Each circular RNA was then transfected into HEK293T cells using MassengerMax (Invitrogen) according to the manufacturer's instructions. 2 pmole of circular RNA was used to transfect one million cells and plated to a 6 well plate. For negative control, vehicle only was used.

To prepare the cell extracts for downstream analysis, transfected cells were collected after 24 and 72 hours by scraping and pelleted by centrifugation. The cell pellets were resuspended in PBS buffer (pH 7.4) with 50 mM sucrose, 0.2 mM PMSF and protease inhibitor cocktail (Thermo Fisher Scientific, 78430). Cells were homogenized by passaging through a fine needle (20×). Sucrose concentration was then increased to 0.25 M and extracts were clarified via centrifugation (14000 g, 10 min, 4° C.). Protein concentrations were normalized using BCA protein assays (ThermoFisher Scientific).

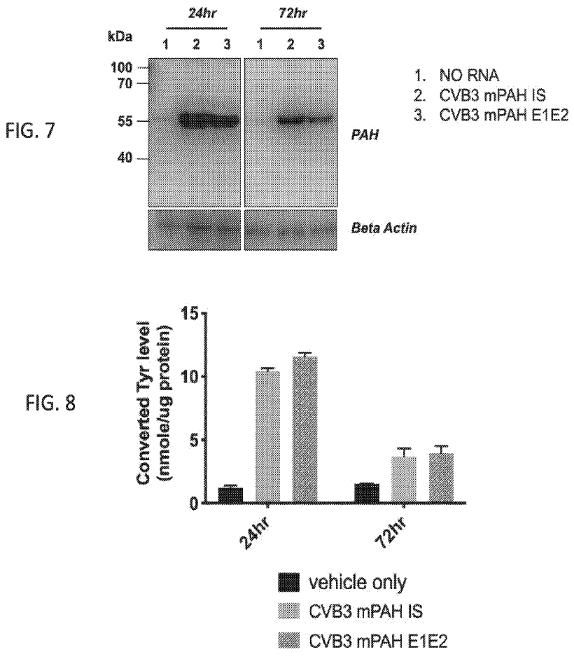

PAH levels was assessed by Western blot. Briefly, 1.5 ug of protein in LDS sample buffer (Invitrogen) was separated on 12% Bis-Tris gel (Invitrogen) and transferred to Nitrocellulose membrane by iBlot2 drying blot system. Protein was detected with rabbit antibodies against PAH (Abcam) and beta actin (Abcam, loading control) as primary antibodies and a horseradish peroxidase-linked anti-rabbit immunoglobulin G as a secondary antibody. Membrane-bound antibodies were detected by enhanced chemiluminescence (Thermo Scientific) using a Imaging system (LI-COR). PAH protein was observed by Western blot and expressed in cells using both circular RNAs tested to a greater extent than with the vehicle only control (FIG. 7).

To measure PAH activity, 10 μg of cell lysate was preincubated with 1 mM L-phenylalanine and 1 mg/ml catalase for 5 mm (30° C.) in 100 mM Na-HEPES (pH 7.3). Ferrous ammonium sulphate was added to a final concentration of 100 uM and incubated for 1 min. The reaction was initiated by adding BH4 (75 uM final concentration) and DTT (2 mM final concentration) and incubated for 2 hours at 30° C. The reaction was halted by incubating the samples at 95° C. for 10 min and clarified via centrifugation (14000 g, 3 min). Tyrosine level converted by PAH from phenylalanine during reaction was measured by Tyrosine assay kit (Sigma, MAK2019) according to the manufacturer's instructions.

As shown in FIG. 8, PAH protein expressed in cells by both circular RNAs tested was functional and able to convert phenylalanine to tyrosine. Tyrosine levels in cells treated with circular RNA was greater than in cells treated with the vehicle only control. PAH expressed from circular RNA bearing the PAH ORF showed significant enzymatic activity, approximately 10 folds higher relative to the vehicle only control. The enzymatic activity shown by in vitro assay was corelated with the expression of PAH protein and sustained up to 3 days.

This example demonstrated successful expression of functional protein in HEK293 cells from circular RNA.

### Example 8: Circular RNA is More Stable in Cells than Linear RNA

This Example demonstrates increased stability of circular RNA expressing a secreted protein when delivered into cells compared with linear RNA.

A non-naturally occurring circular RNA was engineered to express a biologically active secreted protein in cells. As shown in the following Example, circular RNA was detected longer compared to linear RNA encoding the same protein, demonstrating a longer half-life for circular RNA in cells.

In this Example, circular RNA and linear RNA were designed to include an IRES and an ORF encoding Gaussia Luciferase and two spacer elements flanking the IRES-ORF. The circular RNA is generated in vitro as follows: unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

To monitor RNA stability in cells, 5×10³ cells were successfully reverse transfected with a lipid-based transfection reagent (Invitrogen) and 2 nM of linear or circular RNA. Cell lysates were collected to monitor RNA levels by quantitative RT-PCR. Circular RNA levels were analyzed by GLuc-specific QPCR at 6 hrs and 1-4 days post-transfection. In brief, cDNA was generated from cell lysates by random priming using the Power SYBR Green cells to ct kit (ThermoFisher Scientific, cat #4402953) and following manufacturer's instructions. Fold-change was calculated using the Pfaffl method, using β-Actin as housekeeping gene.

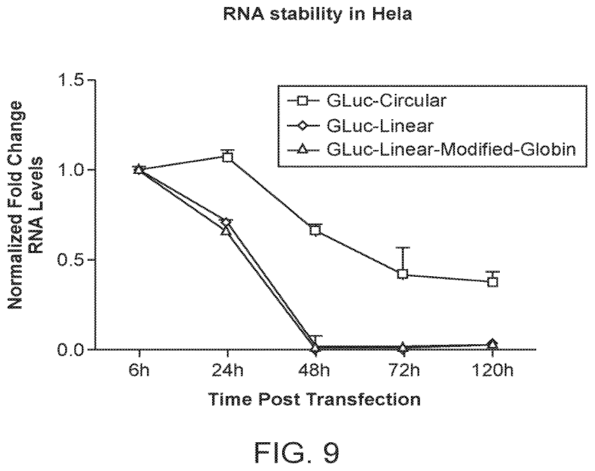

FIG. 9 shows circular RNA is stable more than 4 days (120 h) in HeLa cells, compared to 2 days for linear RNAs.

### Example 9: Circular RNA Less Immunogenic in Cells than Linear RNA

This Example demonstrates less immunogenic response elicited by circular RNA expressing a secreted protein when delivered into cells compared with linear RNA.

A non-naturally occurring circular RNA was engineered to express a biologically active secreted protein in cells. As shown in the following Example, circular RNA induces less expression of the immune response genes RIGI and MDA5 compared to linear RNA encoding the same protein, demonstrating less immunogenicity from circular RNA in cells.

In this Example, circular RNA and linear RNA were designed to include an IRES and an ORF encoding Gaussia Luciferase and two spacer elements flanking the IRES-ORF. The circular RNA was generated in vitro as follows: unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

To monitor RNA stability in cells, 5×10³ cells were successfully reverse transfected with a lipid-based transfection reagent (Invitrogen) and 2 nM of linear or circular RNA. Cell lysates were collected to monitor RNA levels by quantitative RT-PCR. Circular RNA levels were analyzed by GLuc-specific QPCR at 6 hrs and 1-4 days post-transfection. In brief, cDNA was generated from cell lysates by random priming using the Power SYBR Green cells to et kit (ThermoFisher Scientific, cat #4402953) and following manufacturer's instructions. Fold-change was calculated using the Pfaffl method, using β-Actin as housekeeping gene.

The results showed less immunogenicity in cells from circular RNA in HeLa cells compared to linear RNAs.

### Example 10: Circular RNA Less Toxic in Cells than Linear RNA

This Example demonstrates less cell toxicity elicited by circular RNA expressing a secreted protein when delivered into cells compared with linear RNA.

A non-naturally occurring circular RNA was engineered to express a biologically active secreted protein in cells. As shown in the following example, cell growth is less affected when cells are transfected circular RNA when compared to linear RNA encoding the same protein, demonstrating less cytotoxicity from circular RNA in cells.

In this Example, circular RNA and linear RNA were designed to include an IRES and an ORF encoding Gaussia Luciferase and two spacer elements flanking the IRES-ORF. The circular RNA was generated in vitro as follows: unmodified linear RNA was synthesized by in vitro transcription using T7 RNA polymerase from a DNA segment. Transcribed RNA was purified with an RNA purification system (New England Biolabs, Inc.), treated with RNA 5′ Pyrophosphohydrolase (RppH) (New England Biolabs, Inc., M0356) following the manufacturer's instructions, and purified again with the RNA purification system. Splint ligated circular RNA was generated by treatment of the transcribed linear RNA and a DNA splint with T4 RNA ligase 2 (New England Biolabs, Inc., M0239).

To purify the circular RNAs, ligation mixtures were resolved on 4% denaturing PAGE and RNA bands corresponding to each of the circular and linear RNAs were excised. The linear RNAs were purified using the same 4% denaturing PAGE gel. Excised RNA gel fragments (linear or circular) were crushed, and RNA was eluted with gel elution buffer (0.5M NaOAc, 1 mM EDTA and 0.1% SDS) for an hour at 37° C. Supernatant was harvested, and RNA was eluted once again by adding gel elution buffer to the crushed gel and incubated for an hour. Gel debris was removed by centrifuge filters and RNA was precipitated with ethanol.

To monitor cell toxicity in cells, 5×10³ cells were successfully reverse transfected with a lipid-based transfection reagent (Invitrogen) and 2 nM of linear or circular RNA. Cell viability was used as a direct measure of cell toxicity. Bright field imaging as well as ATP production were used to monitor cell viability. Cells were imaged in culture and Cell lysates were collected to monitor ATP levels using a CellTite-Glo kit (Promega) and luminescence was measured following manufacture's instructions.

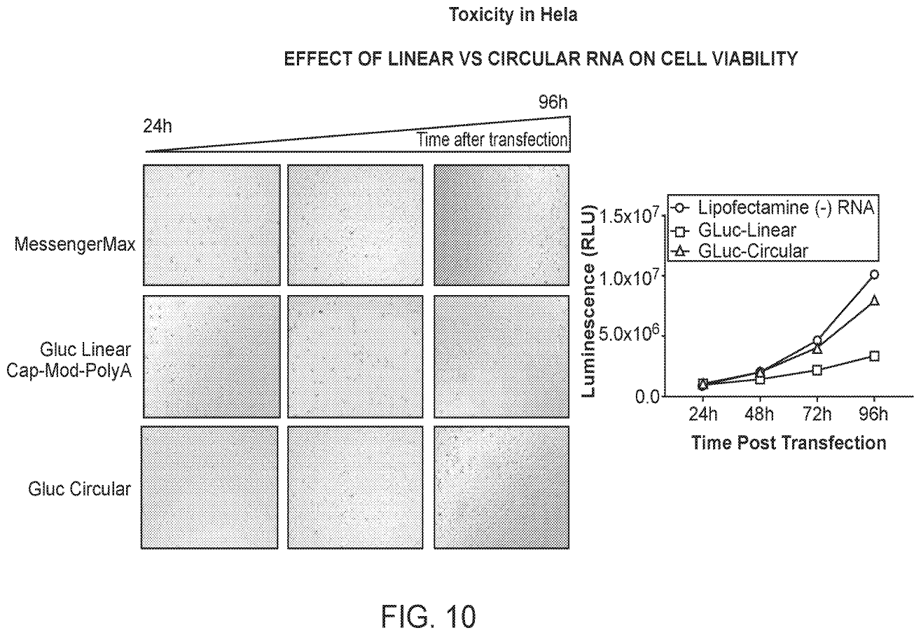

FIG. 10 show less toxicity in cells from circular RNA compared to linear RNAs.

### Example 11: Circular RNA Mediated Delivery Directly into Specific Cell Types

This Example demonstrates the ability to target circular RNA to therapeutically-relevant proteins on a target cell via RNA aptamer sequences contained within the circular RNA.

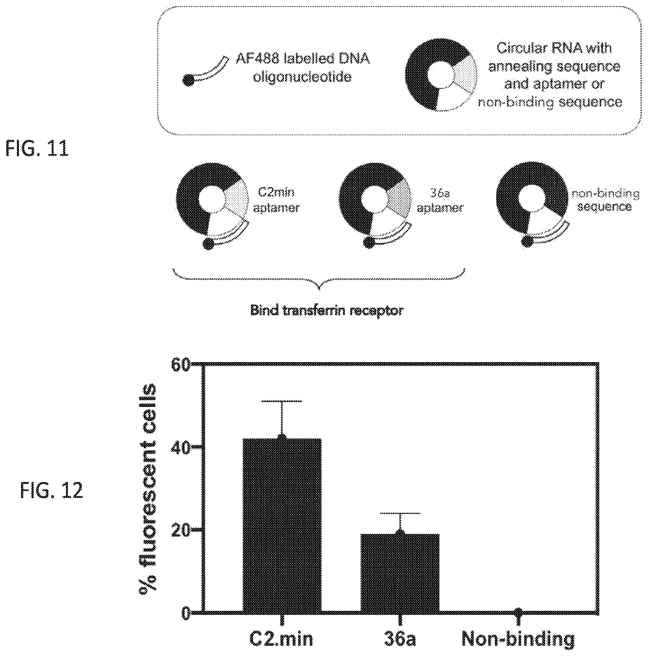

For this Example, circular RNA included either an C2 min aptamer sequence known to bind competitively to the human transferrin receptor (5′-GGG GGA UCA AUC CAA GGG ACC CGG AAA CGC UCC CUU ACA CCC C-3′); or, a 36a (5′-GGG UGA AUG GUU CUA CGA UAA ACG UUA AUG ACC AGC UUA UGG CUG GCA GUU CCU AUA GCA CCC-3′) aptamer sequence known to bind non-competitively to the human transferrin receptor. Circular RNAs were designed to include a spacer region for hybridization of a fluorescent single stranded DNA oligonucleotide for visualization. A control circular RNA including an aptamer sequence that is predicted not to bind to human transferrin receptor was also used. A schematic of these circular RNAs is shown in FIG. 11.

The circular RNA was generated in vitro. Unmodified linear RNA was transcribed in vitro from a DNA template including all the motifs listed above, as well as a T7 RNA polymerase promoter to drive transcription. Transcribed RNA was purified with an RNA cleanup kit (New England Biolabs, T2050), treated with RNA 5′ phosphohydrolase (RppH) (New England Biolabs, M0356) following the manufacturer's instructions, and purified again with an RNA purification column. RppH treated linear RNA was circularized using a splint DNA (5′-TGT TGT GTC TTG GTT GGT-3′ or 5′-TGT TGT GTG TTG GTT GGT-3′) and T4 RNA ligase 2 (New England Biolabs, M0239). Circular RNA was Urea-PAGE purified, eluted in a buffer (0.5 M Sodium Acetate, 0.1% SDS, 1 mM EDTA), ethanol precipitated and resuspended in RNase storage solution (ThermoFisher Scientific, cat #AM7000).

A short single-stranded DNA oligonucleotide with AlexaFluor488 was used to label the aptamer for intracellular visualization (5′-AF488-TGT TGT GTC TTG GTT GGT-3′ or 5′-AF488-TGT TGT GTG TTG GTT GGT-3′, Integrated DNA Technologies, IDT). The fluorescent ssDNA oligonucleotide was added at 3× molar excess over the circular RNA, incubated at 60° C. for 10 minutes followed by a 20 minute incubation at room temperature in the presence of 150 mM KCL. RNA buffer was exchanged to PBS using a Microbiospin column (Biorad).

Circular RNA annealed with the AlexaFluor488-DNA oligonucleotide was added to HeLa cells at 0.1 μM final concentration in 100 μL of Optimem media. After one hour of incubation at 37° C., cells were washed with phosphate-buffered saline solution and transferred to Fluorobrite with DAPI solution. Cells were imaged using an Evos cell imager (ThermoFischer Scientific).

Circular RNA binding to human transferrin was evaluated by fluorescent microscopy. AlexaFluor488 activity was detected inside HeLa cells as punctate fluorescent signals when C2 min and 36a aptamers were contained in the circular RNA (FIG. 12). In contrast, no fluorescent signal was observed for the control circular RNA containing a non-binding aptamer sequence. This indicates aptamer sequences contained within the circular RNA are responsible for internalization via transferrin receptor binding.

This Example demonstrated that RNA aptamer sequences encoded within in circular RNA bind target proteins and can increase uptake into mammalian cells via interaction with the specific surface receptor.

### Example 12: Circular RNA Hybridized to Single-Stranded RNA Oligonucleotides Containing RNA Aptamer Sequences can Target Surface Proteins and Enable Uptake of the Circular RNA

This Example describes targeting of circular RNA to therapeutically relevant proteins on a target cell via RNA aptamer sequences contained in a single-stranded RNA oligonucleotide that hybridizes to the circular RNA.

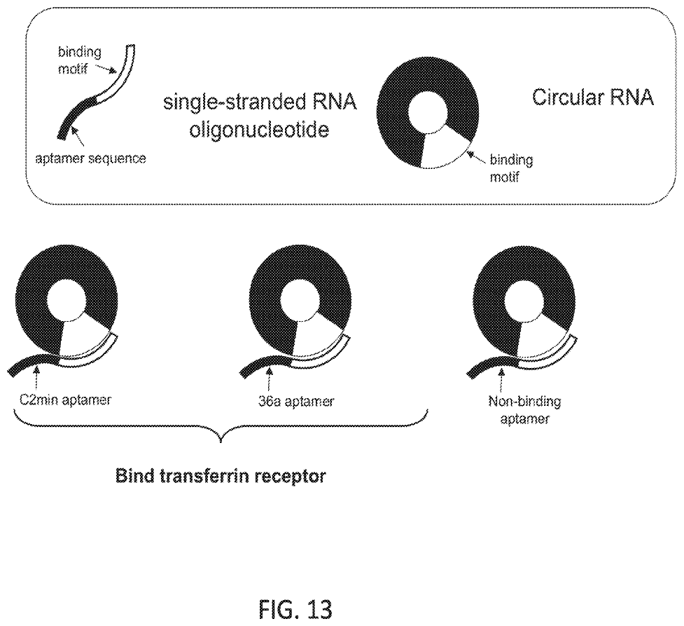

In this Example, the linear single-stranded RNA oligonucleotide includes either an C2 min aptamer sequence known to bind competitively to the human transferrin receptor (5′-GGG GGA UCA AUC CAA GGG ACC CGG AAA CGC UCC CUU ACA CCC C-3′); or, a 36a (5′-GGG UGA AUG GUU CUA CGA UAA ACG UUA AUG ACC AGC UUA UGG CUG GCA GUU CCU AUA GCA CCC-3′) aptamer sequence known to bind non-competitively to the human transferrin receptor. This linear single-stranded RNA oligonucleotide also includes a binding motif for hybridization to the circular RNA. Circular RNAs include the complementary binding region for hybridization of the aptamer-containing single-stranded oligonucleotide as well as an EMCV IRES and Gaussia Luciferase (GLuc) ORF. A control complex is generated using the same circular RNA as described above that hybridizes to a single-stranded linear RNA oligonucleotide including an aptamer sequence that is predicted not to bind to human transferrin receptor. A schematic of these entities is shown in FIG. 13.

The circular RNA is generated in vitro. Unmodified linear RNA is transcribed in vitro from a DNA template including all the motifs listed above, as well as a T7 RNA polymerase promoter to drive transcription. Transcribed RNA is purified with an RNA cleanup kit (New England Biolabs, T2050), treated with RNA 5′ phosphohydrolase (RppH) (New England Biolabs, M0356) following the manufacturer's instructions, and purified again with an RNA purification column. RppH treated linear RNA is circularized using a splint DNA (5′-TGT TGT GTC TTG GTT GGT-3′ or 5′-TGT TGT GTG TTG GTT GGT-3′) and T4 RNA ligase 2 (New England Biolabs, M0239). Circular RNA is Urea-PAGE purified, eluted in a buffer (0.5 M Sodium Acetate, 0.1% SDS, 1 mM EDTA), ethanol precipitated and resuspended in RNase storage solution (ThermoFisher Scientific, cat #AM7000).

In this example, the linear single-stranded RNA oligonucleotide is custom-synthesized by Integrated DNA Technologies (IDT) and contains the aforementioned aptamer sequence and binding motif.

The linear single stranded RNA oligonucleotide (1) is unmodified; or (2) contains 5′-fluoro modifications, as described in Kratschmer et al., (2017) Nucleic Acid Ther. 27(6):335-344; or (3) is modified to include modifications such as a 5′-hydroxyl moiety, or 2′-O-methyl modifications.

The single-stranded RNA oligonucleotide is added at 3× molar excess over the circular, incubated at 60° C. for 10 minutes and then gradually cooled to room temperature in the presence of 150 mM KCL. RNA buffer is exchanged to PBS using a Microbiospin column (Biorad). Annealing is confirmed by agarose gel electrophoresis.

Circular RNA annealed with the aptamer-containing RNA oligonucleotide is added to HeLa cells at 0.1 μM final concentration in 100 μL of Optimem media. Multiple timepoints are studied. After 1 hour, 6 hour, 12 hour, 24 hour and 48 hours of incubation at 37° C., cells are harvested.

Efficiency of delivery for each construct is measured using qRT-PCR. After harvesting, Power SYBR Green Cell to Ct kit (Invitrogen, cat #4402953) is used for lysing cells and reverse transcription according to the manufacturer's instruction. qRT-PCR will be performed with GLuc-specific primers (forward; CCTGAGATTCCTGGGTTCAAG reverse; CTTCTTGAGCAGGTCAGAACA) and iTaq Universal SYBR Green Supermix (Bio-RAD, cat #1725120) and monitored by Real-Time PCR detection system (Bio-RAD).

### Example 13: Isolation and Purification of Circular RNA

This Example demonstrate circular RNA purification.

In certain embodiments, circular RNAs, as described in the previous Examples, may be isolated and purified before expression of the encoded protein products. This Example demonstrates isolation using UREA gel separation. As shown in the following Example, circular RNA was isolated and purified.

CircRNA1 was designed to encode triple FLAG tagged EGF without stop codon (264 nts). It has a Kozak sequence at the start codon for translation initiation (SEQ ID NO: 11). CirRNA2 has identical sequences with circular RNA1 except it has a termination element (triple stop codons) (273 nts, SEQ ID NO: 12). Circular RNA3 was designed to encode triple FLAG tagged EGF flanked by a stagger element (2A sequence), without a termination element (stop codon) (330 nts, SEQ ID NO: 28). CircRNA4 has identical sequences with circular RNA3 except it has a termination element (triple stop codon) (339 nts). CircRNA5 was designed to encode FLAG tagged EGF flanked by a 2A sequence and followed by FLAG tagged nano luciferase (873 nts, SEQ ID NO: 29). CircRNA6 has identical sequence with circular RNA5 except it included a termination element (triple stop codon) between the EGF and nano luciferase genes, and a termination element (triple stop codon) at the end of the nano luciferase sequence (762 nts, SEQ ID NO: 30). CircRNA1, CircRNA2, CircRNA3, CircRNA4, CircRNA5, and CircRNA6 were isolated as described herein.

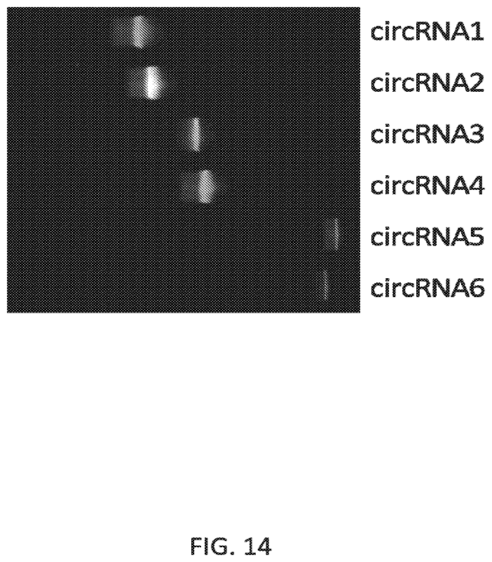

In this Example, linear and circular RNA were generated as described. To purify the circular RNAs, ligation mixtures were resolved on 6% denaturing PAGE and RNA bands corresponding to each of the circular RNAs were excised. Excised RNA gel fragments were crushed and RNA was eluted with 800 μl of 300 mM NaCl overnight. Gel debris was removed by centrifuge filters and RNA was precipitated with ethanol in the presence of 0.3M sodium acetate. Eluted circular RNA was analyzed by 6% denaturing PAGE, see FIG. 14.

Single bands were visualized by PAGE for the circular RNAs having variable sizes.

### Example 14: Circular RNA Demonstrated a Longer Half-Life than Linear RNA in Cells

This Example demonstrates circular RNA delivered into cells and has an increased half-life in cells compared with linear RNA.

A non-naturally occurring circular RNA was engineered to express a biologically active therapeutic protein. As shown in the following Example, circular RNA was present at higher levels compared to its linear RNA counterpart, demonstrating a longer half-life for circular RNA.

In this Example, circular RNA and linear RNA were designed to encode a Kozak, EGF, flanked by a 2A, a stop or no stop sequence (SEQ ID NOs: 9-12). To monitor half-life of RNA in cells, 0.1×10⁶ cells were plated onto each well of a 12 well plate. After 1 day, 1 μg of linear or circular RNA was transfected into each well using a lipid-based transfection reagent (Invitrogen). Twenty-four hours after transfection, total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad). Primer sequences were as follow: Primers for linear or circular RNA, F: ACGACGGTGTGTGCATGTAT, R: TTCCCACCACTTCAGGTCTC; primers for circular RNA, F: TACGCCTGCAACTGTGTTGT, R: TCGATGATCTTGTCGTCGTC.

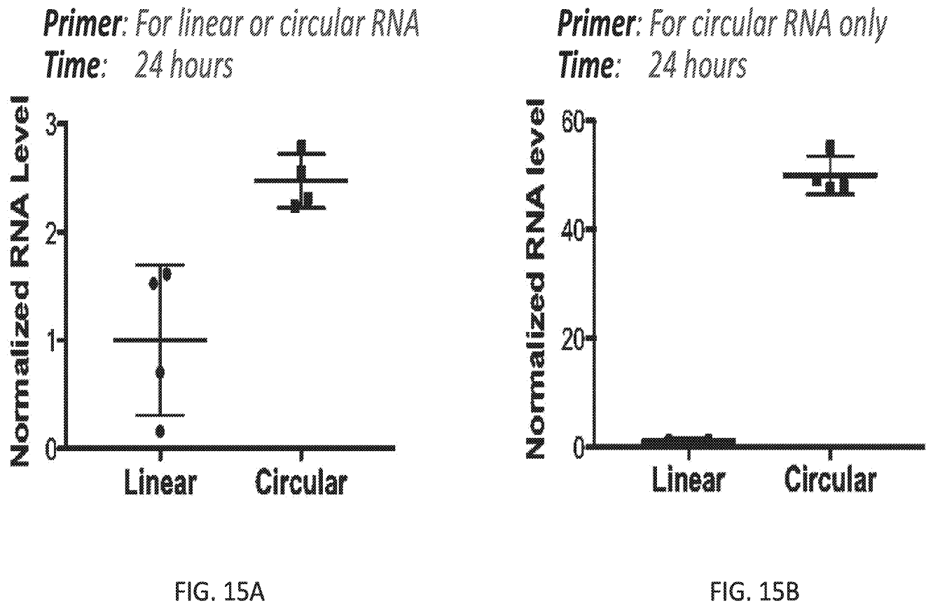

Circular RNA was successfully transfected into 293T cells, as was its linear counterpart. After 24 hours, the circular and linear RNA that remained were measured using qPCR. As shown in FIG. 15A and FIG. 15B, circular RNA was shown to have a longer half-life in cells compared to linear RNA.

### Example 15: Synthetic Circular RNA Demonstrated Reduced Immunogenic Gene Expression in Cells

This Example demonstrates circular RNA engineered to have reduced immunogenicity as compared to a linear RNA.

Circular RNA that encoded a therapeutic protein provided a reduced induction of immunogenic related genes (RIG-I, MDA5, PKA and IFN-beta) in recipient cells, as compared to linear RNA. RIG-I can recognize short 5′ triphosphate uncapped double stranded or single stranded RNA, while MDA5 can recognize longer dsRNAs. RIG-I and MDA5 can both be involved in activating MAVS and triggering antiviral responses. PKR can be activated by dsRNA and induced by interferons, such as IFN-beta. As shown in the following Example, circular RNA was shown to have a reduced activation of an immune related genes in 293T cells than an analogous linear RNA, as assessed by expression of RIG-I, MDA5, PKR and IFN-beta by q-PCR.

The circular RNA and linear RNA were designed to encode either (1) a Kozak, 3×FLAG-EGF sequence with no termination element (SEQ ID NO:9); (2) a Kozak, 3×FLAG-EGF, flanked by a termination element (stop codon) (SEQ ID NO:21); (3) a Kozak, 3×FLAG-EGF, flanked by a 2A sequence (SEQ ID NO:10); or (4) a Kozak, 3×FLAG-EGF sequence flanked by a 2A sequence followed by a termination element (stop codon) (SEQ ID NO:11).

In this Example, the level of innate immune response genes were monitored in cells by plating 0.1×10⁶ cells into each well of a 12 well plate. After 1 day, 1 μg of linear or circular RNA was transfected into each well using a lipid-based transfection reagent (Invitrogen). Twenty-four hours after transfection, total RNA was isolated from cells using a phenol-based extraction reagent (Invitrogen). Total RNA (500 ng) was subjected to reverse transcription to generate cDNA. qRT-PCR analysis was performed using a dye-based quantitative PCR mix (BioRad).

Primer sequences used: Primers for
GAPDH, F:
AGGGCTGCTTTTAACTCTGGT,
R:
CCCCACTTGATTTTGGAGGGA;
RIG-I, F:
TGTGGGCAATGTCATCAAAA,
R:
GAAGCACTTGCTACCTCTTGC;
MDA5, F:
GGCACCATGGGAAGTGATT,
R:
ATTTGGTAAGGCCTGAGCTG;
PKR, F:
TCGCTGGTATCACTCGTCTG,
R:
GATTCTGAAGACCGCCAGAG;
IFN-beta, F:
CTCTCCTGTTGTGCTTCTCC,
R:
GTCAAAGTTCATCCTGTCCTTG.

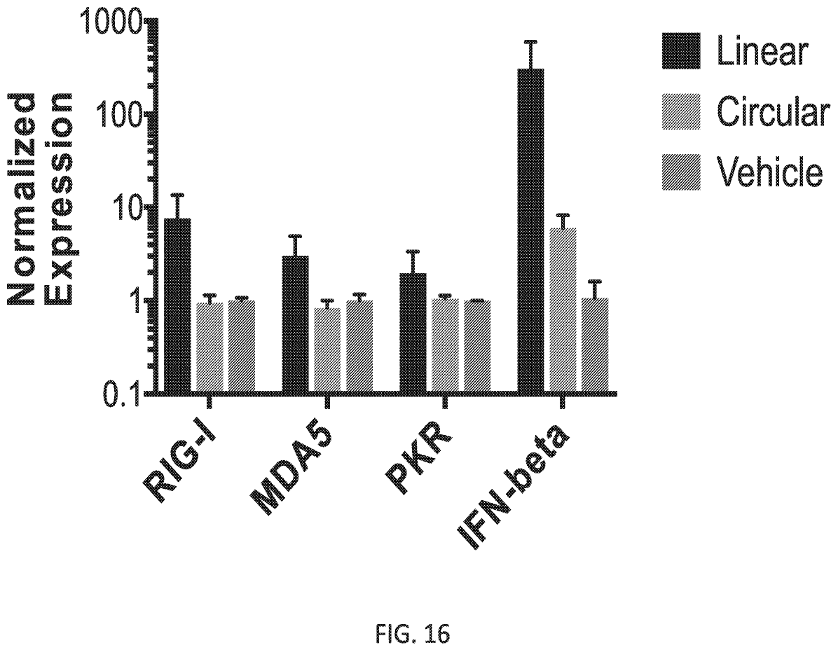

As shown in FIG. 16, qRT-PCR levels of immune related genes from 293T cells transfected with circular RNA showed reduction of RIG-I, MDA5, PKR and IFN-beta as compared to linear RNA transfected cells. Thus, induction of immunogenic related genes in recipient cells was reduced in circular RNA transfected cells, as compared to linear RNA transfected cells.

### Example 16: Increased Expression from Synthetic Circular RNA Via Rolling Circle Translation in Cells

This Example demonstrates increased expression from rolling circle translation of synthetic circular RNA in cells.

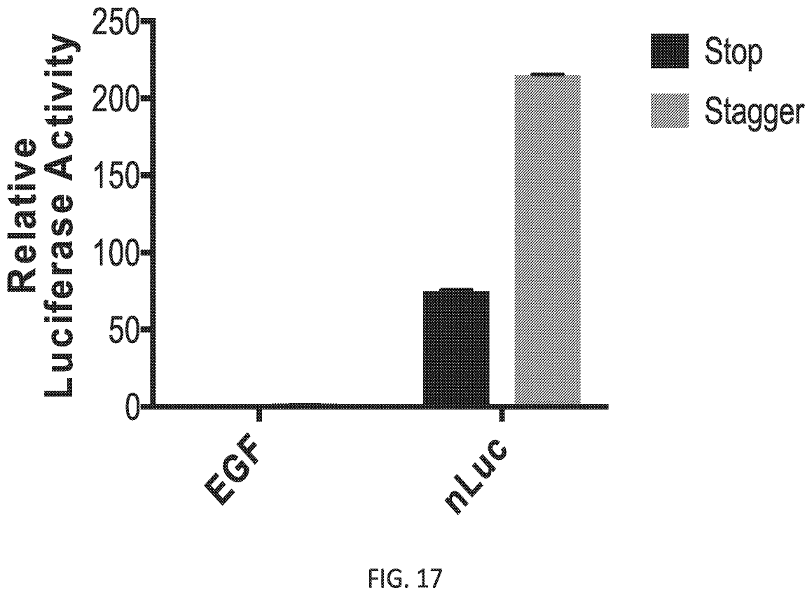

Circular RNAs were designed to include an IRES with a nanoluciferase gene or an EGF negative control gene without a termination element (stop codon). Cells were transfected with EGF negative control (SEQ ID NO:13); nLUC stop (SEQ ID NO:14): EMCV IRES, stagger sequence (2A sequence), 3×FLAG tagged nLUC sequences, stagger sequence (2A sequence), and a stop codon; or nLUC stagger (SEQ ID NO:15): EMCV IRES, stagger sequence (2A sequence), 3×FLAG tagged nLUC sequences, and stagger sequence (2A sequence). As shown in the FIG. 17, both circular RNAs produced translation product having functional luciferase activity.

In this Example, translation of circular RNA was monitored in cells. Specifically, 0.1×10⁶ cells were plated onto each well of a 12 well plate. After 1 day, 300 ng of circular RNA was transfected into each well using a lipid-based transfection reagent (Invitrogen). After 24 hrs, cells were harvested by adding 100 μl of RIPA buffer. Nanoluciferase activity in lysates was measured using a luciferase assay system according to its manufacturer's protocol (Promega).

As shown in FIG. 17, both circular RNAs expressed protein in cells. However, circular RNA with a stagger element, e.g., 2A sequence, that lacks a termination element (stop codon), produced higher levels of protein product having functional luciferase activity than circular RNA with a termination element (stop codon).

### Example 17: Increased Protein Expressed from Circular RNA

This Example demonstrates synthetic circular RNA translation in cells. Additionally, this Example shows that circular RNA produced more expression product of the correct molecular weight than its linear counterpart.

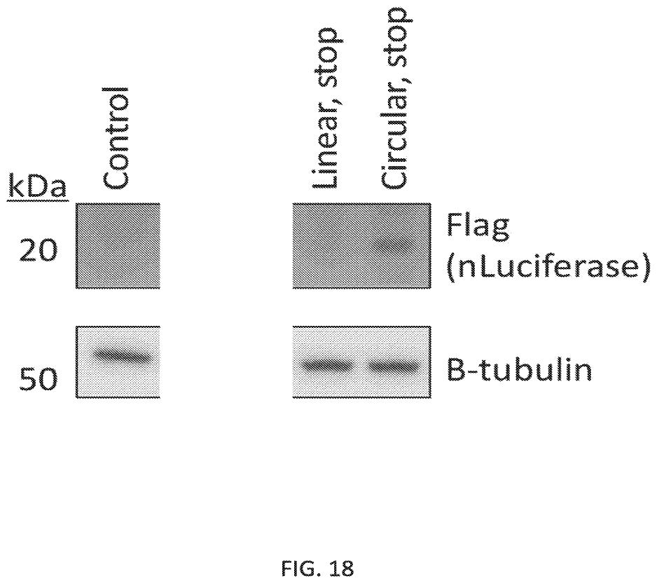

Linear and circular RNAs were designed to include a nanoluciferase gene with a termination element (stop codon). Cells were transfected with vehicle: transfection reagent only; linear nLUC (SEQ ID NO:14): EMCV IRES, stagger element (2A sequence), 3×FLAG tagged nLuc sequences, a stagger element (2A sequence), and termination element (stop codon); or circular nLUC (SEQ ID NO:14): EMCV IRES, stagger element (2A sequence), 3×FLAG tagged nLuc sequences, a stagger element (2A sequence), and a termination element (stop codon). As shown in the FIG. 18, circular RNA produced greater levels of protein having the correct molecular weight as compared to linear RNA.

After 24 hrs, cells were harvested by adding 100 μl of RIPA buffer. After centrifugation at 1400×g for 5 min, the supernatant was analyzed on a 10-20% gradient polyacrylamide/SDS gel.

After being electrotransferred to a nitrocellulose membrane using dry transfer method, the blot was incubated with an anti-FLAG antibody and anti-mouse IgG peroxidase. The blot was visualized with an ECL kit and western blot band intensity was measured by ImageJ.

As shown in FIG. 18, circular RNA was translated into protein in cells. In particular, circular RNA produced higher levels of protein having the correct molecular weight as compared to its linear RNA counterpart.

### Example 18: Persistence of Circular RNA During Cell Division

This Example demonstrates the persistence of circular polyribonucleotide during cell division. A non-naturally occurring circular RNA engineered to include one or more desirable properties may persist in cells through cell division without being degraded. As shown in the following Example, circular RNA expressing Gaussia luciferase (GLuc) was monitored over 72 h days in HeLa cells.

In this Example, a 1307 nt circular RNA included a CVB3 IRES, an ORF encoding Gaussia Luciferase (GLuc), and two spacer elements flanking the IRES-ORF.

Persistence of circular RNA over cell division was monitored in HeLa cells. 5000 cells/well in a 96-well plate were suspension transfected with circular RNA. Bright cell imaging was performed in a Avos imager (ThermoFisher) and cell counts were performed using luminescent cell viability assay (Promega) at 0 h, 24 h, 48 h, 72 h, and 96 h. Gaussia Luciferase enzyme activity was monitored daily as measure of protein expression and gLuc expression was monitored daily in supernatant removed from the wells every 24 h by using the Gaussia Luciferase activity assay (Thermo Scientific Pierce). 50 μl of 1× Gluc substrate was added to 5 μl of plasma to carry out the Gluc luciferase activity assay. Plates were read right after mixing on a luminometer instrument (Promega).

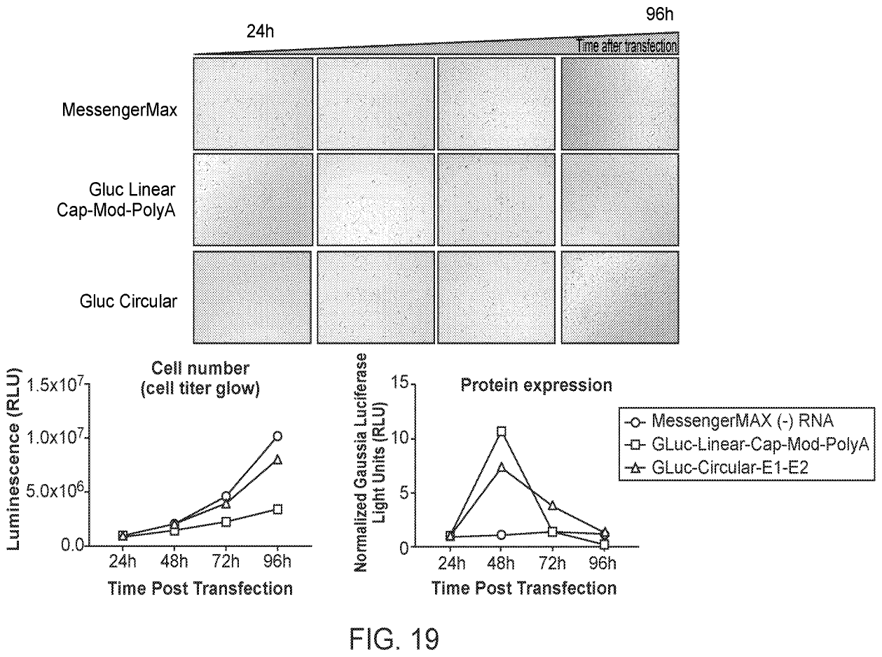

Expression of protein from circular RNA was detected at higher levels than linear RNA in dividing cells (FIG. 19). Cells with circular RNA had higher cell division rates as compared to linear RNA at all timepoints measured. This Example demonstrates increased detection of circular RNA during cell division than its linear RNA counterpart.

### Example 19: Circular RNA Shows Reduced Toxicity Compared to Linear RNA

This Example demonstrates that circular RNA is less toxic than linear RNA.

For this Example, the circular RNA includes an EMCV IRES, an ORF encoding NanoLuc with a 3×FLAG tag and flanked on either side by stagger elements (2A) and a termination element (Stop codon). The circular RNA was generated in vitro and purified as described herein. The linear RNA used in this Example was cap-modified-poly A tailed RNA or cap-unmodified-poly A tailed RNA encoding nLuc with globin UTRs.

To monitor toxicity of RNA in cells, BJ human fibroblast cells were plated onto each well of a 96 well plate. 50 ng of either circular or cap-modified-polyA tailed linear RNA were transfected after zero, forty-eight, and seventy-two hours, using a lipid-based transfection reagent (ThermoFisher) following the manufacturer's recommendations. Bright cell imaging was performed in a Avos imager (ThermoFisher) at 96 h. Total cells per condition were analyzed using ImageJ.

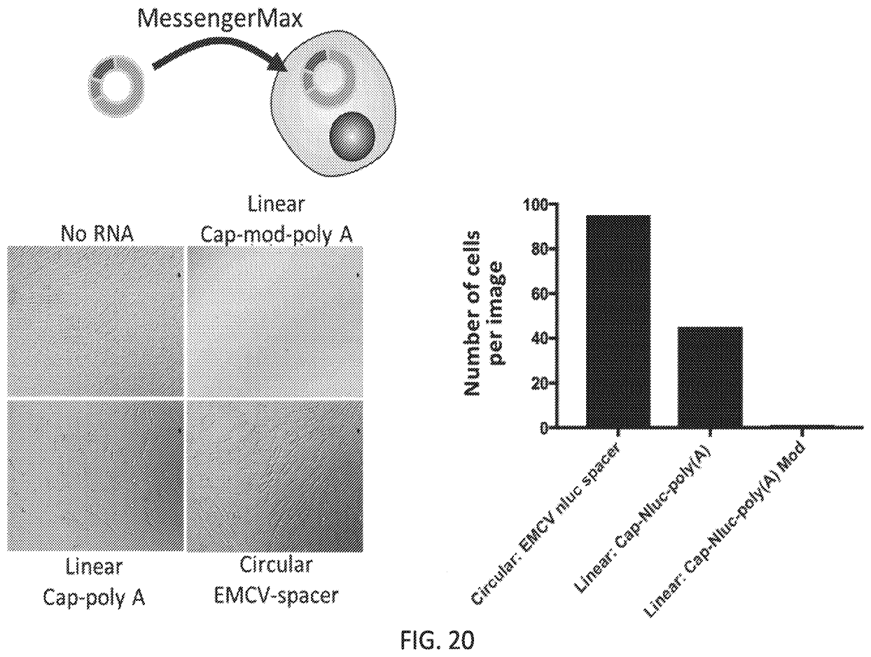

As shown in FIG. 20, transfection of circular RNA demonstrated reduced toxicity compared to linear RNA.

### Example 20: Obtaining Autologous Cells for Non-Viral Circular RNA Cell Therapy

In this Example, cells are obtained for non-viral, circular RNA cell therapy. Therapeutic cell therapy using CAR expression has been demonstrated using autologous T cells. This example demonstrates obtaining autologous T cells for non-viral circular RNA cell therapy.

CAR T cell therapy eligible patients are identified and peripheral blood mononuclear cells (PBMCs) are collected through a leukapheresis procedure. The PBMCs are then cultured under GMP conditions for T cell engineering and expansion. CD8+ Cytotoxic T Cells are isolated from PBMCs using negative selection with immunomagnetic cell separation procedures. Patient T cells are then activated using activated using CD3/CD28 Dynabeads (for 3 days in T cell OpTimizer media) and are ready for electroporation with CAR-encoding mRNA and subsequent infusion into patients.

### Example 21: Obtaining Allogeneic Cells for Non-Viral Circular RNA Cell Therapy

In this Example, cells are obtained for non-viral, circular RNA cell therapy. Therapeutic cell therapy using CAR expression has been demonstrated allogeneic NK cells. This example demonstrates obtaining allogeneic NK cells for non-viral circular RNA cell therapy.

Peripheral blood mononuclear cells (PBMCs) are collected from donors through a leukapheresis procedure. The PBMCs are then cultured under GMP conditions for NK cell engineering and expansion using standard methods (e.g., Shimasaki et al, Cytotherapy, 2012; 14: 830-840 A clinically adaptable method to enhance the cytotoxicity of natural killer cells against B-cell malignancies). Allogeneic NK cells are then are ready for electroporation with CAR-encoding mRNA and subsequent infusion into patients.

### Example 22: In Vitro Circular RNA Production

This example describes in vitro production of a circular RNA.

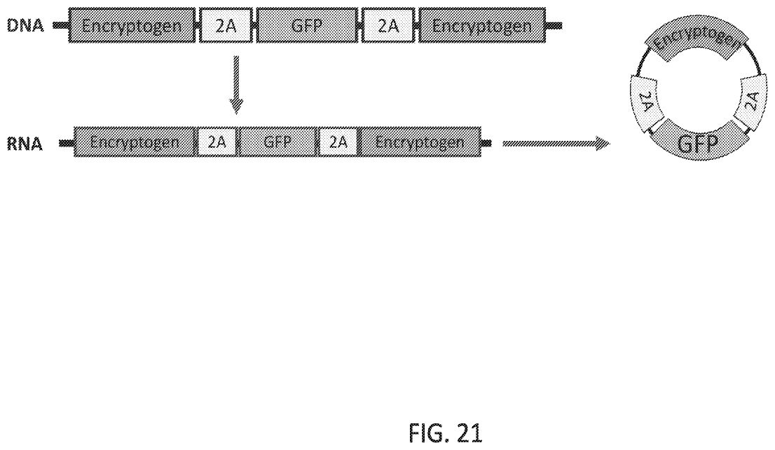

A circular RNA is designed with a start-codon (SEQ ID NO:1), ORF(s) (SEQ ID NO:2), stagger element(s) (SEQ ID NO:3), encryptogen(s) (SEQ ID NO:4), and an IRES (SEQ ID NO:5), shown in FIG. 21. Circularization enables rolling circle translation, multiple open reading frames (ORFs) with alternating stagger elements for discrete ORF expression and controlled protein stoichiometry, encryptogen(s) to attenuate or mitigate RNA immunogenicity, and an optional IRES that targets RNA for ribosomal entry without poly-A sequence.

In this Example, the circular RNA is generated as follows. Unmodified linear RNA is synthesized by in vitro transcription using T7 RNA polymerase from a DNA segment having 5′- and 3′-ZKSCAN1 introns and an ORF encoding GFP linked to 2A sequences. Transcribed RNA is purified with an RNA purification system (QIAGEN), treated with alkaline phosphatase (ThermoFisher Scientific, EF0652) following the manufacturer's instructions, and purified again with the RNA purification system.

Splint ligation circular RNA is generated by treatment of the transcribed linear RNA and a DNA splint using T4 DNA ligase (New England Bio, Inc., M0202M), and the circular RNA is isolated following enrichment with RNase R treatment. RNA quality is assessed by agarose gel or through automated electrophoresis (Agilent).

### Example 23: In Vivo Circular RNA Production, Cell Culture

This example describes in vivo production of a circular RNA.

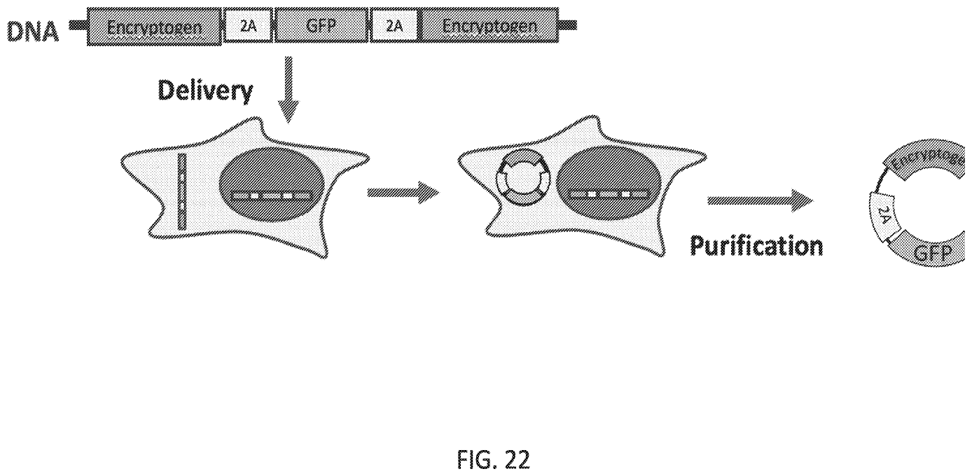

GFP (SEQ ID NO: 2) is cloned into an expression vector, e.g. pcDNA3.1(+) (Addgene) (SEQ ID NO: 6). This vector is mutagenized to induce circular RNA production in cells (SEQ ID NO: 6 and described by Kramer et al 2015), shown in FIG. 22.

HeLa cells are grown at 37° C. and 5% CO₂ in Dulbecco's modified Eagle's medium (DMEM) with high glucose (Life Technologies), supplemented with penicillin-streptomycin and 10% fetal bovine serum. One microgram of the above described expression plasmid is transfected using lipid transfection reagent (Life Technologies), and total RNA from the transfected cells is isolated using a phenol-based RNA isolation reagent (Life Technologies) as per the manufacturer's instructions between 1 hour and 20 days after transfection.

To measure GFP circular RNA and mRNA levels, qPCR reverse transcription using random hexamers is performed. In short, for RT-qPCR HeLa cells' total RNA and RNase R-digested RNA from the same source are used as templates for the RT-PCR. To prepare the cDNAs of GFP mRNAs and circular GFP RNAs, the reverse transcription reactions are performed with a reverse transcriptase (Super-Script II: RNase H; Invitrogen) and random hexamers in accordance with the manufacturer's instruction. The amplified PCR products are analyzed using a 6% PAGE and visualized by ethidium bromide staining. To estimate the enrichment factor, the PCR products are quantified by densitometry (ImageQuant; Molecular Dynamics) and the concentrations of total RNA samples are measured by UV absorbance.

An additional RNA measurement is performed with northern blot analysis. Briefly, whole cell extract was obtained using a phenol based reagent (TRIzol) or nuclear and cytoplasmic protein extracts are obtained by fractionation of the cells with a commercial kit (CelLytic NuCLEAR Extraction Kit, Sigma). To inhibit RNA polymerase II transcription, cells are treated with flavopiridol (1 mM final concentration; Sigma) for 0-6 h at 37° C. For RNase R treatments, 10 mg of total RNA is treated with 20 U of RNase R (Epicentre) for 1 h at 37° C.

Northern blots using oligonucleotide probes are performed as follows. Oligonucleotide probes, PCR primers are designed using standard primer designing tools. T7 promoter sequence is added to the reverse primer to obtain an antisense probe in in vitro transcription reaction. In vitro transcription is performed using T7 RNA polymerase with a DIG-RNA labeling mix according to manufacturer's instruction. DNA templates are removed by DNAs I digestion and RNA probes purified by phenol chloroform extraction and subsequent precipitation. Probes are used at 50 ng/ml. Total RNA (2 μg-10 μg) is denatured using Glyoxal load dye (Ambion) and resolved on 1.2% agarose gel in MOPS buffer. The gel is soaked in 1×TBE for 20 mm and transferred to a Hybond-N+ membrane (GE Healthcare) for 1 h (15 V) using a semi-dry blotting system (Bio-Rad). Membranes are dried and UV-crosslinked (at 265 nm) 1× at 120,000 μJ cm-2. Pre-hybridization is done at 68° C. for 1 h and DIG-labelled in-vitro transcribed RNA probes are hybridized overnight. The membranes are washed three times in 2×SSC, 0.1% SDS at 68° C. for 30 min, followed by three 30 min washes in 0.2×SSC, 0.1% SDS at 68° C. The immunodetection is performed with anti-DIG directly-conjugated with alkaline phosphatase antibodies. Immunoreactive bands are visualized using chemiluminescent alkaline phosphatase substrate (CDP star reagent) and an image detection and quantification system (LAS-4000 detection system).

### Example 24: Preparation of Circular RNA and In Vitro Translation

This example describes gene expression and detection of the gene product from a circular RNA.

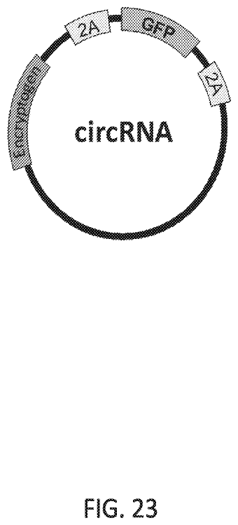

In this Example, the circular RNA is designed with a start-codon (SEQ ID NO:1), a GFP ORF (SEQ ID NO:2), stagger element(s) (SEQ ID NO:3), human-derived encryptogen(s) (SEQ ID NO:4), and with or without an IRES (SEQ ID NO:5), see FIG. 23. In this Example, the circular RNA is generated either in vitro or in cells as described in Example 22 and 23.

The circular RNA is incubated for 5 h or overnight in rabbit reticulocyte lysate (Promega, Fitchburg, WI, USA) at 30° C. The final composition of the reaction mixture includes 70% rabbit reticulocyte lysate, 10 M methionine and leucine, 20 μM amino acids other than methionine and leucine, and 0.8 U/μL RNase inhibitor (Toyobo, Osaka, Japan). Aliquots are taken from the mixture and separated on 10-20% gradient polyacrylamide/sodium dodecyl sulfate (SDS) gels (Atto, Tokyo, Japan). The supernatant is removed and the pellet is dissolved in 2×SDS sample buffer (0.125 M Tris-HCl, pH 6.8, 4% SDS, 30% glycerol, 5% 2-mercaptoethanol, 0.01% bromophenol blue) at 70° C. for 15 min. The hemoglobin protein is removed during this process whereas proteins other than hemoglobin are concentrated.

After centrifugation at 1,400×g for 5 mm, the supernatant is analyzed on 10-20% gradient polyacrylamide/SDS gels. A commercially available standard (BioRad) is used as the size marker. After being electrotransferred to a polyvinylidene fluoride (PVDF) membrane (Millipore) using a semi-dry method, the blot is visualized using a chemiluminescent kit (Rockland).

### Example 25: Stoichiometric Protein Expression from Circular RNA

This example describes the ability of circular RNA to stoichiometrically express of proteins.

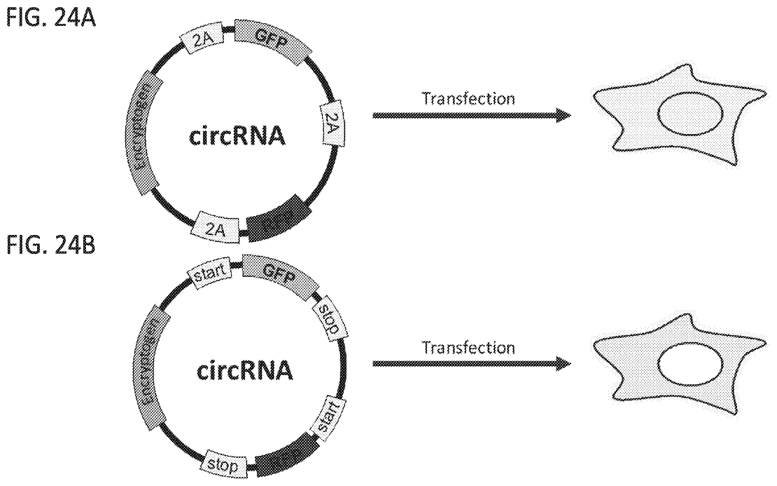

In this Example, one circular RNA is designed to include encryptogens (SEQ ID NO:4) and an ORF encoding GFP (SEQ ID NO: 2) and an ORF encoding RFP (SEQ ID NO: 7) with stagger elements (SEQ ID NO: 3) flanking the GFP and RFP ORFs, see FIG. 24A. Another circular RNA is designed similarly, however instead of flanking 2A sequences it will have a Stop and Start codon in between the GFP and RFP ORFs, see FIG. 24B. The circular RNAs are generated either in vitro or in cells as described in Example 22 and 23.

The circular RNAs are incubated for 5 h or overnight in rabbit reticulocyte lysate (Promega, Fitchburg, WI, USA) at 30° C. The final composition of the reaction mixture includes 70% rabbit reticulocyte lysate, 10 μM methionine and leucine, 20 μM amino acids other than methionine and leucine, and 0.8 U/μL RNase inhibitor (Toyobo, Osaka, Japan). Aliquots are taken from the mixture and separated on 10-20% gradient polyacrylamide/sodium dodecyl sulfate (SDS) gels (Atto, Tokyo, Japan). The supernatant is removed and the pellet is dissolved in 2×SDS sample buffer (0.125 M Tris-HCl, pH 6.8, 4% SDS, 30% glycerol, 5% 2-mercaptoethanol, 0.01% bromophenol blue) at 70° C. for 15 min. The hemoglobin protein is removed during this process whereas proteins other than hemoglobin are concentrated.

After centrifugation at 1,400×g for 5 min, the supernatant is analyzed on 10-20% gradient polyacrylamide/SDS gels. A commercially available standard (BioRad) is used as the size marker. After being electrotransferred to a polyvinylidene fluoride (PVDF) membrane (Millipore) using a semi-dry method, the blot is visualized using a chemiluminescent kit (Rockland).

It is expected that circular RNA with GFP and RFP ORFs not separated by a Stop and start codon will have equal amounts of either protein, while cells treated with the circular RNA including the start and stop codon in between the ORFs will have different amounts of either protein.

Sequence listing

```
SEQ ID NO: 1 (Start Codon)
AUG
SEQ ID NO: 2 (GFP)
EGFP:
atggtgagcaagggcgaggagctgttcaccggggt
ggtgcccatcctggtcgagctggacggcgacgtaa
acggccacaagttcagcgtgtccggcgagggcgag
ggcgatgccacctacggcaagctgaccctgaagtt
catctgcaccaccggcaagctgcccgtgccctggc
ccaccctcgtgaccaccctgacctacggcgtgcag
tgcttcagccgctaccccgaccacatgaagcagca
cgacttcttcaagtccgccatgcccgaaggctacg
tccaggagcgcaccatcttcttcaaggacgacggc
aactacaagacccgcgccgaggtgaagttcgaggg
cgacaccctggtgaaccgcatcgagctgaagggca
tcgacttcaaggaggacggcaacatcctggggcac
aagctggagtacaactacaacagccacaacgtcta
tatcatggccgacaagcagaagaacggcatcaagg
tgaacttcaagatccgccacaacatcgaggacggc
agcgtgcagctcgccgaccactaccagcagaacac
ccccatcggcgacggccccgtgctgctgcccgaca
accactacctgagcacccagtccgccctgagcaaa
gaccccaacgagaagcgcgatcacatggtcctgct
ggagttcgtgaccgccgccgggatcactctcggca
tggacgagctgtacaag
SEQ ID NO: 3 (stagger element)
P2A:
gctactaacttcagcctgctgaagcaggctggcga
cgtggaggagaaccctggacct
T2A:
gagggcaggggaagtctactaacatgcggg
gacgtggaggaaaatcccggccca
E2A:
cagtgtactaattatgctctcttgaaattggctgg
agatgttgagagcaacccaggtccc
Others: F2A, BmCPV2A, BmIFV2A
SEQ ID NO: 4 ZKSCAN
GTAAAAAGAGGTGAAACCTATTATGTGTGAGCAGG
GCACAGACGTTGAAACTGGAGCCAGGAGAAGTATT
GGCAGGCTTTAGGTTATTAGGTGGTTACTCTGTCT
TAAAAATGTTCTGGCTTTCTTCCTGCATCCACTGG
CATACTCATGGTCTGTTTTTAAATATTTTAATTCC
CATTTACAAAGTGATTTACCCACAAGCCCAACCTG
TCTGTCTTCAG
Or
GTAAGAAGCAAGGTTTCATTTAGGGGAAGGGAAAT
GATTCAGGACGAGAGTCTTTGTGCTGCTGAGTGCC
TGTGATGAAGAAGCATGTTAGTcctgggcaacgta
gcgagaccccatctctacaaaaaatagaaaaatta
gccaggtatagtggcgcacacctgtgattccagct
acgcaggaggctgaggtgggaggattgcttgagcc
caggaggttgaggctgcagtgagctgtaatcatgc
cactactccaacctgggcaacacagcaaggaccct
gtctcaaaaGCTACTTACAGAAAAGAATTAggctc
ggcacggtagctcacacctgtaatcccagcacttt
gggaggctgaggcgggcagatcacttgaggtcagg
agtttgagaccagcctggccaacatggtgaaacct
tgtctctactaaaaatatgaaaattagccaggcat
ggtggcacattcctgtaatcccagctactcgggag
gctgaggcaggagaatcacttgaacccaggaggtg
gaggttgcagtaagccgagatcgtaccactgtgct
ctagccttggtgacagagcgagactgtcttaaaaa
aaaaaaaaaaaaaaaaagaattaattaaaaattta
aaaaaaaatgaaaaaaaGCTGCATGCTTGTTTTTT
GTTTTTAGTTATTCTACATTGTTGTCATTATTACC
AAATATTGGGGAAAATACAACTTACAGACCAATCT
CAGGAGTTAAATGTTACTACGAAGGCAAATGAACT
ATGCGTAATGAACCTGGTAGGCATTA
SEQ ID NO: 5 (IRES)
IRES (EMCV):
Acgttactggccgaagccgcttggaataaggccgg
tgtgcgtttgtctatatgttattttccaccatatt
gccgtcttttggcaatgtgagggcccggaaacctg
gccctgtcttcttgacgagcattcctaggggtctt
tcccctctcgccaaaggaatgcaaggtctgttgaa
tgtcgtgaaggaagcagttcctctggaagcttctt
gaagacaaacaacgtctgtagcgaccctttgcagg
cagcggaaccccccacctggcgacaggtgcctctg
cggccaaaagccacgtgtataagatacacctgcaa
aggcggcacaaccccagtgccacgttgtgagttgg
atagttgtggaaagagtcaaatggctctcctcaag
cgtattcaacaaggggctgaaggatgcccagaagg
taccccattgtatgggatctgatctggggcctcgg
tgcacatgattacatgtgtnagtcgaggttaaaaa
acgtctaggccccccgaaccacggggacgtggttt
tcctttgaaaaacacgatgataata
SEQ ID NO: 6 (addgene p3.1 laccase)
pcDNA3.1(+) Laccase2 MCS Exon
Vector sequence 6926 bps
GACGGATCGGGAGATCTCCCGATCCCCTATGGTGC
ACTCTCAGTACAATCTGCTCTGATGCCGCATAGTT
AAGCCAGTATCTGCTCCCTGCTTGTGTGTTGGAGG
TCGCTGAGTAGTGCGCGAGCAAAATTTAAGCTACA
ACAAGGCAAGGCTTGACCGACAATTGCATGAAGAA
TCTGCTTAGGGTTAGGCGTTTTGCGCTGCTTCGCG
ATGTACGGGCCAGATATACGCGTTGACATTGATTA
TTGACTAGTTATTAATAGTAATCAATTACGGGGTC
ATTAGTTCATAGCCCATATATGGAGTTCCGCGTTA
CATAACTTACGGTAAATGGCCCGCCTGGCTGACCG
CCCAACGACCCCCGCCCATTGACGTCAATAATGAC
GTATGTTCCCATAGTAACGCCAATAGGGACTTTCC
ATTGACGTCAATGGGTGGAGTATTTACGGTAAACT
GCCCACTTGGCAGTACATCAAGTGTATCATATGCC
AAGTACGCCCCCTATTGACGTCAATGACGGTAAAT
GGCCCGCCTGGCATTATGCCCAGTACATGACCTTA
TGGGACTTTCCTACTTGGCAGTACATCTACGTATT
AGTCATCGCTATTACCATGGTGATGCGGTTTTGGC
AGTACATCAATGGGCGTGGATAGCGGTTTGACTCA
CGGGGATTTCCAAGTCTCCACCCCATTGACGTCAA
TGGGAGTTTGTTTTGGCACCAAAATCAACGGGACT
TTCCAAAATGTCGTAACAACTCCGCCCCATTGACG
CAAATGGGCGGTAGGCGTGTACGGTGGGAGGTCTA
TATAAGCAGAGCTCTCTGGCTAACTAGAGAACCCA
CTGCTTACTGGCTTATCGAAATTAATACGACTCAC
TATAGGGAGACCCAAGCTGGCTAGCGTTTAAACTT
AAGCTTGGTACCGAGCTCGGATCCACTAGTCCAGT
GTGGTGGAATTCCATTGAGAAATGACTGAGTTCCG
GTGCTCTCAAGTCATTGATCTTTGTCGACTTTTAT
TTGGTCTCTGTAATAACGACTTCAAAAACATTAAA
TTCTGTTGCGAAGCCAGTAAGCTACAAAAAGAAAa
acaagagagaatgctatagtcgtatagtatagtttc
ccgactatctgatacccattacttatctaggggga
atgcgaacccaaaattttatcagttttctcggata
tcgatagatattggggaataaatttaaataaataa
attttgggcgggtttagggcgtggcaaaaagtttt
ttggcaaatcgctagaaatttacaagacttataaa
attatgaaaaaatacaacaaaattttaaacacgtg
ggcgtgacagttttggGcggttttagggcgttaga
gtaggcgaggacagggttacatcgactaggctttg
atcctgatcaagaatatatatactttataccgctt
ccttctacatgttacctatttttcaacgaatctag
tatacctttttactgtacgatttatgggtataaTA
ATAAGCTA
AATCGAGACTAAGttttattgttatatatattttt
tttattttatGCAGAAATTAATTAAACCGGTCCTG
CAGGTGATCAGGCGCGCCGGTTACCGGCCGGCCCC
GCGGAGCGTAAGTATTCAAAATTCCAAAATTTTTT
ACTAGAAATATTCGATTTTTTAATAGGCAGTTTCT
ATACTATTGTATACTATTGtagattcgttgaaaag
tatgtaacaggaagaataaagcataccgaccatgt
aaagtatatatattcttaataaggatcaatagccg
agtcgatctcgccatgtccgtctgtcttattGttt
tattaccgccgagacatcaggaactataaaagcta
gaaggatgagttttagcatacagattctagagaca
aggacgcagagcaagtagttgatccatgctgccac
gctttaactactcaaattgcccaaaactgccatgc
ccacatttttgaactattttcgaaattttttcata
attgtattactcgtgtaaatttccatcaatttgcc
aaaaaactttttgtcacgcgttaacgccctaaagc
cgccaatttggtcacgcccacactattgaGcaatt
atcaaattttttctcattttattccccaatatcta
tcgatatccccgattatgaaattattaaatttcgc
gttcgcattcacactagctgagtaacgagtatctg
atagttggggaaatcgactTATTTTTTATATACAA
TGAAAATGAATTTAATCATATGAATATCGATTATA
GCTTTTTATTTAATATGAATATTTATTTGGGCTTA
AGGTGTAACCTcctcgacataagactcacatggcg
caggcacattgaagacaaaaatactcaTTGTCGGG
TCTCGCACCCTCCAGCAGCACCTAAAATTATGTCT
TCAATTATTGCCAACATTGGAGACACAATTAGTCT
GTGGCACCTCAGGCGGCCGCTCGAGTCTAGAGGGC
CCGTTTAAACCCGCTGATCAGCCTCGACTGTGCCT
TCTAGTTGCCAGCCATCTGTTGTTTGCCCCTCCCC
CGTGCCTTCCTTGACCCTGGAAGGTGCCACTCCCA
CTGTCCTTTCCTAATAAAATGAGGAAATTGCATCG
CATTGTCTGAGTAGGTGTCATTCTATTCTGGGGGG
TGGGGTGGGGCAGGACAGCAAGGGGGAGGATTGGG
AAGACAATAGCAGGCATGCTGGGGATGCGGTGGGC
TCTATGGCTTCTGAGGCGGAAAGAACCAGCTGGGG
CTCTAGGGGGTATCCCCACGCGCCCTGTAGCGGCG
CATTAAGCGCGGCGGGTGTGGTGGTTACGCGCAGC
GTGACCGCTACACTTGCCAGCGCCCTAGCGCCCGC
TCCTTTCGCTTTCTTCCCTTCCTTTCTCGCCACGT
TCGCCGGCTTTCCCCGTCAAGCTCTAAATCGGGGG
CTCCCTTTAGGGTTCCGATTTAGTGCTTTACGGCA
CCTCGACCCCAAAAAACTTGATTAGGGTGATGGTT
CACGTAGTGGGCCATCGCCCTGATAGACGGTTTTT
CGCCCTTTGACGTTGGAGTCCACGTTCTTTAATAG
TGGACTCTTGTTCCAAACTGGAACAACACTCAACC
CTATCTCGGTCTATTCTTTTGATTTATAAGGGATT
TTGCCGATTTCGGCCTATTGGTTAAAAAATGAGCT
GATTTAACAAAAATTTAACGCGAATTAATTCTGTG
GAATGTGTGTCAGTTAGGGTGTGGAAAGTCCCCAG
GCTCCCCAGCAGGCAGAAGTATGCAAAGCATGCAT
CTCAATTAGTCAGCAACCAGGTGTGGAAAGTCCCC
AGGCTCCCCAGCAGGCAGAAGTATGCAAAGCATGC
ATCTCAATTAGTCAGCAACCATAGTCCCGCCCCTA
ACTCCGCCCATCCCGCCCCTAACTCCGCCCAGTTC
CGCCCATTCTCCGCCCCATGGCTGACTAATTTTTT
TTATTTATGCAGAGGCCGAGGCCGCCTCTGCCTCT
GAGCTATTCCAGAAGTAGTGAGGAGGCTTTTTTGG
AGGCCTAGGCTTTTGCAAAAAGCTCCCGGGAGCTT
GTATATCCATTTTCGGATCTGATCAAGAGACAGGA
TGAGGATCGTTTCGCATGATTGAACAAGATGGATT
GCACGCAGGTTCTCCGGCCGCTTGGGTGGAGAGGC
TATTCGGCTATGACTGGGCACAACAGACAATCGGC
TGCTCTGATGCCGCCGTGTTCCGGCTGTCAGCGCA
GGGGCGCCCGGTTCTTTTTGTCAAGACCGACCTGT
CCGGTGCCCTGAATGAACTGCAGGACGAGGCAGCG
CGGCTATCGTGGCTGGCCACGACGGGCGTTCCTTG
CGCAGCTGTGCTCGACGTTGTCACTGAAGCGGGAA
GGGACTGGCTGCTATTGGGCGAAGTGCCGGGGCAG
GATCTCCTGTCATCTCACCTTGCTCCTGCCGAGAA
AGTATCCATCATGGCTGATGCAATGCGGCGGCTGC
ATACGCTTGATCCGGCTACCTGCCCATTCGACCAC
CAAGCGAAACATCGCATCGAGCGAGCACGTACTCG
GATGGAAGCCGGTCTTGTCGATCAGGATGATCTGG
ACGAAGAGCATCAGGGGCTCGCGCCAGCCGAACTG
TTCGCCAGGCTCAAGGCGCGCATGCCCGACGGCGA
GGATCTCGTCGTGACCCATGGCGATGCCTGCTTGC
CGAATATCATGGTGGAAAATGGCCGCTTTTCTGGA
TTCATCGACTGTGGCCGGCTGGGTGTGGCGGACCG
CTATCAGGACATAGCGTTGGCTACCCGTGATATTG
CTGAAGAGCTTGGCGGCGAATGGGCTGACCGCTTC
CTCGTGCTTTACGGTATCGCCGCTCCCGATTCGCA
GCGCATCGCCTTCTATCGCCTTCTTGACGAGTTCT
TCTGAGCGGGACTCTGGGGTTCGAAATGACCGACC
AAGCGACGCCCAACCTGCCATCACGAGATTTCGAT
TCCACCGCCGCCTTCTATGAAAGGTTGGGCTTCGG
AATCGTTTTCCGGGACGCCGGCTGGATGATCCTCC
AGCGCGGGGATCTCATGCTGGAGTTCTTCGCCCAC
CCCAACTTGTTTATTGCAGCTTATAATGGTTACAA
ATAAAGCAATAGCATCACAAATTTCACAAATAAAG
CATTTTTTTCACTGCATTCTAGTTGTGGTTTGTCC
AAACTCATCAATGTATCTTATCATGTCTGTATACC
GTCGACCTCTAGCTAGAGCTTGGCGTAATCATGGT
CATAGCTGTTTCCTGTGTGAAATTGTTATCCGCTC
ACAATTCCACACAACATACGAGCCGGAAGCATAAA
GTGTAAAGCCTGGGGTGCCTAATGAGTGAGCTAAC
TCACATTAATTGCGTTGCGCTCACTGCCCGCTTTC
CAGTCGGGAAACCTGTCGTGCCAGCTGCATTAATG
AATCGGCCAACGCGCGGGGAGAGGCGGTTTGCGTA
TTGGGCGCTCTTCCGCTTCCTCGCTCACTGACTCG
CTGCGCTCGGTCGTTCGGCTGCGGCGAGCGGTATC
AGCTCACTCAAAGGCGGTAATACGGTTATCCACAG
AATCAGGGGATAACGCAGGAAAGAACATGTGAGCA
AAAGGCCAGCAAAAGGCCAGGAACCGTAAAAAGGC
CGCGTTGCTGGCGTTTTTCCATAGGCTCCGCCCCC
CTGACGAGCATCACAAAAATCGACGCTCAAGTCAG
AGGTGGCGAAACCCGACAGGACTATAAAGATACCA
GGCGTTTCCCCCTGGAAGCTCCCTCGTGCGCTCTC
CTGTTCCGACCCTGCCGCTTACCGGATACCTGTCC
GCCTTTCTCCCTTCGGGAAGCGTGGCGCTTTCTCA
TAGCTCACGCTGTAGGTATCTCAGTTCGGTGTAGG
TCGTTCGCTCCAAGCTGGGCTGTGTGCACGAACCC
CCCGTTCAGCCCGACCGCTGCGCCTTATCCGGTAA
CTATCGTCTTGAGTCCAACCCGGTAAGACACGACT
TATCGCCACTGGCAGCAGCCACTGGTAACAGGATT
AGCAGAGCGAGGTATGTAGGCGGTGCTACAGAGTT
CTTGAAGTGGTGGCCTAACTACGGCTACACTAGAA
GAACAGTATTTGGTATCTGCGCTCTGCTGAAGCCA
GTTACCTTCGGAAAAAGAGTTGGTAGCTCTTGATC
CGGCAAACAAACCACCGCTGGTAGCGGTTTTTTTG
TTTGCAAGCAGCAGATTACGCGCAGAAAAAAAGGA
TCTCAAGAAGATCCTTTGATCTTTTCTACGGGGTC
TGACGCTCAGTGGAACGAAAACTCACGTTAAGGGA
TTTTGGTCATGAGATTATCAAAAAGGATCTTCACC
TAGATCCTTTTAAATTAAAAATGAAGTTTTAAATC
AATCTAAAGTATATATGAGTAAACTTGGTCTGACA
GTTACCAATGCTTAATCAGTGAGGCACCTATCTCA
GCGATCTGTCTATTTCGTTCATCCATAGTTGCCTG
ACTCCCCGTCGTGTAGATAACTACGATACGGGAGG
GCTTACCATCTGGCCCCAGTGCTGCAATGATACCG
CGAGACCCACGCTCACCGGCTCCAGATTTATCAGC
AATAAACCAGCCAGCCGGAAGGGCCGAGCGCAGAA
GTGGTCCTGCAACTTTATCCGCCTCCATCCAGTCT
ATTAATTGTTGCCGGGAAGCTAGAGTAAGTAGTTC
GCCAGTTAATAGTTTGCGCAACGTTGTTGCCATTG
CTACAGGCATCGTGGTGTCACGCTCGTCGTTTGGT
ATGGCTTCATTCAGCTCCGGTTCCCAACGATCAAG
GCGAGTTACATGATCCCCCATGTTGTGCAAAAAAG
CGGTTAGCTCCTTCGGTCCTCCGATCGTTGTCAGA
AGTAAGTTGGCCGCAGTGTTATCACTCATGGTTAT
GGCAGCACTGCATAATTCTCTTACTGTCATGCCAT
CCGTAAGATGCTTTTCTGTGACTGGTGAGTACTCA
ACCAAGTCATTCTGAGAATAGTGTATGCGGCGACC
GAGTTGCTCTTGCCCGGCGTCAATACGGGATAATA
CCGCGCCACATAGCAGAACTTTAAAAGTGCTCATC
ATTGGAAAACGTTCTTCGGGGCGAAAACTCTCAAG
GATCTTACCGCTGTTGAGATCCAGTTCGATGTAAC
CCACTCGTGCACCCAACTGATCTTCAGCATCTTTT
ACTTTCACCAGCGTTTCTGGGTGAGCAAAAACAGG
AAGGCAAAATGCCGCAAAAAAGGGAATAAGGGCGA
CACGGAAATGTTGAATACTCATACTCTTCCTTTTT
CAATATTATTGAAGCATTTATCAGGGTTATTGTCT
CATGAGCGGATACATATTTGAATGTATTTAGAAAA
ATAAACAAATAGGGGTTCCGCGCACATTTCCCCGA
AAAGTGCCACCTGACGTC
SEQ ID NO: 7 (RFP)
mCherry:
atggtgagcaagggcgaggaggataacatggccat
catcaaggagttcatgcgcttcaaggtgcacatgg
agggctccgtgaacggccacgagttcgagatcgag
ggcgagggcgagggccgcccctacgagggcaccca
gaccgccaagctgaaggtgaccaagggtggccccc
tgcccttcgcctgggacatcctgtcccctcagttc
atgtacggctccaaggcctacgtgaagcaccccgc
cgacatccccgactacttgaagctgtccttccccg
agggcttcaagtgggagcgcgtgatgaacttcgag
gacggcggcgtggtgaccgtgacccaggactcctc
cctgcaggacggcgagttcatctacaaggtgaagc
tgcgcggcaccaacttcccctccgacggccccgta
atgcagaagaagaccatgggctgggaggcctcctc
cgagcggatgtaccccgaggacggcgccctgaagg
gcgagatcaagcagaggctgaagctgaaggacggc
ggccactacgacgctgaggtcaagaccacctacaa
ggccaagaagcccgtgcagctgcccggcgcctaca
acgtcaacatcaagttggacatcacctcccacaac
gaggactacaccatcgtggaacagtacgaacgcgc
cgagggccgccactccaccggcggcatggacgagc
tgtacaag
Sequence ID 9
Kozak 3XFLAG-EGF nostop (264 bps)
GGGAGCCACCATGGACTACAAGGACGACGACGACA
AGATCATCGACTATAAAGACGACGACGATAAAGGT
GGCGACTATAAGGACGACGACGACAAAGCCATTAA
TAGTGACTCTGAGTGTCCCCTGTCCCACGACGGGT
ACTGCCTCCACGACGGTGTGTGCATGTATATTGAA
GCATTGGACAAGTACGCCTGCAACTGTGTTGTTGG
CTACATCGGGGAGCGCTGTCAGTACCGAGACCTGA
AGTGGTGGGAACTGCGCCT
    5-13: Kozak sequence
14-262: 3XFLAG-EGF
SEQ ID NO: 10
Kozak 3XFLAG-EGF P2A nostop (330 bps)
GGGAGCCACCATGGACTACAAGGACGACGACGACA
AGATCATCGACTATAAAGACGACGACGATAAAGGT
GGCGACTATAAGGACGACGACGACAAAGCCATTAA
TAGTGACTCTGAGTGTCCCCTGTCCCACGACGGGT
ACTGCCTCCACGACGGTGTGTGCATGTATATTGAA
GCATTGGACAAGTACGCCTGCAACTGTGTTGTTGG
CTACATCGGGGAGCGCTGTCAGTACCGAGACCTGA
AGTGGTGGGAACTGCGCGGAAGCGGAGCTACTAAC
TTCAGCCTGCTGAAGCAGGCTGGAGACGTGGAGGA
GAACCCTGGACCTCT
      5-13: Kozak sequence
  14-262: 3XFLAG-EGF
263-328: P2A
SEQ ID NO: 11
Kozak 3XFLAG-EGF nostop (264 bps)
GGGAGCCACCATGGACTACAAGGACGACGACGACA
AGATCATCGACTATAAAGACGACGACGATAAAGGT
GGCGACTATAAGGACGACGACGACAAAGCCATTAA
TAGTGACTCTGAGTGTCCCCTGTCCCACGACGGGT
ACTGCCTCCACGACGGTGTGTGCATGTATATTGAA
GCATTGGACAAGTACGCCTGCAACTGTGTTGTTGG
CTACATCGGGGAGCGCTGTCAGTACCGAGACCTGA
AGTGGTGGGAACTGCGCCT
    5-13: Kozak sequence
14-262: 3XFLAG-EGF
SEQ ID NO: 12
Kozak 3XFLAG-EGF stop (273 bps)
GGGAGCCACCATGGACTACAAGGACGACGACGACA
AGATCATCGACTATAAAGACGACGACGATAAAGGT
GGCGACTATAAGGACGACGACGACAAAGCCATTAA
TAGTGACTCTGAGTGTCCCCTGTCCCACGACGGGT
ACTGCCTCCACGACGGTGTGTGCATGTATATTGAA
GCATTGGACAAGTACGCCTGCAACTGTGTTGTTGG
CTACATCGGGGAGCGCTGTCAGTACCGAGACCTGA
AGTGGTGGGAACTGCGCTGATAGTAACT
      5-13: Kozak sequence
  14-262: 3XFLAG-EGF
263-271: Triple stop codon
SEQ ID NO: 13
EMCV IRES T2A 3XFLAG-EGF P2A nostop
(954 bps)
GGGACCTAACGTTACTGGCCGAAGCCGCTTGGAAC
AAGGCCGGTGTGCGTTTGTCTATATGTTATTTTCC
ACCATATTGCCGTCTTTTGGCAATGTGAGGGCCCG
GAAACCTGGCCCTGTCTTCTTGACGAGCATTCCTA
GGGGTCTTTCCCCTCTCGCCAAAGGAATGCAAGGT
CTGTTGAATGTCGTGAAGGAAGCAGTTCCTCTGGA
AGCTTCTTCAAGACAAACAACGTCTGTAGCGACCC
TTTGCAGGCAGCGGAACCCCCCACCTGGCGACAGG
TGCCTCTGCGGCCAAAAGCCACGTGTATACGATAC
ACCTGCAAAGGCGGCACAACCCCAGTGCCACGTTG
TGAGTTGGATAGTTGTGGAAAGAGTCAAATGGCTC
TCCTCAAGCGTATTCAACAAGGGGCTGAAGGATGC
CCAGAAGGTACCCCATTGTATGGGATCTGATCTGG
GGCCTCGGTGCACATGCTTTACATGTGTTCAGTCG
AGGTTAAAAAACGTCCAGGCCCCCCGAACCACGGG
GACGTGGTTTTCCTTTGAAAAACACGATGATAATA
TGGCCACAACCATGGGCTCCGGCGAGGGCAGGGGA
AGTCTTCTAACATGCGGGGACGTGGAGGAAAATCC
CGGCCCAGACTACAAGGACGACGACGACAAGATCA
TCGACTATAAAGACGACGACGATAAAGGTGGCGAC
TATAAGGACGACGACGACAAAGCCATTAATAGTGA
CTCTGAGTGTCCCCTGTCCCACGACGGGTACTGCC
TCCACGACGGTGTGTGCATGTATATTGAAGCATTG
GACAAGTACGCCTGCAACTGTGTTGTTGGCTACAT
CGGGGAGCGCTGTCAGTACCGAGACCTGAAGTGGT
GGGAACTGCGCGGAAGCGGAGCTACTAACTTCAGC
CTGCTGAAGCAGGCTGGAGACGTGGAGGAGAACCC
TGGACCTCT
    5-574: EMCV IRES
575-637: T2A
638-886: 3XFALG-EGF
887-952: P2A
SEQ ID NO: 14
EMCV T2A 3XFLAG Nluc P2A stop
(1314 nts)
GGGACCTAACGTTACTGGCCGAAGCCGCTTGGAAC
AAGGCCGGTGTGCGTTTGTCTATATGTTATTTTCC
ACCATATTGCCGTCTTTTGGCAATGTGAGGGCCCG
GAAACCTGGCCCTGTCTTCTTGACGAGCATTCCTA
GGGGTCTTTCCCCTCTCGCCAAAGGAATGCAAGGT
CTGTTGAATGTCGTGAAGGAAGCAGTTCCTCTGGA
AGCTTCTTCAAGACAAACAACGTCTGTAGCGACCC
TTTGCAGGCAGCGGAACCCCCCACCTGGCGACAGG
TGCCTCTGCGGCCAAAAGCCACGTGTATACGATAC
ACCTGCAAAGGCGGCACAACCCCAGTGCCACGTTG
TGAGTTGGATAGTTGTGGAAAGAGTCAAATGGCTC
TCCTCAAGCGTATTCAACAAGGGGCTGAAGGATGC
CCAGAAGGTACCCCATTGTATGGGATCTGATCTGG
GGCCTCGGTGCACATGCTTTACATGTGTTCAGTCG
AGGTTAAAAAACGTCCAGGCCCCCCGAACCACGGG
GACGTGGTTTTCCTTTGAAAAACACGATGATAATA
TGGCCACAACCATGGGCTCCGGCGAGGGCAGGGGA
AGTCTTCTAACATGCGGGGACGTGGAGGAAAATCC
CGGCCCAGACTACAAGGACGACGACGACAAGATCA
TCGACTATAAAGACGACGACGATAAAGGTGGCGAC
TATAAGGACGACGACGACAAAGCCATTGTCTTCAC
ACTCGAAGATTTCGTTGGGGACTGGCGACAGACAG
CCGGCTACAACCTGGACCAAGTCCTTGAACAGGGA
GGTGTGTCCAGTTTGTTTCAGAATCTCGGGGTGTC
CGTAACTCCGATCCAAAGGATTGTCCTGAGCGGTG
AAAATGGGCTGAAGATCGACATCCATGTCATCATC
CCGTATGAAGGTCTGAGCGGCGACCAAATGGGCCA
GATCGAAAAAATTTTTAAGGTGGTGTACCCTGTGG
ATGATCATCACTTTAAGGTGATCCTGCACTATGGC
ACACTGGTAATCGACGGGGTTACGCCGAACATGAT
CGACTATTTCGGACGGCCGTATGAAGGCATCGCCG
TGTTCGACGGCAAAAAGATCACTGTAACAGGGACC
CTGTGGAACGGCAACAAAATTATCGACGAGCGCCT
GATCAACCCCGACGGCTCCCTGCTGTTCCGAGTAA
CCATCAACGGAGTGACCGGCTGGCGGCTGTGCGAA
CGCATTCTGGCGGGAAGCGGAGCTACTAACTTCAG
CCTGCTGAAGCAGGCTGGAGACGTGGAGGAGAACCC
TGGACCTTGATAGTAACT
        5-574: EMCV IRES
    575-637: T2A
  638-1237: 3XFLAG Nluc
1238-1303: P2A
1304-1312: Triple stop codon
SEQ ID NO: 15
EMCV T2A 3XFLAG Nluc P2A nostop
(1305 nts)
GGGACCTAACGTTACTGGCCGAAGCCGCTTGGAAC
AAGGCCGGTGTGCGTTTGTCTATATGTTATTTTCC
ACCATATTGCCGTCTTTTGGCAATGTGAGGGCCCG
GAAACCTGGCCCTGTCTTCTTGACGAGCATTCCTA
GGGGTCTTTCCCCTCTCGCCAAAGGAATGCAAGGT
CTGTTGAATGTCGTGAAGGAAGCAGTTCCTCTGGA
AGCTTCTTCAAGACAAACAACGTCTGTAGCGACCC
TTTGCAGGCAGCGGAACCCCCCACCTGGCGACAGG
TGCCTCTGCGGCCAAAAGCCACGTGTATACGATAC
ACCTGCAAAGGCGGCACAACCCCAGTGCCACGTTG
TGAGTTGGATAGTTGTGGAAAGAGTCAAATGGCTC
TCCTCAAGCGTATTCAACAAGGGGCTGAAGGATGC
CCAGAAGGTACCCCATTGTATGGGATCTGATCTGG
GGCCTCGGTGCACATGCTTTACATGTGTTCAGTCG
AGGTTAAAAAACGTCCAGGCCCCCCGAACCACGGG
GACGTGGTTTTCCTTTGAAAAACACGATGATAATA
TGGCCACAACCATGGGCTCCGGCGAGGGCAGGGGA
AGTCTTCTAACATGCGGGGACGTGGAGGAAAATCC
CGGCCCAGACTACAAGGACGACGACGACAAGATCA
TCGACTATAAAGACGACGACGATAAAGGTGGCGAC
TATAAGGACGACGACGACAAAGCCATTGTCTTCAC
ACTCGAAGATTTCGTTGGGGACTGGCGACAGACAG
CCGGCTACAACCTGGACCAAGTCCTTGAACAGGGA
GGTGTGTCCAGTTTGTTTCAGAATCTCGGGGTGTC
CGTAACTCCGATCCAAAGGATTGTCCTGAGCGGTG
AAAATGGGCTGAAGATCGACATCCATGTCATCATC
CCGTATGAAGGTCTGAGCGGCGACCAAATGGGCCA
GATCGAAAAAATTTTTAAGGTGGTGTACCCTGTGG
ATGATCATCACTTTAAGGTGATCCTGCACTATGGC
ACACTGGTAATCGACGGGGTTACGCCGAACATGAT
CGACTATTTCGGACGGCCGTATGAAGGCATCGCCG
TGTTCGACGGCAAAAAGATCACTGTAACAGGGACC
CTGTGGAACGGCAACAAAATTATCGACGAGCGCCT
GATCAACCCCGACGGCTCCCTGCTGTTCCGAGTAA
CCATCAACGGAGTGACCGGCTGGCGGCTGTGCGAA
CGCATTCTGGCGGGAAGCGGAGCTACTAACTTCAG
CCTGCTGAAGCAGGCTGGAGACGTGGAGGAGAACC
CTGGACCTCT
        5-574: EMCV IRES
    575-637: T2A
  638-1237: 3XFLAG Nluc
1238-1303: P2A
SEQ ID NO: 16
CD19 CAR ORF:
ATGGCTCTGCCTGTGACAGCTCTGCTGCTGCCTCT
GGCCCTGCTGCTGCATGCTGCCAGACCTGACATCC
AGATGACACAGACTACATCCTCCCTGTCTGCCTCT
CTGGGAGACAGAGTCACCATCAGTTGCAGGGCAAG
TCAGGACATTAGTAAATATTTAAATTGGTATCAGC
AGAAACCAGATGGAACTGTTAAACTCCTGATCTAC
CATACATCAAGATTACACTCAGGAGTCCCATCAAG
GTTCAGTGGCAGTGGGTCTGGAACAGATTATTCTC
TCACCATTAGCAACCTGGAGCAAGAAGATATTGCC
ACTTACTTTTGCCAACAGGGTAATACGCTTCCGTA
CACGTTCGGAGGGGGGACTAAGTTGGAAATAACAG
GCTCCACCTCTGGATCCGGCAAGCCCGGATCTGGC
GAGGGATCCACCAAGGGCGAGGTGAAACTGCAGGA
GTCAGGACCTGGCCTGGTGGCGCCCTCACAGAGCC
TGTCCGTCACATGCACTGTCTCAGGGGTCTCATTA
CCCGACTATGGTGTAAGCTGGATTCGCCAGCCTCC
ACGAAAGGGTCTGGAGTGGCTGGGAGTAATATGGG
GTAGTGAAACCACATACTATAATTCAGCTCTCAAA
TCCAGACTGACCATCATCAAGGACAACTCCAAGAG
CCAAGTTTTCTTAAAAATGAACAGTCTGCAAACTG
ATGACACAGCCATTTACTACTGTGCCAAACATTAT
TACTACGGTGGTAGCTATGCTATGGACTACTGGGG
TCAAGGAACCTCAGTCACCGTCTCCTCAGCGTTCG
TGCCGGTCTTCCTGCCAGCGAAGCCCACCACGACG
CCAGCGCCGCGACCACCAACACCGGCGCCCACCAT
CGCGTCGCAGCCCCTGTCCCTGCGCCCAGAGGCGT
GCCGGCCAGCGGCGGGGGGCGCAGTGCACACGAGG
GGGCTGGACTTCGCCTGTGATATCTACATCTGGGC
GCCCTTGGCCGGGACTTGTGGGGTCCTTCTCCTGT
CACTGGTTATCACCCTTTACTGCAACCACAGGAAC
CGTTTCTCTGTTGTTAAACGGGGCAGAAAGAAGCT
CCTGTATATATTCAAACAACCATTTATGAGACCAG
TACAAACTACTCAAGAGGAAGATGGCTGTAGCTGC
CGATTTCCAGAAGAAGAAGAAGGAGGATGTGAACT
GAGAGTGAAGTTCAGCAGGAGCGCAGACGCCCCCG
CGTACCAGCAGGGCCAGAACCAGCTCTATAACGAG
CTCAATCTAGGACGAAGAGAGGAGTACGATGTTTT
GGACAAGAGACGTGGCCGGGACCCTGAGATGGGGG
GAAAGCCGAGAAGGAAGAACCCTCAGGAAGGCCTG
TACAATGAACTGCAGAAAGATAAGATGGCGGAGGC
CTACAGTGAGATTGGGATGAAAGGCGAGCGCCGGA
GGGGCAAGGGGCACGATGGCCTTTACCAGGGTCTC
AGTACAGCCACCAAGGACACCTACGACGCCCTTCAC
ATGCAGGCCCTGCCCCCTCGCTAA
SEQ ID NO: 17
CVB3 IRES:
TTAAAACAGCCTGTGGGTTGATCCCACCCACAGGC
CCATTGGGCGCTAGCACTCTGGTATCACGGTACCT
TTGTGCGCCTGTTTTATACCCCCTCCCCCAACTGT
AACTTAGAAGTAACACACACCGATCAACAGTCAGC
GTGGCACACCAGCCACGTTTTGATCAAGCACTTCT
GTTACCCCGGACTGAGTATCAATAGACTGCTCACG
CGGTTGAAGGAGAAAGCGTTCGTTATCCGGCCAAC
TACTTCGAAAAACCTAGTAACACCGTGGAAGTTGC
AGAGTGTTTCGCTCAGCACTACCCCAGTGTAGATC
AGGTCGATGAGTCACCGCATTCCCCACGGGCGACC
GTGGCGGTGGCTGCGTTGGCGGCCTGCCCATGGGG
AAACCCATGGGACGCTCTAATACAGACATGGTGCG
AAGAGTCTATTGAGCTAGTTGGTAGTCCTCCGGCC
CCTGAATGCGGCTAATCCTAACTGCGGAGCACACA
CCCTCAAGCCAGAGGGCAGTGTGTCGTAACGGGCA
ACTCTGCAGCGGAACCGACTACTTTGGGTGTCCGT
GTTTCATTTTATTCCTATACTGGCTGCTTATGGTG
ACAATTGAGAGATCGTTACCATATAGCTATTGGAT
TGGCCATCCGGTGACTAATAGAGCTATTATATATC
CCTTTGTTGGGTTTATACCACTTAGCTTGAAAGAG
GTTAAAACATTACAATTCATTGTTAAGTTGAATACA
GCAAA
SEQ ID NO: 18
Human alpha globin 5′ UTR:
ACTCTTCTGGTCCCCACAGACTCAGAGAGAACCCACC
SEQ ID NO: 19
Human alpha globin 3′ UTR:
GCTGGAGCCTCGGTAGCCGTTCCTCCTGCCCGCTG
GGCCTCCCAACGGGCCCTCCTCCCCTCCTTGCACC
GGCCCTTCCTGGTCTTTGAATAAAGTCTGAGTGGG
CAGCA
SEQ ID NO: 20
C2 min sequence with annealing region
5′-CACACAACA GGGGGAUCAAUCCAAGGGACCC
GGAAACGCUCCCUUACACCCC ACCAACCAA-3′
SEQ ID NO: 21
Non-binding sequence with annealing region
5′-CACACAACA GGCGUAGUGAUUAUGAAUCGUG
UGCUAAUACACGCC ACCAACCAA-3′
SEQ ID NO: 22
36a sequence with annealing region
5′-GACACAACAGGGUGAAUGGUUCUACGAUAAAC
GUUAAUGACCAGCUUAUGGCUGGCAGUU CCUAUA
GCACCC ACCAACCAA-3′
SEQ ID NO: 23
Enhanced green fluorescent protein
DNA template
AGCAAGGGCGAGGAGCTGTTCACCGGGGTGGTGCC
CATCCTGGTCGAGCTGGACGGCGACGTAAACGGCC
ACAAGTTCAGCGTGTCCGGCGAGGGCGAGGGCGAT
GCCACCTACGGCAAGCTGACCCTGAAGTTCATCTG
CACCACCGGCAAGCTGCCCGTGCCCTGGCCCACCC
TCGTGACCACCCTGACCTACGGCGTGCAGTGCTTC
AGCCGCTACCCCGACCACATGAAGCAGCACGACTT
CTTCAAGTCCGCCATGCCCGAAGGCTACGTCCAGG
AGCGCACCATCTTCTTCAAGGACGACGGCAACTAC
AAGACCCGCGCCGAGGTGAAGTTCGAGGGCGACAC
CCTGGTGAACCGCATCGAGCTGAAGGGCATCGACT
TCAAGGAGGACGGCAACATCCTGGGGCACAAGCTG
GAGTACAACTACAACAGCCACAACGTCTATATCAT
GGCCGACAAGCAGAAGAACGGCATCAAGGTGAACT
TCAAGATCCGCCACAACATCGAGGACGGCAGCGTG
CAGCTCGCCGACCACTACCAGCAGAACACCCCCAT
CGGCGACGGCCCCGTGCTGCTGCCCGACAACCACT
ACCTGAGCACCCAGTCCGCCCTGAGCAAAGACCCC
AACGAGAAGCGCGATCACATGGTCCTGCTGGAGTT
CGTGACCGCCGCCGGGATCACTCTCGGCATGGACG
AGCTGTACAAG
SEQ ID NO: 24
CVB3 mPAH IS
GGGAAUAGCCGAAAAACAAAAAACAAAAAAAACAA
AAAAAAAACCAAAAAAACAAAACACAUUAAAACAG
CCUGUGGGUUGAUCCCACCCACAGGCCCAUUGGGC
GCUAGCACUCUGGUAUCACGGUACCUUUGUGCGCC
UGUUUUAUACCCCCUCCCCCAACUGUAACUUAGAA
GUAACACACACCGAUCAACAGUCAGCGUGGCACAC
CAGCCACGUUUUGAUCAAGCACUUCUGUUACCCCG
GACUGAGUAUCAAUAGACUGCUCACGCGGUUGAAG
GAGAAAGCGUUCGUUAUCCGGCCAACUACUUCGAA
AAACCUAGUAACACCGUGGAAGUUGCAGAGUGUUU
CGCUCAGCACUACCCCAGUGUAGAUCAGGUCGAUG
AGUCACCGCAUUCCCCACGGGCGACCGUGGCGGUG
GCUGCGUUGGCGGCCUGCCCAUGGGGAAACCCAUG
GGACGCUCUAAUACAGACAUGGUGCGAAGAGUCUA
UUGAGCUAGUUGGUAGUCCUCCGGCCCCUGAAUGC
GGCUAAUCCUAACUGCGGAGCACACACCCUCAAGC
CAGAGGGCAGUGUGUCGUAACGGGCAACUCUGCAG
CGGAACCGACUACUUUGGGUGUCCGUGUUUCAUUU
UAUUCCUAUACUGGCUGCUUAUGGUGACAAUUGAG
AGAUCGUUACCAUAUAGCUAUUGGAUUGGCCAUCC
GGUGACUAAUAGAGCUAUUAUAUAUCCCUUUGUUG
GGUUUAUACCACUUAGCUUGAAAGAGGUUAAAACA
UUACAAUUCAUUGUUAAGUUGAAUACAGCAAAAUG
GCAGCUGUUGUCCUGGAGAACGGAGUCCUGAGCAG
AAAACUCUCAGACUUUGGGCAGGAAACAAGUUACA
UCGAAGACAACUCCAAUCAAAAUGGUGCUGUAUCU
CUGAUAUUCUCACUCAAAGAGGAAGUUGGUGCCCU
GGCCAAGGUCCUGCGCUUAUUUGAGGAGAAUGAGA
UCAACCUGACACACAUUGAAUCCAGACCUUCUCGU
UUAAACAAAGAUGAGUAUGAGUUUUUCACCUAUCU
GGAUAAGCGUAGCAAGCCCGUCCUGGGCAGCAUCA
UCAAGAGCCUGAGGAACGACAUUGGUGCCACUGUC
CAUGAGCUUUCCCGAGACAAGGAAAAGAACACAGU
GCCCUGGUUCCCAAGGACCAUUCAGGAGCUGGACA
GAUUCGCCAAUCAGAUUCUCAGCUAUGGAGCCGAA
CUGGAUGCAGACCACCCAGGCUUUAAAGAUCCUGU
GUACCGGGCGAGACGAAAGCAGUUUGCUGACAUUG
CCUACAACUACCGCCAUGGGCAGCCCAUUCCUCGG
GUGGAAUACACAGAGGAGGAGAGGAAGACCUGGGG
AACGGUGUUCAGGACUCUGAAGGCCUUGUAUAAAA
CACAUGCCUGCUACGAGCACAACCACAUCUUCCCU
CUUCUGGAAAAGUACUGCGGUUUCCGUGAAGACAA
CAUCCCGCAGCUGGAAGAUGUUUCUCAGUUUCUGC
AGACUUGUACUGGUUUCCGCCUCCGUCCUGUUGCU
GGCUUACUGUCGUCUCGAGAUUUCUUGGGUGGCCU
GGCCUUCCGAGUCUUCCACUGCACACAGUACAUUA
GGCAUGGAUCUAAGCCCAUGUACACACCUGAACCU
GAUAUCUGUCAUGAACUCUUGGGACAUGUGCCCUU
GUUUUCAGAUAGAAGCUUUGCCCAGUUUUCUCAGG
AAAUUGGGCUUGCAUCGCUGGGGGCACCUGAUGAG
UACAUUGAGAAACUGGCCACAAUUUACUGGUUUAC
UGUGGAGUUUGGGCUUUGCAAGGAAGGAGAUUCUA
UAAAGGCAUAUGGUGCUGGGCUCUUGUCAUCCUUU
GGAGAAUUACAGUACUGUUUAUCAGACAAGCCAAA
GCUCCUGCCCCUGGAGCUAGAGAAGACAGCCUGCC
AGGAGUAUACUGUCACAGAGUUCCAGCCCCUGUAC
UAUGUGGCCGAGAGUUUCAAUGAUGCCAAGGAGAA
AGUGAGGACUUUUGCUGCCACAAUCCCCCGGCCCU
UCUCCGUUCGCUAUGACCCCUACACUCAAAGGGUU
GAGGUCCUGGACAAUACUCAGCAGUUGAAGAUUUU
AGCUGACUCCAUUAAUAGUGAGGUUGGAAUCCUUU
GCCAUGCCCUGCAGAAAAUAAAGUCAUGAAAAAAAC
AAAAAACAAAACGGCUAUU
SEQ ID NO: 25
CVB3 mPAH E1E2
GGGAAAATCCGTTGACCTTAAACGGTCGTGTGGGT
TCAAGTCCCTCCACCCCCACGCCGGAAACGCAATA
GCCGAAAAACAAAAAACAAAAAAAACAAAAAAAAA
ACCAAAAAAACAAAACACATTAAAACAGCCTGTGG
GTTGATCCCACCCACAGGCCCATTGGGCGCTAGCA
CTCTGGTATCACGGTACCTTTGTGCGCCTGTTTTA
TACCCCCTCCCCCAACTGTAACTTAGAAGTAACAC
ACACCGATCAACAGTCAGCGTGGCACACCAGCCAC
GTTTTGATCAAGCACTTCTGTTACCCCGGACTGAG
TATCAATAGACTGCTCACGCGGTTGAAGGAGAAAG
CGTTCGTTATCCGGCCAACTACTTCGAAAAACCTA
GTAACACCGTGGAAGTTGCAGAGTGTTTCGCTCAG
CACTACCCCAGTGTAGATCAGGTCGATGAGTCACC
GCATTCCCCACGGGCGACCGTGGCGGTGGCTGCGT
TGGCGGCCTGCCCATGGGGAAACCCATGGGACGCT
CTAATACAGACATGGTGCGAAGAGTCTATTGAGCT
AGTTGGTAGTCCTCCGGCCCCTGAATGCGGCTAAT
CCTAACTGCGGAGCACACACCCTCAAGCCAGAGGG
CAGTGTGTCGTAACGGGCAACTCTGCAGCGGAACC
GACTACTTTGGGTGTCCGTGTTTCATTTTATTCCT
ATACTGGCTGCTTATGGTGACAATTGAGAGATCGT
TACCATATAGCTATTGGATTGGCCATCCGGTGACT
AATAGAGCTATTATATATCCCTTTGTTGGGTTTAT
ACCACTTAGCTTGAAAGAGGTTAAAACATTACAAT
TCATTGTTAAGTTGAATACAGCAAAATGGCAGCTG
TTGTCCTGGAGAACGGAGTCCTGAGCAGAAAACTC
TCAGACTTTGGGCAGGAAACAAGTTACATCGAAGA
CAACTCCAATCAAAATGGTGCTGTATCTCTGATAT
TCTCACTCAAAGAGGAAGTTGGTGCCCTGGCCAAG
GTCCTGCGCTTATTTGAGGAGAATGAGATCAACCT
GACACACATTGAATCCAGACCTTCTCGTTTAAACA
AAGATGAGTATGAGTTTTTCACCTATCTGGATAAG
CGTAGCAAGCCCGTCCTGGGCAGCATCATCAAGAG
CCTGAGGAACGACATTGGTGCCACTGTCCATGAGC
TTTCCCGAGACAAGGAAAAGAACACAGTGCCCTGG
TTCCCAAGGACCATTCAGGAGCTGGACAGATTCGC
CAATCAGATTCTCAGCTATGGAGCCGAACTGGATG
CAGACCACCCAGGCTTTAAAGATCCTGTGTACCGG
GCGAGACGAAAGCAGTTTGCTGACATTGCCTACAA
CTACCGCCATGGGCAGCCCATTCCTCGGGTGGAAT
ACACAGAGGAGGAGAGGAAGACCTGGGGAACGGTG
TTCAGGACTCTGAAGGCCTTGTATAAAACACATGC
CTGCTACGAGCACAACCACATCTTCCCTCTTCTGG
AAAAGTACTGCGGTTTCCGTGAAGACAACATCCCG
CAGCTGGAAGATGTTTCTCAGTTTCTGCAGACTTG
TACTGGTTTCCGCCTCCGTCCTGTTGCTGGCTTAC
TGTCGTCTCGAGATTTCTTGGGTGGCCTGGCCTTC
CGAGTCTTCCACTGCACACAGTACATTAGGCATGG
ATCTAAGCCCATGTACACACCTGAACCTGATATCT
GTCATGAACTCTTGGGACATGTGCCCTTGTTTTCA
GATAGAAGCTTTGCCCAGTTTTCTCAGGAAATTGG
GCTTGCATCGCTGGGGGCACCTGATGAGTACATTG
AGAAACTGGCCACAATTTACTGGTTTACTGTGGAG
TTTGGGCTTTGCAAGGAAGGAGATTCTATAAAGGC
ATATGGTGCTGGGCTCTTGTCATCCTTTGGAGAAT
TACAGTACTGTTTATCAGACAAGCCAAAGCTCCTG
CCCCTGGAGCTAGAGAAGACAGCCTGCCAGGAGTA
TACTGTCACAGAGTTCCAGCCCCTGTACTATGTGG
CCGAGAGTTTCAATGATGCCAAGGAGAAAGTGAGG
ACTTTTGCTGCCACAATCCCCCGGCCCTTCTCCGT
TCGCTATGACCCCTACACTCAAAGGGTTGAGGTCC
TGGACAATACTCAGCAGTTGAAGATTTTAGCTGAC
TCCATTAATAGTGAGGTTGGAATCCTTTGCCATGC
CCTGCAGAAAATAAAGTCATGAAAAAAACAAAAAA
CAAAACGGCTATTATGCGTTACCGGCGAGACGCTAC
GGACTT
SEQ ID NO: 26
CVB3 IRES
TTAAAACAGCCTGTGGGTTGATCCCACCCACAGGC
CCATTGGGCGCTAGCACTCTGGTATCACGGTACCT
TTGTGCGCCTGTTTTATACCCCCTCCCCCAACTGT
AACTTAGAAGTAACACACACCGATCAACAGTCAGC
GTGGCACACCAGCCACGTTTTGATCAAGCACTTCT
GTTACCCCGGACTGAGTATCAATAGACTGCTCACG
CGGTTGAAGGAGAAAGCGTTCGTTATCCGGCCAAC
TACTTCGAAAAACCTAGTAACACCGTGGAAGTTGC
AGAGTGTTTCGCTCAGCACTACCCCAGTGTAGATC
AGGTCGATGAGTCACCGCATTCCCCACGGGCGACC
GTGGCGGTGGCTGCGTTGGCGGCCTGCCCATGGGG
AAACCCATGGGACGCTCTAATACAGACATGGTGCG
AAGAGTCTATTGAGCTAGTTGGTAGTCCTCCGGCC
CCTGAATGCGGCTAATCCTAACTGCGGAGCACACA
CCCTCAAGCCAGAGGGCAGTGTGTCGTAACGGGCA
ACTCTGCAGCGGAACCGACTACTTTGGGTGTCCGT
GTTTCATTTTATTCCTATACTGGCTGCTTATGGTG
ACAATTGAGAGATCGTTACCATATAGCTATTGGAT
TGGCCATCCGGTGACTAATAGAGCTATTATATATC
CCTTTGTTGGGTTTATACCACTTAGCTTGAAAGAG
GTTAAAACATTACAATTCATTGTTAAGTTGAATAC
AGCAAA
SEQ ID NO: 27
mPAH (Phenylalanine Hydroxylase, mouse)
ATGGCAGCTGTTGTCCTGGAGAACGGAGTCCTGAG
CAGAAAACTCTCAGACTTTGGGCAGGAAACAAGTT
ACATCGAAGACAACTCCAATCAAAATGGTGCTGTA
TCTCTGATATTCTCACTCAAAGAGGAAGTTGGTGC
CCTGGCCAAGGTCCTGCGCTTATTTGAGGAGAATG
AGATCAACCTGACACACATTGAATCCAGACCTTCT
CGTTTAAACAAAGATGAGTATGAGTTTTTCACCTA
TCTGGATAAGCGTAGCAAGCCCGTCCTGGGCAGCA
TCATCAAGAGCCTGAGGAACGACATTGGTGCCACT
GTCCATGAGCTTTCCCGAGACAAGGAAAAGAACAC
AGTGCCCTGGTTCCCAAGGACCATTCAGGAGCTGG
ACAGATTCGCCAATCAGATTCTCAGCTATGGAGCC
GAACTGGATGCAGACCACCCAGGCTTTAAAGATCC
TGTGTACCGGGCGAGACGAAAGCAGTTTGCTGACA
TTGCCTACAACTACCGCCATGGGCAGCCCATTCCT
CGGGTGGAATACACAGAGGAGGAGAGGAAGACCTG
GGGAACGGTGTTCAGGACTCTGAAGGCCTTGTATA
AAACACATGCCTGCTACGAGCACAACCACATCTTC
CCTCTTCTGGAAAAGTACTGCGGTTTCCGTGAAGA
CAACATCCCGCAGCTGGAAGATGTTTCTCAGTTTC
TGCAGACTTGTACTGGTTTCCGCCTCCGTCCTGTT
GCTGGCTTACTGTCGTCTCGAGATTTCTTGGGTGG
CCTGGCCTTCCGAGTCTTCCACTGCACACAGTACA
TTAGGCATGGATCTAAGCCCATGTACACACCTGAA
CCTGATATCTGTCATGAACTCTTGGGACATGTGCC
CTTGTTTTCAGATAGAAGCTTTGCCCAGTTTTCTC
AGGAAATTGGGCTTGCATCGCTGGGGGCACCTGAT
GAGTACATTGAGAAACTGGCCACAATTTACTGGTT
TACTGTGGAGTTTGGGCTTTGCAAGGAAGGAGATT
CTATAAAGGCATATGGTGCTGGGCTCTTGTCATCC
TTTGGAGAATTACAGTACTGTTTATCAGACAAGCC
AAAGCTCCTGCCCCTGGAGCTAGAGAAGACAGCCT
GCCAGGAGTATACTGTCACAGAGTTCCAGCCCCTG
TACTATGTGGCCGAGAGTTTCAATGATGCCAAGGA
GAAAGTGAGGACTTTTGCTGCCACAATCCCCCGGC
CCTTCTCCGTTCGCTATGACCCCTACACTCAAAGG
GTTGAGGTCCTGGACAATACTCAGCAGTTGAAGAT
TTTAGCTGACTCCATTAATAGTGAGGTTGGAATCC
TTTGCCATGCCCTGCAGAAAATAAAGTCATGA
SEQ ID NO: 28
Kozak 3XFLAG-EGF P2A nostop (330 bps)
GGGAGCCACCATGGACTACAAGGACGACGACGACA
AGATCATCGACTATAAAGACGACGACGATAAAGGT
GGCGACTATAAGGACGACGACGACAAAGCCATTAA
TAGTGACTCTGAGTGTCCCCTGTCCCACGACGGGT
ACTGCCTCCACGACGGTGTGTGCATGTATATTGAA
GCATTGGACAAGTACGCCTGCAACTGTGTTGTTGG
CTACATCGGGGAGCGCTGTCAGTACCGAGACCTGA
AGTGGTGGGAACTGCGCGGAAGCGGAGCTACTAAC
TTCAGCCTGCTGAAGCAGGCTGGAGACGTGGAGGA
GAACCCTGGACCTCT
      5-13: Kozak sequence
  14-262: 3XFLAG-EGF
263-328: P2A
SEQ ID NO: 29
Kozak 1XFLAG-EGF T2A 1XFLAG-Nluc
P2A nostop (873 bps)
GGGAGCCACCATGGACTACAAGGACGACGACGACA
AGATCATCAATAGTGACTCTGAGTGTCCCCTGTCC
CACGACGGGTACTGCCTCCACGACGGTGTGTGCAT
GTATATTGAAGCATTGGACAAGTACGCCTGCAACT
GTGTTGTTGGCTACATCGGGGAGCGCTGTCAGTAC
CGAGACCTGAAGTGGTGGGAACTGCGCGGCTCCGG
CGAGGGCAGGGGAAGTCTTCTAACATGCGGGGACG
TGGAGGAAAATCCCGGCCCAGACTATAAGGACGAC
GACGACAAAATCATCGTCTTCACACTCGAAGATTT
CGTTGGGGACTGGCGACAGACAGCCGGCTACAACC
TGGACCAAGTCCTTGAACAGGGAGGTGTGTCCAGT
TTGTTTCAGAATCTCGGGGTGTCCGTAACTCCGAT
CCAAAGGATTGTCCTGAGCGGTGAAAATGGGCTGA
AGATCGACATCCATGTCATCATCCCGTATGAAGGT
CTGAGCGGCGACCAAATGGGCCAGATCGAAAAAAT
TTTTAAGGTGGTGTACCCTGTGGATGATCATCACT
TTAAGGTGATCCTGCACTATGGCACACTGGTAATC
GACGGGGTTACGCCGAACATGATCGACTATTTCGG
ACGGCCGTATGAAGGCATCGCCGTGTTCGACGGCA
AAAAGATCACTGTAACAGGGACCCTGTGGAACGGC
AACAAAATTATCGACGAGCGCCTGATCAACCCCGA
CGGCTCCCTGCTGTTCCGAGTAACCATCAACGGAG
TGACCGGCTGGCGGCTGTGCGAACGCATTCTGGCG
GGAAGCGGAGCTACTAACTTCAGCCTGCTGAAGCA
GGCTGGAGACGTGGAGGAGAACCCTGGACCTCT
      5-13: Kozak sequence
  14-202: 1XFLAG-EGF
203-265: T2A
266-805: 1XFLAG-Nluc
806-871: P2A
SEQ ID NO: 30
Kozak 1XFLAG-EGF stop 1XFLAG-Nluc
stop (762 bps)
GGGAGCCACCATGGACTACAAGGACGACGACGACA
AGATCATCAATAGTGACTCTGAGTGTCCCCTGTCC
CACGACGGGTACTGCCTCCACGACGGTGTGTGCAT
GTATATTGAAGCATTGGACAAGTACGCCTGCAACT
GTGTTGTTGGCTACATCGGGGAGCGCTGTCAGTAC
CGAGACCTGAAGTGGTGGGAACTGCGCTGATAGTA
AGACTATAAGGACGACGACGACAAAATCATCGTCT
TCACACTCGAAGATTTCGTTGGGGACTGGCGACAG
ACAGCCGGCTACAACCTGGACCAAGTCCTTGAACA
GGGAGGTGTGTCCAGTTTGTTTCAGAATCTCGGGG
TGTCCGTAACTCCGATCCAAAGGATTGTCCTGAGC
GGTGAAAATGGGCTGAAGATCGACATCCATGTCAT
CATCCCGTATGAAGGTCTGAGCGGCGACCAAATGG
GCCAGATCGAAAAAATTTTTAAGGTGGTGTACCCT
GTGGATGATCATCACTTTAAGGTGATCCTGCACTA
TGGCACACTGGTAATCGACGGGGTTACGCCGAACA
TGATCGACTATTTCGGACGGCCGTATGAAGGCATC
GCCGTGTTCGACGGCAAAAAGATCACTGTAACAGG
GACCCTGTGGAACGGCAACAAAATTATCGACGAGC
GCCTGATCAACCCCGACGGCTCCCTGCTGTTCCGA
GTAACCATCAACGGAGTGACCGGCTGGCGGCTGTG
CGAACGCATTCTGGCGTGATAGTAACT
      5-13: Kozak sequence
  14-202: 1XFLAG-EGF
203-211: Triple stop codon
212-751: 1XFLAG-Nluc
752-760: Triple stop codon
```

1-59. (canceled)

60. A method of producing a therapeutic immune cell, comprising:
contacting an isolated immune cell ex vivo with a circular polyribonucleotide encoding a protein of interest, wherein the protein of interest is transiently expressed in the immune cell.

61. The method of claim 60, wherein the immune cell is selected from a T cell, a natural killer (NK) cell, a macrophage, or a dendritic cell.

62. The method of claim 60, wherein the circular polyribonucleotide comprises a self-splicing intron.

63. The method of claim 60, wherein the method excludes stable genetic modification of the immune cell.

64. The method of claim 60, wherein the method excludes viral transduction steps.

65. The method of claim 60, wherein the method excludes DNA amplification steps.

66. A method of producing a therapeutic cell composition, comprising:
(a) contacting isolated cells ex vivo with a circular polyribonucleotide encoding a protein of interest;
(b) culturing the cells of step (a) to permit expression of the protein of interest; and
(c) recovering the cells of step (b), thereby making a therapeutic cell composition.

67. The method of claim 66, wherein the cells of step (c) are washed prior to administration.

68. The method of claim 66, wherein expression of the protein of interest is transient.

69. The method of claim 66, wherein the circular polyribonucleotide is degraded during culturing of the cells.

70. The method of claim 66, wherein the circular polyribonucleotide encodes a therapeutic protein.

71. The method of claim 66, wherein the protein of interest is an antigen receptor.

72. The method of claim 66, wherein the protein of interest is not an antigen receptor.

73. The method of claim 72, wherein the protein of interest is an immune-modulatory protein.

74. The method of claim 72, wherein the immune-modulatory protein is a cytokine or an intracellular signaling regulator.

75. A method of administering a therapeutic cell composition, comprising administering cells produced according to claim 66, wherein the cells are administered after degradation of at least a portion of the circular polyribonucleotide used to produce the cells.

76. The method of claim 75, wherein the protein of interest remains functional in the administered cells.

77. The method of claim 75, wherein administration occurs after a defined period following exposure of the cells to the circular polyribonucleotide.

78. The method of claim 75, wherein the administered cell composition is free of exogenous nucleic acids.


SEQUENCE LISTING (56 sequences)

SEQ ID NO: 1 - skipped

SEQ ID NO: 2 - DNA, 717 bp, synthetic construct
  source 1..717: mol_type = other DNA; note = /note="Description of Artificial Sequence: Synthetic polynucleotide"
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 3 - DNA, 57 bp, synthetic construct
  source 1..57: mol_type = other DNA; note = /note="Description of Artificial Sequence: Synthetic oligonucleotide"
gctactaacttcagcctgctgaagcaggctggcgacgtggaggagaaccctggacct

SEQ ID NO: 4 - DNA, 221 bp, synthetic construct
  source 1..221: mol_type = other DNA; note = /note="Description of Artificial Sequence: Synthetic polynucleotide"
gtaaaaagaggtgaaacctattatgtgtgagcagggcacagacgttgaaactggagccaggagaagtattggcaggctttaggttattaggtggttactctgtcttaaaaatgttctggctttcttcctgcatccactggcatactcatggtctgtttttaaatattttaattcccatttacaaagtgatttacccacaagcccaacctgtctgtcttcag

SEQ ID NO: 5 - DNA, 552 bp, Encephalomyocarditis virus
  source 1..552: mol_type = genomic DNA
acgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataata

SEQ ID NO: 6 - DNA, 6926 bp, synthetic construct
  source 1..6926: mol_type = other DNA; note = /note="Description of Artificial Sequence: Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattccattgagaaatgactgagttccggtgctctcaagtcattgatctttgtcgacttttatttggtctctgtaataacgacttcaaaaacattaaattctgttgcgaagccagtaagctacaaaaagaaaaaacaagagagaatgctatagtcgtatagtatagtttcccgactatctgatacccattacttatctagggggaatgcgaacccaaaattttatcagttttctcggatatcgatagatattggggaataaatttaaataaataaattttgggcgggtttagggcgtggcaaaaagttttttggcaaatcgctagaaatttacaagacttataaaattatgaaaaaatacaacaaaattttaaacacgtgggcgtgacagttttgggcggttttagggcgttagagtaggcgaggacagggttacatcgactaggctttgatcctgatcaagaatatatatactttataccgcttccttctacatgttacctatttttcaacgaatctagtatacctttttactgtacgatttatgggtataataataagctaaatcgagactaagttttattgttatatatattttttttattttatgcagaaattaattaaaccggtcctgcaggtgatcaggcgcgccggttaccggccggccccgcggagcgtaagtattcaaaattccaaaattttttactagaaatattcgattttttaataggcagtttctatactattgtatactattgtagattcgttgaaaagtatgtaacaggaagaataaagcatttccgaccatgtaaagtatatatattcttaataaggatcaatagccgagtcgatctcgccatgtccgtctgtcttattgttttattaccgccgagacatcaggaactataaaagctagaaggatgagttttagcatacagattctagagacaaggacgcagagcaagtttgttgatccatgctgccacgctttaactttctcaaattgcccaaaactgccatgcccacatttttgaactattttcgaaattttttcataattgtattactcgtgtaaatttccatcaatttgccaaaaaactttttgtcacgcgttaacgccctaaagccgccaatttggtcacgcccacactattgagcaattatcaaattttttctcattttattccccaatatctatcgatatccccgattatgaaattattaaatttcgcgttcgcattcacactagctgagtaacgagtatctgatagttggggaaatcgacttattttttatatacaatgaaaatgaatttaatcatatgaatatcgattatagctttttatttaatatgaatatttatttgggcttaaggtgtaacctcctcgacataagactcacatggcgcaggcacattgaagacaaaaatactcattgtcgggtctcgcaccctccagcagcacctaaaattatgtcttcaattattgccaacattggagacacaattagtctgtggcacctcaggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 7 - DNA, 708 bp, synthetic construct
  source 1..708: mol_type = other DNA; note = /note="Description of Artificial Sequence: Synthetic polynucleotide"
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 8 - DNA, 796 bp, synthetic construct
  source 1..796: mol_type = other DNA; note = /note="Description of Artificial Sequence: Synthetic polynucleotide"
gtaagaagcaaggtttcatttaggggaagggaaatgattcaggacgagagtctttgtgctgctgagtgcctgtgatgaagaagcatgttagtcctgggcaacgtagcgagaccccatctctacaaaaaatagaaaaattagccaggtatagtggcgcacacctgtgattccagctacgcaggaggctgaggtgggaggattgcttgagcccaggaggttgaggctgcagtgagctgtaatcatgccactactccaacctgggcaacacagcaaggaccctgtctcaaaagctacttacagaaaagaattaggctcggcacggtagctcacacctgtaatcccagcactttgggaggctgaggcgggcagatcacttgaggtcaggagtttgagaccagcctggccaacatggtgaaaccttgtctctactaaaaatatgaaaattagccaggcatggtggcacattcctgtaatcccagctactcgggaggctgaggcaggagaatcacttgaacccaggaggtggaggttgcagtaagccgagatcgtaccactgtgctctagccttggtgacagagcgagactgtcttaaaaaaaaaaaaaaaaaaaaaagaattaattaaaaatttaaaaaaaaatgaaaaaaagctgcatgcttgttttttgtttttagttattctacattgttgtcattattaccaaatattggggaaaatacaacttacagaccaatctcaggagttaaatgttactacgaaggcaaatgaactatgcgtaatgaacctggtaggcatta

SEQ ID NO: 9 - DNA, 264 bp, synthetic construct
  source 1..264: mol_type = other DNA; note = /note="Description of Artificial Sequence: Synthetic polynucleotide"
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcct

SEQ ID NO: 10 - DNA, 330 bp, synthetic construct
  source 1..330: mol_type = other DNA; note = /note="Description of Artificial Sequence: Synthetic polynucleotide"
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggaagcggagctactaacttcagcctgctgaagcaggctggagacgtggaggagaaccctggacctct

SEQ ID NO: 11 - DNA, 264 bp, synthetic construct; identical to SEQ ID NO: 9
  source 1..264: mol_type = other DNA; note = /note="Description of Artificial Sequence: Synthetic polynucleotide"

SEQ ID NO: 12 - DNA, 273 bp, synthetic construct
  source 1..273: mol_type = other DNA; note = /note="Description of Artificial Sequence: Synthetic polynucleotide"
gggagccaccatggactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgctgatagtaact

SEQ ID NO: 13 - DNA, 954 bp, synthetic construct
  source 1..954: mol_type = other DNA; note = /note="Description of Artificial Sequence: Synthetic polynucleotide"
gggacctaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggaagcggagctactaacttcagcctgctgaagcaggctggagacgtggaggagaaccctggacctct

SEQ ID NO: 14 - DNA, 1314 bp, synthetic construct
  source 1..1314: mol_type = other DNA; note = /note="Description of Artificial Sequence: Synthetic polynucleotide"
gggacctaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggaccttgatagtaact

SEQ ID NO: 15 - DNA, 1305 bp, synthetic construct
  source 1..1305: mol_type = other DNA; note = /note="Description of Artificial Sequence: Synthetic polynucleotide"
gggacctaacgttactggccgaagccgcttggaacaaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttcaagacaaacaacgtctgtagcgaccctttgcaggcagcggaaccccccacctggcgacaggtgcctctgcggccaaaagccacgtgtatacgatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgttcagtcgaggttaaaaaacgtccaggccccccgaaccacggggacgtggttttcctttgaaaaacacgatgataatatggccacaaccatgggctccggcgagggcaggggaagtcttctaacatgcggggacgtggaggaaaatcccggcccagactacaaggacgacgacgacaagatcatcgactataaagacgacgacgataaaggtggcgactataaggacgacgacgacaaagccattgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctct

SEQ ID NO: 16 - DNA, 1530 bp, synthetic construct
  source 1..1530: mol_type = other DNA; note = /note="Description of Artificial Sequence: Synthetic polynucleotide"
atggctctgcctgtgacagctctgctgctgcctctggccctgctgctgcatgctgccagacctgacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcgttcgtgccggtcttcctgccagcgaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaccacaggaaccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 17 - DNA, 741 bp, Human coxsackievirus B3
  source 1..741: mol_type = genomic DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 18 - DNA, 37 bp, Homo sapiens
  source 1..37: mol_type = genomic DNA
actcttctggtccccacagactcagagagaacccacc

SEQ ID NO: 19 - DNA, 110 bp, Homo sapiens
  source 1..110: mol_type = genomic DNA
gctggagcctcggtagccgttcctcctgcccgctgggcctcccaacgggccctcctcccctccttgcaccggcccttcctggtctttgaataaagtctgagtgggcagca

SEQ ID NO: 20 - RNA, 61 nt, synthetic construct
  source 1..61: mol_type = other RNA; note = /note="Description of Artificial Sequence: Synthetic oligonucleotide"
cacacaacagggggatcaatccaagggacccggaaacgctcccttacaccccaccaaccaa

SEQ ID NO: 21 - RNA, 54 nt, synthetic construct
  source 1..54: mol_type = other RNA; note = /note="Description of Artificial Sequence: Synthetic oligonucleotide"
cacacaacaggcgtagtgattatgaatcgtgtgctaatacacgccaccaaccaa

SEQ ID NO: 22 - RNA, 81 nt, synthetic construct
  source 1..81: mol_type = other RNA; note = /note="Description of Artificial Sequence: Synthetic oligonucleotide"
gacacaacagggtgaatggttctacgataaacgttaatgaccagcttatggctggcagttcctatagcacccaccaaccaa

SEQ ID NO: 23 - DNA, 711 bp, synthetic construct
  source 1..711: mol_type = other DNA; note = /note="Description of Artificial Sequence: Synthetic polynucleotide"
agcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 24 - RNA, 2190 nt, synthetic construct
  source 1..2190: mol_type = other RNA; note = /note="Description of Artificial Sequence: Synthetic polynucleotide"
gggaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggcagctgttgtcctggagaacggagtcctgagcagaaaactctcagactttgggcaggaaacaagttacatcgaagacaactccaatcaaaatggtgctgtatctctgatattctcactcaaagaggaagttggtgccctggccaaggtcctgcgcttatttgaggagaatgagatcaacctgacacacattgaatccagaccttctcgtttaaacaaagatgagtatgagtttttcacctatctggataagcgtagcaagcccgtcctgggcagcatcatcaagagcctgaggaacgacattggtgccactgtccatgagctttcccgagacaaggaaaagaacacagtgccctggttcccaaggaccattcaggagctggacagattcgccaatcagattctcagctatggagccgaactggatgcagaccacccaggctttaaagatcctgtgtaccgggcgagacgaaagcagtttgctgacattgcctacaactaccgccatgggcagcccattcctcgggtggaatacacagaggaggagaggaagacctggggaacggtgttcaggactctgaaggccttgtataaaacacatgcctgctacgagcacaaccacatcttccctcttctggaaaagtactgcggtttccgtgaagacaacatcccgcagctggaagatgtttctcagtttctgcagacttgtactggtttccgcctccgtcctgttgctggcttactgtcgtctcgagatttcttgggtggcctggccttccgagtcttccactgcacacagtacattaggcatggatctaagcccatgtacacacctgaacctgatatctgtcatgaactcttgggacatgtgcccttgttttcagatagaagctttgcccagttttctcaggaaattgggcttgcatcgctgggggcacctgatgagtacattgagaaactggccacaatttactggtttactgtggagtttgggctttgcaaggaaggagattctataaaggcatatggtgctgggctcttgtcatcctttggagaattacagtactgtttatcagacaagccaaagctcctgcccctggagctagagaagacagcctgccaggagtatactgtcacagagttccagcccctgtactatgtggccgagagtttcaatgatgccaaggagaaagtgaggacttttgctgccacaatcccccggcccttctccgttcgctatgacccctacactcaaagggttgaggtcctggacaatactcagcagttgaagattttagctgactccattaatagtgaggttggaatcctttgccatgccctgcagaaaataaagtcatgaaaaaaacaaaaaacaaaacggctatt

SEQ ID NO: 25 - DNA, 2282 bp, synthetic construct
  source 1..2282: mol_type = other DNA; note = /note="Description of Artificial Sequence: Synthetic polynucleotide"
gggaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggcagctgttgtcctggagaacggagtcctgagcagaaaactctcagactttgggcaggaaacaagttacatcgaagacaactccaatcaaaatggtgctgtatctctgatattctcactcaaagaggaagttggtgccctggccaaggtcctgcgcttatttgaggagaatgagatcaacctgacacacattgaatccagaccttctcgtttaaacaaagatgagtatgagtttttcacctatctggataagcgtagcaagcccgtcctgggcagcatcatcaagagcctgaggaacgacattggtgccactgtccatgagctttcccgagacaaggaaaagaacacagtgccctggttcccaaggaccattcaggagctggacagattcgccaatcagattctcagctatggagccgaactggatgcagaccacccaggctttaaagatcctgtgtaccgggcgagacgaaagcagtttgctgacattgcctacaactaccgccatgggcagcccattcctcgggtggaatacacagaggaggagaggaagacctggggaacggtgttcaggactctgaaggccttgtataaaacacatgcctgctacgagcacaaccacatcttccctcttctggaaaagtactgcggtttccgtgaagacaacatcccgcagctggaagatgtttctcagtttctgcagacttgtactggtttccgcctccgtcctgttgctggcttactgtcgtctcgagatttcttgggtggcctggccttccgagtcttccactgcacacagtacattaggcatggatctaagcccatgtacacacctgaacctgatatctgtcatgaactcttgggacatgtgcccttgttttcagatagaagctttgcccagttttctcaggaaattgggcttgcatcgctgggggcacctgatgagtacattgagaaactggccacaatttactggtttactgtggagtttgggctttgcaaggaaggagattctataaaggcatatggtgctgggctcttgtcatcctttggagaattacagtactgtttatcagacaagccaaagctcctgcccctggagctagagaagacagcctgccaggagtatactgtcacagagttccagcccctgtactatgtggccgagagtttcaatgatgccaaggagaaagtgaggacttttgctgccacaatcccccggcccttctccgttcgctatgacccctacactcaaagggttgaggtcctggacaatactcagcagttgaagattttagctgactccattaatagtgaggttggaatcctttgccatgccctgcagaaaataaagtcatgaaaaaaacaaaaaacaaaacggctattatgcgttaccggcgagacgctacggactt

SEQ ID NO: 26 - DNA, 741 bp, Human coxsackievirus B3; identical to SEQ ID NO: 17
  source 1..741: mol_type = genomic DNA

SEQ ID NO: 27 - DNA, 1362 bp, Mus sp.
  source 1..1362: mol_type = genomic DNA
atggcagctgttgtcctggagaacggagtcctgagcagaaaactctcagactttgggcaggaaacaagttacatcgaagacaactccaatcaaaatggtgctgtatctctgatattctcactcaaagaggaagttggtgccctggccaaggtcctgcgcttatttgaggagaatgagatcaacctgacacacattgaatccagaccttctcgtttaaacaaagatgagtatgagtttttcacctatctggataagcgtagcaagcccgtcctgggcagcatcatcaagagcctgaggaacgacattggtgccactgtccatgagctttcccgagacaaggaaaagaacacagtgccctggttcccaaggaccattcaggagctggacagattcgccaatcagattctcagctatggagccgaactggatgcagaccacccaggctttaaagatcctgtgtaccgggcgagacgaaagcagtttgctgacattgcctacaactaccgccatgggcagcccattcctcgggtggaatacacagaggaggagaggaagacctggggaacggtgttcaggactctgaaggccttgtataaaacacatgcctgctacgagcacaaccacatcttccctcttctggaaaagtactgcggtttccgtgaagacaacatcccgcagctggaagatgtttctcagtttctgcagacttgtactggtttccgcctccgtcctgttgctggcttactgtcgtctcgagatttcttgggtggcctggccttccgagtcttccactgcacacagtacattaggcatggatctaagcccatgtacacacctgaacctgatatctgtcatgaactcttgggacatgtgcccttgttttcagatagaagctttgcccagttttctcaggaaattgggcttgcatcgctgggggcacctgatgagtacattgagaaactggccacaatttactggtttactgtggagtttgggctttgcaaggaaggagattctataaaggcatatggtgctgggctcttgtcatcctttggagaattacagtactgtttatcagacaagccaaagctcctgcccctggagctagagaagacagcctgccaggagtatactgtcacagagttccagcccctgtactatgtggccgagagtttcaatgatgccaaggagaaagtgaggacttttgctgccacaatcccccggcccttctccgttcgctatgacccctacactcaaagggttgaggtcctggacaatactcagcagttgaagattttagctgactccattaatagtgaggttggaatcctttgccatgccctgcagaaaataaagtcatga

SEQ ID NO: 28 - DNA, 330 bp, synthetic construct; identical to SEQ ID NO: 10
  source 1..330: mol_type = other DNA; note = /note="Description of Artificial Sequence: Synthetic polynucleotide"

SEQ ID NO: 29 - DNA, 873 bp, synthetic construct
  source 1..873: mol_type = other DNA; note = /note="Description of Artificial Sequence: Synthetic polynucleotide"
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgcggctccggcgagggcaggggaagtcttctaacatgcggggacgtggaggaaaatcccggcccagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggaagcggagctactaacttcagcctgctgaagcaggctggagacgtggaggagaaccctggacctct

SEQ ID NO: 30 - DNA, 762 bp, synthetic construct
  source 1..762: mol_type = other DNA; note = /note="Description of Artificial Sequence: Synthetic polynucleotide"
gggagccaccatggactacaaggacgacgacgacaagatcatcaatagtgactctgagtgtcccctgtcccacgacgggtactgcctccacgacggtgtgtgcatgtatattgaagcattggacaagtacgcctgcaactgtgttgttggctacatcggggagcgctgtcagtaccgagacctgaagtggtgggaactgcgctgatagtaagactataaggacgacgacgacaaaatcatcgtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtgatagtaact

SEQ ID NO: 31 - DNA, 54 bp, synthetic construct
  source 1..54: mol_type = other DNA; note = /note="Description of Artificial Sequence: Synthetic oligonucleotide"
gagggcaggggaagtctactaacatgcggggacgtggaggaaaatcccggccca

SEQ ID NO: 32 - DNA, 60 bp, synthetic construct
  source 1..60: mol_type = other DNA; note = /note="Description of Artificial Sequence: Synthetic oligonucleotide"
cagtgtactaattatgctctcttgaaattggctggagatgttgagagcaacccaggtccc

SEQ ID NO: 33 - protein, 8 aa, synthetic construct
  VARIANT 2: note = /replace="Ile"
  VARIANT 4: note = MOD_RES - Any amino acid
  SITE 1..8: note = /note="Variant residues given in the sequence have no preference with respect to those in the annotations for variant positions"
  source 1..8: note = /note="Description of Artificial Sequence: Synthetic peptide"
DVEXNPGP

SEQ ID NO: 34 - DNA, 20 bp, synthetic construct
  source 1..20: mol_type = other DNA; note = /note="Description of Artificial Sequence: Synthetic oligonucleotide"
ggctattcccaatagccgtt

SEQ ID NO: 35 - DNA, 30 bp, synthetic construct
  source 1..30: mol_type = other DNA; note = /note="Description of Artificial Sequence: Synthetic oligonucleotide"
gtttttcggctattcccaatagccgttttg

SEQ ID NO: 36 - DNA, 32 bp, synthetic construct
  source 1..32: mol_type = other DNA; note = /note="Description of Artificial Sequence: Synthetic oligonucleotide"
gtcaacggattttcccaagtccgtagcgtctc

SEQ ID NO: 37 - RNA, 43 nt, synthetic construct
  source 1..43: mol_type = other RNA; note = /note="Description of Artificial Sequence: Synthetic oligonucleotide"
gggggatcaatccaagggacccggaaacgctcccttacacccc

SEQ ID NO: 38 - RNA, 63 nt, synthetic construct
  source 1..63: mol_type = other RNA; note = /note="Description of Artificial Sequence: Synthetic oligonucleotide"
gggtgaatggttctacgataaacgttaatgaccagcttatggctggcagttcctatagcaccc

SEQ ID NO: 39 - DNA, 18 bp, synthetic construct
  source 1..18: mol_type = other DNA; note = /note="Description of Artificial Sequence: Synthetic oligonucleotide"
tgttgtgtcttggttggt

SEQ ID NO: 40 - DNA, 18 bp, synthetic construct
  source 1..18: mol_type = other DNA; note = /note="Description of Artificial Sequence: Synthetic oligonucleotide"
tgttgtgtgttggttggt

SEQ ID NO: 41 - DNA, 21 bp, synthetic construct
  source 1..21: mol_type = other DNA; note = /note="Description of Artificial Sequence: Synthetic primer"
cctgagattcctgggttcaag

SEQ ID NO: 42 - DNA, 21 bp, synthetic construct
  source 1..21: mol_type = other DNA; note = /note="Description of Artificial Sequence: Synthetic primer"
cttcttgagcaggtcagaaca

SEQ ID NO: 43 - DNA, 20 bp, synthetic construct
  source 1..20: mol_type = other DNA; note = /note="Description of Artificial Sequence: Synthetic primer"
acgacggtgtgtgcatgtat

SEQ ID NO: 44 - DNA, 20 bp, synthetic construct
  source 1..20: mol_type = other DNA; note = /note="Description of Artificial Sequence: Synthetic primer"
ttcccaccacttcaggtctc

SEQ ID NO: 45 - DNA, 20 bp, synthetic construct
  source 1..20: mol_type = other DNA; note = /note="Description of Artificial Sequence: Synthetic primer"
tacgcctgcaactgtgttgt

SEQ ID NO: 46 - DNA, 20 bp, synthetic construct
  source 1..20: mol_type = other DNA; note = /note="Description of Artificial Sequence: Synthetic primer"
tcgatgatcttgtcgtcgtc

SEQ ID NO: 47 - DNA, 21 bp, synthetic construct
  source 1..21: mol_type = other DNA; note = /note="Description of Artificial Sequence: Synthetic primer"
agggctgcttttaactctggt

SEQ ID NO: 48 - DNA, 21 bp, synthetic construct
  source 1..21: mol_type = other DNA; note = /note="Description of Artificial Sequence: Synthetic primer"
ccccacttgattttggaggga

SEQ ID NO: 49 - DNA, 20 bp, synthetic construct
  source 1..20: mol_type = other DNA; note = /note="Description of Artificial Sequence: Synthetic primer"
tgtgggcaatgtcatcaaaa

SEQ ID NO: 50 - DNA, 21 bp, synthetic construct
  source 1..21: mol_type = other DNA; note = /note="Description of Artificial Sequence: Synthetic primer"
gaagcacttgctacctcttgc

SEQ ID NO: 51 - DNA, 19 bp, synthetic construct
  source 1..19: mol_type = other DNA; note = /note="Description of Artificial Sequence: Synthetic primer"
ggcaccatgggaagtgatt

SEQ ID NO: 52 - DNA, 20 bp, synthetic construct
  source 1..20: mol_type = other DNA; note = /note="Description of Artificial Sequence: Synthetic primer"
atttggtaaggcctgagctg

SEQ ID NO: 53 - DNA, 20 bp, synthetic construct
  source 1..20: mol_type = other DNA; note = /note="Description of Artificial Sequence: Synthetic primer"
tcgctggtatcactcgtctg

SEQ ID NO: 54 - DNA, 20 bp, synthetic construct
  source 1..20: mol_type = other DNA; note = /note="Description of Artificial Sequence: Synthetic primer"
gattctgaagaccgccagag

SEQ ID NO: 55 - DNA, 20 bp, synthetic construct
  source 1..20: mol_type = other DNA; note = /note="Description of Artificial Sequence: Synthetic primer"
ctctcctgttgtgcttctcc

SEQ ID NO: 56 - DNA, 22 bp, synthetic construct
  source 1..22: mol_type = other DNA; note = /note="Description of Artificial Sequence: Synthetic primer"
gtcaaagttcatcctgtccttg